SEQUENCE LISTING (16 sequences)

SEQ ID NO: 1 - protein, 393 aa, Homo sapiens [sequence omitted]

SEQ ID NO: 2 - protein, 173 aa, Unknown
  Description of Unknown PIK3CA sequence
MQPFSIPVQITLQGSRRRQGRTAFPASGKKRETDYSDGDPLDVHKRLPSSAGEDRAVMLGFAMMGFSVLMFFLLGTTILKPFMLSIQREESTCTAIHTDIMDDWLDCAFTCGVHCHGQGKYPCLQVFVNLSHPGQKALLHYNEEAVQINPKRDVTDCRVKEKQTLTVSDEHKQ

SEQ ID NO: 3 - protein, 1068 aa, Unknown
  Description of Unknown PIK3CA sequence
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 4 - DNA, 3008 bp, Unknown
  Description of Unknown Akt1 sequence
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 5 - protein, 1342 aa, Unknown
  Description of Unknown HER3 sequence
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 6 - DNA, 4721 bp, Unknown
  Description of Unknown HER3 sequence
ctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggggggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggggtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctc

SEQ ID NO: 7 - DNA, 19 bp, Artificial Sequence
  Description of Artificial Sequence Synthetic oligonucleotide
  Description of Combined DNA/RNA Molecule Synthetic oligonucleotide
ggaaccugucuccacaaag

SEQ ID NO: 8 - DNA, 19 bp, Artificial Sequence
  Description of Artificial Sequence Synthetic oligonucleotide
  Description of Combined DNA/RNA Molecule Synthetic oligonucleotide
gaagaaugcagguuuaaua

SEQ ID NO: 9 - DNA, 48 bp, Artificial Sequence
  Description of Artificial Sequence Synthetic oligonucleotide
  CDS (1)..(48)
ggcctggagaagctgggtatcttcgtcaagaccgtgacggagggtggt

SEQ ID NO: 10 - protein, 16 aa, Artificial Sequence
  Description of Artificial Sequence Synthetic peptide
GLEKLGIFVKTVTEGG

SEQ ID NO: 11 - DNA, 34 bp, Artificial Sequence
  Description of Artificial Sequence Synthetic oligonucleotide
ggcctggagaagctgggtattgacggagggtggt

SEQ ID NO: 12 - DNA, 14 bp, Artificial Sequence
  Description of Artificial Sequence Synthetic oligonucleotide
cttcgtcaagaccg

SEQ ID NO: 13 - DNA, 46 bp, Artificial Sequence
  Description of Artificial Sequence Synthetic oligonucleotide
ggcctggagaagctgggtatcttcgtcaagactgacggagggtggt

SEQ ID NO: 14 - DNA, 47 bp, Artificial Sequence
  Description of Artificial Sequence Synthetic oligonucleotide
ggcctggagaagctgggtatcttcgtcaagaccggacggagggtggt

SEQ ID NO: 15 - DNA, 11603 bp, Artificial Sequence
  Description of Artificial Sequence Synthetic polynucleotide
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccgccggcaccggtgatcagttatctagactacttgtcgtcgtcgtccttgtagtcgatgtcgtggtccttgtagtcgccgtcgtggtccttgtagtcggcgccaccgcctccaaccactttgtacaagaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgactaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggggggcgtaaacgccgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgatacagtagaaattacagaaactttatcacgtttagtaagtatagaggctgaaaatccagatgaagccgaacgacttgtaagagaaaagtataagagttgtgaaattgttcttgatgcagatgattttcaggactatgacactagcgtatatgaataggtagatgtttttattttgtcacacaaaaaagaggctcgcacctctttttcttatttctttttatgatttaatacggcattgaggacaatagcgagtaggctggatacgacgattccgtttgagaagaacatttggaaggctgtcggtcgactaagttggcagcatcacccgaagaacatttggaaggctgtcggtcgactacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaacttgtggtaccggtgtatacgggaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgaattaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaacgcgcgaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaacgcgaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagtttttaaagaaattgtatttgttaaatatgtactacaaacttagtagt

SEQ ID NO: 16 - DNA, 11612 bp, Artificial Sequence
  Description of Artificial Sequence Synthetic polynucleotide
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaactacaaaaacaaattacaaaaattcaaaattttcgggtttattacagggacagcagagatccagtttatcgacttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccgccggcaccggtgatcagttatctagactaaaccactttgtacaagaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgactaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggggggcgtaaacgccgcgtggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgatacagtagaaattacagaaactttatcacgtttagtaagtatagaggctgaaaatccagatgaagccgaacgacttgtaagagaaaagtataagagttgtgaaattgttcttgatgcagatgattttcaggactatgacactagcgtatatgaataggtagatgtttttattttgtcacacaaaaaagaggctcgcacctctttttcttatttctttttatgatttaatacggcattgaggacaatagcgagtaggctggatacgacgattccgtttgagaagaacatttggaaggctgtcggtcgactaagttggcagcatcacccgaagaacatttggaaggctgtcggtcgactacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaacttgtggcgccaccgcctcccttgtcgtcgtcgtccttgtagtcgatgtcgtggtccttgtagtcgccgtcgtggtccttgtagtccatggtggcggtaccggtgtatacgggaattctttacgagggtaggaagtggtacggaaagttggtataagacaaaagtgttgtggaattgaagtttactcaaaaaatcagcactcttttataggcgccctggtttacataagcaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgaggtgataattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcatcgaattaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaacgcgcgaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaacgcgaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggtttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagtttttaaagaaattgtatttgttaaatatgtactacaaacttagtagt

(19) United States
(12) Patent Application Publication
(10) Pub. No.: US 2024/0321457 A1
(43) Pub. Date: Sep. 26, 2024

(54) SYSTEMS FOR AND METHODS OF TREATMENT SELECTION

(71) Applicant: THE REGENTS OF THE UNIVERSITY OF CALIFORNIA, Oakland, CA (US)

(72) Inventor: Nevan J. KROGAN, San Francisco, CA (US)

(21) Appl. No.: 18/032,153

(22) PCT Filed: Oct. 14, 2021
(86) PCT No.: PCT/US21/55100

§ 371 Date: Apr. 14, 2023

Related U.S. Application Data
(60) Provisional application No. 63/091,924, filed on Oct. 14, 2020.

(51) Int. Cl. G16H 50/30; G01N 33/574; G01N 33/68; G16H 10/20; G16H 10/40; G16H 20/10

(52) U.S. Cl. CPC G16H 50/30; G01N 33/57484; G01N 33/6848; G16H 10/20; G16H 10/40; G16H 20/10

(57) ABSTRACT

The disclosure relates to a system comprising software that identifies drug targets and predicts responsiveness of cancer subjects to certain disease modifying drugs. Embodiments of the disclosure include methods comprising calculating a differential interaction score (DIS), correlating the DIS with the likelihood that a dysfunctional protein-protein interaction is the causal agent of a hyperproliferative disorder, identifying a drug target based on the causal agent, evaluating a therapeutic specific to the drug target, thereby restoring and/or alleviating dysfunction within the protein network, identifying a subject responsive to a hyperproliferative disorder treatment based upon the causal agent, and monitoring the subject's response to the hyperproliferative disorder treatment.

### CROSS-REFERENCE TO RELATED APPLICATIONS

This application claims the benefit of U.S. Application No. 63/091,924, filed on Oct. 14, 2020, the contents of which are hereby incorporated by reference in their entirety.

### STATEMENT REGARDING FEDERALLY SPONSORED RESEARCH

This invention was made with government support under grant number U54 CA209891 awarded by the National Institutes of Health (NIH). The government has certain rights in the invention.

### REFERENCE TO AN ELECTRONIC SEQUENCE LISTING

The contents of the electronic sequence listing (UCAL021US_SeqListing.txt; size: 69,509 bytes; and date of creation: Dec. 15, 2023) is herein incorporated by reference in its entirety.

### FIELD OF INVENTION

The disclosure relates to a system comprising software that identifies drug targets and predicts responsiveness of cancer subjects to certain disease modifying drugs. Embodiments of the disclosure include methods comprising calculating a differential interaction score (DIS), correlating the DIS with the likelihood that a dysfunctional protein-protein interaction is the causal agent of a hyperproliferative disorder, identifying a drug target based on the causal agent, evaluating a therapeutic specific to the drug target, thereby restoring and/or alleviating dysfunction within the protein network, identifying a subject responsive to a hyperproliferative disorder treatment based upon the causal agent, and monitoring the subject's response to the hyperproliferative disorder treatment.

### BACKGROUND

Genome sequencing efforts over the past decade have profiled the genetic landscape of thousands of patient tumors and solidified the concept of cancer as a highly heterogeneous disease (Biankin et al., 2012; Cancer Genome Atlas, Network, 2012, 2015; Cancer Genome Atlas Research, Network, 2008, 2011; Hoadley et al., 2018; Robinson et al., 2015; Stephens et al., 2012). Evidence from these efforts has also revealed that nearly every human gene is altered in cancer, presenting an overwhelming degree of complexity that has limited the power of connecting individual alterations with cancer patient phenotypes. As a consequence, the field has begun to interpret this heterogeneous genetic landscape in the context of hallmark cancer pathways, with the hypothesis that rare individual alterations among a population converge on more commonly altered protein networks and signaling cascades (Hanahan and Weinberg, 2011; Hanahan et al., 2000; Krogan et al., 2015; Vogelstein et al., 2004). As such, a fundamental component of many cancer genome analyses has been the summarization of genetic alterations in the context of well-characterized cancer pathway diagrams (Biankin et al., 2012; Cancer Genome Atlas, Network, 2012, 2015; Cancer Genome Atlas Research, Network, 2008, 2011; Li et al., 2014; Stephens et al., 2012).

To further facilitate such interpretation, powerful network biology approaches have been developed to bridge the gap between genetic alterations and phenotypes. In such approaches, protein network knowledge is used to aggregate individual tumor mutations and, on the basis of altered networks, predict patient survival and response to therapy (Akavia et al., 2010; Cerami et al., 2010; Consequences and Consortium, Pathway Analysis working group of the International Cancer Genome, 2015; Drier et al., 2013; Hofree et al., 2013; Horn et al., 2018; Leiserson et al., 2015; Li et al., 2016; Paczkowska et al., 2020; Paull et al., 2013; Reyna et al., 2020). However, an important factor in the utility of such network-based approaches is a strong reliance on existing databases of molecular interactions. Publicly available human molecular networks have been populated primarily by systematic efforts to determine protein-protein interactions (PPIs) using large-scale yeast two-hybrid screening (Luck et al., 2020; Rolland et al., 2014) or, more recently, affinity purification-mass spectrometry (AP-MS) (Hein et al., 2015; Huttlin et al., 2015, 2017). The vast majority of PPIs in such databases have been collected either without human cellular context (yeast two-hybrid) or in workhorse cell lines such as HEK293T embryonic kidney cells that lack cancer context. Importantly, there is a growing recognition that such PPIs can vary significantly across cellular contexts (Huttlin et al., 2020). Thus, the generation and incorporation of cancer-specific physical and functional networks may represent a critical component to interpret and predict cancer biology and its clinical outcomes (Krogan et al., 2015).

Breast cancer (BC) is the most common malignancy in women and the second leading cause of cancer-related death in the United States (American Cancer Society, 2019; Anp et al., 2020; Society, 2019), where an estimated 276,480 women and 2,620 men will be newly diagnosed in 2020 (Anp et al., 2020). The disease has been divided into different subtypes, based largely on the presence or absence of three key proteins: estrogen receptor (ER), progesterone receptor (PR), and human epidermal growth factor receptor 2 (HER2/ERBB2). Despite this and much additional heterogeneity at the molecular level, the majority of BC patients are treated using untailored chemotherapy or hormone therapies. Therefore, an urgent need is to develop targeted therapies matched to the specific molecular alterations in a tumor, with the goal of achieving better efficacy and avoiding unnecessary treatment.

HNSCC is a cancer affecting squamous mucosal epithelial cells in the oral cavity, pharynx, and larynx, estimated to be the sixth most common malignancy worldwide (Riaz et al., 2014). The primary causes of HNSCC are carcinogen exposure (e.g., alcohol and tobacco) or infection by the human papillomavirus (HPV). Despite a wealth of data detailing the genetic alterations in this tumor type (Cancer Genome Atlas Network, 2015), only two types of targeted therapies are presently available (Riaz et al., 2014). Therefore, HNSCC also presents a unique opportunity to apply emerging, quantitative, systems approaches to identify new diagnostic subtypes and therapeutic targets.

Network approaches can also be used to further our understanding of existing chemotherapeutic targets, such as PIK3CA, the most commonly mutated oncogene in HNSCC. PIK3CA encodes p110alpha (p110α), the catalytic subunit of phosphatidylinositol 3-kinase (P1I3K), and is a potent mediator of cellular signaling. It interacts with both intracellular small GTPases (e.g., RAS proteins) as well as receptor kinases (e.g., EGFR) to regulate downstream signaling via both the MAPK/ERK pathway and the Akt/mTOR pathway. Hyperactivation of this pathway is a hallmark of numerous tumor types and can be directly attributed to either amplification or mutation of the PIK3CA gene (Bailey et al., 2018). The majority of PIK3CA mutations reside in the helical (E542K and E545K) and kinase domains (H1047R) and have been studied extensively. For example, the H1047R mutation enhances the association of PI3K with the cell membrane, allowing it to bypass the requirement of association with RAS (Zhao and Vogt, 2008). Meanwhile, helical domain mutants (E545K, E542K) disrupt the interaction of p110α with its auto-inhibitory p85 subunits (PIK3R1/2/3), leading to increased kinase activation (Carson et al., 2008; Miled et al., 2007; Shekar et al., 2005). The functions of the remaining non-canonical mutations are less clear. While some have previously been profiled for oncogenic activity (Dogruluk et al., 2015; Lui et al., 2013; Rudd et al., 2011), much remains to be learned about how these mutants regulate PIK3CA function.

Accordingly, there remains a need for methods and systems for facilitating interpretation of cancer biology, predicting clinical outcomes, and developing treatment strategies.

### SUMMARY OF EMBODIMENTS

Advances in DNA sequencing technology have enabled the widespread analysis of breast tumor genomes, creating a catalog of genetic mutations that may initiate or drive tumor progression (Cancer Genome Atlas, Network, 2012; Stephens et al., 2012). In addition to common mutations in well-known cancer genes, such as TP53 and PIK3CA, breast cancers harbor many additional mutations, each of which are seen rarely across the patient population (Cancer Genome Atlas, Network, 2012; Stephens et al., 2012). A key question is how these less common alterations, dispersed across a multitude of genes, elicit pathologic consequences, and patient outcomes. An important answer may lie in understanding how individual gene mutations converge on multi-gene functional modules, including the signaling pathways orchestrating cell proliferation and apoptosis and DNA repair complexes (Cho et al., 2016; Creixell et al., 2015; Hofree et al., 2013; Knijnenburg et al., 2018; Leiserson et al., 2015; Paczkowska et al., 2020; Reyna et al., 2020; Sanchez-Vega et al., 2018; Wood et al., 2007).

PIK3CA and AKT activating mutations and copy-number amplifications are frequently found in many cancer types including BC (Brugge et al., 2007; Carpten et al., 2007; Fruman et al., 2017; Vivanco and Sawyers, 2002; Yuan and Cantley, 2008), indicating that the PI3K/AKT pathway is a key signaling module for cancer cell proliferation, and thus an attractive target for therapeutic intervention (McCubrey et al., 2012; Pal et al., 2010; Yap et al., 2011). Given its substantial role in tumorigenesis, however, how this signaling pathway is regulated by other proteins rather than mutations and/or alterations in the PIK3CA and AKT genes still remains largely unknown.

BRCA1 is a major hereditary cancer susceptibility gene (Futreal et al., 1994; Miki et al., 1994) that plays critical roles in DNA repair by homologous recombination (HR) (Prakash et al.; Venkitaraman, 2014) in addition to other processes, such as regulation of transcription, RNA splicing and cell cycle (Hatchi et al., 2015; Hill et al., 2014; Mullan et al., 2006; Savage et al., 2014). BRCA1 carries out its functions in concert with other proteins (Li and Greenberg, 2012; Moynahan and Jasin, 2010; Prakash et al.; Yun and Hiom, 2009), leading to many studies of BRCA1-containing complexes and their roles in DNA repair (Escribano-Diaz et al., 2013; Hill et al., 2014; Kim et al., 2007a; Liu et al., 2007; Wang et al., 2009, 2000; Wu et al., 1996; Yu et al., 2003). To date, many of these findings have been based on either immunoprecipitation with antibodies against the WT BRCA1 protein or interrogation of pairwise protein interactions with the yeast two-hybrid system. Moreover, these analyses were done exclusively using WT BRCA1 protein and did not capture how different mutations in BRCA1 might affect its protein interactions.

To broadly enable a pathway understanding of cancer, a prerequisite is to generate general and comprehensive maps of cancer molecular networks in relevant malignant and premalignant cell contexts. Here, affinity purification combined with mass spectrometry (AP-MS) is used to catalog protein-protein interactions (PPIs) for 40 proteins significantly altered in BC, including multi-dimensional measurements across mutant and normal protein isoforms and across cancerous and non-cancerous cellular contexts. The resulting interaction landscape reveals many PPIs that are private to a specific cell type or distinct between wild-type (WT) and mutant proteins, thereby providing a framework to understand how PPI networks are re-wired by tumor cell states. Finally, analysis of these multi-dimensional interaction maps in the context of the I-SPY 2 clinical trial (Barker et al., 2009) identifies key proteins and protein complexes with promise as biomarkers of therapeutic response.

Systematic affinity purification and tandem mass spectrometry (AP-MS) experiments were also conducted to map protein networks in the context of head and neck squamous cell carcinoma (HNSCC). Specifically, a comparative AP-MS analysis across 3 cell lines is presented for 31 genes frequently altered in HNSCC, including 16 PIK3CA mutations.

Without wishing to be bound by theory, these results demonstrate that mapping of protein networks in cancer cells reveals novel mechanisms of cancer pathogenesis, instructs the selection of therapeutic targets, and informs which point mutations in the tumor are most likely to respond to treatment.

The present disclosure therefore relates to methods of identifying a therapeutic target for a hyperproliferative disorder treatment, the method comprising: (a) compiling genetic data about a population of subjects that has a mutation candidate that causes a hyperproliferative disorder; (b) performing a mass spectrometry analysis on a sample associated with the hyperproliferative disorder to identify dysfunctional protein-protein interactions associated with the hyperproliferative disorder; (c) calculating a differential interaction score (DIS); (d) correlating the DIS with the likelihood that the dysfunctional protein-protein interaction is a causal agent of the hyperproliferative disorder, wherein if the DIS score is above a first threshold, then the causal agent is selected as a therapeutic target for the hyperproliferative disorder treatment, and wherein if the DIS score is below the first threshold, then the causal agent is not selected as a therapeutic target for the hyperproliferative disorder treatment.

The disclosure further relates to methods of identifying a therapeutic target for a hyperproliferative disorder treatment, the method comprising: (a) calculating a differential interaction score (DIS); and (b) correlating the DIS with a likelihood that a dysfunctional protein-protein interaction is a causal agent of the hyperproliferative disorder, wherein if the DIS score is above a first threshold, then the causal agent is selected as a therapeutic target for the hyperproliferative disorder treatment, and wherein if the DIS score is below the first threshold, then the causal agent is not selected as a therapeutic target for the hyperproliferative disorder treatment.

The disclosure further relates to methods of identifying a therapeutic for treating a hyperproliferative disorder, the method comprising screening a candidate compound for binding with, or activity against a therapeutic target, wherein the therapeutic target was identified via a disclosed method.

The disclosure further relates to methods of predicting a likelihood that a hyperproliferative disorder is responsive to a therapeutic, the method comprising: (a) compiling genetic data about a population of subjects that has a mutation candidate that causes a hyperproliferative disorder; (b) performing a mass spectrometry analysis on a sample associated with the hyperproliferative disorder to identify dysfunctional protein-protein interactions associated with the hyperproliferative disorder; (c) calculating a differential interaction score (DIS); (d) correlating the DIS with the likelihood that the dysfunctional protein-protein interaction is the causal agent of the hyperproliferative disorder; and (e) selecting a therapeutic for treating the hyperproliferative disorder based upon the causal agent.

The disclosure further relates to methods of identifying a subject likely to respond to a hyperproliferative disorder treatment, the method comprising: (a) compiling genetic data about a population of subjects that has a mutation candidate that causes a hyperproliferative disorder, wherein the population of subjects includes the subject; (b) performing a mass spectrometry analysis on a sample associated with the hyperproliferative disorder to identify dysfunctional protein-protein interactions associated with the hyperproliferative disorder; (c) calculating a differential interaction score (DIS); and (d) correlating the DIS with the likelihood that the dysfunctional protein-protein interaction is a causal agent of the hyperproliferative disorder.

The disclosure further relates to methods of identifying a subject likely to respond to a hyperproliferative disorder treatment, the method comprising: (a) calculating a differential interaction score (DIS); and (b) correlating the DIS with a likelihood that a dysfunctional protein-protein interaction is a causal agent of the hyperproliferative disorder, wherein if the DIS score is above a first threshold, then the subject is likely to respond to a hyperproliferative disorder treatment based upon the causal agent, and wherein if the DIS score is below the first threshold, then the subject is not likely to respond to the hyperproliferative disorder treatment based upon the causal agent.

The disclosure further relates to methods of predicting a likelihood that a subject does or does not respond to a hyperproliferative disorder treatment, the method comprising: (a) compiling genetic data about a population of subjects that has a mutation candidate that causes a hyperproliferative disorder, wherein the population of subjects includes the subject; (b) performing a mass spectrometry analysis on a sample associated with the hyperproliferative disorder to identify dysfunctional protein-protein interactions associated with the hyperproliferative disorder; (c) calculating a differential interaction score (DIS); (d) correlating the DIS with the likelihood that the dysfunctional protein-protein interaction is the causal agent of the hyperproliferative disorder; and (e) selecting a cancer treatment for the subject based upon the causal agent.

The disclosure further relates to computer program products encoded on a computer-readable storage medium, wherein the computer program product comprises instructions for: (a) performing a mass spectrometry analysis on a sample from a subject that has a mutation candidate that causes a hyperproliferative disorder; (b) identifying dysfunctional protein-protein interactions associated with the hyperproliferative disorder; and (c) calculating a differential interaction score (DIS).

The disclosure further relates to systems for identifying a protein interaction network in a subject, the system comprising: (a) a processor operable to execute programs; (b) a memory associated with the processor; (c) a database associated with said processor and said memory; and (d) a program stored in the memory and executable by the processor, the program being operable for: (i) performing a mass spectrometry analysis on a sample from a subject that has a mutation candidate that causes a hyperproliferative disorder; (ii) identifying dysfunctional protein-protein interactions associated with the hyperproliferative disorder; and (iii) calculating a differential interaction score (DIS).

The disclosure further relates to methods of treating a cancer in a subject having a genetic alteration in Akt signaling, the method comprising administering to the subject a pharmaceutically effective amount of an Akt inhibitor, wherein the subject was previously identified as being in need of treatment by: (a) performing a mass spectrometry analysis on a sample from the subject; (b) identifying dysfunctional protein-protein interactions associated with the hyperproliferative disorder; and (c) calculating a differential interaction score (DIS).

The disclosure further relates to methods of treating a cancer in a subject having a genetic alteration in HER3 expression, the method comprising administering to the subject a pharmaceutically effective amount of a HER3 inhibitor, wherein the subject was previously identified as being in need of treatment by: (a) performing a mass spectrometry analysis on a sample from the subject; (b) identifying dysfunctional protein-protein interactions associated with the hyperproliferative disorder; and (c) calculating a differential interaction score (DIS).

The disclosure further relates to methods of selecting a hyperproliferative disorder treatment for a subject in need thereof, the method comprising: (a) identifying genetic data from the subject in need of treatment; (b) comparing the genetic data from the subject to a compilation of genetic data from population of subjects that has a mutation candidate that causes a hyperproliferative disorder, wherein the population of subjects includes the subject in need thereof; (c) performing a mass spectrometry analysis on a sample from the subject associated with the hyperproliferative disorder to identify dysfunctional protein-protein interactions associated with the hyperproliferative disorder; (d) calculating a differential interaction score (DIS); (e) correlating the DIS with the likelihood that the dysfunctional protein-protein interaction is a causal agent of the hyperproliferative disorder; and (f) selecting a hyperproliferative disorder treatment for the subject based upon the causal agent.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several embodiments and together with the description serve to explain the principles of the invention.

FIG. 1A and FIG. 1B show representative data illustrating protein-protein interaction filtering using compass and SAINTexpress algorithms.

FIG. 2A-D show representative data illustrating an overview of high-confidence PPIs from three breast cell lines.

FIG. 3A-E show representative data illustrating an overview of protein-protein interaction mapping in breast epithelial cells.

FIG. 4A-I show representative data illustrating a differential interaction analysis of the BC-specific interactome.

FIG. 5 shows representative data illustrating that knockdown of AKT reduces its phosphorylation on S473.

FIG. 6A-I show representative data illustrating a comparative interactome analysis of WT and mutant proteins.

FIG. 7A-E show representative data illustrating a quantitative analysis of the effect of mutations on the BRCA1 interactome.

FIG. 8A-C show representative data illustrating that pathogenic mutations in the BRCT domain of BRCA1 disrupt the interaction with HR proteins.

FIG. 9A-G show representative data illustrating that Spinophilin interacts with BRCA1 and regulates DNA damage response via dephosphorylation.

FIG. 10A-E show representative data illustrating that knockout of PPP1R9B (encoding Spinophilin) upregulates phosphorylation on many DNA repair proteins.

FIG. 11A-H show representative data illustrating an experimental design and workflow for mapping of the head and neck cancer interactions.

FIG. 12A-C show representative data illustrating an overview of HC-PPI detection.

FIG. 13A-E show representative data illustrating a differential interaction analysis of the HNSCC specific interactome.

FIG. 14 shows representative data illustrating the properties of differentially interacting proteins.

FIG. 15A-I show representative data illustrating that an HNSCC-specific FGFR3:Daple interaction mediates activation of cell migratory proteins.

FIG. 16A-C show representative data illustrating that HNSCC-specific FGFR3:Daple interaction mediates activation of Gαi.

FIG. 17A-D show representative data illustrating a quantitative interactome analysis of common missense mutations in HNSCC.

FIG. 18 shows representative heatmap displaying the PPI regulation between WT and mutant baits for all PPIs found commonly in 2 or more cell lines.

FIG. 19A-G show representative data illustrating that PIK3CA mutant interactome.

FIG. 20A-C show representative data illustrating the PIK3CA mutant interactome and cellular response to CDX3379 treatment.

FIG. 21A-E show representative data illustrating in vivo targeting of HER3 in the context of different PIK3CA mutants.

FIG. 22 and FIG. 23 show representative plasmid maps, SEQ ID NO: 15 and SEQ ID NO:16 respectively, including the nucleic acid sequence with annotations. See also paragraph [0175].

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF EMBODIMENTS

Before the present systems and methods are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean.

According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

Ranges provided herein are understood to include all individual integer values and all subranges within the ranges.

As used herein, the terms “cancer patient,” “individual diagnosed with cancer,” and “individual suspected of having cancer” all refer to an individual who has been diagnosed with cancer, has been given a probable diagnosis of cancer, or an individual who has positive PET scans but otherwise lacks major symptoms of cancer and is without a clinical diagnosis of cancer.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the subject in need thereof is a human seeking prevention of cancer. In some embodiments, the subject in need thereof is a human diagnosed with cancer. In some embodiments, the subject in need thereof is a human seeking treatment for cancer. In some embodiments, the subject in need thereof is a human undergoing treatment for cancer.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods or compositions of matter wherein the sample is taken from a mammal or non-human mammal. The present disclosure relates to any of the methods or compositions of matter wherein the sample is taken from a human or non-human primate.

As used herein, the term “predicting” refers to making a finding that an individual has a significantly enhanced probability or likelihood of benefiting from and/or responding to a chemotherapeutic treatment. In some embodiments, the chemotherapeutic treatment is administration of an Akt modulator. In some embodiments, the chemotherapeutic treatment is administration of a HER3 inhibitor.

A “score” is a numerical value that may be assigned or generated after normalization of the value based upon the presence, absence, or quantity of dysfunctional protein-protein interactions associated with a hyperproliferative disorder. In some embodiments, the score is normalized in respect to a control data value.

As used herein, the term “stratifying” refers to sorting individuals into different classes or strata based on the features of cancer. For example, stratifying a population of individuals with breast cancer involves assigning the individuals on the basis of the severity of the disease (e.g., stage 0, stage 1, stage, 2, stage 3, etc.).

As used herein, the term “subject,” “individual,” or “patient,” used interchangeably, means any animal, including mammals, such as mice, rats, other rodents, rabbits, dogs, cats, swine, cattle, sheep, horses, or primates, such as humans. In some embodiments, the subject is a human seeking treatment for cancer. In some embodiments, the subject is a human diagnosed with cancer. In some embodiments, the subject is a human suspected of having cancer. In some embodiments, the subject is a healthy human being.

As used herein, the term “threshold” refers to a defined value by which a normalized score can be categorized. By comparing to a preset threshold, a normalized score can be classified based upon whether it is above or below the preset threshold.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the term “therapeutic” means an agent utilized to treat, combat, ameliorate, prevent, or improve an unwanted condition or disease of a patient.

A “therapeutically effective amount” or “effective amount” of a composition is a predetermined amount calculated to achieve the desired effect, i.e., to treat, combat, ameliorate, prevent, or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way.

A therapeutically effective amount of compounds of embodiments of the present disclosure is typically an amount such that when it is administered in a physiologically tolerable excipient composition, it is sufficient to achieve an effective systemic concentration or local concentration in the tissue.

The term “hyperproliferative disorder” refers to a disease or disorder characterized by abnormal proliferation, abnormal growth, abnormal senescence, abnormal quiescence, or abnormal removal of cells in an organism, and includes all forms ofhyperplasias, neoplasias, and cancer. In some embodiments, the hyperproliferative disease is a cancer derived from the gastrointestinal tract or urinary system. In some embodiments, a hyperproliferative disease is a cancer of the adrenal gland, bladder, bone, bone marrow, brain, spine, breast, cervix, gall bladder, ganglia, gastrointestinal tract, stomach, colon, heart, kidney, liver, lung, muscle, ovary, pancreas, parathyroid, penis, prostate, salivary glands, skin, spleen, testis, thymus, thyroid, or uterus. In some embodiments, the term hyperproliferative disease is a cancer chosen from: lung cancer, bone cancer, CMML, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, testicular, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The terms “identical” or “percent identity” or “homology” in the context of two or more nucleic acids, as used herein, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art.

These include, but are not limited to, BLAST, ALIGN, Megalign, BestFit, GCG Wisconsin Package, and variations thereof. In some embodiments, two nucleic acids of the invention are substantially identical, meaning they have at least about 70%, at least about 75%, at least about 80%, at least about 85%, at least about 90%, and in some embodiments at least about 95%, 96%, 97%, 98%, 99% nucleotide or amino acid residue sequence identity, when compared and aligned for maximum correspondence, as measured using a sequence comparison algorithm or by visual inspection. In some embodiments, identity exists over a region of the sequences that is at least about 10, at least about 20, at least about 40-60 nucleotides, at least about 60-80 nucleotides or any integral value therebetween. In some embodiments, identity exists over a longer region than 60-80 nucleotides, such as at least about 80-100 nucleotides, and in some embodiments the sequences are substantially identical over the full length of the sequences being compared.

### Methods of Developing Protein-Protein Interaction Maps and Identifying Dysfunctional Protein-Protein Interactions

In some embodiments, the disclosure relates to methods of developing a protein-protein interaction map, the method comprising compiling genetic data about a population of subjects that has a mutation candidate that causes a hyperproliferative disorder. In some embodiments, the method further comprises performing a mass spectrometry analysis on a sample associated with the hyperproliferative disorder, thereby identifying dysfunctional protein-protein interactions associated with the hyperproliferative disorder.

In some embodiments, disclosed are methods of identifying a dysfunctional protein-protein interaction, the method comprising: (a) identifying genetic data from a subject in need of hyperproliferative disorder treatment; (b) comparing the genetic data from the subject to a compilation of genetic data from a population of subjects that has a mutation candidate that causes a hyperproliferative disorder; and (c) performing a mass spectrometry analysis on a sample from the subject in need of hyperproliferative disorder treatment, thereby identifying dysfunctional protein-protein interactions associated with the hyperproliferative disorder. In some embodiments, the method further comprises: (d) calculating a differential interaction score (DIS). In some embodiments, the method further comprises: (e) correlating the DIS with the likelihood that the dysfunctional protein-protein interaction is a causal agent of the hyperproliferative disorder. In some embodiments, the method further comprises: (f) selecting a hyperproliferative disorder treatment for the subject based upon the causal agent. In some embodiments, the step of identifying the genetic information from a subject comprises sequencing the genetic information from a biopsy or a sample obtained from the subject.

In some embodiments, the sample is a population of cells. For example, in some embodiments, the population of cells are cancer cells.

In some embodiments, the mass spectrometry analysis is performed on a plurality of samples. In further embodiments, each sample is a different population of cells. Thus, for example, the cells can be cancer cells or non-cancerous cells. In still further embodiments, each sample is the same population of cells (e.g., cancer cells, non-cancerous cells).

In some embodiments, the mass spectrometry analysis is performed on a plurality of samples, wherein calculating comprises calculating a SAINTexpress algorithm score for each sample, and averaging the SAINTexpress algorithm scores.

In some embodiments, the hyperproliferative disorder is a cancer. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, head and neck cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma). In further embodiments, the cancer is breast cancer of head and neck cancer. In still further embodiments, the cancer is breast cancer. In yet further embodiments, the cancer is head and neck cancer.

In some embodiments, the method further comprises harvesting samples with a functional bioassay. In a further embodiment, the functional bioassay is an animal model comprising growth of transformed cell lines.

In some embodiments, the dysfunctional protein-protein interaction is one or more of a D1:PI3K interaction or a FGFR3: Daple interaction. In some embodiments, the dysfunctional protein-protein interaction is one or more of a BPIFA1: PIK3CA interaction, a S100A3: Akt interaction, a SCGB2A1: PIK3CA interaction, or a Spinophilin: BRCA1 interaction.

### Methods of Identifying Therapeutic Targets and of Screening for and Evaluating Therapeutics

In some embodiments, the disclosure relates to methods of identifying a therapeutic target for a hyperproliferative disorder treatment, the method comprising: (a) compiling genetic data about a population of subjects that has a mutation candidate that causes a hyperproliferative disorder; (b) performing a mass spectrometry analysis on a sample associated with the hyperproliferative disorder to identify dysfunctional protein-protein interactions associated with the hyperproliferative disorder; (c) calculating a differential interaction score (DIS); (d) correlating the DIS with the likelihood that the dysfunctional protein-protein interaction is a causal agent of the hyperproliferative disorder, wherein if the DIS score is above a first threshold, then the causal agent is selected as a therapeutic target for the hyperproliferative disorder treatment, and wherein if the DIS score is below the first threshold, then the causal agent is not selected as a therapeutic target for the hyperproliferative disorder treatment. In some embodiments, the methods further comprise selecting the treatment of a subject.

In some embodiments, the disclosure relates to methods of identifying a therapeutic target for a hyperproliferative disorder treatment, the method comprising: (a) calculating a differential interaction score (DIS); and (b) correlating the DIS with a likelihood that a dysfunctional protein-protein interaction is a causal agent of the hyperproliferative disorder, wherein if the DIS score is above a first threshold, then the causal agent is selected as a therapeutic target for the hyperproliferative disorder treatment, and wherein if the DIS score is below the first threshold, then the causal agent is not selected as a therapeutic target for the hyperproliferative disorder treatment.

In some embodiments, the disclosure relates to methods of identifying a therapeutic for treating a hyperproliferative disorder, the method comprising screening a candidate compound for binding with, or activity against a therapeutic target, wherein the therapeutic target was identified via a disclosed method.

In some embodiments, the disclosure relates to methods of predicting a likelihood that a hyperproliferative disorder is responsive to a therapeutic, the method comprising: (a) compiling genetic data about a population of subjects that has a mutation candidate that causes a hyperproliferative disorder; (b) performing a mass spectrometry analysis on a sample associated with the hyperproliferative disorder to identify dysfunctional protein-protein interactions associated with the hyperproliferative disorder; (c) calculating a differential interaction score (DIS); (d) correlating the DIS with the likelihood that the dysfunctional protein-protein interaction is the causal agent of the hyperproliferative disorder; and (e) selecting a therapeutic for treating the hyperproliferative disorder based upon the causal agent.

In some embodiments, the sample is a population of cells. For example, in some embodiments, the population of cells are cancer cells.

In some embodiments, the mass spectrometry analysis is performed on a plurality of samples. In further embodiments, each sample is a different population of cells. Thus, for example, the cells can be cancer cells or non-cancerous cells. In still further embodiments, each sample is the same population of cells (e.g., cancer cells, non-cancerous cells).

In some embodiments, the calculating comprises calculating one or more of a SAINTexpress algorithm score and a CompPASS algorithm score. In a further embodiment, the calculating comprises calculating the SAINTexpress algorithm score. In a still further embodiment, the calculating comprises calculating the CompPASS algorithm score. In yet further embodiments, the calculating comprises calculating the SAINTexpress algorithm score and the CompPASS algorithm score.

Methods of using SAINTexpress algorithms are known by those of skill in the art. See, e.g., Teo, et al. (2014) J Proteomics 100: 37-43. As further described herein, a SAINTexpress algorithm can be used for PPI confidence scoring. In various aspects, PPI scoring can be performed separately for each cell line.

In some embodiments, the SAINTexpress algorithm score is calculated by a formula:

$$P ( {X}_{ij} \vert \text{*)} = {π}_{T} P ( {X}_{ij} \vert {λ}_{ij} ) + ( 1 - {π}_{T} ) P ( {X}_{ij} \vert {κ}_{ij} ) \qquad ( 1 )$$

wherein Xᵢⱼ is the spectral count for a prey protein i identified in a purification of bait j;
wherein λᵢⱼ is the mean count from a Poisson distribution representing true interaction;
wherein κᵢⱼ is the mean count from a Poisson distribution representing false interaction;
wherein π_{T} is the proportion of true interactions in the data; and wherein dot notation represents all relevant model parameters estimated from the data for the pair of prey i and bait j.

Methods of using CompPASS algorithms are known by those of skill in the art. See, e.g., Huttlin, et al. (2015) Cell 162: 425-440; and Sowa, et al. (2009) Cell 138: 389-403. As further described herein, a CompPASS algorithm can be used for PPI confidence scoring. In various aspects, PPI scoring can be performed separately for each cell line.

In some embodiments, the CompPASS algorithm score is calculated by calculating the Z-score, the S-score, the D-score, and the WD-score, as further described herein.

In some embodiments, the DIS is calculated for a cancer cell line or a plurality of cancer cell lines and also calculated for a normal cell line. The DIS for the cancer cell line or the plurality of cancer cell lines is then compared to the DIS for the normal cell line. If the DIS for the cancer cell line or the plurality of cancer cell lines is greater than the DIS for the normal cell line, the DIS is assigned a positive (+) sign. If the DIS for the cancer cell line or the plurality of cancer cell lines is less than the DIS for the normal cell line, the DIS is assigned a negative (−) sign. Thus, a positive DIS represents a PPI that is enriched in a cancer cell line or a plurality of cancer cell lines, and a negative DIS represents a PPI that is depleted in a cancer cell line or a plurality of cancer cell lines.

In some embodiments, the DIS is calculated by a first formula:

$${DIS}_{A} ( b , p ) = {S}_{C 1} ( b , p ) \times {S}_{C 2} ( b , p ) \times [ 1 - {S}_{C 3} ( b , p ) ]$$

wherein DIS_{A}(b,p) is the DIS for each PPI (b, p) that is conserved in a first cell line and a second cell line, but not shared by a third cell line; wherein S_{C1}(b,p) is the probability of a PPI being present in the first cell line; wherein S_{C2}(b,p) is the probability of a PPI being present in the second cell line; and wherein S_{c3}(b,p) is the probability of a PPI being present in the third cell line; and a second formula:

$${DIS}_{B} ( b , p ) = [ 1 - {S}_{C 1} ( b , p ) ] \times [ 1 - {S}_{C 2} ( b , p ) ] \times {S}_{C 3} ( b , p$$

wherein DIS_{B}(b,p) is the DIS score for each PPI (b, p) that is conserved in the third cell line, but not shared by the first cell line and the second cell line; wherein a (+) sign is assigned if DIS_{A}(b,p)>DIS_{B}(b,p); and wherein a (−) sign is assigned if DIS_{A}(b,p)<DIS_{B}(b,p).

In some embodiments, the DIS is calculated by a first formula:

$${DIS}_{cancer} ( b , p ) = {S}_{C 1} ( b , p ) \times {S}_{C 2} ( b , p ) \times [ 1 - {S}_{N} ( b , p ) ]$$

wherein DIS_{cancer}(b,p) is the DIS for each PPI (b, p) that is conserved across a cancer cell line, but not shared by a normal cell line; wherein S_{C1}(b,p) is the probability of a PPI being present in a first cancer cell line; wherein S_{C2}(b,p) is the probability of a PPI being present in a second cancer cell line; and wherein S_{N}(b,p) is the probability of a PPI being present in a normal cell line; and a second formula:

$${DIS}_{normal} ( b , p ) = [ 1 - {S}_{C 1} ( b , p ) ] \times [ 1 - {S}_{C 2} ( b , p ) ] \times {S}_{N} ( b , p )$$

wherein DISₙₒᵣₘₐₗ(b,p) is the DIS score for each PPI (b, p) that is present in a normal cell line, but depleted in a cancer cell line; and assigning a (+) sign if DIS_{cancer}(b,p)>DISₙₒᵣₘₐₗ(b,p) and assigning a (−) sign if DIS_{cancer}(b,p)<DISₙₒᵣₘₐₗ(b,p).

In some embodiments, the DIS is an average of a SAINTexpress algorithm score and a CompPASS algorithm score. In some further embodiments, the DIS is a SAINTexpress algorithm score.

In some embodiments, the DIS ranges from 0.0 to 1.0. Thus, in various embodiments, the DIS ranges from 0.0 to 0.9, from 0.0 to 0.8, from 0.0 to 0.7, from 0.0 to 0.6, from 0.0 to 0.5, from 0.0 to 0.4, from 0.0 to 0.3, from 0.0 to 0.2, from 0.0 to 0.1, from 0.1 to 1.0, from 0.2 to 1.0, from 0.3 to 1.0, from 0.4 to 1.0, from 0.5 to 1.0, from 0.6 to 1.0, from 0.7 to 1.0, from 0.8 to 1.0, from 0.9 to 1.0, from 0.1 to 0.9, from 0.2 to 0.8, from 0.3 to 0.7, or from 0.4 to 0.6.

In some embodiments, a DIS of 0.5 or greater than 0.5 indicates that the dysfunctional protein-protein interaction is likely a causal agent of the hyperproliferative disorder. Thus, in various embodiments, a DIS of greater than 0.5, greater than 0.6, greater than 0.7, greater than 0.8, or greater than 0.9 indicates that the dysfunctional protein-protein interaction is likely a causal agent of the hyperproliferative disorder. In some embodiments, a DIS of 0.5 or greater than 0.5 indicates that the dysfunctional protein-protein interaction is likely a causal agent of the hyperproliferative disorder, and, therefore, indicates that the causal agent should be selected as a therapeutic target for a hyperproliferative disorder treatment.

In some embodiments, a DIS of 0.5 or less than 0.5 indicates that the dysfunctional protein-protein interaction is not likely a causal agent of the hyperproliferative disorder. Thus, in various embodiments, a DIS of less than 0.5, less than 0.4, less than 0.3, less than 0.2, or less than 0.1 indicates that the dysfunctional protein-protein interaction is not likely a causal agent of the hyperproliferative disorder. In some embodiments, a DIS of 0.5 or less than 0.5 indicates that the dysfunctional protein-protein interaction is not likely a causal agent of the hyperproliferative disorder, and, therefore, indicates that the causal agent should not be selected as a therapeutic target for a hyperproliferative disorder treatment.

In some embodiments, the mass spectrometry analysis is performed on a plurality of samples, wherein calculating comprises calculating a SAINTexpress algorithm score for each sample, and averaging the SAINTexpress algorithm scores.

In some embodiments, the hyperproliferative disorder is a cancer. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, head and neck cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma). In further embodiments, the cancer is breast cancer of head and neck cancer. In still further embodiments, the cancer is breast cancer. In yet further embodiments, the cancer is head and neck cancer.

In some embodiments, the method further comprises harvesting samples with a functional bioassay. In a further embodiment, the functional bioassay is an animal model comprising growth of transformed cell lines.

In some embodiments, the subject is a mammal. In some embodiments, the mammal is a human.

In some embodiments, the subject has been diagnosed with a need for treatment of the hyperproliferative disorder prior to the administering step.

In some embodiments, the method further comprises identifying a therapeutic target for a hyperproliferative disorder treatment. In a further embodiment, the therapeutic target is identified as a hyperproliferative disorder treatment if the DIS score is 0.5 or greater than 0.5.

Thus, in various embodiments, the subject is identified as being likely to respond to a cancer treatment if the DIS score is greater than 0.5, greater than 0.6, greater than 0.7, greater than 0.8, or greater than 0.9.

In some embodiments, the target is identified as being unlikely to offer a therapeutic benefit as a hyperproliferative disorder treatment if the DIS score is 0.5 or less than 0.5.

Thus, in various embodiments, the target is identified as being unlikely to offer a therapeutic benefit as a hyperproliferative disorder treatment if the DIS score is less than 0.5, less than 0.4, less than 0.3, less than 0.2, or less than 0.1.

In some embodiments, the mutation candidate is one or more genes having a mutant protein sequence, wherein the gene is selected from TP53, CDKN2A, PIK3CA, TP63, FADD, SOX2, RHOA, CCND1, EGFR, CASP8, NFE2L2, MAPK1, MYC, PTEN, KEAP1, CUL3, E2F1, FBXW7, PTPRT, GFGR1, RB1, IGF1R, HRAS, TRAF3, TGFBR2, ERBB2, FGFR3, HLA-A, NRAS, STAT3, and XPC. In some embodiments, the mutation candidate is one or more genes having a mutant protein sequence, wherein the gene is selected from PIK3CA, TP53, MTDG, AKT3, CDH1, ERBB2, GATA3, TSPYL5, PTEN, RB1, BRIP1, CBFB, RAF51C, FOXA1, PALB2, ARID1A, ESR1, STK11, CDKN1B, MSH2, AKT1, AKT2, BRCA1, CHEK2, RPA2, EGFR, RAD51D, CASP8, CCND3, CTCF, MLH1, SMARCB1, XPC, SCUBE2, TBX3, XRN2, EZH2, FANCC, HRAS, or SMARCD1.

In some embodiments, the gene is TP53, PIK3CA, NFE212, MAPK1, FBXW7, or HRAS. In some embodiments, the gene is AKT1, AKT3, BRCA1, BRIP1, CDH1, CHEK2, HRAS, MTDH, PALB2, PIK3CA, or TP53.

In some embodiments, the gene is NFE2L2 and the mutant protein sequence is E79K or E79Q, wherein the gene is HRAS and the mutant protein sequence is G12D, wherein the gene is TP53 and the mutant protein sequence is R248W or R273H, wherein the gene is MAPK1 and the mutant protein sequence is E322K, or wherein the gene is FBXW7 and the mutant protein sequence is R505G.

In some embodiments, the gene is AKT1 and the mutant protein sequence is E17K, wherein the gene is AKT3 and the mutant protein sequence is E17K, wherein the gene is BRIP1 and the mutant protein sequence is A745T, wherein the gene is CDH1 and the mutant protein sequence is E243K, wherein the gene is CHEK2 and the mutant protein sequence is 1100deIC or K373E, wherein the gene is HRAS and the mutant protein sequence is G12D, wherein the gene is MTDH and the mutant protein sequence is A78S, wherein the gene is PALB2 and the mutant protein sequence is E837K, or wherein the gene is TP53 and the mutant protein sequence is R175H, R248W, or R273H.

In some embodiments, the gene is PIK3CA and the mutant protein sequence is R88Q, E110DeI, K111N, K111E, V344G, G363A, E453K, E542K, E545K, E545G, E726K, C971R, G1007R, M1043V, H1047L, or H1047R. In some embodiments, the gene is PIK3CA and the mutant protein sequence is E545K, M1043V, or H1047R. In some embodiments, the gene is BRCA1 and the mutant protein sequence is I16A, C61G, R71G, Δexon11, S1655F, 5832insC, or M1775R.

In some embodiments, the dysfunctional protein-protein interaction is one or more of a D1:PI3K interaction or a FGFR3: Daple interaction. In some embodiments, the dysfunctional protein-protein interaction is one or more of a BPIFA1: PIK3CA interaction, a S100A3: Akt interaction, a SCGB2A1: PIK3CA interaction, or a Spinophilin: BRCA1 interaction.

In some embodiments, the causal agent is HER3. In some embodiments, the causal agent is Akt.

In some embodiments, the method further comprises selecting a therapeutic target for treating a hyperproliferative disorder in a subject based upon the causal agent. In some embodiments, the method further comprises screening a candidate compound for binding with, or activity against, the therapeutic target. In some embodiments, the method further comprises selecting a candidate compound as a therapeutic for treating a hyperproliferative disorder. In some embodiments, the candidate compound is selected from a database of known treatments for the dysfunctional protein-protein interaction.

In some embodiments, the hyperproliferative disorder treatment comprises administration of a HER3 inhibitor. Examplers of HER3 inhibitors include, but are not limited to, lapatinib, erlotinib, gefitinib, afatinib, neratinib, CDX-3379, U-31402, HMBD-001, MCLA-128, KBP-5209, Poziotinib, Varlitinib, FCN-411, Elgemtumab, Sirotinib, vaccines to target Her3 for solid tumors, AV2103, AV2103, ETBX-031, MP-EV-20, MP-EV-20/1959, and oligonucleotides to inhibit EGFR, ERBB2, and ERBB3. Additional exemplary HER3 inhibitors are described in US 2018/0362443 A1, U.S. Pat. No. 10,383,878 B2, US 2019/0300624 A1, WO 2018/182420 A1, WO 2015/007219 A1, U.S. Pat. No. 8,735,551 B2, U.S. Pat. No. 10,507,209 B2, U.S. Pat. No. 9,956,222 B2, U.S. Pat. No. 10,487,143 B2, WO 2018/233511 A1, CN106692969A, US 2020/0147193 A1, U.S. Pat. No. 9,346,889 B2, WO 2020/099235 A1, US 2019/0201552 A1, US 2018/0105815 A1, and US 2020/0157542 A1. In some embodiments, the HER3 inhibitor is CDX3379.

In some embodiments, the hyperproliferative disorder is head and neck cancer, wherein the mutation candidate is a mutant PIK3CA, wherein the causal agent is HER3, and wherein the hyperproliferative disorder treatment comprises administration of a HER3 inhibitor.

In some embodiments, the hyperproliferative disorder treatment comprises administration of an Akt inhibitor. Examples of Akt inhibitors include, but are not limited to, MK-2206, AZD5363, GSK690693, GDC-0068, GSK2141795, GSK2110183, AT7867, CCT128930, BAY1125976, perifosine, and AKT inhibitor III.

In some embodiments, the Akt modulator is a PIK3CA modulator. Examples of PIK3CA modulators include, but are not limited to, Alpelisib, Copanlisib hydrochloride, GDC-0077, Bimiralisib, Fimepinostat, Serabelisib, HHCYH-33, omipalisib, and PQR-514.

In some embodiments, the hyperproliferative disorder is breast cancer, wherein the mutation candidate is a mutant PIK3CA or a mutant BRCA1, wherein the causal agent is Akt, and wherein the hyperproliferative disorder treatment comprises administration of an Akt inhibitor.

### Methods of Identifying and Monitoring a Subject's Responsiveness to a Hyperproliferative Disorder Treatment

In some embodiments, the disclosure relates to methods of identifying a subject likely to respond to a hyperproliferative disorder treatment, the method comprising: (a) compiling genetic data about a population of subjects that has a mutation candidate that causes a hyperproliferative disorder, wherein the population of subjects includes the subject; (b) performing a mass spectrometry analysis on a sample associated with the hyperproliferative disorder to identify dysfunctional protein-protein interactions associated with the hyperproliferative disorder; (c) calculating a differential interaction score (DIS); and (d) correlating the DIS with the likelihood that the dysfunctional protein-protein interaction is a causal agent of the hyperproliferative disorder.

In some embodiments, the disclosure relates to methods of identifying a subject likely to respond to a hyperproliferative disorder treatment, the method comprising: (a) calculating a differential interaction score (DIS); and (b) correlating the DIS with a likelihood that a dysfunctional protein-protein interaction is a causal agent of the hyperproliferative disorder, wherein if the DIS score is above a first threshold, then the subject is likely to respond to a hyperproliferative disorder treatment based upon the causal agent, and wherein if the DIS score is below the first threshold, then the subject is not likely to respond to the hyperproliferative disorder treatment based upon the causal agent. In some embodiments, the method further comprises: (a) compiling genetic data about a population of subjects comprising the subject, wherein the population of subjects has a mutation candidate that causes the hyperproliferative disorder; and (b) performing a mass spectrometry analysis on a sample associated with the hyperproliferative disorder to identify dysfunctional protein-protein interactions associated with the hyperproliferative disorder.

In some embodiments, disclosed are methods of predicting a likelihood that a subject does or does not respond to a hyperproliferative disorder treatment, the method comprising: (a) compiling genetic data about a population of subjects that has a mutation candidate that causes a hyperproliferative disorder, wherein the population of subjects includes the subject; (b) performing a mass spectrometry analysis on a sample associated with the hyperproliferative disorder to identify dysfunctional protein-protein interactions associated with the hyperproliferative disorder; (c) calculating a differential interaction score (DIS); (d) correlating the DIS with the likelihood that the dysfunctional protein-protein interaction is the causal agent of the hyperproliferative disorder; and (e) selecting a cancer treatment for the subject based upon the causal agent. In some embodiments, the method further comprises: (f) comparing the DIS score to a first threshold; and (g) classifying the subject as being likely to respond to a hyperproliferative disorder treatment, wherein each of steps (f) and (g) are performed after step (c), and wherein the first threshold is calculated relative to a first control dataset.

In some embodiments, disclosed are methods of treating a cancer in a subject having a genetic alteration in Akt signaling, the method comprising administering to the subject a pharmaceutically effective amount of an Akt inhibitor, wherein the subject was previously identified as being in need of treatment by: (a) performing a mass spectrometry analysis on a sample from the subject; (b) identifying dysfunctional protein-protein interactions associated with the cancer; and (c) calculating a differential interaction score (DIS). In some embodiments, the cancer is head and neck cancer.

In some embodiments, disclosed are methods of treating a cancer in a subject having a genetic alteration in HER3 expression, the method comprising administering to the subject a pharmaceutically effective amount of a HER3 inhibitor, wherein the subject was previously identified as being in need of treatment by: (a) performing a mass spectrometry analysis on a sample from the subject; (b) identifying dysfunctional protein-protein interactions associated with the cancer; and (c) calculating a differential interaction score (DIS). In some embodiments, the cancer is breast cancer.

In some embodiments, disclosed are methods of selecting a hyperproliferative disorder treatment for a subject in need thereof, the method comprising: (a) identifying genetic data from the subject in need of treatment; (b) comparing the genetic data from the subject to a compilation of genetic data from population of subjects that has a mutation candidate that causes a hyperproliferative disorder, wherein the population of subjects includes the subject in need thereof; (c) performing a mass spectrometry analysis on a sample from the subject associated with the hyperproliferative disorder to identify dysfunctional protein-protein interactions associated with the hyperproliferative disorder; (d) calculating a differential interaction score (DIS); (e) correlating the DIS with the likelihood that the dysfunctional protein-protein interaction is a causal agent of the hyperproliferative disorder; and (f) selecting a hyperproliferative disorder treatment for the subject based upon the causal agent. In some embodiments, the step of identifying the genetic information from a subject comprises sequencing the genetic information from a biopsy or sample obtained from the subject.

In some embodiments, the sample is a population of cells. For example, in some embodiments, the population of cells are cancer cells.

In some embodiments, the mass spectrometry analysis is performed on a plurality of samples. In further embodiments, each sample is a different population of cells. Thus, for example, the cells can be cancer cells or non-cancerous cells. In still further embodiments, each sample is the same population of cells (e.g., cancer cells, non-cancerous cells).

In some embodiments, the calculating comprises calculating one or more of a SAINTexpress algorithm score and a CompPASS algorithm score. In a further embodiment, the calculating comprises calculating the SAINTexpress algorithm score. In a still further embodiment, the calculating comprises calculating the CompPASS algorithm score. In yet further embodiments, the calculating comprises calculating the SAINTexpress algorithm score and the CompPASS algorithm score.

Methods of using SAINTexpress algorithms are known by those of skill in the art.

See, e.g., Teo, et al. (2014) J Proteomics 100: 37-43. As further described herein, a SAINTexpress algorithm can be used for PPI confidence scoring. In various aspects, PPI scoring can be performed separately for each cell line.

In some embodiments, the SAINTexpress algorithm score is calculated by a formula:

$$P ( {X}_{ij} \vert \text{*)} = {π}_{T} P ( {X}_{ij} \vert {λ}_{ij} ) + ( 1 - {π}_{T} ) P ( {X}_{ij} \vert {κ}_{ij} ) \qquad ( 1 )$$

wherein Xᵢⱼ is the spectral count for a prey protein i identified in a purification of bait j; wherein λᵢⱼ is the mean count from a Poisson distribution representing true interaction; wherein κᵢⱼ is the mean count from a Poisson distribution representing false interaction; wherein π_{T} is the proportion of true interactions in the data; and wherein dot notation represents all relevant model parameters estimated from the data for the pair of prey i and bait j.

Methods of using CompPASS algorithms are known by those of skill in the art. See, e.g., Huttlin, et al. (2015) Cell 162: 425-440; and Sowa, et al. (2009) Cell 138: 389-403. As further described herein, a CompPASS algorithm can be used for PPI confidence scoring. In various aspects, PPI scoring can be performed separately for each cell line.

In some embodiments, the CompPASS algorithm score is calculated by calculating the Z-score, the S-score, the D-score, and the WD-score, as further described herein.

In some embodiments, the DIS is calculated for a cancer cell line or a plurality of cancer cell lines and also calculated for a normal cell line. The DIS for the cancer cell line or the plurality of cancer cell lines is then compared to the DIS for the normal cell line. If the DIS for the cancer cell line or the plurality of cancer cell lines is greater than the DIS for the normal cell line, the DIS is assigned a positive (+) sign. If the DIS for the cancer cell line or the plurality of cancer cell lines is less than the DIS for the normal cell line, the DIS is assigned a negative (−) sign. Thus, a positive DIS represents a PPI that is enriched in a cancer cell line or a plurality of cancer cell lines, and a negative DIS represents a PPI that is depleted in a cancer cell line or a plurality of cancer cell lines.

In some embodiments, the DIS is calculated by a first formula:

$${DIS}_{A} ( b , p ) = {S}_{C 1} ( b , p ) \times {S}_{C 2} ( b , p ) \times [ 1 - {S}_{C 3} ( b , p ) ]$$

wherein DIS_{A}(b,p) is the DIS for each PPI (b, p) that is conserved in a first cell line and a second cell line, but not shared by a third cell line; wherein S_{C1}(b,p) is the probability of a PPI being present in the first cell line; wherein S_{C2}(b,p) is the probability of a PPI being present in the second cell line; and wherein S_{c3}(b,p) is the probability of a PPI being present in the third cell line; and a second formula:

$${DIS}_{B} ( b , p ) = [ 1 - {S}_{C 1} ( b , p ) ] \times [ 1 - {S}_{C 2} ( b , p ) ] \times {S}_{C 3} ( b , p$$

wherein DIS_{B}(b,p) is the DIS score for each PPI (b, p) that is conserved in the third cell line, but not shared by the first cell line and the second cell line; wherein a (+) sign is assigned if DIS_{A}(b,p)>DIS_{B}(b,p); and wherein a (−) sign is assigned if DIS_{A}(b,p)<DIS_{B}(b,p).

In some embodiments, the DIS is calculated by a first formula:

$${DIS}_{cancer} ( b , p ) = {S}_{C 1} ( b , p ) \times {S}_{C 2} ( b , p ) \times [ 1 - {S}_{N} ( b , p ) ]$$

wherein DIS_{cancer}(b,p) is the DIS for each PPI (b, p) that is conserved across a cancer cell line, but not shared by a normal cell line; wherein S_{C1}(b,p) is the probability of a PPI being present in a first cancer cell line; wherein S_{C2}(b,p) is the probability of a PPI being present in a second cancer cell line; and wherein S_{N}(b,p) is the probability of a PPI being present in a normal cell line; and a second formula:

$${DIS}_{normal} ( b , p ) = [ 1 - {S}_{C 1} ( b , p ) ] \times [ 1 - {S}_{C 2} ( b , p ) ] \times {S}_{N} ( b , p )$$

wherein DISₙₒᵣₘₐₗ(b,p) is the DIS score for each PPI (b, p) that is present in a normal cell line, but depleted in a cancer cell line; and assigning a (+) sign if DIS_{cancer}(b,p)>DISₙₒᵣₘₐₗ(b,p) and assigning a (−) sign if DIS_{cancer}(b,p)<DISₙₒᵣₘₐₗ(b,p).

In some embodiments, the DIS is an average of a SAINTexpress algorithm score and a CompPASS algorithm score. In some further embodiments, the DIS is a SAINTexpress algorithm score.

In some embodiments, the DIS ranges from about 0.0 to about 1.0. Thus, in various embodiments, the DIS ranges from about 0.0 to about 0.9, from about 0.0 to about 0.8, from about 0.0 to about 0.7, from about 0.0 to about 0.6, from about 0.0 to about 0.5, from about 0.0 to about 0.4, from 0.0 to 0.3, from 0.0 to 0.2, from 0.0 to 0.1, from 0.1 to 1.0, from 0.2 to 1.0, from 0.3 to 1.0, from 0.4 to 1.0, from 0.5 to 1.0, from 0.6 to 1.0, from 0.7 to 1.0, from 0.8 to 1.0, from about 0.9 to about 1.0, from about 0.1 to about 0.9, from about 0.2 to about 0.8, from about 0.3 to about 0.7, or from about 0.4 to about 0.6.

In some embodiments, a DIS of about 0.5 or greater than about 0.5 indicates that the dysfunctional protein-protein interaction is likely a causal agent of the hyperproliferative disorder. Thus, in various embodiments, a DIS of greater than about 0.5, greater than about 0.6, greater than about 0.7, greater than about 0.8, or greater than about 0.9 indicates that the dysfunctional protein-protein interaction is likely a causal agent of the hyperproliferative disorder.

In some embodiments, a DIS of 0.5 or less than 0.5 indicates that the dysfunctional protein-protein interaction is not likely a causal agent of the hyperproliferative disorder. Thus, in various embodiments, a DIS of less than 0.5, less than 0.4, less than 0.3, less than 0.2, or less than 0.1 indicates that the dysfunctional protein-protein interaction is likely a causal agent of the hyperproliferative disorder.

In some embodiments, the mass spectrometry analysis is performed on a plurality of samples, wherein calculating comprises calculating a SAINTexpress algorithm score for each sample, and averaging the SAINTexpress algorithm scores.

In some embodiments, the hyperproliferative disorder is a cancer. Examples of cancers include, but are not limited to, a sarcoma, a carcinoma, a hematological cancer, a solid tumor, breast cancer, cervical cancer, gastrointestinal cancer, colorectal cancer, brain cancer, skin cancer, head and neck cancer, prostate cancer, ovarian cancer, thyroid cancer, testicular cancer, pancreatic cancer, liver cancer, endometrial cancer, melanoma, a glioma, leukemia, lymphoma, chronic myeloproliferative disorder, myelodysplastic syndrome, myeloproliferative neoplasm, non-small cell lung carcinoma, and plasma cell neoplasm (myeloma). In further embodiments, the cancer is breast cancer of head and neck cancer. In still further embodiments, the cancer is breast cancer. In yet further embodiments, the cancer is head and neck cancer.

In some embodiments, the method further comprises harvesting samples with a functional bioassay. In a further embodiment, the functional bioassay is an animal model comprising growth of transformed cell lines.

In some embodiments, the subject is a mammal. In some embodiments, the mammal is a human.

In some embodiments, the subject has been diagnosed with a need for treatment of the hyperproliferative disorder prior to the administering step.

In some embodiments, the method further comprises identifying a subject in need of treatment of the hyperproliferative disorder. In a further embodiment, the subject is identified as being likely to respond to a cancer treatment if the DIS score is 0.5 or greater than 0.5. Thus, in various embodiments, the subject is identified as being likely to respond to a cancer treatment if the DIS score is greater than 0.5, greater than 0.6, greater than 0.7, greater than 0.8, or greater than 0.9.

In some embodiments, the subject is identified as being unlikely to respond to a cancer treatment if the DIS score is 0.5 or less than 0.5. Thus, in various embodiments, the subject is identified as being unlikely to respond to a cancer treatment if the DIS score is less than about 0.5, less than about 0.4, less than about 0.3, less than about 0.2, or less than about 0.1.

In some embodiments, the mutation candidate is one or more genes having a mutant protein sequence, wherein the gene is selected from TP53, CDKN2A, PIK3CA, TP63, FADD, SOX2, RHOA, CCND1, EGFR, CASP8, NFE2L2, MAPK1, MYC, PTEN, KEAP1, CUL3, E2F1, FBXW7, PTPRT, GFGR1, RB1, IGF1R, HRAS, TRAF3, TGFBR2, ERBB2, FGFR3, HLA-A, NRAS, STAT3, and XPC. In some embodiments, the mutation candidate is one or more genes having a mutant protein sequence, wherein the gene is selected from PIK3CA, TP53, MTDG, AKT3, CDH1, ERBB2, GATA3, TSPYL5, PTEN, RB1, BRIP1, CBFB, RAF51C, FOXA1, PALB2, ARID1A, ESR1, STK11, CDKN1B, MSH2, AKT1, AKT2, BRCA1, CHEK2, RPA2, EGFR, RAD51D, CASP8, CCND3, CTCF, MLH1, SMARCB1, XPC, SCUBE2, TBX3, XRN2, EZH2, FANCC, HRAS, or SMARCD1.

In some embodiments, the gene is TP53, PIK3CA, NFE212, MAPK1, FBXW7, or HRAS. In some embodiments, the gene is AKT1, AKT3, BRCA1, BRIP1, CDH1, CHEK2, HRAS, MTDH, PALB2, PIK3CA, or TP53.

In some embodiments, the gene is NFE2L2 and the mutant protein sequence is E79K or E79Q, wherein the gene is HRAS and the mutant protein sequence is G12D, wherein the gene is TP53 and the mutant protein sequence is R248W or R273H, wherein the gene is MAPK1 and the mutant protein sequence is E322K, or wherein the gene is FBXW7 and the mutant protein sequence is R505G.

In some embodiments, the gene is AKT1 and the mutant protein sequence is E17K, wherein the gene is AKT3 and the mutant protein sequence is E17K, wherein the gene is BRIP1 and the mutant protein sequence is A745T, wherein the gene is CDH1 and the mutant protein sequence is E243K, wherein the gene is CHEK2 and the mutant protein sequence is 1100deIC or K373E, wherein the gene is HRAS and the mutant protein sequence is G12D, wherein the gene is MTDH and the mutant protein sequence is A78S, wherein the gene is PALB2 and the mutant protein sequence is E837K, or wherein the gene is TP53 and the mutant protein sequence is R175H, R248W, or R273H.

In some embodiments, the gene is PIK3CA and the mutant protein sequence is R88Q, E110DeI, K111N, K111E, V344G, G363A, E453K, E542K, E545K, E545G, E726K, C971R, G1007R, M1043V, H1047L, or H1047R. In some embodiments, the gene is PIK3CA and the mutant protein sequence is E545K, M1043V, or H1047R. In some embodiments, the gene is BRCA1 and the mutant protein sequence is I16A, C61G, R71G, Δexon11, S1655F, 5832insC, or M1775R. The nucleic acid sequence of TP53 is found

| | | |
|---|---|---|
| TP53 | NC_000017.11 | NM_000546.5 |

The amino acid sequence of PT53 is:

(SEQ ID NO: 1)
MEEPQSDPSVEPPLSQETFSDLWKLLPENNVLSPLPSQAMDDLM
LSPDDIEQWFTEDPGPDEAPRMPEAAPPVAPAPAAPTPAAPAPA
PSWPLSSSVPSQKTYQGSYGFRLGFLHSGTAKSVTCTYSPALNK
MFCQLAKTCPVQLWVDSTPPPGTRVRAMAIYKQSQHMTEVVRRC
PHHERCSDSDGLAPPQHLIRVEGNLRVEYLDDRNTFRHSVVVPY
EPPEVGSDCTTIHYNYMCNSSCMGGMNRRPILTIITLEDSSGNL
LGRNSFEVRVCACPGRDRRTEEENLRKKGEPHHELPPGSTKRAL
PNNTSSSPQPKKKPLDGEYFTLQIRGRERFEMFRELNEALELKD
AQAGKEPGGSRAHSSHLKSKKGQSTSRHKKLMFKTEGPDSD
The amino acid sequence of PIK3CA is:
(SEQ ID NO: 2)
MQPFSIPVQITLQGSRRRQGRTAFPASGKKRETDYSDGDPLDVH
KRLPSSAGEDRAVMLGFAMMGFSVLMFFLLGTTILKPFMLSIQR
EESTCTAIHTDIMDDWLDCAFTCGVHCHGQGKYPCLQVFVNLSH
PGQKALLHYNEEAVQINPKRDVTDCRVKEKQTLTVSDEHKQ

In some embodiments, the amino acid sequence of PIK3CA (contiguous) is:

(SEQ ID NO: 3)
MPPRPSSGELWGIHLMPPRILVECLLPNGMIVTLECLREATLIT
IKHELFKEARKYPLHQLLQDESSYIFVSVTQEAEREEFFDETRR
LCDLRLFQPFLKVIEPVGNREEKILNREIGFAIGMPVCEFDMVK
DPEVQDFRRNILNVCKEAVDLRDLNSPHSRAMYVYPPNVESSPE
LPKHIYNKLDKGQIIVVIWVIVSPNNDKQKYTLKINHDCVPEQV
IAEAIRKKTRSMLLSSEQLKLCVLEYQGKYILKVCGCDEYFLEK
YPLSQYKYIRSCIMLGRMPNLMLMAKESLYSQLPMDCFTMPSYS
RRISTATPYMNGETSTKSLWVINSALRIKILCATYVNVNIRDID
KIYVRTGIYHGGEPLCDNVNTQRVPCSNPRWNEWLNYDIYIPDL
PRAARLCLSICSVKGRKGAKEEHCPLAWGNINLFDYTDTLVSGK
MALNLWPVPHGLEDLLNPIGVTGSNPNKETPCLELEFDWFSSVV
KFPDMSVIEEHANWSVSREAGFSYSHAGLSNRLARDNELRENDK
EQLKAISTRDPLSEITEQEKDFLWSHRHYCVTIPEILPKLLLSV
KWNSRDEVAQMYCLVKDWPPIKPEQAMELLDCNYPDPMVRGFAV
RCLEKYLTDDKLSQYLIQLVQVLKYEQYLDNLLVRFLLKKALTN
QRIGHFFFWHLKSEMHNKTVSQRFGLLLESYCRACGMYLKHLNR
QVEAMEKLINLTDILKQEKKDETQKVQMKFLVEQMRRPDFMDAL
QGFLSPLNPAHQLGNLRLEECRIMSSAKRPLWLNWENPDIMSEL
LFQNNEIIFKNGDDLRQDMLTLQIIRIMENIWQNQGLDLRMLPY
GCLSIGDCVGLIEVVRNSHTIMQIQCKGGLKGALQFNSHTLHQW
LKDKNKGEIYDAAIDLFTRSCAGYCVATFILGIGDRHNSNIMVK
DDGQLFHIDFGHFLDHKKKKFGYKRERVPFVLTQDFLIVISKGA
QECTKTREFERFQEMCYKAYLAIRQHANLFINLFSMMLGSGMPE
LQSFDDIAYIRKTLALDKTEQEALEYFMKQMNDAHHGGWTTKMD
WIFHTIKQHALN

The nucleic acid sequence of Akt11 is:

| | (SEQ ID NO: 4) |
|---|---|
| 1 | taattatggg tctgtaacca ccctggactg ggtgctcctc actgacggac ttgtctgaac |
| 61 | ctctctttgt ctccagcgcc cagcactggg cctggcaaaa cctgagacgc ccggtacatg |
| 121 | ttggccaaat gaatgaacca gattcagacc ggcaggggcg ctgtggttta ggaggggcct |
| 181 | ggggtttctc ccaggaggtt tttgggcttg cgctggaggg ctctggactc ccgtttgcgc |
| 241 | cagtggcctg catcctggtc ctgtcttcct catgtttgaa tttctttgct ttcctagtct |
| 301 | ggggagcagg gaggagccct gtgccctgtc ccaggatcca tgggtaggaa caccatggac |
| 361 | agggagagca aacggggcca tctgtcacca ggggcttagg gaaggccgag ccagcctggg |
| 421 | tcaaagaagt caaaggggct gcctggagga ggcagcctgt cagctggtgc atcagaggct |
| 481 | gtggccaggc cagctgggct cggggagcgc cagcctgaga ggagcgcgtg agcgtcgcgg |
| 541 | gagcctcggg caccatgagc gacgtggcta ttgtgaagga gggttggctg cacaaacgag |
| 601 | gggagtacat caagacctgg cggccacgct acttcctcct caagaatgat ggcaccttca |
| 661 | ttggctacaa ggagcggccg caggatgtgg accaacgtga ggctcccctc aacaacttct |
| 721 | ctgtggcgca gtgccagctg atgaagacgg agcggccccg gcccaacacc ttcatcatcc |
| 781 | gctgcctgca gtggaccact gtcatcgaac gcaccttcca tgtggagact cctgaggagc |
| 841 | gggaggagtg gacaaccgcc atccagactg tggctgacgg cctcaagaag caggaggagg |
| 901 | aggagatgga cttccggtcg ggctcaccca gtgacaactc aggggctgaa gagatggagg |
| 961 | tgtccctggc caagcccaag caccgcgtga ccatgaacga gtttgagtac ctgaagctgc |
| 1021 | tgggcaaggg cactttcggc aaggtgatcc tggtgaagga gaaggccaca ggccgctact |
| 1081 | acgccatgaa gatcctcaag aaggaagtca tcgtggccaa ggacgaggtg gcccacacac |
| 1141 | tcaccgagaa ccgcgtcctg cagaactcca ggcacccctt cctcacagcc ctgaagtact |
| 1201 | ctttccagac ccacgaccgc ctctgctttg tcatggagta cgccaacggg ggcgagctgt |
| 1261 | tcttccacct gtcccgggag cgtgtgttct ccgaggaccg ggcccgcttc tatggcgctg |
| 1321 | agattgtgtc agccctggac tacctgcact cggagaagaa cgtggtgtac cgggacctca |
| 1381 | agctggagaa cctcatgctg gacaaggacg ggcacattaa gatcacagac ttcgggctgt |
| 1441 | gcaaggaggg gatcaaggac ggtgccacca tgaagacctt ttgcggcaca cctgagtacc |
| 1501 | tggcccccga ggtgctggag gacaatgact acggccgtgc agtggactgg tgggggctgg |
| 1561 | gcgtggtcat gtacgagatg atgtgcggtc gcctgccctt ctacaaccag gaccatgaga |
| 1621 | agctttttga gctcatcctc atggaggaga tccgcttccc gcgcacgctt ggtcccgagg |
| 1681 | ccaagtcctt gctttcaggg ctgctcaaga aggaccccaa gcagaggctt ggcgggggct |
| 1741 | ccgaggacgc caaggagatc atgcagcatc gcttctttgc cggtatcgtg tggcagcacg |
| 1801 | tgtacgagaa gaagctcagc ccacccttca agccccaggt cacgtcggag actgacacca |
| 1861 | ggtattttga tgaggagttc acggcccaga tgatcaccat cacaccacct gaccaagatg |
| 1921 | acagcatgga gtgtgtggac agcgagcgca ggccccactt cccccagttc tcctactcgg |
| 1981 | ccagcggcac ggcctgaggc ggcggtggac tgcgctggac gatagcttgg agggatggag |
| 2041 | aggcggcctc gtgccatgat ctgtatttaa tggtttttat ttctcgggtg catttgagag |
| 2101 | aagccacgct gtcctctcga gcccagatgg aaagacgttt ttgtgctgtg ggcagcaccc |
| 2161 | tcccccgcag cggggtaggg aagaaaacta tcctgcgggt tttaatttat ttcatccagt |
| 2221 | ttgttctccg ggtgtggcct cagccctcag aacaatccga ttcacgtagg gaaatgttaa |
| 2281 | ggacttctgc agctatgcgc aatgtggcat tggggggccg ggcaggtcct gcccatgtgt |
| 2341 | cccctcactc tgtcagccag ccgccctggg ctgtctgtca ccagctatct gtcatctctc |
| 2401 | tggggccctg ggcctcagtt caacctggtg gcaccagatg caacctcact atggtatgct |
| 2461 | ggccagcacc ctctcctggg ggtggcaggc acacagcagc cccccagcac taaggccgtg |
| 2521 | tctctgagga cgtcatcgga ggctgggccc ctgggatggg accagggatg ggggatgggc |
| 2581 | cagggtttac ccagtgggac agaggagcaa ggtttaaatt tgttattgtg tattatgttg |
| 2641 | ttcaaatgca ttttgggggt ttttaatctt tgtgacagga aagccctccc ccttcccctt |
| 2701 | ctgtgtcaca gttcttggtg actgtcccac cgggagcctc cccctcagat gatctctcca |
| 2761 | cggtagcact tgaccttttc gacgcttaac ctttccgctg tcgccccagg ccctccctga |
| 2821 | ctccctgtgg gggtggccat ccctgggccc ctccacgcct cctggccaga cgctgccgct |
| 2881 | gccgctgcac cacggcgttt ttttacaaca ttcaacttta gtatttttac tattataata |
| 2941 | taatatggaa ccttccctcc aaattcttca ataaaagttg cttttcaaaa aaaaaaaaaa |
| 3001 | aaaaaaaa |

The HER3 amino acid sequence is

| | (SEQ ID NO: 5) |
|---|---|
| 1 | mrandalqvl gllfslargs evgnsqavcp gtlnglsvtg daenqyqtly klyercevvm |
| 61 | gnleivltgh nadlsflqwi revtgyvlva mnefstlplp nlrvvrgtqv ydgkfaifvm |
| 121 | lnyntnssha lrqlrltqlt eilsggvyie kndklchmdt idwrdivrdr daeivvkdng |
| 181 | rscppchevc kgrcwgpgse dcqtltktic apqcnghcfg pnpnqcchde caggcsgpqd |
| 241 | tdcfacrhfn dsgacvprcp qplvynkltf qlepnphtky qyggvcvasc phnfvvdqts |
| 301 | cvracppdkm evdknglkmc epcgglcpka cegtgsgsrf qtvdssnidg fvnctkilgn |
| 361 | ldflitglng dpwhkipald peklnvfrtv reitgylniq swpphmhnfs vfsnlttigg |
| 421 | rslynrgfsl limknlnvts lgfrslkeis agriyisanr qlcyhhslnw tkvlrgptee |
| 481 | rldikhnrpr rdcvaegkvc dplcssggcw gpgpgqclsc rnysrggvcv thcnflngep |
| 541 | refaheaecf schpecqpme gtatcngsgs dtcaqcahfr dgphcvsscp hgvlgakgpi |
| 601 | ykypdvqnec rpchenctqg ckgpelqdcl gqtlvligkt hltmaltvia glvvifmmlg |
| 661 | gtflywrgrr iqnkramrry lergesiepl dpsekankvl arifketelr klkvlgsgvf |
| 721 | gtvhkgvwip egesikipvc ikviedksgr qsfqavtdhm laigsldhah ivrllglcpg |
| 781 | sslqlvtqyl plgslldhvr qhrgalgpql llnwgvqiak gmyyleehgm vhrnlaarnv |
| 841 | llkspsqvqv adfgvadllp pddkqllyse aktpikwmal esihfgkyth qsdvwsygvt |
| 901 | vwelmtfgae pyaglrlaev pdllekgerl aqpqictidv ymvmvkcwmi denirptfke |
| 961 | laneftrmar dpprylvikr esgpgiapgp ephgltnkkl eevelepeld ldldleaeed |
| 1021 | nlatttlgsa lslpvgtlnr prgsqsllsp ssgympmnqg nlgescqesa vsgssercpr |
| 1081 | pvslhpmprg clasessegh vtgseaelqe kvsmcrsrsr srsprprgds ayhsqrhsll |
| 1141 | tpvtplsppg leeedvngyv mpdthlkgtp ssregtlssv glssvlgtee ededeeyeym |
| 1201 | nrrrrhspph pprpssleel gyeymdvgsd lsaslgstqs cplhpvpimp tagttpdedy |
| 1261 | eymnrqrdgg gpggdyaamg acpaseqgye emrafqgpgh qaphvhyarl ktlrsleatd |
| 1321 | safdnpdywh srlfpkanaq rt |
| The nucleotide sequence of HER3 is: | |
| (SEQ ID NO: 6) | |
| | ctccga ggtgggcaac tctcaggcag tgtgtcctgg gactctgaat |
| | ggcctgagtg tgaccggcga tgctgagaac caataccaga cactgtacaa gctctacgag |
| | aggtgtgagg tggtgatggg gaaccttgag attgtgctca cgggacacaa tgccgacctc |
| | tccttcctgc agtggattcg agaagtgaca ggctatgtcc tcgtggccat gaatgaattc |
| | tctactctac cattgcccaa cctccgcgtg gtgcgaggga cccaggtcta cgatgggaag |
| | tttgccatct tcgtcatgtt gaactataac accaactcca gccacgctct gcgccagctc |
| | cgcttgactc agctcaccga gattctgtca gggggtgttt atattgagaa gaacgataag |
| | ctttgtcaca tggacacaat tgactggagg gacatcgtga gggaccgaga tgctgagata |
| | gtggtgaagg acaatggcag aagctgtccc ccctgtcatg aggtttgcaa ggggcgatgc |
| 781 | tggggtcctg gatcagaaga ctgccagaca ttgaccaaga ccatctgtgc tcctcagtgt |
| 841 | aatggtcact gctttgggcc caaccccaac cagtgctgcc atgatgagtg tgccgggggc |
| 901 | tgctcaggcc ctcaggacac agactgcttt gcctgccggc acttcaatga cagtggagcc |
| 961 | tgtgtacctc gctgtccaca gcctcttgtc tacaacaagc taactttcca gctggaaccc |
| 1021 | aatccccaca ccaagtatca gtatggagga gtttgtgtag ccagctgtcc ccataacttt |
| 1081 | gtggtggatc aaacatcctg tgtcagggcc tgtcctcctg acaagatgga agtagataaa |
| 1141 | aatgggctca agatgtgtga gccttgtggg ggactatgtc ccaaagcctg tgagggaaca |
| 1201 | ggctctggga gccgcttcca gactgtggac tcgagcaaca ttgatggatt tgtgaactgc |
| 1261 | accaagatcc tgggcaacct ggactttctg atcaccggcc tcaatggaga cccctggcac |
| 1321 | aagatccctg ccctggaccc agagaagctc aatgtcttcc ggacagtacg ggagatcaca |
| 1381 | ggttacctga acatccagtc ctggccgccc cacatgcaca acttcagtgt tttttccaat |
| 1441 | ttgacaacca ttggaggcag aagcctctac aaccggggct tctcattgtt gatcatgaag |
| 1501 | aacttgaatg tcacatctct gggcttccga tccctgaagg aaattagtgc tgggcgtatc |
| 1561 | tatataagtg ccaataggca gctctgctac caccactctt tgaactggac caaggtgctt |
| 1621 | cgggggccta cggaagagcg actagacatc aagcataatc ggccgcgcag agactgcgtg |
| 1681 | gcagagggca aagtgtgtga cccactgtgc tcctctgggg gatgctgggg cccaggccct |
| 1741 | ggtcagtgct tgtcctgtcg aaattatagc cgaggaggtg tctgtgtgac ccactgcaac |
| 1801 | tttctgaatg gggagcctcg agaatttgcc catgaggccg aatgcttctc ctgccacccg |
| 1861 | gaatgccaac ccatgggggg cactgccaca tgcaatggct cgggctctga tacttgtgct |
| 1921 | caatgtgccc attttcgaga tgggccccac tgtgtgagca gctgccccca tggagtccta |
| 1981 | ggtgccaagg gcccaatcta caagtaccca gatgttcaga atgaatgtcg gccctgccat |
| 2041 | gagaactgca cccaggggtg taaaggacca gagcttcaag actgtttagg acaaacactg |
| 2101 | gtgctgatcg gcaaaaccca tctgacaatg gctttgacag tgatagcagg attggtagtg |
| 2161 | attttcatga tgctgggcgg cacttttctc tactggcgtg ggcgccggat tcagaataaa |
| 2221 | agggctatga ggcgatactt ggaacggggt gagagcatag agcctctgga ccccagtgag |
| 2281 | aaggctaaca aagtcttggc cagaatcttc aaagagacag agctaaggaa gcttaaagtg |
| 2341 | cttggctcgg gtgtctttgg aactgtgcac aaaggagtgt ggatccctga gggtgaatca |
| 2401 | atcaagattc cagtctgcat taaagtcatt gaggacaaga gtggacggca gagttttcaa |
| 2461 | gctgtgacag atcatatgct ggccattggc agcctggacc atgcccacat tgtaaggctg |
| 2521 | ctgggactat gcccagggtc atctctgcag cttgtcactc aatatttgcc tctgggttct |
| 2581 | ctgctggatc atgtgagaca acaccggggg gcactggggc cacagctgct gctcaactgg |
| 2641 | ggagtacaaa ttgccaaggg aatgtactac cttgaggaac atggtatggt gcatagaaac |
| 2701 | ctggctgccc gaaacgtgct actcaagtca cccagtcagg ttcaggtggc agattttggt |
| 2761 | gtggctgacc tgctgcctcc tgatgataag cagctgctat acagtgaggc caagactcca |
| 2821 | attaagtgga tggcccttga gagtatccac tttgggaaat acacacacca gagtgatgtc |
| 2881 | tggagctatg gtgtgacagt ttgggagttg atgaccttcg gggcagagcc ctatgcaggg |
| 2941 | ctacgattgg ctgaagtacc agacctgcta gagaaggggg agcggttggc acagccccag |
| 3001 | atctgcacaa ttgatgtcta catggtgatg gtcaagtgtt ggatgattga tgagaacatt |
| 3061 | cgcccaacct ttaaagaact agccaatgag ttcaccagga tggcccgaga cccaccacgg |
| 3121 | tatctggtca taaagagaga gagtgggcct ggaatagccc ctgggccaga gccccatggt |
| 3181 | ctgacaaaca agaagctaga ggaagtagag ctggagccag aactagacct agacctagac |
| 3241 | ttggaagcag aggaggacaa cctggcaacc accacactgg gctccgccct cagcctacca |
| 3301 | gttggaacac ttaatcggcc acgtgggagc cagagccttt taagtccatc atctggatac |
| 3361 | atgcccatga accagggtaa tcttgggggg tcttgccagg agtctgcagt ttctgggagc |
| 3421 | agtgaacggt gcccccgtcc agtctctcta cacccaatgc cacggggatg cctggcatca |
| 3481 | gagtcatcag aggggcatgt aacaggctct gaggctgagc tccaggagaa agtgtcaatg |
| 3541 | tgtagaagcc ggagcaggag ccggagccca cggccacgcg gagatagcgc ctaccattcc |
| 3601 | cagcgccaca gtctgctgac tcctgttacc ccactctccc cacccgggtt agaggaagag |
| 3661 | gatgtcaacg gttatgtcat gccagataca cacctcaaag gtactccctc ctcccgggaa |
| 3721 | ggcacccttt cttcagtggg tctcagttct gtcctgggta ctgaagaaga agatgaagat |
| 3781 | gaggagtatg aatacatgaa ccggaggaga aggcacagtc cacctcatcc ccctaggcca |
| 3841 | agttcccttg aggagctggg ttatgagtac atggatgtgg ggtcagacct cagtgcctct |
| 3901 | ctgggcagca cacagagttg cccactccac cctgtaccca tcatgcccac tgcaggcaca |
| 3961 | actccagatg aagactatga atatatgaat cggcaacgag atggaggtgg tcctgggggt |
| 4021 | gattatgcag ccatgggggc ctgcccagca tctgagcaag ggtatgaaga gatgagagct |
| 4081 | tttcaggggc ctggacatca ggccccccat gtccattatg cccgcctaaa aactctacgt |
| 4141 | agcttagagg ctacagactc tgcctttgat aaccctgatt actggcatag caggcttttc |
| 4201 | cccaaggcta atgcccagag aacgtaactc ctgctccctg tggcactcag ggagcattta |
| 4261 | atggcagcta gtgcctttag agggtaccgt cttctcccta ttccctctct ctcccaggtc |
| 4321 | ccagcccctt ttccccagtc ccagacaatt ccattcaatc tttggaggct tttaaacatt |
| 4381 | ttgacacaaa attcttatgg tatgtagcca gctgtgcact ttcttctctt tcccaacccc |
| | aggaaaggtt ttccttattt tgtgtgcttt cccagtccca ttcctcagct tcttcacagg |
| | cactcctgga gatatgaagg attactctcc atatcccttc ctctcaggct cttgactact |
| | tggaactagg ctcttatgtg tgcctttgtt tcccatcaga ctgtcaagaa gaggaaaggg |
| | aggaaaccta gcagaggaaa gtgtaatttt ggtttatgac tcttaacccc ctagaaagac |
| | agaagcttaa aatctgtgaa gaaagaggtt aggagtagat attgattact atcataattc |
| | agcacttaac tatgagccag gcatcatact aaacttcacc tacattatct cacttagtcc |
| | tttatcatcc ttaaaacaat tctgtgacat acatattatc tcattttaca caaagggaag |
| | tcgggcatgg tggctcatgc ctgtaatctc agcactttgg gaggctgagg cagaaggatt |
| | acctgaggca aggagtttga gaccagctta gccaacatag taagaccccc atctc |

In some embodiments, the dysfunctional protein-protein interaction is one or more of a D1:PI3K interaction or a FGFR3: Daple interaction. In some embodiments, the dysfunctional protein-protein interaction is one or more of a BPIFA1: PIK3CA interaction, a S100A3: Akt interaction, a SCGB2A1: PIK3CA interaction, or a Spinophilin: BRCA1 interaction.

In some embodiments, the causal agent is HER3 or a dysfunction in HER3 due to a mutation. In some embodiments, the causal agent is Akt or a dysfunction of Akt due to a mutation

In some embodiments, the method further comprises selecting a hyperproliferative disorder treatment for the subject based upon the causal agent. In some embodiments, the step of selecting a hyperproliferative disorder treatment comprises selecting a treatment from a database of known treatments for the dysfunctional protein-protein interaction.

In some embodiments, the hyperproliferative disorder treatment comprises administration of a HER3 inhibitor. Examplers of HER3 inhibitors include, but are not limited to, lapatinib, erlotinib, gefitinib, afatinib, neratinib, CDX-3379, U-31402, HMBD-001, MCLA-128, KBP-5209, Poziotinib, Varlitinib, FCN-411, Elgemtumab, Sirotinib, vaccines to target Her3 for solid tumors, AV2103, AV2103, ETBX-031, MP-EV-20, MP-EV-20/1959, and oligonucleotides to inhibit EGFR, ERBB2, and ERBB3. Additional exemplary HER3 inhibitors are described in US 2018/0362443 A1, U.S. Pat. No. 10,383,878 B2, US 2019/0300624 A1, WO 2018/182420 A1, WO 2015/007219 A1, U.S. Pat. No. 8,735,551 B2, U.S. Pat. No. 10,507,209 B2, U.S. Pat. No. 9,956,222 B2, U.S. Pat. No. 10,487,143 B2, WO 2018/233511 A1, CN106692969A, US 2020/0147193 A1, U.S. Pat. No. 9,346,889 B2, WO 2020/099235 A1, US 2019/0201552 A1, US 2018/0105815 A1, and US 2020/0157542 A1. In some embodiments, the HER3 inhibitor is CDX3379.

In some embodiments, the hyperproliferative disorder is head and neck cancer, wherein the mutation candidate is a mutant PIK3CA, wherein the causal agent is HER3, and wherein the hyperproliferative disorder treatment comprises administration of a HER3 inhibitor.

In some embodiments, the hyperproliferative disorder treatment comprises administration of an Akt inhibitor. Examples of Akt inhibitors include, but are not limited to, MK-2206, AZD5363, GSK690693, GDC-0068, GSK2141795, GSK2110183, AT7867, CCT128930, BAY1125976, perifosine, and AKT inhibitor III.

In some embodiments, the Akt modulator is a PIK3CA modulator. Examples of PIK3CA modulators include, but are not limited to, Alpelisib, Copanlisib hydrochloride, GDC-0077, Bimiralisib, Fimepinostat, Serabelisib, HHCYH-33, omipalisib, and PQR-514.

In some embodiments, the hyperproliferative disorder is breast cancer, wherein the mutation candidate is a mutant PIK3CA or a mutant BRCA1, wherein the causal agent is Akt, and wherein the hyperproliferative disorder treatment comprises administration of an Akt inhibitor.

### Systems

The above-described methods can be implemented in any of numerous ways. For example, the embodiments may be implemented using a computer program product (i.e., software), hardware, software, or a combination thereof. When implemented in software, the software code can be executed on any suitable processor or collection of processors, whether provided in a single computer or distributed among multiple computers.

Thus, in some embodiments, the disclosure relates to computer program products encoded on a computer-readable storage medium, wherein the computer program product comprises instructions for: (a) performing a mass spectrometry analysis on a sample from a subject that has a mutation candidate that causes a hyperproliferative disorder; (b) identifying dysfunctional protein-protein interactions associated with the hyperproliferative disorder; and (c) calculating a differential interaction score (DIS). In some embodiments, the computer program product further comprises instructions for correlating the DIS with the likelihood that the dysfunctional protein-protein interaction is a causal agent of the hyperproliferative disorder. In some embodiments, the computer program product further comprises instructions for: (d) comparing the DIS score to a first threshold; and (e) classifying the subject as being likely to respond to a hyperproliferative disorder treatment, wherein each of steps (d) and (e) are performed after step (c), and wherein the first threshold is calculated relative to a first control dataset.

In some embodiments, the disclosure relates to systems comprising a disclosed computer program product, and one or more of: (a) a processor operable to execute programs; and (b) a memory associated with the processor.

In some embodiments, the disclosure relates to systems for identifying a protein interaction network in a subject, the system comprising: (a) a processor operable to execute programs; (b) a memory associated with the processor; (c) a database associated with said processor and said memory; and (d) a program stored in the memory and executable by the processor, the program being operable for: (i) performing a mass spectrometry analysis on a sample from a subject that has a mutation candidate that causes a hyperproliferative disorder; (ii) identifying dysfunctional protein-protein interactions associated with the hyperproliferative disorder; and (iii) calculating a differential interaction score (DIS).

Without wishing to be bound by theory, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output.

Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks, or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. The disclosure also relates to a computer readable storage medium comprising executable instructions. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Computer-implemented embodiments of the disclosure relate to methods of determining a subject likely to respond to cancer disease-modifying agents comprising steps of: (e) comparing the first normalized score to a first threshold relative to a first control dataset of a sample and comparing a second normalized score to a second threshold relative to a control dataset of the sample; and (f) classifying the subject as being likely to respond to a chemotherapeutic treatment based upon results of comparing of step (e) relative to the first and/or second threshold; wherein each of steps (e) and (f) are performed after step (d).

In some embodiments, the disclosure relates to a system that comprises at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a Local Area Network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method steps and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification have been labeled as circuits, in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or the like.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing. In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Functions, operations, components and/or features described herein with reference to one or more embodiments, may be combined with, or may be utilized in combination with, one or more other functions, operations, components and/or features described herein with reference to one or more other embodiments, or vice versa.

The disclosure relates to a computer program product comprising instructions to calculate DIS in connection with a structure of an oncoprotein. In some embodiments, the computer program product comprises instructions for any of the steps identified in the disclosure. In some embodiments, the disclosure relates to a method of imaging a structure of a protein associated with a hyperproliferative disorder, the method comprising: (a) identifying a nucleic acid sequence or protein sequence associated with a hyperproliferative disorder (b) calculating a DIS score associated with the nucleic acid sequence or protein sequence; and (c) creating an image of the structure of the protein based upon the DIS using a system disclosed herein, the image being displayed on a display operably connected to a controller comprising a computer program product disclosed herein.

In some embodiments, the disclosure relates to methods of imaging a protein, the method comprising: (a) identifying a first protein that co-localizes with a first host protein in one or a plurality of bioassays; (b) calculating a differential interaction score (DIS) corresponding to the first protein in a sample; and (c) predicting the three-dimensional structure of the first protein by integrating the DIS score into a fit. In some embodiments, the first protein is isolated in vitro from a sample. In some embodiments, the sample is from a cell extract or subject. In some embodiments, the first protein is mutated as compared to a wild-type or endogenous, unmutated sequence. In some embodiments, the method is a computer-implemented method performed on a system disclosed herein, comprising instructions for execution of the DIS calculation.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

All referenced journal articles, patents, and other publications are incorporated by reference herein in their entireties.

### EXAMPLES

Representative examples of the disclosed methods and systems are illustrated in the following non-limiting methods and examples.

### Example 1. The Protein Interaction Landscape of Breast Cancer

### Experimental Model and Subject Details

### Cloning and Cell Line Generation

Complementary DNAs (cDNA) of each bait were obtained from human ORFeome collection (v8.1) or Addgene [pcDNA6-ARID1A (#39311), pcDNA3-Casp8 (#11817), hEcadherin-pcDNA3 (#45769), pDONR223_EGFR_WT (#81926), pDONR221-Spinophilin (#87123)]. In case that cDNAs of canonical isoforms were not available, they were synthesized using gBlock fragments (IDT, Genewiz). These cDNAs were cloned using the Gateway Cloning System (Life Technologies) into a doxycycline-inducible N-term or C-term 3×FLAG-tagged vector modified to be Gateway compatible from the pLVX-Puro vector (Clontech). Point mutant baits were generated via site-directed mutagenesis. All expression vectors were full-sequence verified.

### Cell Culture, Lentivirus Production, and Stable Cell Line Generation

A MDA-MB-231 (ATTC, HTB-26) were maintained in DMEM and Ham's F-12 50/50 (Corning) supplemented with 10% fetal bovine serum (Gibco) and 1% Penicillin/Streptomycin (Corning). MCF10A (ATCC CRL-10317) cells were maintained in DMEM and Ham's F-12 50/50 supplemented with 20% horse serum (Gibco), EGF (PeproTech), Hydrocortisone (Sigma-Aldrich), Cholera toxin (Sigma-Aldrich), Insulin (Sigma-Aldrich) and 1% Penicillin/Streptomycin. HEK293T (ATCC, CRL-3216), MCF7 (ATCC, HTB-22) and U2OS-GFP reporter cell lines (gifts from Dr. Stark at City of Hope National Medical Center) were maintained in DMEM supplemented with 10% fetal bovine serum (Gibco) and 1% Penicillin-Streptomycin. All cells were cultured at 37° C. in a humidified atmosphere with 5% CO₂.

One day prior to transfection, 5.0 million HEK293T cells were plated in a 15 cm dish. Lentivirus was produced for each protein by using 5 μg of expression vector, 3.33 μg of Gag-Pol-Tat-Rev packaging vector (pJH045 from Judd Hultquist) and VSV-G (pJH046 from Judd Hultquist) mixed with 30 μL of PolyJet DNA Transfection Reagent (SignaGen) in serum free DMEM. DNA complexes were incubated at RT for 25 min and added dropwise to HEK293T cells. After 72 hrs, the lentivirus containing supernatant from infected HEK293T cells was centrifuged at 400×g for 5 min to pellet any debris. The supernatant was filtered through a 0.45 μm PVDF filter. Virions were let to aggregate and precipitate in PEG-6000 (8.5% final) and NaCl (0.3 M final) at 4° C. for 4-8 h. Virions were pelleted by spinning at 3500 rpm for 20 min at 4° C. The pellet was then resuspended in DPBS for a final volume between 800 to 1000 μL and stored at −80° C. until use.

Stable cell lines were generated by transducing a 10 cm plate at 80% confluency with 200 μL of precipitated lentivirus for 24 hrs. Transduced cells were selected with 2.5 μg/mL of puromycin.

### Cell Lysis and Affinity Purification

Three independent biological replicates of cells were plated in 10 cm dishes. For doxycycline-inducible gene expression, cells were induced at 40-50% confluence with 1 μg/mL doxycycline for 40 hrs. To prepare cell extracts, a 10 cm dish was washed with 1 mL of ice-cold PBS and lysed in 300 μL of S150 lysis buffer (50 mM Tris, pH 7.5, 150 mM NaCl, 1 mM EDTA, 0.5% NP-40, 1 mM DTT, 1× Protease and Phosphatase Inhibitor Cocktail and 125 U Benzonase/mL) using freeze thaw method—5 min on dry ice, followed by 30-45 see thaw in 37° C. water bath with agitation. Cell lysates were clarified by spinning at 13,000×g for 15 min at 4° C. A 20 μL aliquot was saved for western blot.

For FLAG purification, 25 μL of bead slurry was washed twice with 1 mL of S150 buffer. Supernatants were incubated with Anti-FLAG M2 magnetic beads (M8823, Sigma-Aldrich) or Anti-V5 magnetic beads (M167-11, MBL International) overnight at 4° C. with rotation. The beads were washed one time with 1 mL of S150 buffer containing 0.1% NP40 followed by two washes in detergent free S150 buffer.

To perform on bead digestion, magnetic beads were resuspended in 15 μL of freshly prepared 8 M urea with 50 mM Tris, pH 9.0, 1 mM DTT and 1 μg LysC and incubated for 1 hr at 37° C. Supernatant was incubated with 3 mM iodoacetamide (IAA) in the dark at room temperature (RT) for 45 min. Quenching IAA with 3 mM DTT for 15 min at RT was followed by another incubation for 1 hr at RT with shaking. Samples were diluted 4-fold by 50 mM

Tris, pH 8.0 to bring final concentration of urea to 2 M and digested with 1 μg trypsin at 37° C. overnight. Samples were acidified with 10% trifluoroacetic acid (TFA) to final 0.5% (pH<2) and desalted using Nest Tips C18. Tips were conditioned with 80% acetonitrile, 0.1% TFA and sequentially equilibrated three times with 0.1% TFA before applying samples. Bound peptides were sequentially rinsed three times with 0.1% TFA and eluted with 50% acetonitrile and 0.25% formic acid (FA). Eluted peptides were dried under vacuum centrifugation and resuspended in 3% ACN and 0.1% FA prior to mass spectrometry.

### Global Endogenous Protein Abundance Analysis

Following cell lysis, protein concentration was determined using Bradford assay. IAA was added to each sample to a final concentration of 10 mM, and samples were incubated in the dark at room temperature for 30 min.

Excess IAA was quenched by the addition of dithiothreitol to 10 mM, followed by incubation in the dark at room temperature for 30 min. Samples were then diluted with 0.1 M ammonium bicarbonate, pH 8.0 to a final urea concentration of 2 M. Trypsin (Promega) was added at a 1:100 (enzyme: protein) ratio and digested overnight at 37° C. with rotation. Following digestion, 10% TFA was added to each sample to a final pH˜2. Samples were desalted under vacuum using Sep Pak C18 cartridges (Waters). Each cartridge was activated with 1 mL 80% acetonitrile (ACN)/0.1% TFA, then equilibrated three times with 1 mL of 0.1% TFA. Following sample loading, cartridges were washed four times with 1 mL of 0.1% TFA, and samples were eluted four times with 0.5 mL 50% ACN/0.25% FA. 20 μg of each sample was kept for protein abundance measurements, and the remainder was used for phosphopeptide enrichment. Samples were dried by vacuum centrifugation.

### Mass Spectrometry Data Acquisition and Analysis

For AP-MS experiments, samples were resuspended in 15 μL of MS loading buffer (4% formic acid, 2% acetonitrile) and 2 μL were separated by a reversed-phase gradient over a nanoflow 75 μm ID×25 cm long picotip column packed with 1.9 μM C18 particles (Dr. Maisch). Peptides were directly injected over the course of a 75 min acquisition into a Q-Exactive Plus mass spectrometer (Thermo), or over the course of a 90 min acquisition into a Orbitrap Elite mass spectrometer. For analysis of endogenous protein abundances in parental cell lines, ˜500 ng of peptides was separated over a 180 min gradient using the same column as for AP-MS experiments, and directly injected into a Q-Exactive Plus mass spectrometer. Raw MS data were searched against the uniprot canonical isoforms of the human proteome (downloaded Mar. 21, 2018), and using the default settings in MaxQuant (version 1.6.2.10), with a match-between-runs enabled (Cox and Mann, 2008). Peptides and proteins were filtered to 1% false discovery rate in MaxQuant, and identified proteins were then subjected to protein-protein interaction scoring. To quantify changes in interactions between WT and mutant baits, or differences in endogenous protein abundances between parental cell lines, a label free quantification approach was used, in which statistical analysis was performed using MSstats (Choi et al., 2014) from within the artMS R-package. All raw data files and search results are available from the Pride partner ProteomeXchange repository under the PXD019639 identifier (Vizcaino et al. 2014; Perez-Riverol et al. 2019).

### Protein-Protein Interaction Scoring

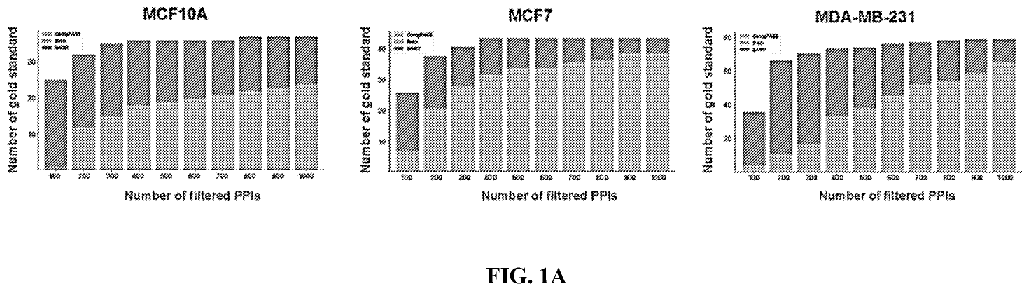

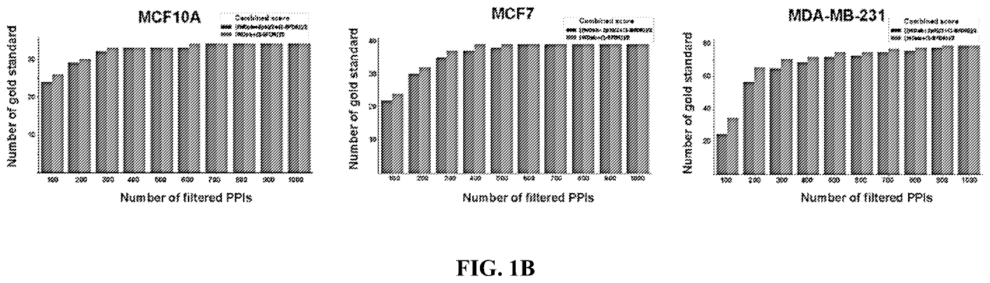

Protein spectral counts as determined by MaxQuant search results were used for PPI confidence scoring by both SAINTexpress (version 3.6.1) (Teo et al., 2014b) and CompPASS (version 0.0.0.9000) (Huttlin et al., 2015b; Sowa et al., 2009b). All PPI scoring was performed separately for each cell line. For SAINTexpress, control samples in which bait protein was not induced by doxycycline were used. For CompPASS, a stats table representing all no dox-induced samples (at least one per each bait) and WT baits was used. When recovery rates of known PPIs (gold standard) from public databases (CORUM, BioPlex2, and BioGRID low throughput and multivalidated) were monitored by varying thresholds of key metrics of each algorithm (WD per bait percentile for compPASS and BFDR for SAINTexpress, respectively), it is noticeable that CompPASS and SAINTexpress are complementary to each other, in that the best gold standard PPI recovery could be obtained when the PPIs from each algorithm are combined (FIG. 1A and FIG. 1B). Therefore, a PPI score was defined on a zero to 1 scale, wherein WD per bait percentile and (1-BFDR) were equally weighted: PPI score=[WD per bait percentile+(1−BFDR)]/2. The PPIs were filtered with PPI score≥0.9.

Referring to FIG. 1A, a comparison of PPIs filtered by compPASS and SAINTexpress, respectively, is shown. For each cell line, two sorted lists of PPIs were created based on the WD per bait percentile derived from compPASS score and SAINTexpress BFDR. The numbers of gold standard PPIs among top x PPIs (x=100, 200, . . . , 1,000) in each sorted list were compared. Top shaded portion indicates the gold standard PPIs recovered by SAINTexpress, bottom shaded portion indicates the ones recovered by compPASS, and middle shaded portion indicates the gold standard PPI recovered by both. Combination of compPASS and SAINTexpress best accommodate gold standard PPIs.

Referring to FIG. 1B, a comparison of PPI score calculated using compPASS and SAINTexpress scores is shown. Left bars indicate the number of gold standard PPIs recovered using a PPI score calculated using WD per bait percentile (WDpb) and Z per bait percentile (Zpb) from compPASS, and BFDR from SAINTexpress. Right bars indicate the recovery of gold standard PPIs based on a PPI score that only considers WDpb and BFDR. The comparison reveals that WDpb and BFDR are the most deterministic factors in recovering gold standard PPIs from each cell line.

### Correspondence Between Interaction Uniqueness and Expression Abundance Analysis

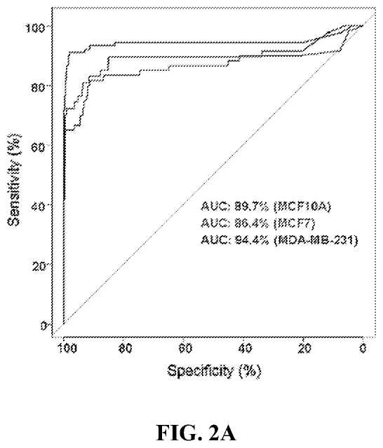

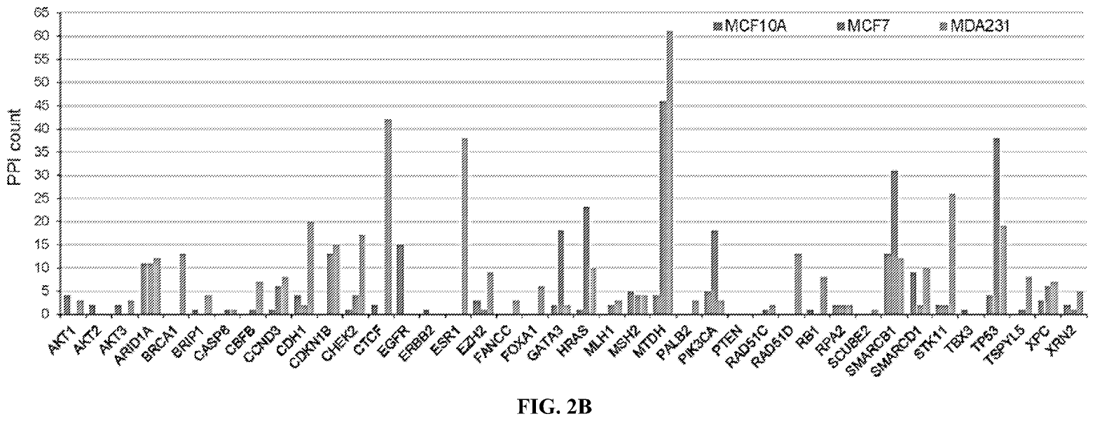

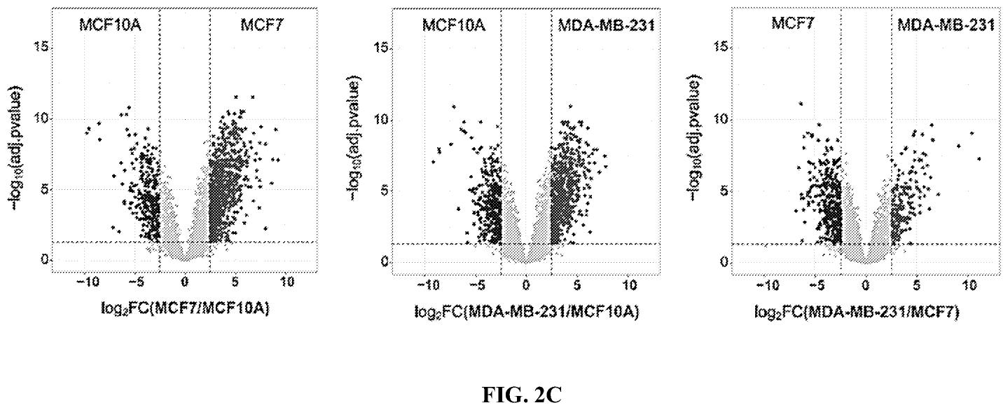

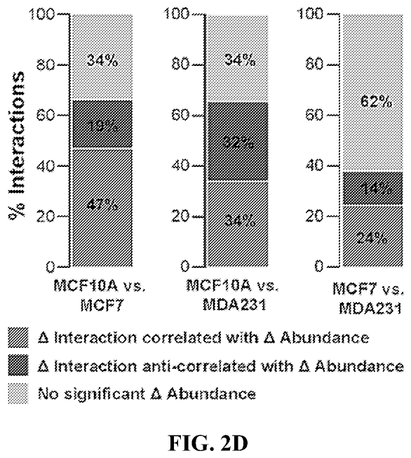

For each cell line comparison, shared baits were identified. For each bait, unique preys were extracted and their corresponding global abundance log 2FC was annotated. Only preys with a detected measurement in the global abundance analysis were included. Next, the fraction of preys (unique in one cell line or another in binding to a certain bait) with a correlated (gain in interaction=increase in abundance, and vice versa) or anticorrelated (gain in interaction=decrease in abundance, and vice versa) significant change [abs(log 2FC)>1 & adjusted p-value<0.05] in global abundance was calculated (see FIG. 2D).

Referring to FIG. 2D, the percentage of unique interactions (preys) for the same bait between cell lines with a correlated (bottom) or anticorrelated (middle) significant change (≥2-fold change, adjusted p-value<0.05) in abundance. Top indicates unique interactions with no significant change in abundance. Only preys detected by global abundance mass spectrometry analysis are considered.

### Differential Interaction Score Calculation

An important goal of cancer therapy is to identify drug targets that are cancer specific, and are applicable across many patients. As such, comparing PPIs across cell lines to prioritize those that were shared between cancer cell lines, but absent from the MCF10A non-tumorigenic cell line, was of interest. Unfortunately, a simple overlap analysis of BC PPIs identified within each cell line does not faithfully represent whether a given PPI is shared or unique in all cases. The reason for this is that to establish a finite list of BC PPIs, one must establish a threshold for such classification. This threshold strikes a balance between maximizing sensitivity for true interactions, while minimizing the inclusion of erroneous false positive interaction partners, which are often due to non-specific binding to the beads. However, it can also be the case that real PPIs do not meet this threshold (false negatives).

To compare PPIs across cell lines, a method for calculating a differential interaction score (DIS) and a corresponding false discovery rate (FDR) was developed using AP-MS data across multiple cell lines. This approach uses the SAINTexpress score (Teo et al., 2014b), which is the probability of a PPI being bonafide in a single cell line computed using a mixture of distribution modeling spectral counts of true and false interactions. The probabilities based on the analysis of a single cell line can then be used to calculate a differential interaction score between PPIs present in cancer cells and normal cells. A cancer-specific differential interaction score was defined as the probability of the PPI being present in a cancer cell line but absent in the normal cell line. Let Sc(p1, p2) be the SAINTexpress score of a PPI denoted as (p1, p2) in a cell line c. Given that PPIs are independent events across different cell lines, the differential interaction score is computed for each (p1, p2) as the product of the probability of a bonafide PPI in one cell line and the probability of the PPI being false in the other cell lines, which can be denoted as follows:

$${DIS}_{MCF 7} ( {p}_{1} , {p}_{2} ) = {S}_{MCF 7} ( {p}_{1} , {p}_{2} ) \times ( 1 - {S}_{MDA - MB - 231} ( {p}_{1} , {p}_{2} ) ) \times ( 1 - {S}_{MCF 10 A} ( {p}_{1} , {p}_{2} ) ) and {DIS}_{MDA - MB - 231} ( {p}_{1} , {p}_{2} ) = {S}_{MDA - MB - 231} ( {p}_{1} , {p}_{2} ) \times ( 1 - {S}_{MCF 7} ( {p}_{1} , {p}_{2} ) ) \times ( 1 - {S}_{MCF 10 A} ( {p}_{1} , {p}_{2} ) ) and {DIS}_{MCF 10 A} ( {p}_{1} , {p}_{2} ) = {S}_{MCF 10 A} ( {p}_{1} , {p}_{2} ) \times ( 1 - {S}_{MDA - MB - 231} ( {p}_{1} , {p}_{2} ) ) \times ( 1 - {S}_{MCF 7} ( {p}_{1} , {p}_{2} ) )$$

For all differential interaction scores that were calculated, the Bayesian false discovery rate (BFDR) estimates at all possible thresholds (p*) were computed as follows:

$$FDR ( {p}^{*} ) = \frac{{\sum }_{i , i} ( 1 - DIS ( {p}_{i} , {p}_{j} ) ) \times I ( DIS ( {p}_{i} , {p}_{j} ) > {p}^{*} )}{{\sum }_{i , j} I ( DIS ( {p}_{i} , {p}_{j} ) > {p}^{*} )} , \\ where I \{ A \} is 1 when A is True and 0 otherwise .$$

### Permutation Test

A permutation test was performed in which genes were drawn from the list of all genes detected in the global protein abundance analysis of the parental cell lines. The null distribution of the average number of samples with variation was learned from 10,000 random gene lists of equal size to the set of interacting partners. This permutation test was performed individually for non-synonymous mutations, CNVs, and mRNA expression. The information for observed variation of each gene is collected from the TCGA BC cohort (firehose legacy).

### IAS Network

The integrated associated stringency (IAS) network was derived from integration of five major types of protein pairwise relationships recorded in public databases: (1) physical protein-protein interaction; (2) mRNA co-expression; (3) protein co-expression; (4) co-dependence (correlation of cell line growth upon gene knockouts); and (5) sequence-based relationships. A broad survey created a compendium of 127 network features used as inputs to a random forest regression model, trained to best recover the proximity of protein pairs in the Gene Ontology (GO). The final IAS score, ranging from 0 to 1, quantifies all pairwise associations among 19035 human proteins. In this study, stringent protein interactions were displayed with IAS>0.3 when the IAS network was used in figures.

### Peptide Phosphorylation Assay

This assay uses a set of peptide sequences that are derived from computationally curated biological targets of kinases' substrates deposited in PhosphoAtlas (Chen and Coppé, 2012; Olow et al., 2016). Peptides (total 453 peptides from 237 proteins) individually allocated to separate wells in a series of 384-well plates serve as phosphorylatable probes in a large-scale ATP-consumption biochemical assay handled by automated liquid dispensing instruments. For each experimental run, the average value of ATP concentration in sample-containing wells was used for internal normalization to calculate the phosphorylation activity per peptide as the difference in ATP consumption between each peptide-derived read out and the internal mean. For the current study, the analysis of peptide phosphorylation profiles measured in Spinophilin knockout cells was focused on. To prepare protein extracts to run on the assay platform, cells at ˜85% confluency were washed three times with cold PBS and lysed with freshly prepared 1× cell lysis buffer (1 ml per 3×106 cells) (10× Cell Lysis Buffer, Cell Signaling; cat #9803) complemented with 1× of Halt Protease & Phosphatase (100×, ThermoScientific; cat #1861281). Cell lysates were collected and spun down at 14,000 rpm for 15 min at 4° C. and supernatants stored at −80° C.

### In-Cell Western Blot Assay

Four independent siRNAs per target gene were purchased from Dharmacon (siGENOME SMARTpool) in Echocompatible 384-well plates (Labcyte #PP-0200) and resuspended in 20 uL nuclease-free water. For the assay, 4 pmol of siRNAs were aliquoted into each well of a black walled clear bottom 96-well plates (Corning #3904) avoiding edges using a Labcyte Echo 525. Plates were then stored at −80° C. On the day of the experiment, plates were thawed for 0.5-1 hour at room temperature, centrifuged at 1000 rpm for 5 minutes, and reconstituted with 20 uL of nuclease free water (Ambion #AM9938) on a rotator for 30 minutes. Transfection reagent was prepared using 0.1% RNAiMax (Invitrogen #13778150) and 20% Optimem (Gibco #31985062) for a seeding density of 4,000 cells per well; reagent was allowed to sit for 10 minutes at room temperature before adding 40 uL to each well and incubated for an additional 20 minutes. Cells grown to a confluency of 80% were lifted using 0.25% Trypsin (BioUltra #V611×), counted, and 4000 cells were seeded per well in a 140 uL volume, resulting in 200 uL total volume for each well. Cells were incubated in a standard incubator at 37° C. and 5% CO₂ for 48 hours. Following the 48-hour incubation, growth media was aspirated and cells were fixed using 50 uL per well of 4% paraformaldehyde solution (Thermo Fisher #PI28908) for 15 minutes. Cells were permeabilized using 50 uL 1:100 dilution of Triton X-100 (Sigma #9002-93-1) in 1×PBS for 30 minutes, then incubated in a 2× blocking solution (2% BSA in 1×PBS) at room temperature for 2 hours. Next, blocking buffer was removed and replaced with 50 uL 1× primary antibody per well, prepared by diluting Total AKT (mouse; Cell Signaling Technologies #2920S) and pAKT S473 (rabbit; Cell Signaling Technologies #4060S) at 1:800 dilution in 1× blocking buffer (1% BSA in 1×PBS). Cells were incubated in 1× primary antibody solution overnight at 4° C. The next morning, cells were washed with 1× wash buffer (250 uL Tween-20 in 50 mL 1×PBS) and incubated for 2 hours in the dark at room temperature with 1× secondary antibody solution containing 1:1000 dilution (in 1% BSA) of anti-mouse (926-32210) and anti-rabbit (926-32211) near-infrared antibodies. Cells were washed using 1× wash buffer and resuspended in 100 uL PBS for fluorescence detection using an LiCOR Odyssey plate scanner (9140). Wavelengths for the antibodies were set to 680 nm for anti-rabbit and 800 nm for anti-mouse. To measure cell viability, PBS was aspirated and cells were stained with 50 uL Janus Green Stain (Abcam #ab111622) for 5 minutes at room temperature. Cells were washed using ultrapure water and lysed with 100 ul 0.5m HCl shaking at 400 rpm for 10 minutes. A standard microplate spectrophotometer was used to measure OD 595 nm.

### Co-Immunoprecipitation and Western Blot Analysis

Cell extracts were prepared using the same protocol as described in the Cell lysis and affinity purification. To ensure the same amount of proteins for each sample, supernatant was quantified by Bradford protein assay prior incubation with the beads. After overnight incubation with beads at 4° C., as previously described, proteins were eluted from the beads by boiling in 2×SDS Sample Buffer (Alfa Aesar) diluted in S150 buffer and stored at −20° C.

For immunoblots, samples were loaded onto 7.5% Mini-PROTEAN® TGX™ Precast Protein Gel (Bio-Rad). After gel electrophoresis, the samples were transferred to a membrane with Trans-Blot Turbo Transfer System (BioRad). Membranes were blocked with 5% Milk TBST for 1 h at RT and incubated in the blocking solution overnight at 4° C. with the indicated antibodies. The incubation was followed by washing with TBST and 1 hr incubation at RT with secondary antibodies. Bands were detected using an ECL chemiluminescence detection method with KwikQuant Ultra Digital ECL-solution, KwikQuant™ Imager and analyzed with KwikQuant Image Manager Software (all Kindle Biosciences, LLC).

### DSB GFP Reporter Assay

U2OS cells were reverse transfected by plating 2×105 cells in antibiotic-free media in a 12 well plate. Each well already contained preformed transfection complexes with 20 pmol siRNA and 3.6 μL Lipofectamine RNAiMAX Reagent (Invitrogen) in Opti-MEM used according to the manufacturer's protocol. After 20 hrs, 2×105 cells were transferred to 6 well plates and left to recover until the next day. Transient I-SceI transfection was performed 48 hrs post initial reverse transfection. 1.92 μg I-SceI expression vector, prepared by Mini or Midi Kit (Qiagen), was used along with 24 pmol siRNA and 8.64 μL Lipofectamine 2000 Transfection Reagent (Invitrogen) in Opti-MEM according to the manufacturer's protocol. Cells were incubated with transfection complexes for 3 hrs at 37° C. followed by gentle washing and addition of fresh growth media with antibiotics.

### Flow Cytometric Analysis

Approximately 72 hrs after I-SceI transfection, cells were trypsinized, washed with PBS, fixed in 1% formaldehyde and transferred to V-bottom 96-well plates. DNA repair activity was assessed by a quantification of the percentages of GFP+ cells using the Attune NxT Flow Cytometer (ThermoFisher), and analyzed using FlowJo software (FlowJo, LLC). Experiments were performed in triplicates and error bars expressed as standard deviation (SD).

### Western Blot Analysis

Protein extracts were performed as described previously. After Bradford analysis, samples were boiled in 1×SDS Sample Buffer, before proceeding with gel electrophoresis and protein transfer onto a membrane. To detect the protein of interest, the membranes were incubated with indicated antibodies.

### I-SPY 2 TRIAL: Patients, Data, and Analysis

This correlative study involved 375 (MK2206 arm: 94; veliparib/carboplatin (VC) arm: 71; Ctr: 210) women with high-risk stage II and III early breast cancer who were enrolled in the multicenter, multi-arm, neo-adjuvant I-SPY 2 TRIAL (NCT01042379; IND 105139) (Barker et al., 2009). Detailed descriptions of the design, eligibility, and study assessments in the I-SPY 2 trial have been reported previously, including the efficacy of investigational agents VC (Rugo et al., 2016) and MK-2206 (Chien et al., 2020). I-SPY 2 TRIAL patients are randomized either to the control arm [paclitaxel followed by doxorubicin/cyclophosphamide; T→AC; plus trastuzumab (and later pertuzumab) if HER2+] or one of the active experimental arms. The investigational agent MK2206 was active in the trial from September 2012 to May 2014. MK2206 arm patients received MK2206 plus standard chemotherapy (n=94; M+T→AC), with trastuzumab if HER2+. 72 HER2-patients were randomized to the VC arm from May 2010 to July 2012, and treated with veliparib and carboplatin in addition to standard taxane/anthracycline chemotherapy (VC+T→AC) (Rugo et al., 2016). All patients signed informed consent to allow research on and use of their biospecimen samples (Chien et al., 2020; Rugo et al., 2016). Pre-treatment tumor samples were assayed using Agilent 44K (32627) or 32K (15746) expression arrays; and these data were combined into a single gene-level dataset after batch-adjusting using ComBat (Johnson et al., 2007). In the pre-specified analysis plan as previously summarized (Wolf et al., 2017; Wulfkuhle et al., 2018), logistic regression is used to assess association with pCR in the control and experimental-arm treated populations individually. Relative biomarker performance between arms (biomarker×treatment interaction) is assessed using a logistic model (pCR˜treatment+biomarker+treatment×biomarker). Analysis is also performed adjusting for HR/HER2 (binary) status (pCR-treatment+biomarker+treatment:biomarker+HR+HER2). Markers were analyzed individually; p-values are descriptive.

### Results

### Protein-Protein Interaction Mapping of Breast Cancer Drivers

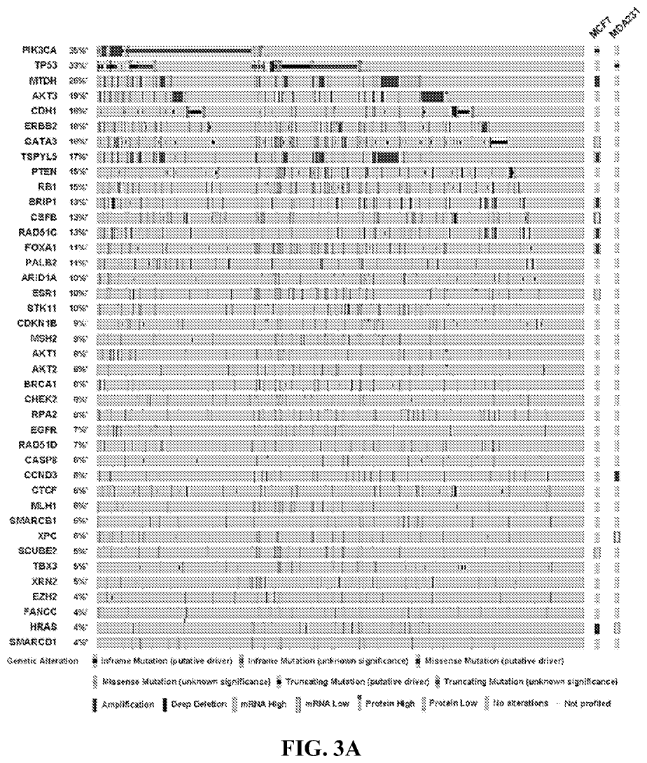

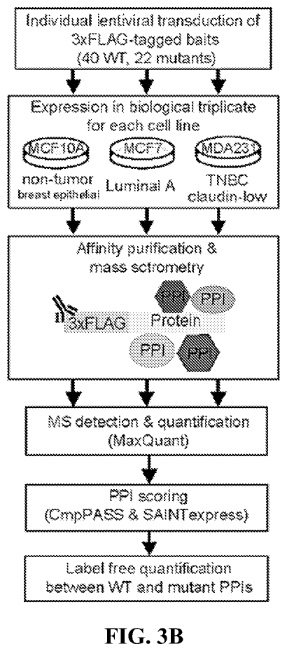

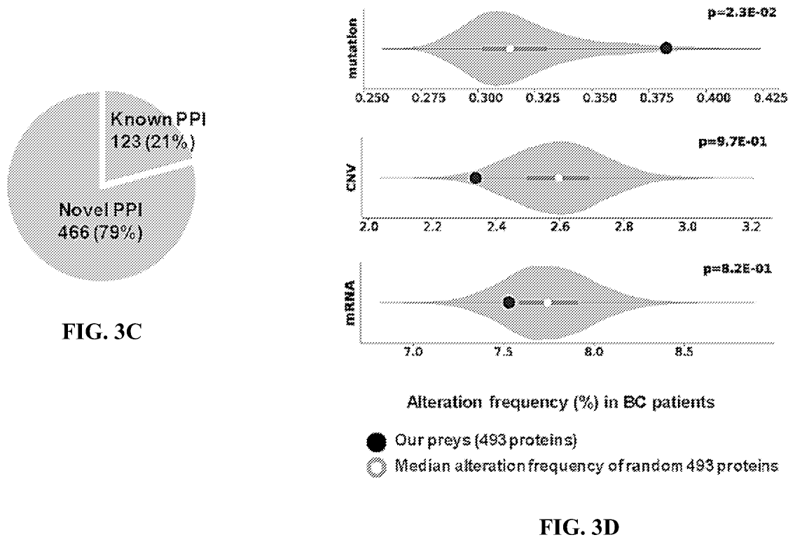

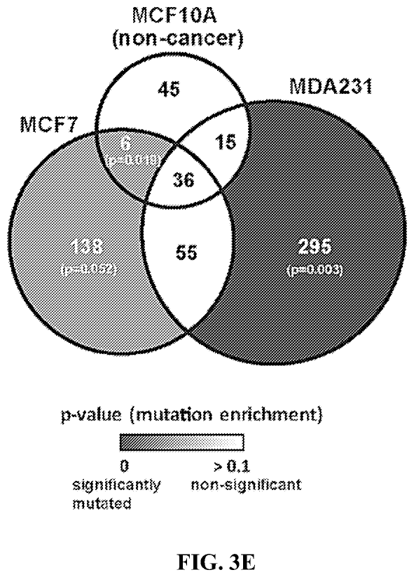

A panel of genes that are associated with molecular alterations in BC were collected, and the list (Cancer Genome Atlas, Network, 2012; Stephens et al., 2012) was used to guide the selection of 40 proteins for generation of PPI networks. The selected targets included proteins with well-known roles in BC (e.g., TP53, PIK3CA, CDH1, and BRCA1) as well as less-well appreciated proteins with recurrent mutations (e.g., CHEK2) (Beca et al., 2017; Chen et al., 2017; Epping et al., 2011; Fuqua et al., 2014; Goldberg et al., 2017; Harkness et al., 2015; Hoenerhoff et al., 2009; Lin et al., 2014; Mimori et al., 2002; Morales et al., 2016; Thompson et al., 2012; Tokunaga et al., 2014; Zheng et al., 2011). This list was inclusive, as 93% of BC tumors in TCGA harbor an alteration in one or more of these 40 genes (FIG. 3A).

Referring to FIG. 3A, the gene alteration frequencies from the breast invasive carcinoma (TCGA Firehose Legacy) dataset for the 40 genes selected as AP-MS baits in this study are shown. Each patient is represented by a grey box that is colored based on the occurrence and type of alteration(s) observed in that patient. In total, 93% (1028 out of 1108) of BC patients have non-synonymous mutation, chromosomal copy-number alteration (CNA), or mRNA/protein expression alteration in one or more of these 40 genes. Existing gene alterations in MCF7 and MDA-MB-231 are shown in the right.

Three breast cell lines derived from human mammary epithelium were selected: MCF7 (ER+, luminal A subtype), MDA-MB-231 (ER-, PR-, HER2-triple-negative TN subtype), and MCF10A (non-tumorigenic mammary epithelial cells). These particular cell lines were selected because they have been shown to replicate therapeutically relevant responses found in BC tumors (Iorio et al., 2016), their RNA profiles are highly correlated with those of BC tumors (Yu et al., 2019), and ER+ and TN subtypes together account for approximately 90% of BC patients (Santagata et al., 2014). It was reasoned that comparing protein networks among ER+, TN, and nontumorigenic models would allow study of how PPI networks are altered between normal and tumorigenic backgrounds as well as influenced by different mammary epithelial lineages.

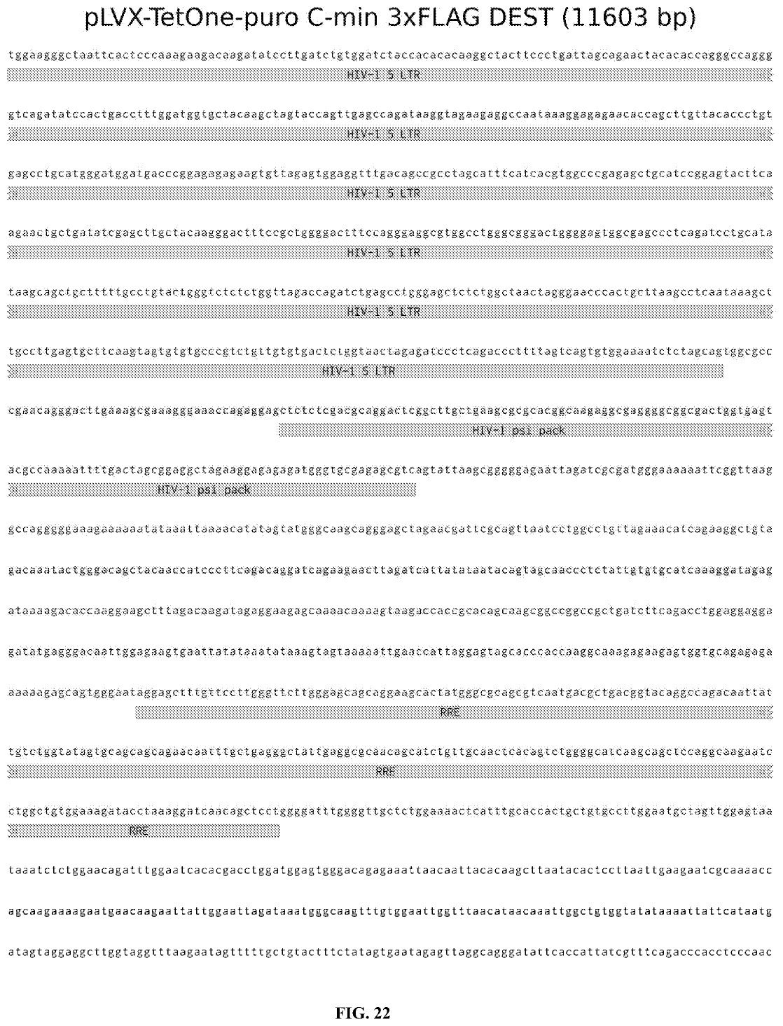

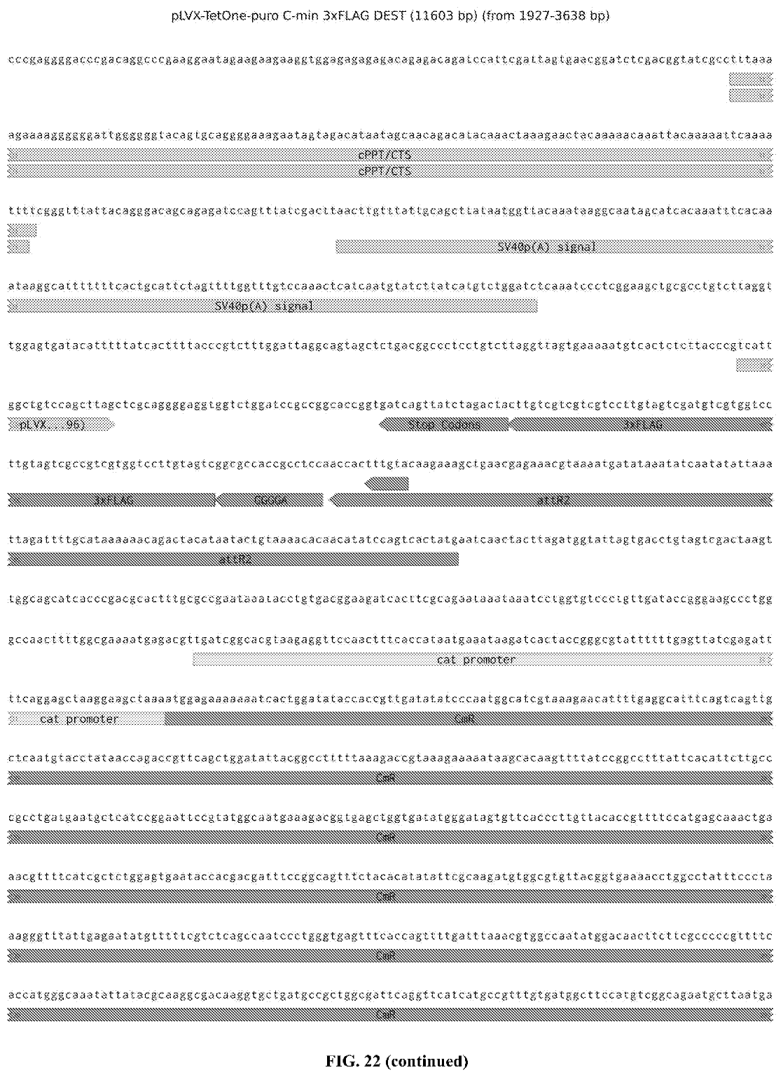

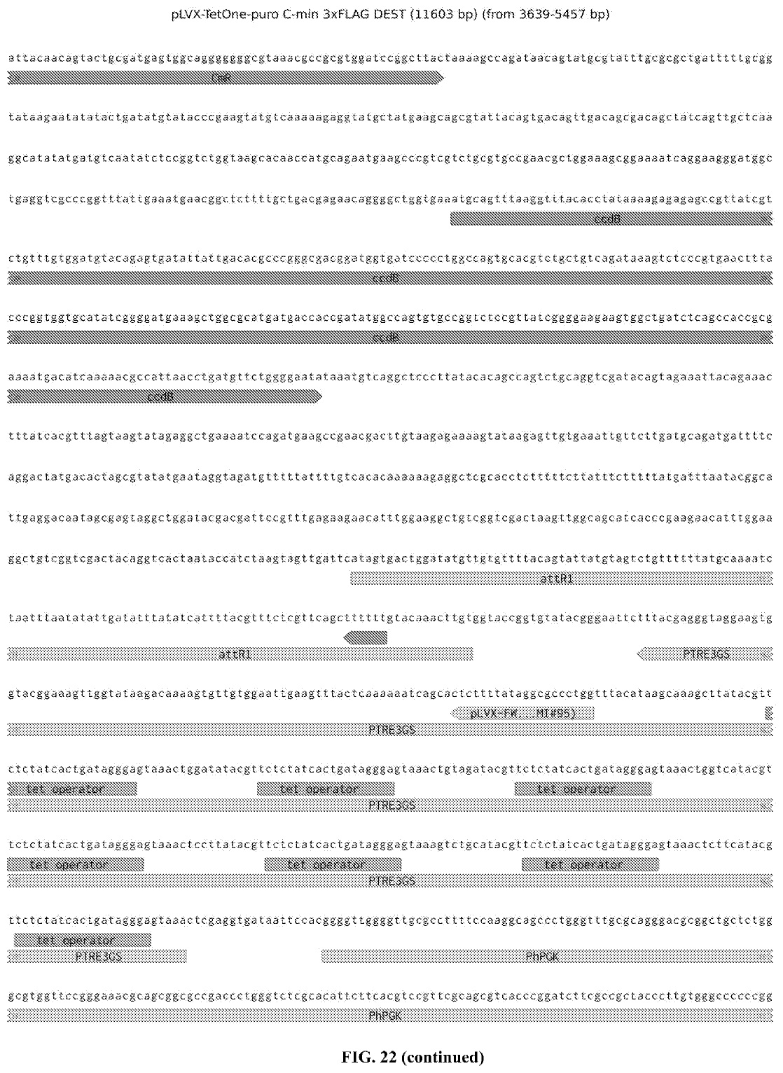

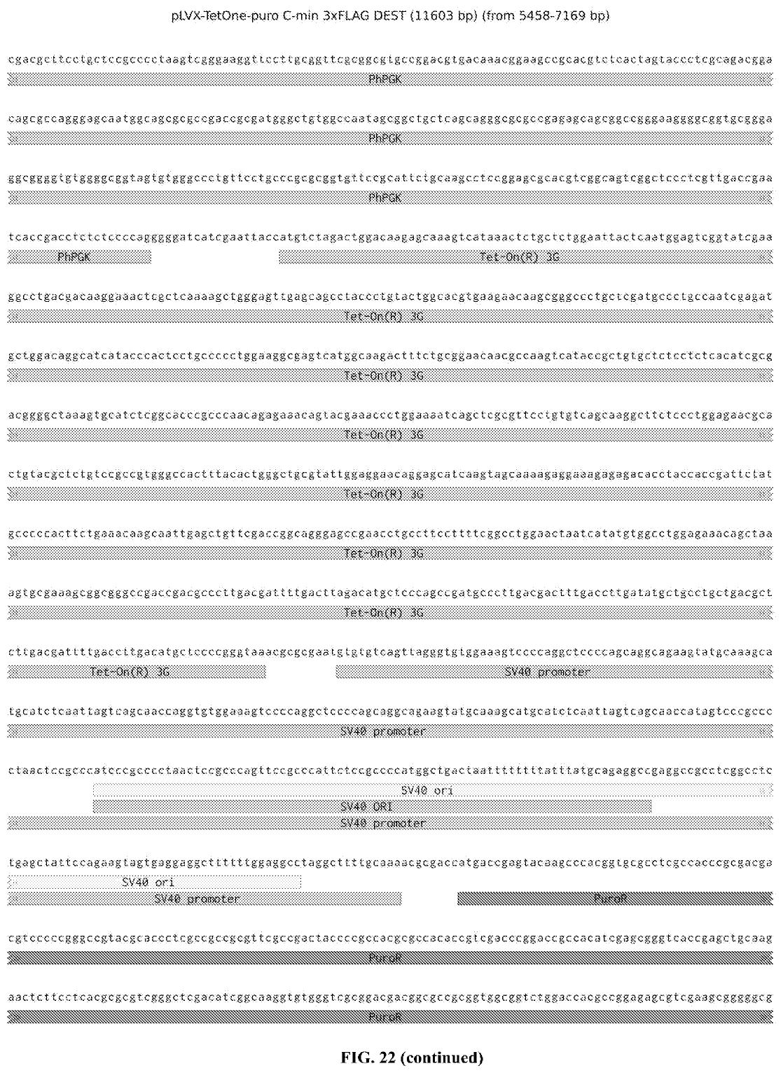

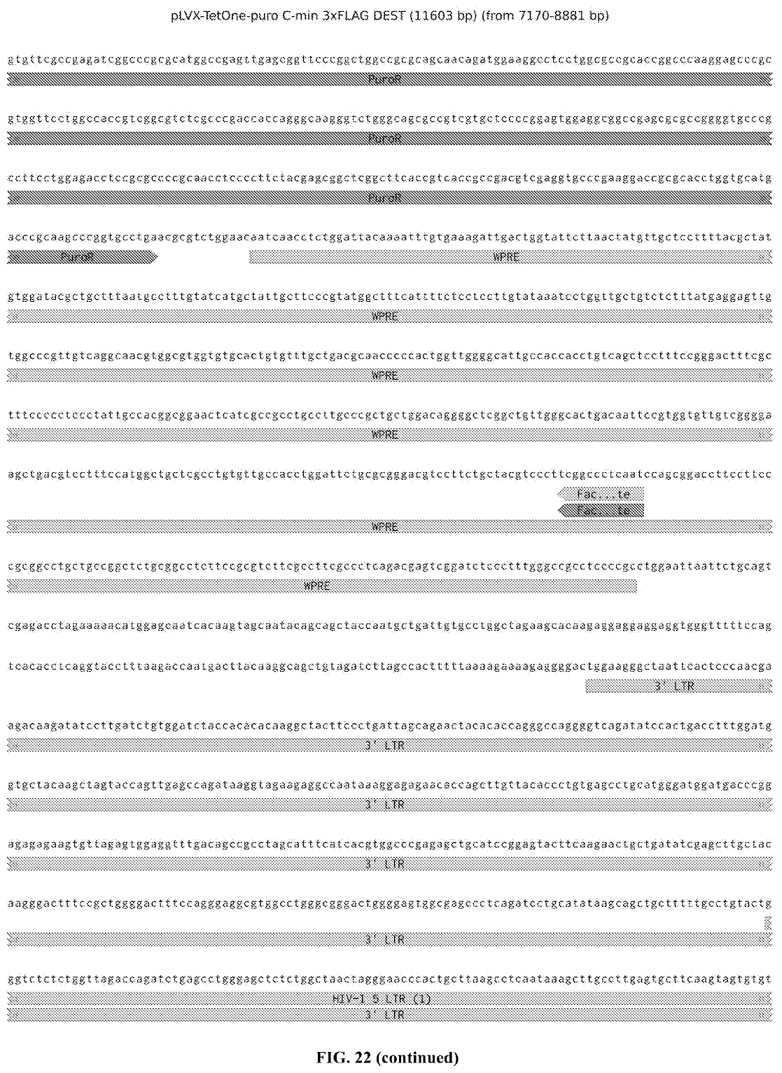

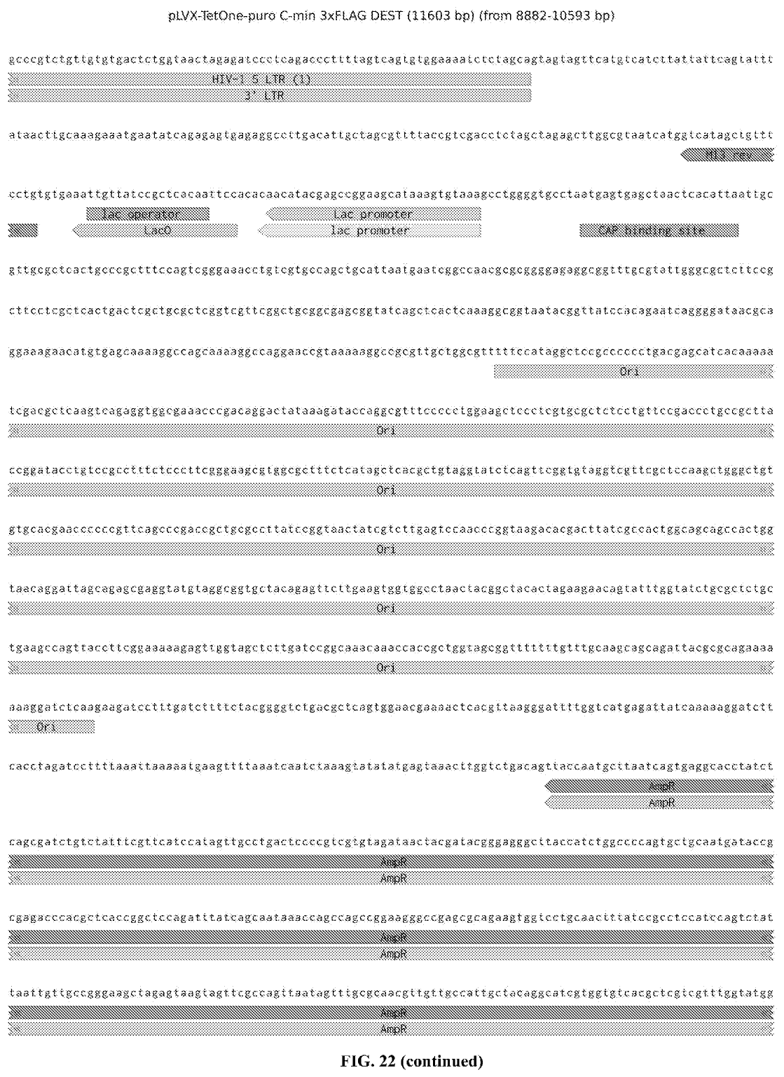

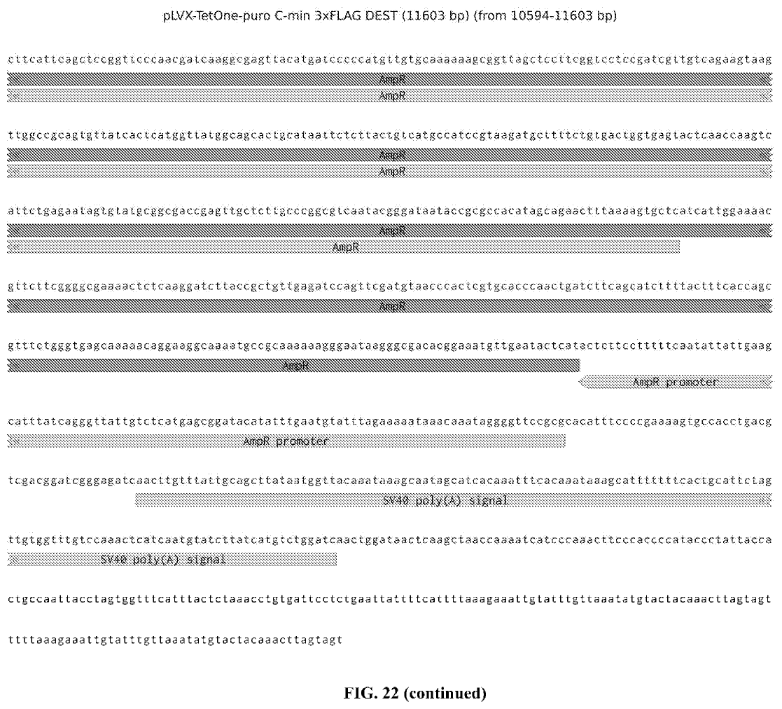

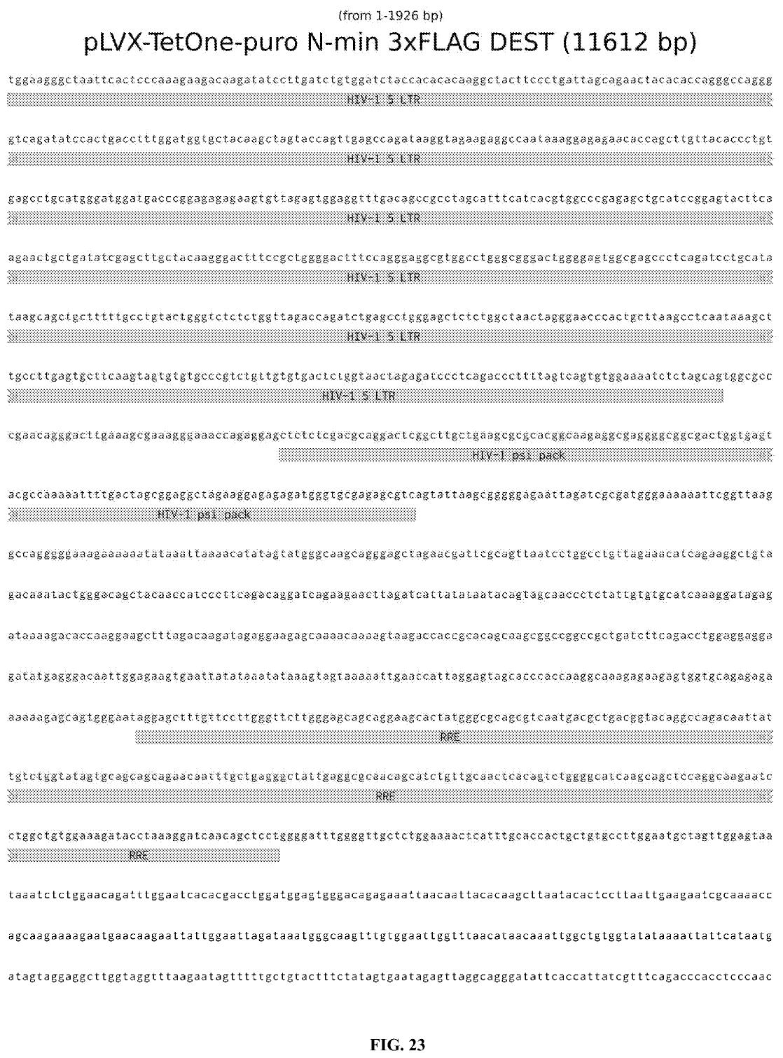

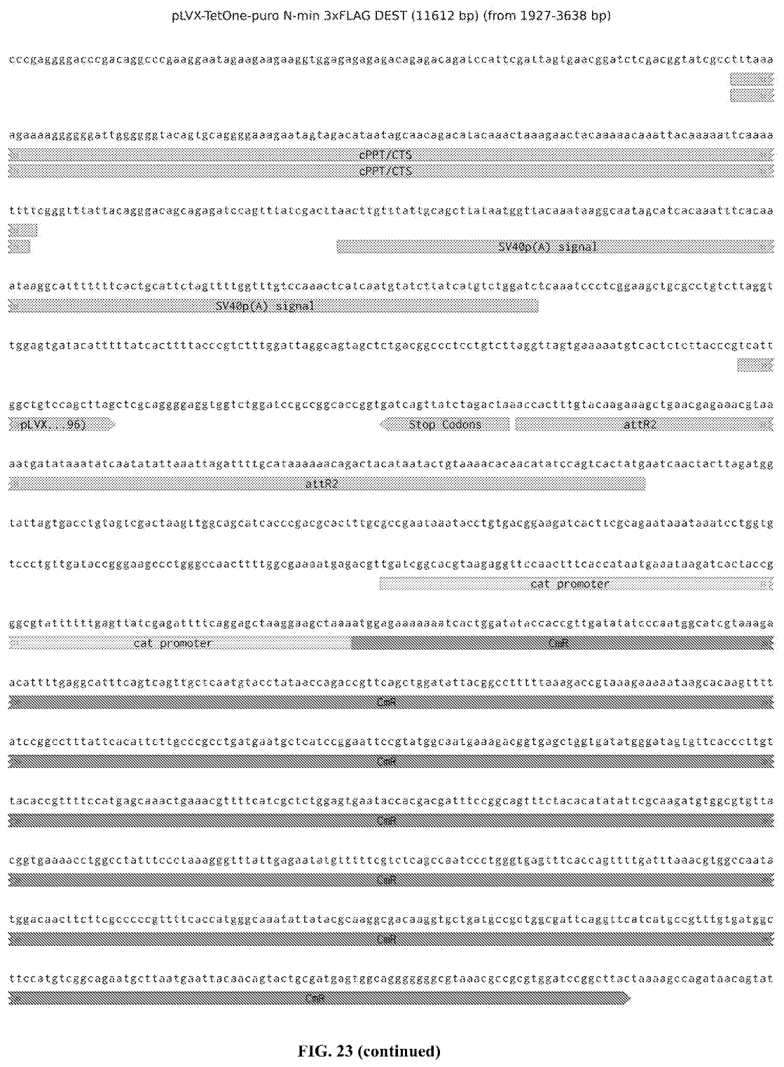

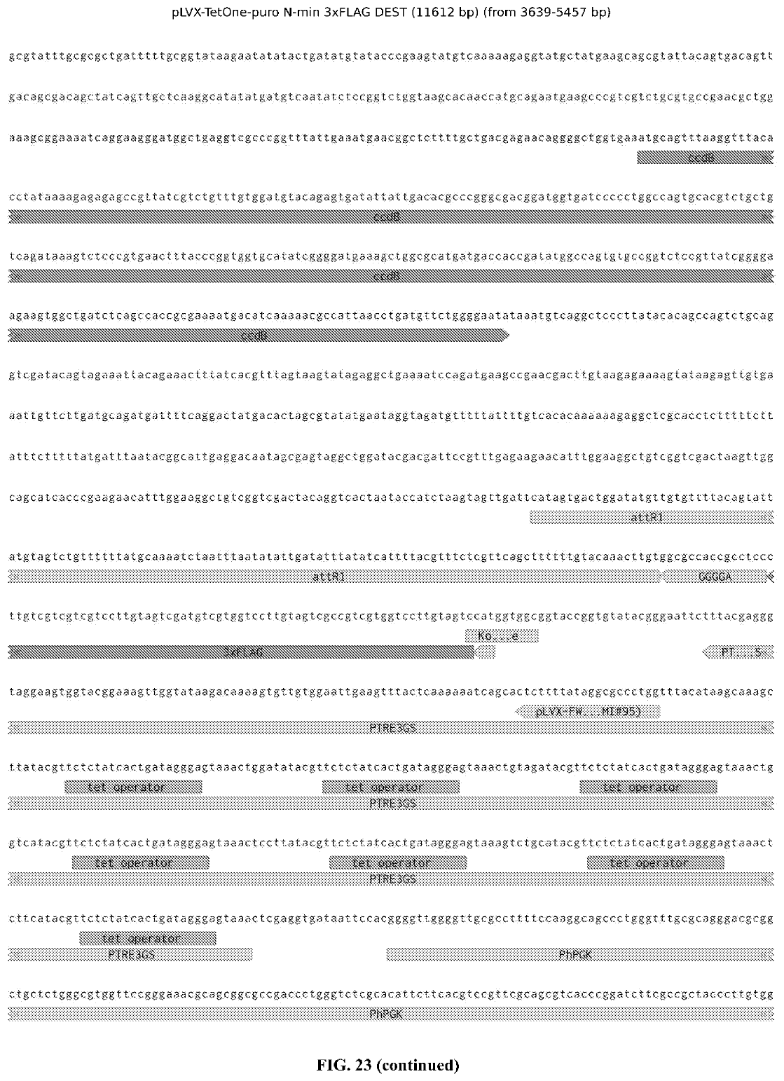

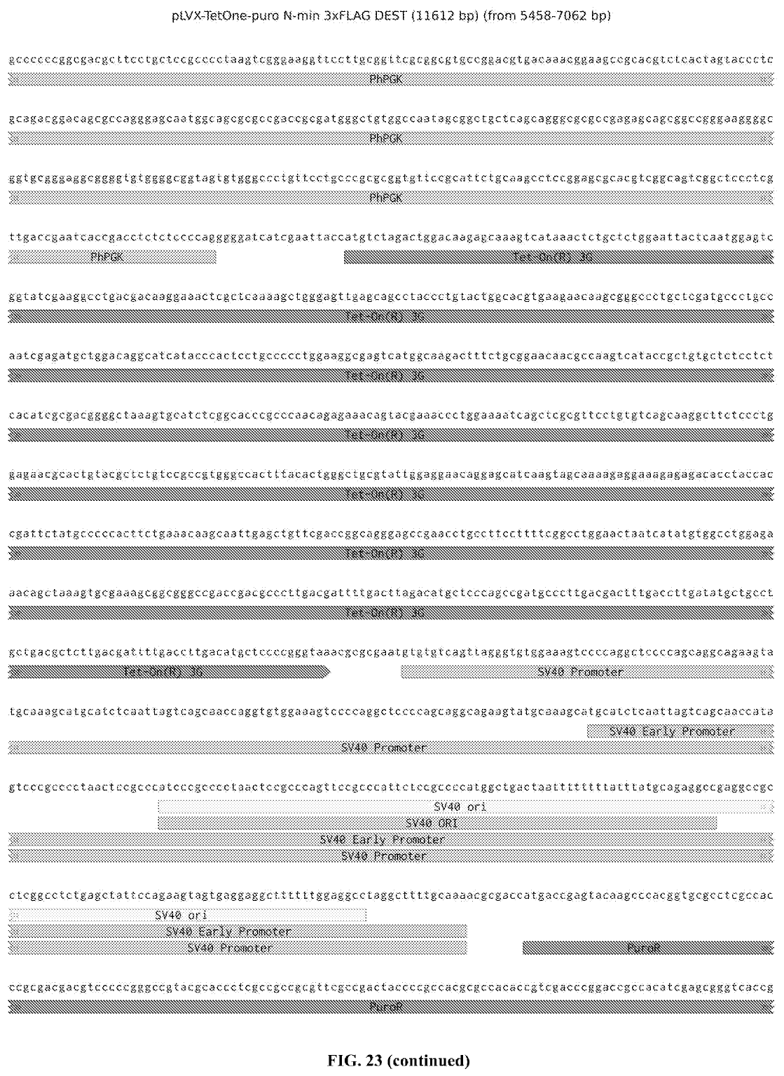

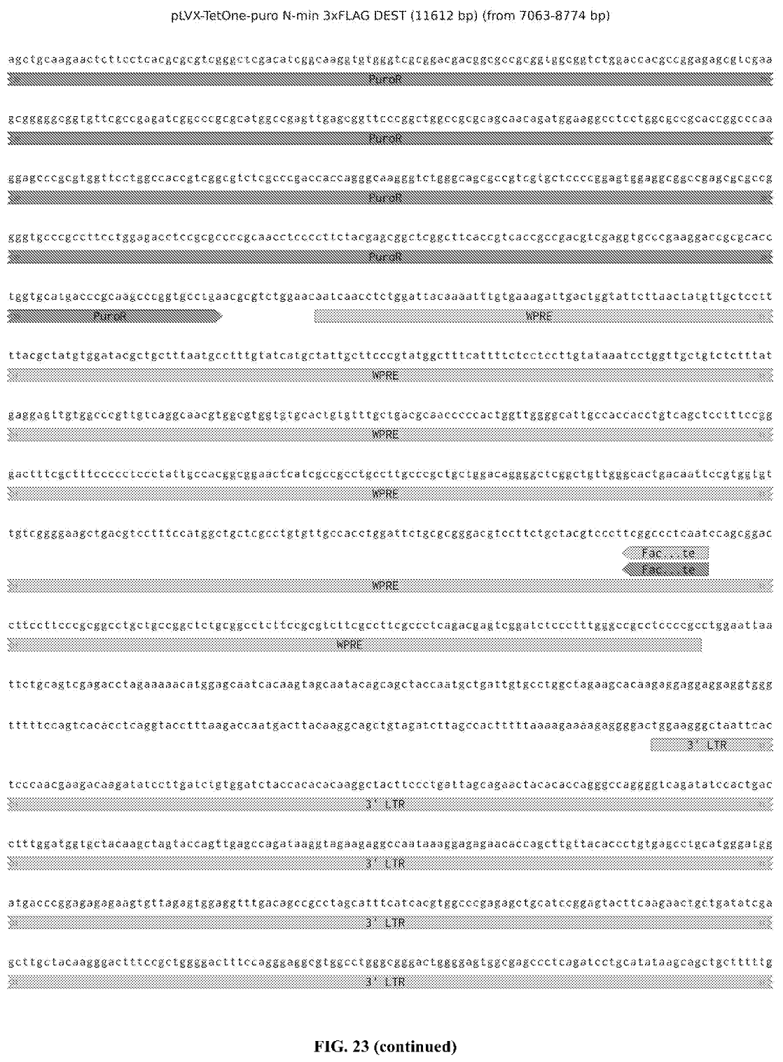

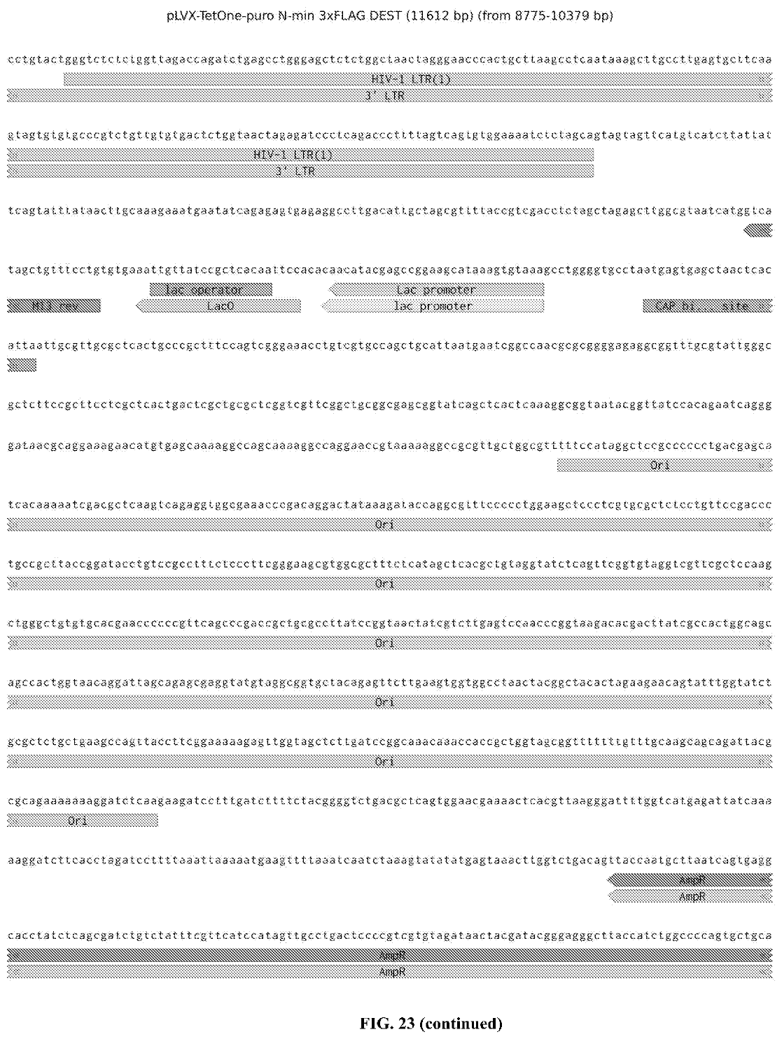

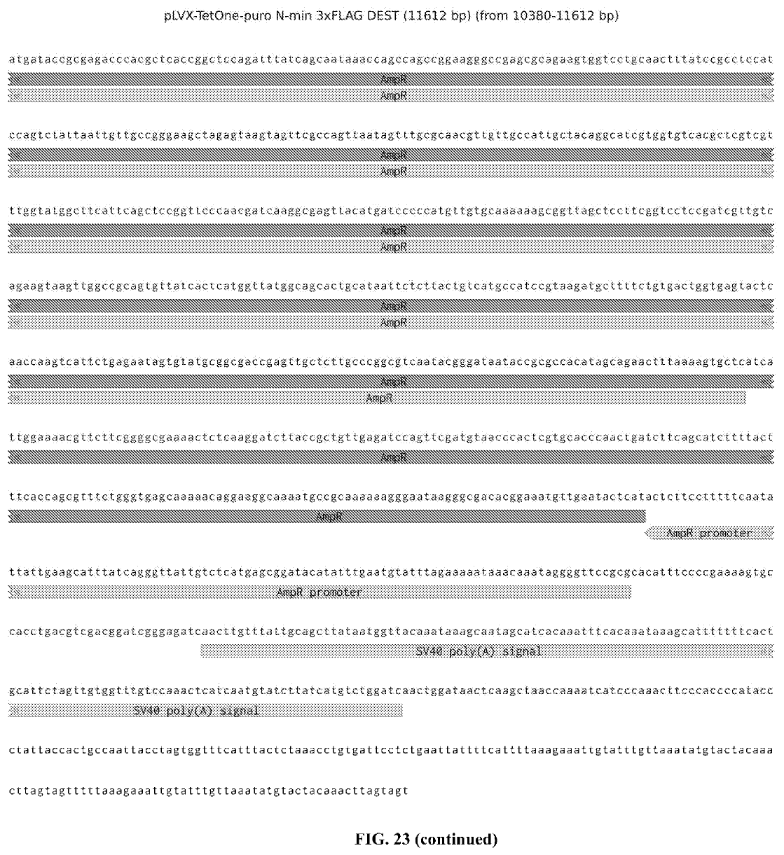

To generate PPI maps, “bait” proteins were cloned into triple FLAG-tagged lentiviral vectors, individually transduced into each cell line and expressed in biological triplicate via a doxycycline inducible promoter (FIG. 3B). See also FIG. 22 and FIG. 23. Cells were harvested after approximately 40 hr doxycycline-induction, and anti-FLAG tag-based affinity purification was performed followed by mass spectrometry to detect interacting “prey” proteins in an unbiased manner. Two PPI scoring algorithms were employed to quantify high-confidence interacting proteins: compPASS (Huttlin et al., 2015a; Sowa et al., 2009a) and SAINTexpress (Teo et al., 2014a). The AP-MS data was independently analyzed using these two algorithms and monitored recovery rates of known PPIs from public databases (serving as gold standards) by varying the thresholds of key metrics in each algorithm (WD per bait percentile for compPASS and BFDR for SAINTexpress, respectively). Without wishing to be bound by theory, it was found that the best gold-standard PPI recovery was obtained when data from both algorithms were combined (FIG. 1A and FIG. 1B). Using this approach, a total of 589 high-confidence PPIs involving 493 prey proteins were identified (FIGS. 2A, 2B, and 3C).

Referring to FIG. 2A, receiver operating characteristic (ROC) curve illustrating high recovery of gold standards (sensitivity) are shown.

Referring to FIG. 2B, the number of high-confidence PPIs per cell line for each bait are shown.

Referring to FIG. 3B, the experimental workflow in which each bait was expressed in biological triplicate in 3 cell lines and subjected to AP-MS analysis is shown.

Referring to FIG. 3C, the majority (79%) of the high-confidence PPIs identified in this study are not represented in a panel of public PPI databases (CORUM, BioPlex 2.0, or BioGRID low throughput & multivalidated).

Collectively, 79% of the BC PPIs identified were not previously reported in protein-protein interaction databases (CORUM, BioPlex 2.0, or BioGRID low throughput and multivalidated) (FIG. 3C). Without wishing to be bound by theory, the high percentage of novel interactions may reflect cell type-specific PPIs as nearly all systematic protein-protein interaction analyses to date have been performed in HEK293T or HeLa cell lines (Hein et al., 2015; Huttlin et al., 2015a, 2017, 2020). This study is the first to collect large-scale human PPI data in cell line contexts physiologically relevant to breast cancer.

PPIs often suggest functional relationships among proteins that work together to accomplish a specific cellular process. Previously, a significant enrichment of frequently mutated proteins was found in large PPI repositories (Bouhaddou et al., 2019; Creixell et al., 2015; Eckhardt et al., 2018; Hofree et al., 2013; Leiserson et al., 2015; Paczkowska et al., 2020; Reyna et al., 2020). Similarly, it was investigated whether the BC PPI network showed enrichment for three major types of alterations—non-synonymous mutations, chromosomal CNVs, and mRNA expression alterations—documented in the BC TCGA cohort. Accordingly, the average frequency of each alteration was calculated for prey proteins detected in the PPIs, compared to background expectation (STAR Methods and FIG. 2C). It was observed that BC-associated mutations were significantly enriched in BC PPIs, but that CNVs and mRNA expression alterations were not (FIG. 3D). Specific enrichment of mutations was found in preys detected in either of the two cancer cell lines (MCF7, MDA-MB-231) but not in the preys of non-cancerous MCF10A cells (FIG. 3E). Without wishing to be bound by theory, this result supports the notion that the interaction partners of frequently mutated cancer proteins are also under positive pressure for mutations.

Referring to FIG. 2C, volcano plots displaying the differential abundance of endogenous proteins between cell lines used in this study are shown. Colored data points (left and right sides) indicate proteins that have ≤5.6-fold difference in protein abundance between cell lines and an adjusted p-value<0.05.

Referring to FIG. 3D, the frequency of non-synonymous mutations, chromosomal CNVs, or mRNA expression alterations of 10,000 random size-matched permutations taken from the list of genes detected in the global protein abundance analysis is shown. The white circle indicates the median of the random sampling, and the grey bar represents ±1 standard deviation. The frequency of alterations found in the prey retrieved in our PPI dataset is indicated in the solid colored.

Referring to FIG. 3E, a Venn diagram illustrating the overlap of PPIs (PPI score≥0.9) across the 3 cell lines is shown. PPI score is an average of the PPI confidence scores calculated from compPASS and SAINTexpress. The frequency of non-synonymous mutations of the prey genes in each sector of the Venn diagram was compared to those of 10,000 random size-matched permutations as in FIG. 3D. The p-values for mutation enrichment in each prey set were shown in a shaded scale, where a stronger gray represents more significant mutation enrichment.

Key Resources Table I.

| Reagent | Source | Identifier |
|---|---|---|
| Antibodies | | Cat# |
| pSTK11 | Cell | 3482 |
| | Signaling | |
| | Techologies | |
| STK11 | Cell | 3050 |
| | Signaling | |
| | Techologies | |
| pAMPK | Cell | 2535 |
| | Signaling | |
| | Techologies | |
| AMPK | Cell | 5832 |
| | Signaling | |
| | Techologies | |
| pSIKs | Abcam | Ab199474 |
| SIK1 | Thermo | PA5-42799 |
| | Fisher | |
| | Scientific | |
| Phospho-AKT | Cell | 4060S |
| (Ser473)(D9E) | Signaling | |
| XP Rabbit mAb | Techologies | |
| Akt (pan)(40D4) | Cell | 2920S |
| Mouse mAb | Signaling | |
| | Techologies | |
| Goat Anti-Mouse | LI-COR | 926-32210 |
| Secondary | | |
| Antibody 800CW | | |
| LI-COR | | |
| Goat Anti-Rabbit | LI-COR | 926-32211 |
| Secondary | | |
| Antbidoy 800CW | | |
| LI-COR | | |
| BRCA1 (D-9) | Santa Cruz | sc-6954 |
| | Biotechnology | |
| BARD1 (E-11) | Santa Cruz | sc-74559 |
| | Biotechnology | |
| RBBP8 | Santa Cruz | sc-271339 |
| | Biotechnology | |
| Spinophilin/ | Santa Cruz | sc-373974 |
| Neurabin-II (D-7) | Biotechnology | |
| UIMC1 | Abcam | ab-124763 |
| BRIP1 | Abcam | ab-180853 |
| MLH1 | Abcam | ab-92312 |
| Beta-Tubulin | Sigma- | T8328 |
| | Aldrich | |
| Actin (13E5) | Cell | 4970 |
| | Signaling | |
| | Techologies | |
| Anti-Mouse | Cell | 7076 |
| | Signaling | |
| | Techologies | |
| Anti-Rabbit | Cell | 7074 |
| | Signaling | |
| | Techologies | |
| Cell Culture | | |
| Media | | Cat# |
| High glucose | Corning | MT10017CV |
| Dulbecco's | | |
| modified Eagle's | | |
| medium (DMEM) | | |
| DMEM and | Corning | MT10092CV |
| Ham's F-12 50/50 | | |
| Chemicals, | | |
| Drugs, Peptides, | | |
| and Enzymes | | Cat# |
| Fetal Bovine | Gibco | A3160502 |
| Serum | | |
| Penicillin/ | Corning | MT30002C1 |
| Streptomycin | | |
| Gag-Pol-Tat-Rev | | pJH045 |
| VSV-G | | pJH046 |
| PolyJet DNA | SigmaGen | SL100688 |
| Transfection | Laboratories | |
| Reagent | | |
| 8.5% PEG-6000 | Sigma- | 528877 |
| | Millipore | |
| Puromycin | Sigma | P8833 |
| Blasticidin S HCI | Gibco | R21001 |
| Doxycycline | Selleckchem | S4163 |
| 100X Halt™ | Thermo | 78443 |
| Protease and | Scientific | |
| Phosphatase | | |
| Inhibitor Single- | | |
| Use Cocktail, | | |
| EDTA-Free | | |
| Benzonase | Sigma | E1014-25KU |
| LysC | Wako | 129-02543 |
| | Chemicals | |
| Iodacetamide | BioUltra | I1149 |
| (IAA) | | |
| Trypsin | Promega | V611X |
| 6X SDS Sample | Alfa Aesar | J61337-AD |
| Buffer | | |
| Hydrocortisone | Sigma- | HO888-1G |
| | Aldrich | |
| Insulin Solution | Sigma- | 501656853 |
| Human 19278 | Aldrich | |
| Animal-Free | PeproTech | 10781-696 (EA) |
| Recombinant | | |
| Human EGF | | |
| Cholera Toxin | Sigma- | C-8052 |
| | Aldrich | |
| Janus Green Stan | Abcam | Ab111622 |
| Invitrogen | Ambion | AM9938 |
| Nuclease-Free | | |
| Water | | |
| 16% | Thermo | PI28908 |
| Paraformaldehyde | Fisher | |
| Triton X-100 | Sigma- | 9002-93-1 |
| | Aldrich | |
| Lipofectamine2000 | Invitrogen | 11668019 |
| Lipfectamine | Invitrogen | 13778150 |
| RNAiMAX | | |
| Opt-mem | Gibco | 31985070 |
| Reduced Serum | | |
| Laboratory | | |
| equipment and | | |
| beads | | Cat# |
| 0.45 μM PVDF | Millipore | MM_NF_SLHV033RS |
| filter | | |
| Nest Tips C18 | The Nest | SUM SS18V |
| | Group | |
| Anti-FLAG M2 | Sigma- | M8823 |
| magnetic beads | Aldrich | |
| Anti-V5 magentic | MBL | M167-11 |
| beads | International | |
| Ni-NTA Magnetic | Qiagen | 36111 |
| Agarose Beads | | |
| Micro Bio-Spin | Bio-Rad | 7326204 |
| chromatography | | |
| Columns | | |
| 7.5% Mini- | Bio-Rad | 4561024 |
| PROTEAN® | | |
| TGX™ Precast | | |
| Protein Gel | | |
| Trans-Blot Turbo | Bio-Rad | |
| Transfer System | | |
| KiwkQuant Ultra | Kindle | R1002 |
| Digital ECL- | Biosciences, | |
| solution | LLC | |
| KwikQuant™ | Kindle | |
| Imager | Biosciences, | |
| | LLC | |
| KwikQuant Image | Kindle | |
| Manager Software | Biosciences, | |
| | LLC | |
| Echo Qualified | Dharmacon | cat# PP-0200 |
| Labcyte 384-well | | |
| platesCorning | | |
| Clear-bottom 96 | Corning | CLS3904 |
| Well Plates | | |
| Li-Cor Odyssey | Li-Cor | 9140 |
| CLx | | |
| Deposited Data | | Cat# |
| Raw MS files and | Proteome | Identifier: PXD019639 |
| MaxQuant search | Xchange | Username: reviewer58609@ebi.ac.uk |
| files | Pride | Password: vymSibLr |
| | partner | |
| | Repository | |
| Experimental | | |
| Models: Cell | | |
| Lines | | Cat# |
| HEK293T | ATCC | CRL-3216 |
| U2OS | Gunn and | Jeremy Stark's laboratory |
| | Stark, 2012 | |
| MDA-MB-231 | ATCC | HTB-26 |
| MCF7 | ATCC | HTB-22 |
| MCF10A | ATCC | CRL-10317 |
| siRNA | | Cat# |
| AKT1- | Dharmacon | M-003000-03-0005 |
| siGENOME | | |
| SMARTpool | | |
| AKT2- | Dharmacon | M-003001-02-0005 |
| siGENOME | | |
| SMARTpool | | |
| AKT3- | Dharmacon | M-003002-02-0005 |
| siGENOME | | |
| SMARTpool | | |
| S100A3- | Dharmacon | M-011767-00-0005 |
| siGENOME | | |
| SMARTpool | | |
| KRT32- | Dharmacon | M-011310-01-0005 |
| siGENOME | | |
| SMARTpool | | |
| PIK3CA- | Dharmacon | M-003018-03-0005 |
| siGENOME | | |
| SMARTpool | | |
| PTEN- | Dharmacon | M-003023-02-0005 |
| siGENOME | | |
| SMARTpool | | |
| BPIFA1- | Dharmacon | M-008613-00-0005 |
| siGENOME | | |
| SMARTpool | | |
| BPIFB1- | Dharmacon | M-010095-01-0005 |
| siGENOME | | |
| SMARTpool | | |
| SCGB2A1- | Dharmacon | M-019606-01-0005 |
| siGENOME | | |
| SMARTpool | | |
| PRR4- | Dharmacon | M-012367-02-0005 |
| siGENOME | | |
| SMARTpool | | |
| MUC5B- | Dharmacon | M-184282-00-0005 |
| siGENOME | | |
| SMARTpool | | |
| ZG16B- | Dharmacon | M-015971-01-0005 |
| siGENOME | | |
| SMARTpool | | |
| ANXA1- | Dharmacon | M-011161-01-0005 |
| siGENOME | | |
| SMARTpool | | |
| IRS1- | Dharmacon | M-003015-01-0005 |
| siGENOME | | |
| SMARTpool | | |
| APOA1- | Dharmacon | M-010994-00-0005 |
| siGENOME | | |
| SMARTpool | | |
| LTF- | Dharmacon | M-19661-01-0005 |
| siGENOME | | |
| SMARTpool | | |
| PIK3R1- | Dharmacon | M-003020-04-0005 |
| siGENOME | | |
| SMARTpool | | |
| PIGR- | Dharmacon | M-017729-00-0005 |
| siGENOME | | |
| SMARTpool | | |
| PIP- | Dharmacon | M-004904-00-0005 |
| siGENOME | | |
| SMARTpool | | |
| NTC#1- | Dharmacon | D-001210-02-05 |
| siGENOME | | |
| NTC#2- | Dharmacon | D-001210-04-05 |
| siGENOME | | |
| Spinophilin #4- | Dharmacon | D-014932-02-0010 |
| siGENOME | | |
| Spinophilin #5- | Dharmacon | D-014932-03-0010 |
| siGENOME | | |
| BRCA-296- | Dharmacon | CTM-554665 |
| siGENOME | (Anantha et | (GGAACCUGUCUCCACAAA GdTdT) |
| | al., 2017) | (SEQ ID NO: 7) |
| TP53BP1- | Dharmacon | M-003548-01-0005 |
| siGENOME | | |
| SMARTpool | | |
| BRCA2-1949- | Dharmacon | CTM-566149 |
| siGENOME | (Anatha et | (gaagaaugcagguuuaau adTdT) |
| | al., 2017) | (SEQ ID NO: 8) |
| Software and | | |
| Algorithms | | |
| FlowJo v106.1 | FlowJo, | https://www.flowjo.com/ |
| | LLC | |
| Attune NxT Flow | ThermoFisher | |
| Cytometer | | |
| Li-Cor Imaging | Li-Cor | https://www.licor.com/bio/ |
| Studio Software | | image-studio/ |
| atMS | Bioconductor | https://www.bioconductor/ |
| | | org/packages/release/bioc/ |
| | | html/artMS.html |
| MSstats | Bioconductor | https://www.bioconductor.org/ |
| | | packages/release/bioc/html/ |
| | | MSstats.html |
| Max Quant | Jurgen Cox | https://www.maxquant.org/ |
| (version | Lab | |
| 1.6.2.10) | | |
| CompPASS | Github | https://github.com/dnusinow/ |
| (version | | cRomppass/blob/master/R/ |
| 0.0.0.9000) | | comppass.R |
| SAINTexpress | Sourceforge | https://sourceforge.net/ |
| (version 3.6.1) | | projects/saint-apms/files/ |
| InstantClue | | http://www.instantclue. |
| | | unikoeln.de/ |

Out of 589 PPIs identified, 81% were not shared with other cell lines, reflecting high cell type-specificity of PPIs in different genetic contexts (FIG. 3E). It was speculated that differential protein abundance across cell lines might provide one explanation for cell type-specific PPIs. However, while some changes in interaction could be explained by changes in protein abundance, many cases were also found with the opposite behavior, in which a gain in interaction was observed with a concomitant decrease in protein abundance (FIG. 2D).

### Cell Type-Specific Interactions Reveal Novel Modulator of AKT

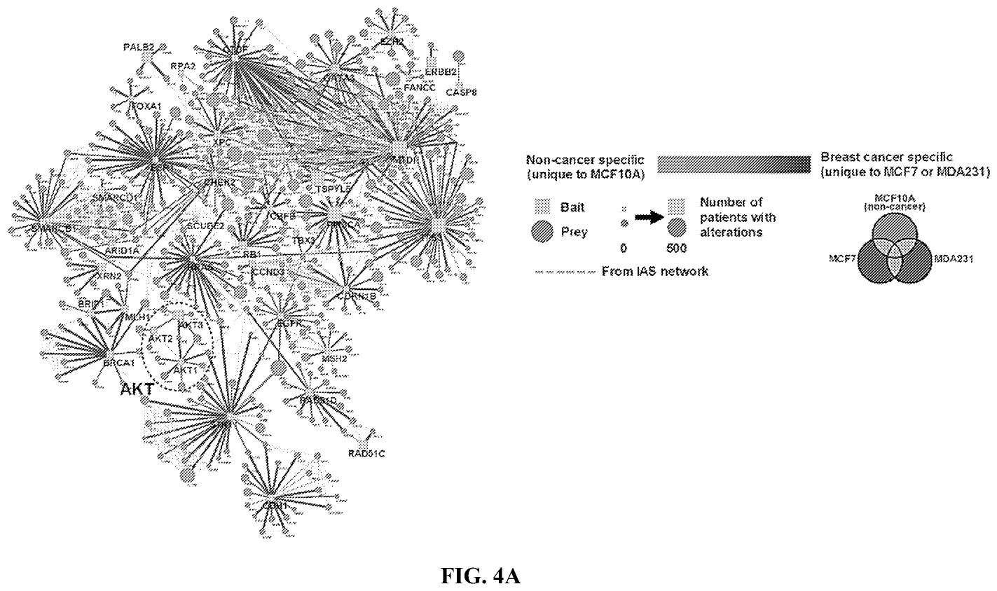

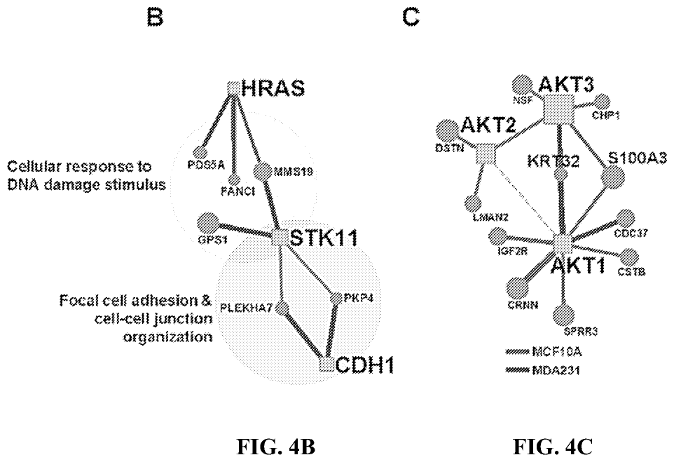

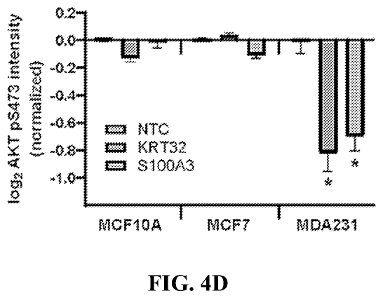

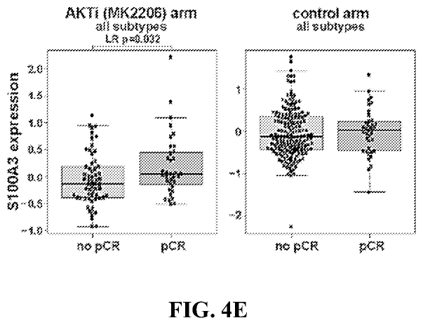

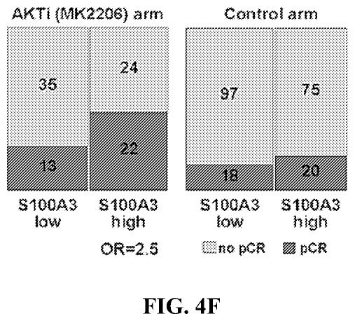

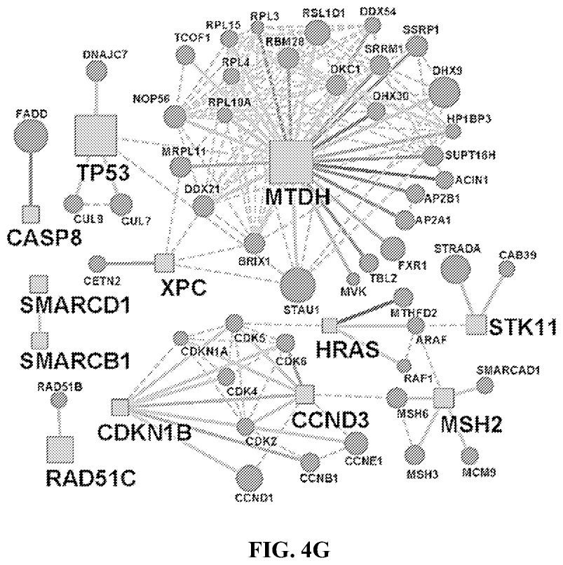

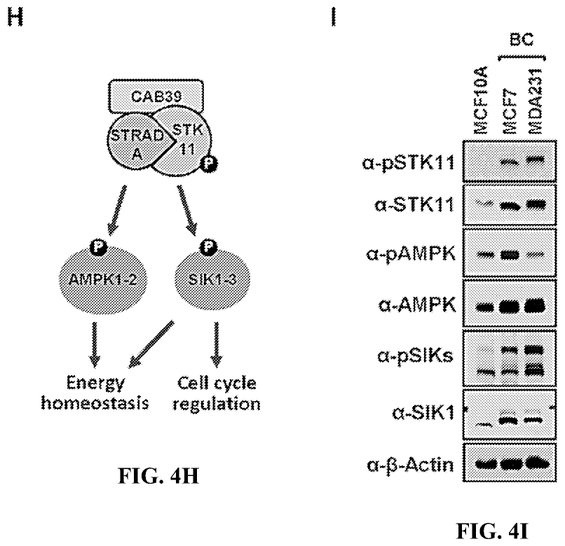

To compare PPIs across cell lines, a cancer-specific differential interaction score (DIS) was defined as the probability of the PPI being present in a cancer cell line (either MCF7 or MDA-MB-231) but absent in the normal cell line (MCF10A, Key Resources Table I). The results of this differential scoring analysis were used to visualize the entire BC PPI network showing PPIs that are (1) private to a cancer cell line, (2) private to non-cancerous MCF10A cells, or (3) conserved in the two cancer cell lines but absent in the non-cancerous context (FIG. 4A).

Referring to FIG. 4A, an interactome of the union of all high-confidence PPIs detected across all cell lines is shown. Edges are colored based on their differential interaction, with darker edges representing PPIs that are enriched to BC cell lines (unique to either MDA-MB-231 or MCF7) as compared to MCF10A cells (shown in teal edges). Dotted line represents the physical protein-protein association (validated in other studies) with high Integrated Association Stringency score. AKT subnetwork is outlined in a dotted circle.

Among interactions private to a cancer cell line, it was found that the HRAS proto-oncogene and the tumor suppressor kinase STK11 (also known as LKB1) interact with a set of DNA damage response (DDR) proteins (PDS5A, FANCI, MMS19, GPS1) in MCF7 and MDA-MB-231 cells, respectively (FIG. 4B). Given the previous observations that silencing of HRAS and STK11 lead to defective DNA repair and genome instability (Grabocka et al., 2014), these interactions may provide insights into direct effectors by which HRAS and STK11 modulate DDR. STK11 also interacted with cell adhesion factors in MCF10A cells (PLEKHA7 and PKP4, FIG. 4B), consistent with its role in cell autonomous polarization (Baas et al., 2004; Forcet et al., 2005; Zhang et al., 2008) and actin filament assembly at the cellular leading edge (Xu et al., 2010). Interestingly, CDH1 but not STK11 was found to interact with these same proteins in MDA-MB-231 cells. CDH1 plays critical roles as a master regulator of cell-cell adhesion via adherens junctions, cell polarity and cell migration (Brieher and Yap, 2013; van Roy and Berx, 2008), and abrogation of CDH1 expression is a hallmark of the epithelial-to-mesenchymal transition (Canel et al., 2013). The observed interaction patterns suggest that STK11 may contribute to cell polarity and focal adhesion via a physical interaction with PLEKHA7 and PKP4, but that it requires the cellular ability to form adherens junctions. This may explain the lack of interaction of STK11 with PLEKHA7 and PKP4 in MDA-MB-231, which do not normally express CDH1 due to promoter hypermethylation (Lombaerts et al., 2006; Tate et al., 2012).

Referring to FIG. 4B, PPIs connecting HRAS, STK11, and CDH1 are shown. HRAS and STK11 have several interactors including MMS19 in BC cells involved in cellular response to DNA damage stimulus. STK11 and CDH1 interact with PKP4 and PLEKHA7 in a cell type-specific manner, implying differential regulation of cell adhesion and cell-cell junction in non-BC and BC cells.

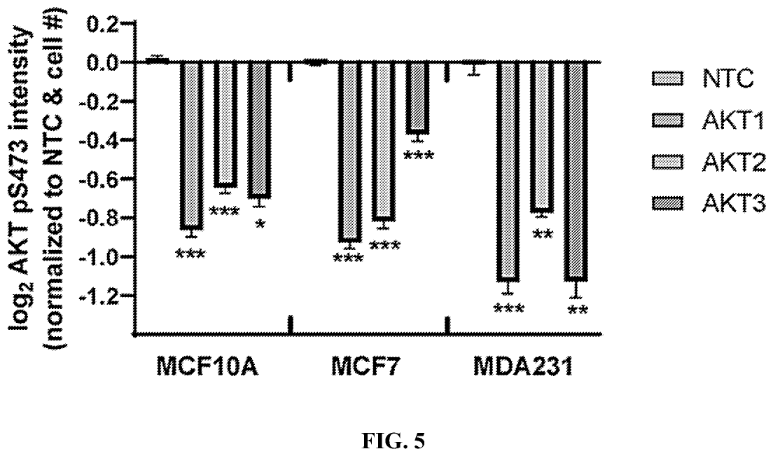

The cell-line specific analysis also revealed contextual interactions with AKT, the central signaling kinase frequently deregulated in BC and many other types of human cancers (Guerrero-Zotano et al., 2016; Manning and Cantley, 2007; Manning and Toker, 2017; Vivanco and Sawyers, 2002). In particular, AKT1 and its paralog AKT3 were both found to interact with S100 Calcium Binding Protein A3 (S100A3) and keratin KRT32, specifically in MDA-MB-231 cells (FIG. 4C). To probe the role of these proteins in regulating AKT kinase activities, small interfering RNA (siRNA)-mediated knockdown of S100A3 and KRT32 was performed, and phosphorylation of AKT at S473, a proxy of AKT activation (Alessi et al., 1996, 1997; Sarbassov et al., 2005; Stokoe et al., 1997), was monitored. Depletion of S100A3 and KRT32 significantly reduced normalized phospho-AKT (pAKT) levels in MDA-MB-231 but not in the other two cell lines (FIG. 4D). In contrast, knockdown of AKT1, AKT2, or AKT3 led to the expected reduction of pAKT in all three cell lines (FIG. 5). Without wishing to be bound by theory, these results indicate that S100A3 and KRT32 are functionally relevant, cell-type specific activators of the AKT pathway. One plausible explanation for the cell type-specificity of S100A3 is higher protein expression in MDA-MB-231 cells (>4-fold), a trend also observed in RNA-seq studies (Papatheodorou et al., 2020).

Referring to FIG. 4C, AKT (AKT1/2/3) subnetwork has many cell type-specific interactors. AKT1 and AKT3 interact with S100A3 and KRT32 in MDA-MB-231 cells.

Referring to FIG. 4D, small interfering RNA-mediated knockdown of S100A3 and KRT32 significantly reduces phospho-AKT (S473) level in MDA-MB-231 cells. pAKT level was normalized to non-targeting control (NTC) and cell numbers as well as total AKT level. * p-value<0.05.

Referring to FIG. 5, small interfering RNA-mediated knockdown of AKT1, AKT2, and AKT3 reduces phospho-AKT (S473) level in all three cell lines analyzed. pAKT (S473) intensity was normalized to non-targeting control (NTC) and cell numbers. *** pvalue<0.001, ** p-value<0.01, * p-value<0.05.

These results were also analyzed in the context of I-SPY 2, a neoadjuvant, adaptive clinical platform trial for high risk early stage breast cancer (Barker et al., 2009). It was found that patients who achieved pathologic complete response (pCR) to the pan-AKT allosteric inhibitor MK2206 (Chien et al., 2020) had pre-treatment tumors with significantly higher S100A3 mRNA expression than those of non-responding patients (p=0.03, FIG. 4E). In contrast, tumors in the control arm receiving only standard chemotherapy did not show any significant difference in S100A3 expression between responsive and non-responsive groups. Thus, high S100A3 expression may provide a biomarker of AKT pathway activation predictive of response to AKT-targeted therapy (Odds Ratio 2.5, FIG. 4F). As high S100A3 expression positively regulates AKT activity, such regulation may create a dependence on the AKT pathway akin to oncogene addiction.

Referring to FIG. 4E, a box plot shows that the patient group (enrolled in the I-SPY 2 clinical trial) with pathologic complete response (pCR) to MK2206 (pan-AKT inhibitor) had pre-treatment tumors with significantly higher S100A3 mRNA expression (Likelihood ratio (LR) p-value=0.032) than those of non-responding patients. In the control arm, there is no difference in S100A3 expression between pCR and no pCR groups.

Referring to FIG. 4F, a mosaic plot shows that BC patients who did pCR to MK2206 had 2.5 times more likely had higher mRNA expression of S100A3 in their pre-treatment tumors (Odds Ratio=2.5). In the control arm, there is no significant difference in pCR between low and high S100A3 expression groups. Numbers in each block represent the patient sample size. Column width indicates the relative proportion of the S100A3 low and high expression group on the patient population.

### Interactions Conserved Across BC Contexts

A number of PPIs were commonly observed in both MCF7 and MDA-MB-231 BC cells but not in a non-cancerous tissue context (FIG. 4G). Notably, this group of prey proteins was not as frequently mutated in BC as those distinct to one of the two cancer cell lines (FIG. 3E), although some clearly play central roles in cancer cell proliferation. For instance, protein interactions among cyclins (e.g., CCND3), cyclin-dependent kinases (CDKs 2,4,5,6) and CDK inhibitors (CDKN1B) were seen predominantly in BC but not normal cells. These interactions may reflect dysregulated activation of CDKs and uncontrolled cell cycle progression in BC (Malumbres and Barbacid, 2001; Santo et al., 2015), providing the rationale for CDK4/6-targeted therapy (Hamilton and Infante, 2016; Lim et al., 2016; Niu et al., 2019).

Referring to FIG. 4G, high-confidence PPIs that are commonly detected only in two cancer cell lines (MDA-MB-231 and MCF7) but not in non-cancerous MCF10A cells are shown. Node and edge styles and colors as seen in FIG. 4A.

It was also found that STK11 interacts with STRADA and CAB39 (also known as M025) preferentially in the two cancer cell lines (FIG. 4G). STRADA and CAB39 form a heterotrimeric complex with STK11 (Baas et al., 2003, 2004; Zeqiraj et al., 2009a) to properly position the activation loop of STK11 in an active conformation (Zeqiraj et al., 2009b), enabling STK11 to phosphorylate and activate downstream kinases, including AMP-activated protein kinases (AMPKs) and salt-inducible kinases (SIKs) involved in energy homeostasis and cell cycle regulation (FIG. 4H) (Alessi et al., 2006; Hardie et al., 2013; Hollstein et al., 2019). The increased associations of STK11 with STRADA and CAB39 suggested that STK11 activity is generally increased in cancer. Consistent with this speculation, it was found that both total and activated STK11 (phosphorylated at Ser428) are more abundant in MCF7 and MDA-MB-231 than in MCF10A cells (FIG. 4I). Furthermore, phosphorylation of STK11 downstream targets, including SIK1, SIK2, SIK3, and AMPK, was higher in the two BC cell lines (FIG. 4I). Increased STK11 activity may support cellular fitness by balancing energy production with anabolic metabolism, as previously seen in hepatocellular carcinoma (Lee et al., 2015).

Referring to FIG. 4H, STK11 forms a heterotrimeric complex with CAB39 and STRADA to activate its kinase activity and phosphorylate downstream kinases including AMPK and SIK for regulating energy homeostasis and cell cycle.

Referring to FIG. 4I, STK11 kinase activity was monitored by measuring total and phosphorylation levels of its known downstream substrates (AMPK and SIKs) as well as itself. The following phospho-epitopes were detected by antibodies: pSTK11 (pS428), pAMPKu (pT172), pSIKs [pSIK1 (pT182), pSIK2 (pT175), pSIK3 (pT163)].

### Comparative Network Analysis Between Wild-Type and Mutant Cancer Proteins

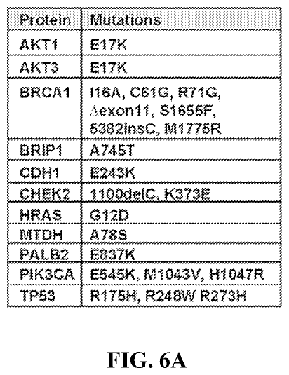

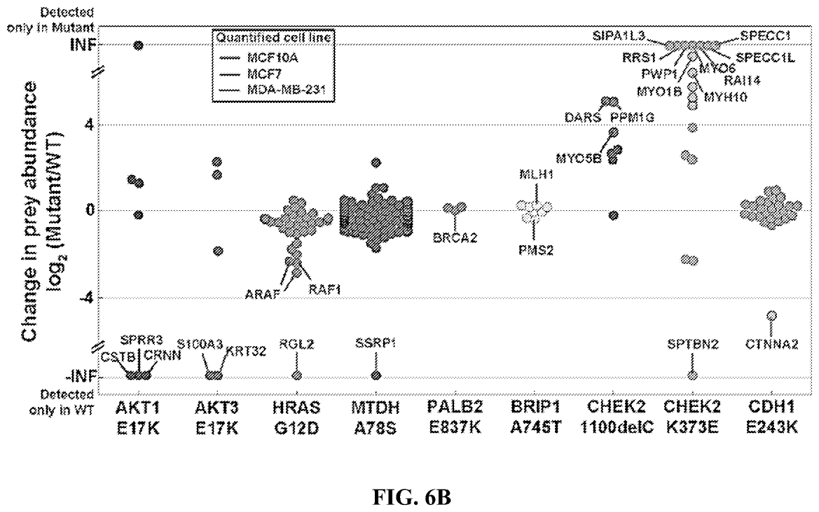

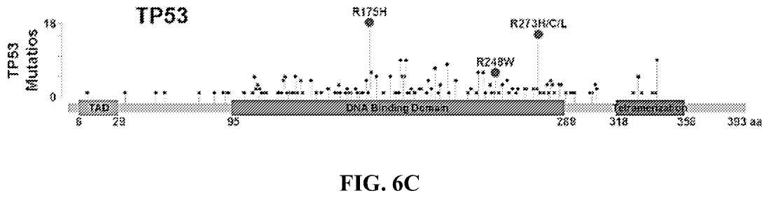

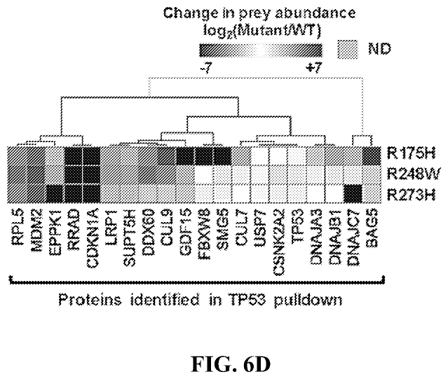

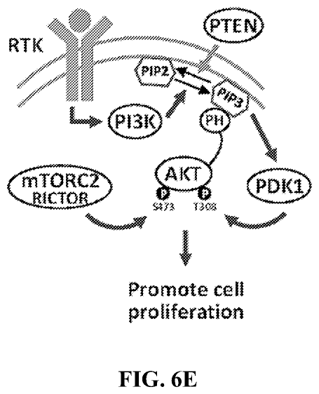

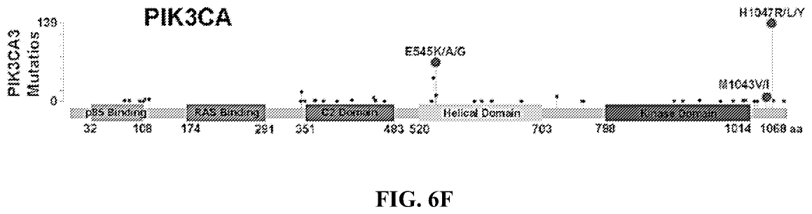

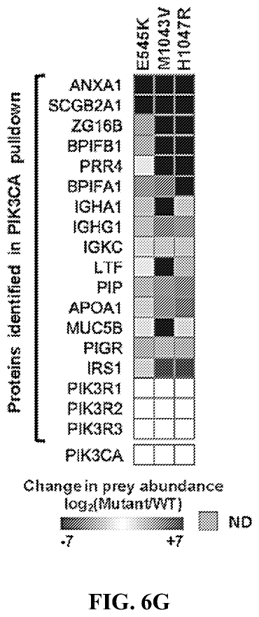

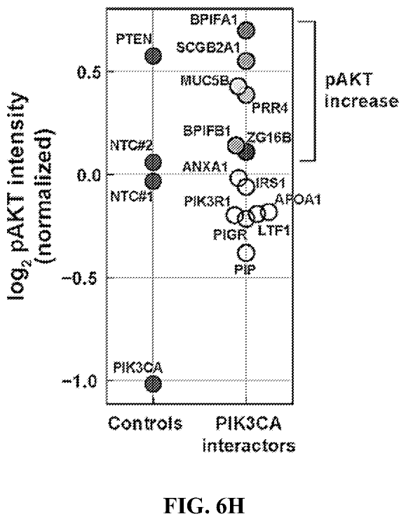

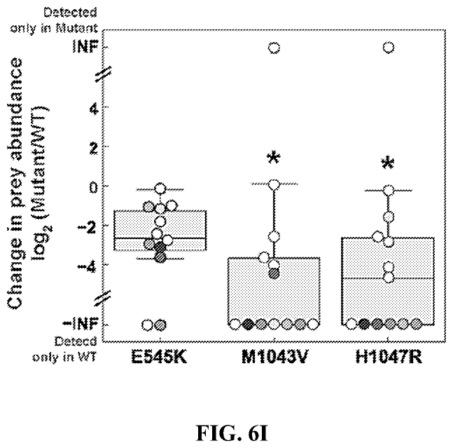

Many BC proteins are recurrently mutated in tumors, but how these mutations affect and re-wire PPIs has not been extensively analyzed. 11 proteins with frequent or known pathogenic mutations in BC were selected, and AP-MS was performed on both the WT and mutant isoforms to quantitatively measure changes in PPIs (FIG. 6A). The E17K mutation of AKT1 and AKT3 is an activating mutation that causes constitutive membrane association of AKT kinases (Carpten et al., 2007; Davies et al., 2008; Landgraf et al., 2008; Lindhurst et al., 2011; Rudolph et al., 2016). Intriguingly, while WT AKT1 was found to interact with CSTB, CRNN, and SPRR3, the AKT1 E17K mutant was not. Similarly, the aforementioned S100A3 and KRT32 exclusively interacted with WT AKT3 but not the E17K mutant (FIG. 6B). Without wishing to be bound by theory, these results indicate that a conformational change of AKT may be induced by the E17K mutation in the pleckstrin homology domain. Alternatively, the resulting constitutive membrane localization of AKT may significantly affect its pattern of PPIs. Overexpression of CRNN and SPRR3 has been noted to activate AKT (Cho et al., 2010; Li et al., 2019), similar to the roles of S100A3 and KRT32 (FIG. 4D). Without wishing to be bound by theory, the results here indicate these proteins activate the AKT pathway through direct interaction with AKT1 in a manner that is disrupted by AKT mutation.

Referring to FIG. 6A, proteins analyzed for both WT and mutant forms are listed.

Referring to FIG. 6B, changes in abundance of high-confidence preys between each mutant and the corresponding WT protein were quantified. Each dot represents an individual PPI. Highly differential PPIs between WT and mutant are annotated, with the line color representing the cell line from which that PPI was quantified.

The CHEK2 1100delC and K373E mutations are associated with cancer predisposition (Apostolou and Papasotiriou, 2017; Kumar and Bose, 2017), and both mutations disrupt CHEK2 kinase activity (Higashiguchi et al., 2016; Kumar and Bose, 2017). It was found that CHEK2 proteins with either of these mutations show a marked increase in abundance of their interacting preys (FIG. 6B), most of which participate in the actin cytoskeleton, including MYO5B, MYO6, MYH10, SIPA1L3 and SPECCI (Gene Ontology Biological Process GO:0015629, FDR=9.09×10-4). These two mutations may abrogate nuclear localization of CHEK2, allowing it to interact with cytoskeleton proteins in the cytoplasm. Interestingly, expression of the CHEK2 1100delC mutant induces cell migration and invasion in gastric cancer cells (Hong et al., 2017), suggesting that association of CHEK2 mutants with actin cytoskeleton proteins may contribute to cellular invasion and metastasis.

Due to the high prevalence of TP53 mutations in BC patients, three of the most common mutations were selected for quantitative AP-MS analysis (FIG. 6C). It was found that all three mutations abolished the interaction with CDKN1A and MDM2, as previously reported (Kim et al., 2017; Li et al., 2011; Schulz-Heddergott and Moll, 2018). In addition, it was found that these mutations disrupt the interaction with RRAD (FIG. 6D), a Ras-related small GTPase (Reynet and Kahn, 1993) associated with poor prognosis in lung and breast cancers when lowly expressed (Suzuki et al., 2007). Cancer cells, often under hypoxic conditions, preferentially utilize glycolysis (Cairns et al., 2011; Warburg, 1956), such that TP53 has been known to repress glycolysis for tumor suppression (Aylon and Oren, 2011; Feng and Levine, 2010; Gottlieb and Vousden, 2010). As one of the mechanisms of this suppression, TP53 transcriptionally activates RRAD, which in turn represses glycolysis through inhibition of GLUT1 translocation to the plasma membrane (Zhang et al., 2014). Without wishing to be bound by theory, these results suggest that TP53 may also regulate RRAD via direct protein interaction, and that the three oncogenic TP53 mutations abolish this regulatory function in BC cells. In addition to RRAD, several other PPIs were differentially affected by these TP53 mutations. For example, interactions with DnaJ/Hsp40 family proteins (DNAJA3, DNAJB1, and DNAJC7) and BAG5 were significantly increased in the R175H conformational mutant but not in the two DNA contact mutants (R248W and R273H) (FIG. 6D), consistent with the role of these proteins in protecting conformational TP53 mutants from degradation, thus promoting mutant TP53 accumulation (Parrales et al., 2016; Qi et al., 2014; Yue et al., 2016).

Referring to FIG. 6C, a lollipop plot representing the location of TP53 mutations and the number of BC patients bearing a given TP53 mutation from TCGA (Firehose Legacy) study is shown. TAD, transactivation domain; Tetramerization, tetramerization domain.

Referring to FIG. 6D, relative quantification of the abundance of high-confidence TP53 preys observed in MDA-MB-231 cells is shown. Preys detected only in WT are represented in deep blue while preys detected only in mutants are in deep red. All three TP53 mutants were expressed and detected at a similar level. The interaction of TP53 with CDKN1A and MDM2 are strongly diminished in all three pathogenic TP53 mutants tested as previously known. ND, not detected.

### Novel Regulators of PIK3CA Signaling

Activation of PIK3CA via receptor tyrosine kinase (RTK) or oncogenic mutations leads to membrane recruitment and activation of AKT (FIG. 6E) (Alessi et al. 1996; Alessi et al. 1997; Stokoe et al. 1997; Sarbassov et al. 2005). In BC, mutations at E545 and H1047 residues are most frequently found (FIG. 6F). Using AP-MS, 20 prey proteins that interact with PIK3CA were identified, 18 of which were observed only in MCF7 cells. These patterns may reflect activation of PI3K signaling due to PIK3CA E545K mutation, which is present in MCF7. Of the 18 proteins, only 4 (IRS1 and PIK3R1/2/3) were previously known interactors. Of note, it was found that many of these novel PPIs are significantly decreased, and in some cases completely abolished, by different PIK3CA mutations (FIG. 6G). To determine whether these PIK3CA interactors modulate the PI3K/AKT pathway, whether depletion of each target by siRNA affects downstream AKT activation was tested by measuring cellular phospho-AKT (pS473) levels in an in-cell western blot assay (Chen et al., 2005). Non-targeting control siRNAs (NTCs) as well as siRNAs targeting PIK3CA (a positive regulator) and PTEN (a negative regulator) were included as controls (Breuleux et al., 2009; Brognard et al., 2007; Cantley and Neel, 1999; He et al., 2008; Sarbassov et al., 2005; Uche and Kane, 2016). As expected, knockdown of PIK3CA in MCF7 cells significantly diminished pAKT signal, while knockdown of PTEN augmented it (FIG. 6H). Intriguingly, knockdown of the PIK3CA interactors BPIFA1 and SCGB2A1 (also named SPLUNC1 and Mammaglobin-B, respectively) increased pAKT activity to a degree higher than or the same as the PTEN knockdowns, suggesting that these two proteins are negative regulators of the PI3K/AKT pathway (FIG. 6H). Importantly, proteins for which knockdown increased pAKT (especially BPIFA1, BPIFB1, PRR4, and ZG16B) showed significantly reduced interactions with kinase domain mutants of PIK3CA (M1043V and H1047R) compared to a helical domain mutant (E545K) (FIG. 6I). Without wishing to be bound by theory, these results imply that mutations in the kinase domain may relieve PIK3CA from negative regulation by multiple factors.

Referring to FIG. 6E, an overview of the receptor tyrosine kinase (RTK)-PI3K signaling cascade leading to the phosphorylation (T308 and S473) and activation of AKT pathway is shown.

Referring to FIG. 6F, a lollipop plot representing the sites of PIK3CA mutations and the number of BC patients bearing a given PIK3CA mutation from TCGA (Firehose Legacy) study is shown.

Referring to FIG. 6G, relative quantification of the abundance of high-confidence preys observed from pull-down of PIK3CA (WT and mutants) in MCF7 cells is shown. Same color scheme as in FIG. 6D.

Referring to FIG. 6H, the level of AKT S463 phosphorylation (as proxy of activation) was measured upon siRNA-mediated knockdown of PIK3CA interacting preys and control genes (PTEN and PIK3CA). The intensity of AKT pS473 was normalized to non-targeting control (NTC), total AKT, and cell counts. Proteins whose knockdown increases pAKT are labeled in multiple colors.

Referring to FIG. 6I, changes in the abundance of PIK3CA interacting proteins from each PIK3CA mutant pull-down are represented as box plots, in which the proteins that increase pAKT upon knockdown are labeled as in H. Association of these proteins with M1043V and H1047R mutants is significantly abolished, compared to E545K mutant. * p-value<0.05.

### Effect of Pathogenic Mutations on the BRCA1 Interactome

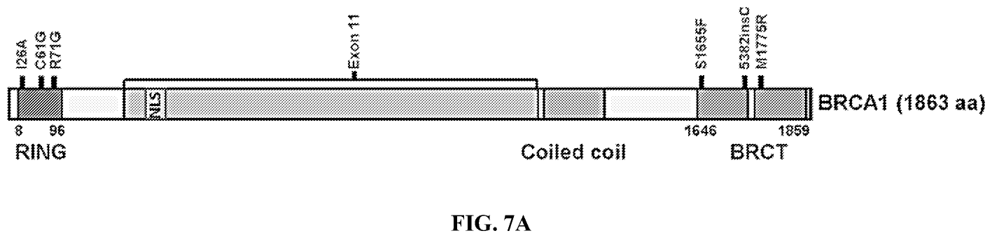

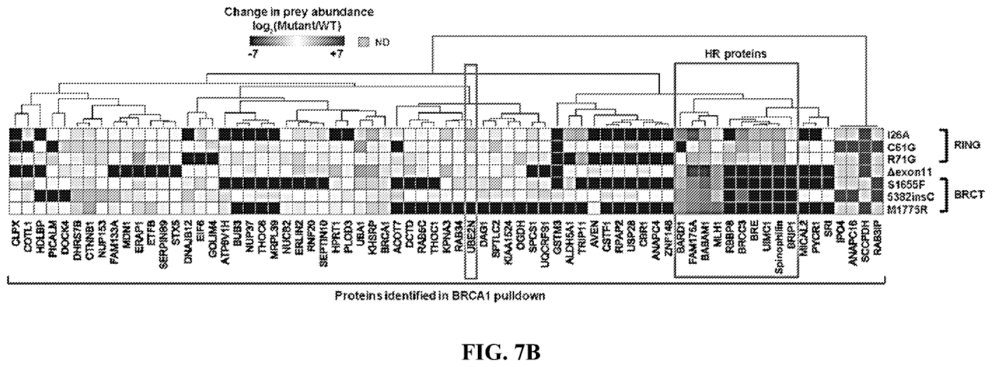

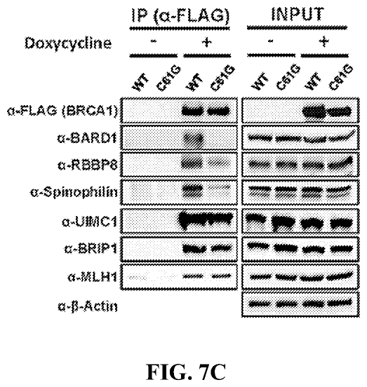

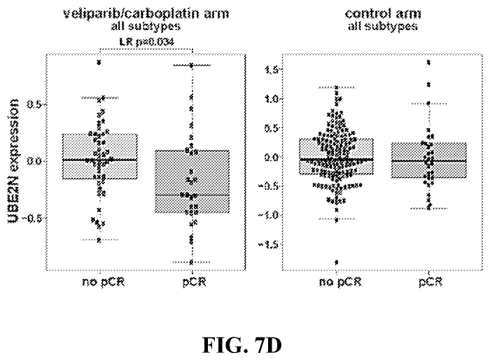

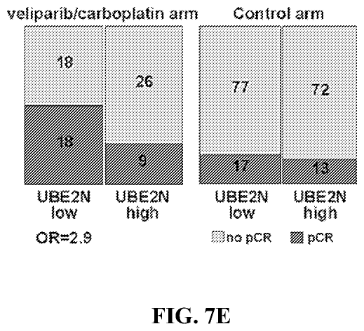

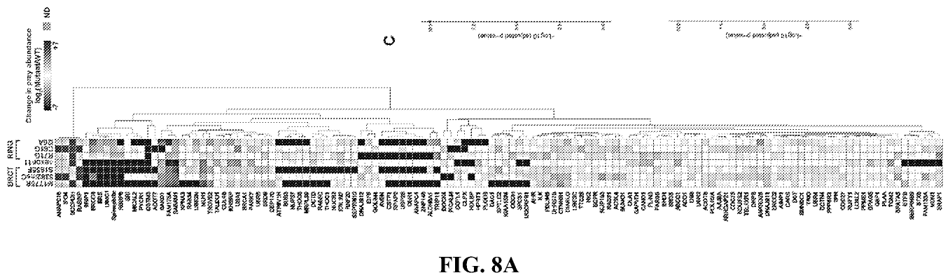

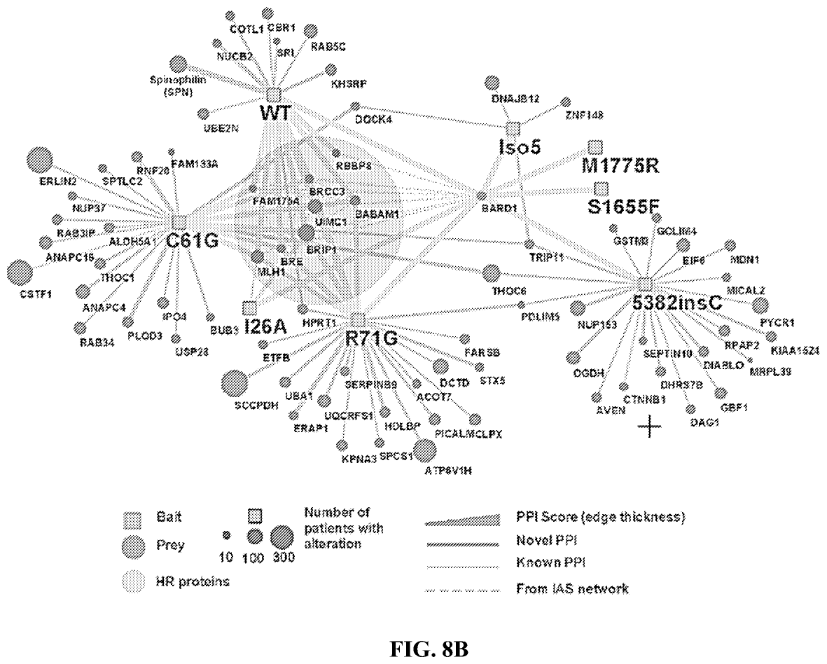

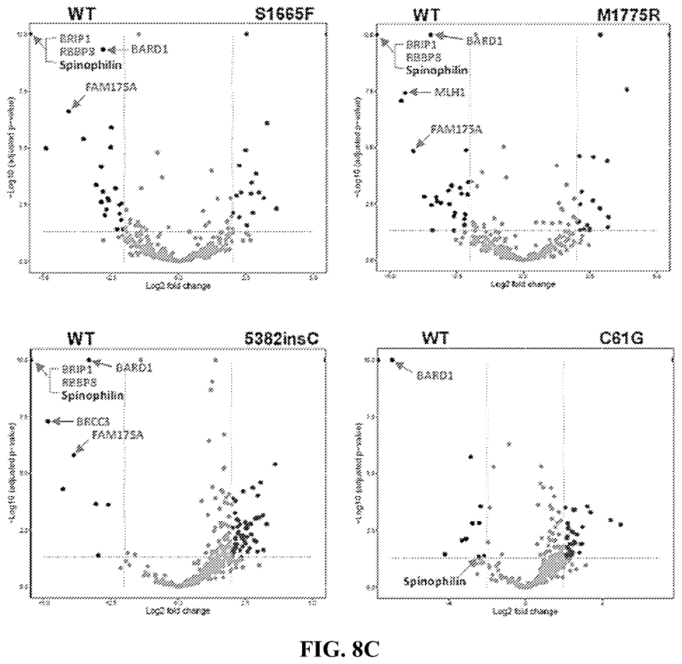

To comprehensively catalog the BRCA1 interactome and how pathogenic BRCA1 mutations alter these interaction profiles, AP-MS was performed on WT and pathogenic variants reported in cancer patients, including C61G and R71G in the N-terminal RING domain (Drost et al., 2011; Górski et al., 2000; Vega et al., 2001) and 51655F, 5382insC and M1775R in the C-terminal tandem BRCT domain (Anantha et al., 2017; Clapperton et al., 2004; Dever et al., 2011; Levy-Lahad et al., 1997) (FIG. 7A). Given that alternative splicing in cancer often generates BRCA1 isoforms lacking exon 11, which confers residual HR activity and therapeutic resistance (Wang et al., 2016a), this isoform was also included in the analysis. The I26A separation of function mutation in the RING domain, which abrogates E3 ubiquitin ligase activity but retains BARD1 binding, was also analyzed (Shakya et al., 2011). The expression of these BRCA1 proteins was induced in all three breast cell lines; however, only MDA-MB-231 cells (harboring the TP53 R280K mutation) supported ectopic 3×FLAG-BRCA1 expression. These observations were consistent with previous studies, which have shown that ectopic overexpression of BRCA1 (both WT and mutants) is not stably maintained without a compensatory TP53 mutation (Arizti et al., 2000; Buller et al., 2001; Holstege et al., 2009; McAllister and Wiseman, 2002). AP-MS experiments in MDAMB-231 cells identified 128 high-confidence interactions from 8 BRCA1 constructs (WT and 7 mutants, PPI score≥0.65, FIG. 8A); of these interacting proteins, 70 showed ≥8-fold change (FIG. 7B and FIG. 8B).

Referring to FIG. 7A, functional domains in the BRCA1 gene and the location of mutations analyzed by AP-MS are shown.

Referring to FIG. 7B, relative quantification of the abundance of prey proteins (PPI score≥0.65, >8-fold change) identified by BRCA1 AP-MS in MDA-MB-231 cells is shown. All prey abundance values were normalized by 3×FLAG-tagged BRCA1 levels in their respective AP-MS experiments. Preys detected only in WT are represented in deep blue while preys detected only in mutants are in deep red. A group of proteins involved in HR repair (boxed in green) are clustered together, wherein RING domain and BRCT domain BRCA1 mutants show distinct PPI abundance profiles. Spinophilin has not previously been known to have a function relevant to HR repair. UBE2N is boxed in sky blue. ND, not detected.

Referring to FIG. 8A, relative quantification of the abundance of prey proteins (PPI score≥0.65) identified by BRCA1 AP-MS in MDAMB-231 cells is shown. Preys detected only in WT are represented in deep blue while preys detected only in mutants are in deep red. ND, not detected.

Referring to FIG. 8B, PPIs across all BRCA1 proteins analyzed (WT and 7 mutants) are visualized in a network view. A selective set of prey proteins (PPI score≥0.65, ≥8-fold change) is shown. Proteins playing crucial roles in DNA repair by homologous recombination are circled in pink.

These data revealed a number of previously unidentified BRCA1-interacting proteins, along with known interactors, many of which were differentially affected by mutations in different domains of BRCA1. For example, HR proteins previously known to interact with BRCA1 (including BRIP1, RBBP8, and UIMC1) (Clapperton et al., 2004; Kim et al., 2007b; Sobhian et al., 2007; Yu and Chen, 2004) had a similar pattern of interaction loss (boxed in green in FIG. 7B) associated with BRCT domain mutants (S1655F, 5382insC, M1775R), whereas RING domain mutants (I26A, C61G, R71G) maintained these interactions. In a separate pattern, it was found that the C61G RING domain mutant abolishes interaction with BARD1 (FIG. 7B), as previously reported (Nelson and Holt, 2010; Wu et al., 1996). Several interactions could be confirmed by co-IP/western blot analysis (FIG. 7C). Without wishing to be bound by theory, these results suggest that RING domain mutants are hypomorphic and may retain some BRCA1 functions, which could explain at least in part why the BRCA1 C61G variant is only moderately sensitive to cisplatin and poly (ADP-ribose) polymerase (PARP) inhibitors and becomes readily resistant to these drugs (Drost et al., 2016; Wang et al., 2016b).

Referring to FIG. 7C, PPIs of selected proteins with BRCA1 (WT or C61G mutant) were confirmed by co-immunoprecipitation with antiFLAG antibody followed by western blot analysis.

A ubiquitin E2-conjugating enzyme, UBE2N (also known as UBC13), was found to interact with WT BRCA1, but to a lesser degree with mutant forms of BRCA1 (PPI score<0.6) (boxed in sky blue in FIG. 7B). For example, consistent with reports from yeast two-hybrid studies (Christensen et al., 2007), a six-fold reduction in UBE2N associated with the I26A mutant compared to WT was found, suggesting that UBE2N interacts with BRCA1 through the RING domain. Notably, the M1775R BRCT domain mutation also dramatically reduced the interaction with UBE2N (FIG. 7B), suggesting that M1775 residue in the BRCTs domain may also contribute to the interaction with UBE2N, although the underlying mechanism is currently unclear. Depletion of UBE2N shows HR defects including altered RAD51 filament formation and E3 Ub ligase function of BRCA1 (Zhao et al., 2007), indicating a critical role of UBE2N in HR repair.

Consistent with the cell line models, it was found that baseline UBE2N mRNA expression was significantly lower in I-SPY 2 BC patients who achieved pCR to the PARP-inhibitor (veliparib)/carboplatin (Rugo et al., 2016) in comparison to non-responsive patients (p=0.034, FIG. 7D). In contrast, BC tumors in the control arm did not show any significant difference in UBE2N expression between pCR and no pCR groups. Without wishing to be bound by theory, these results indicate that expression of UBE2N may serve as a biomarker of response to PARP inhibitors and other DNA repair targeted therapies (Odds Ratio=2.9, FIG. 7E).

Referring to FIG. 7D, a box plot shows that the patient group (enrolled in the I-SPY 2 clinical trial) with pCR to veliparib (PARP inhibitor) and carboplatin (VC) had pre-treatment tumors with significantly lower UBE2N mRNA expression (LR p-value=0.034) than those of non-responding patients. In contrast, BC patient tumors in the control arm did not show any significant difference in UBE2N expression between pCR and no pCR groups.

Referring to FIG. 7E, a mosaic plot shows that BC patients who did pCR to VC in addition to standard chemotherapy had 2.9 times more likely had lower mRNA expression of UBE2N in their pre-treatment tumors (Odds Ratio=2.9). In the control arm, there is no significant difference in pCR between low and high UBE2N expression groups. Numbers in each block represent the patient sample size. Column width indicates the relative proportion of the UBE2N low and high expression group on the patient population

### Spinophilin is a Novel BRCA1-Interacting Protein

Another protein interacting with BRCA1 in a mutation-dependent manner was Spinophilin (encoded by PPP1R9B), a known neuronal scaffolding protein that regulates synaptic transmission through its ability to target protein phosphatase 1 (PP1) to dendritic spines where it inactivates glutamate receptors (Allen et al., 1997; Feng et al., 2000; Sarrouilhe et al., 2006). Binding of Spinophilin to BRCA1 was unanticipated, and it was abolished by BRCT domain mutations similar to the pattern observed earlier for HR proteins (FIG. 7B). MSstats analysis of differential interactions between BRCA1 wild-type and BRCT domain mutants demonstrated that an intact BRCT domain is required for the BRCA1-Spinophilin interaction (FIG. 8C). Such recognition of the BRCT domain has been previously reported for BRCA1 interactions with FAM175A, BRIP1, and RBBP8 (Clapperton et al., 2004; Leung and Glover, 2011; Wu et al., 2016; Yu and Chen, 2004). Spinophilin was previously observed but unexplored in a systematic analysis of proteins interacting with the BRCT domain of BRCA1 (Woods et al., 2012).

Referring to FIG. 8C, volcano plots show proteins that differentially interact with BRCA1 between WT and mutants. The BRCT domain mutations tested (S1655F, M1775R, 5382insC) completely abolish the interaction of BRCA1 with Spinophilin as well as BRIP1 and RBBP8, while C61G mutation in the RING domain abrogates the interaction with BARD1. Colored data points indicate proteins that have ≤4-fold difference in protein interaction between WT and mutants and an adjusted p-value<0.05.

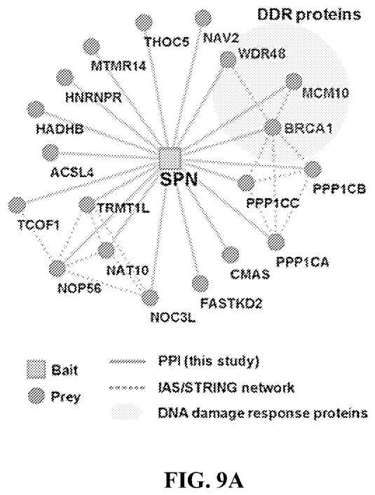

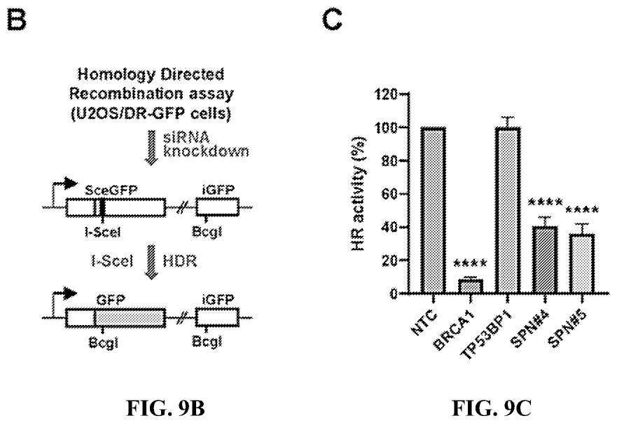

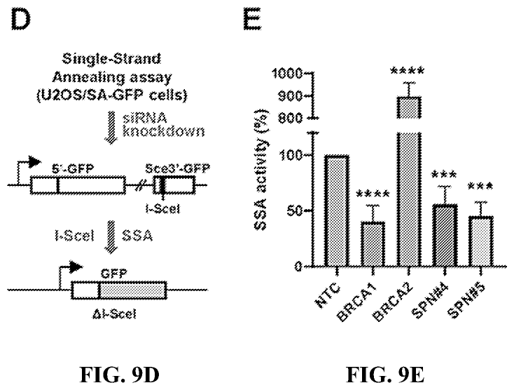

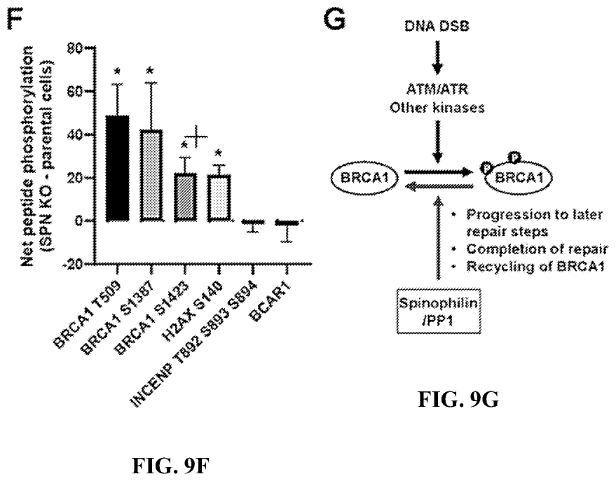

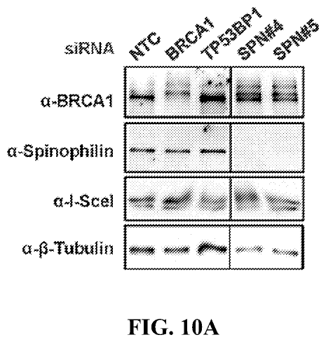

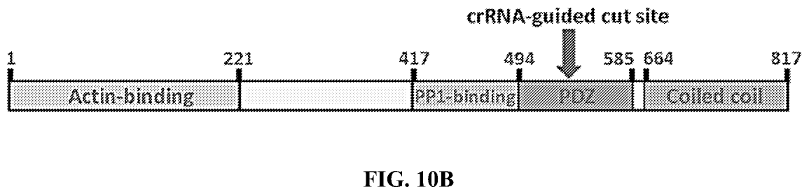

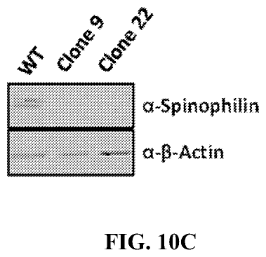

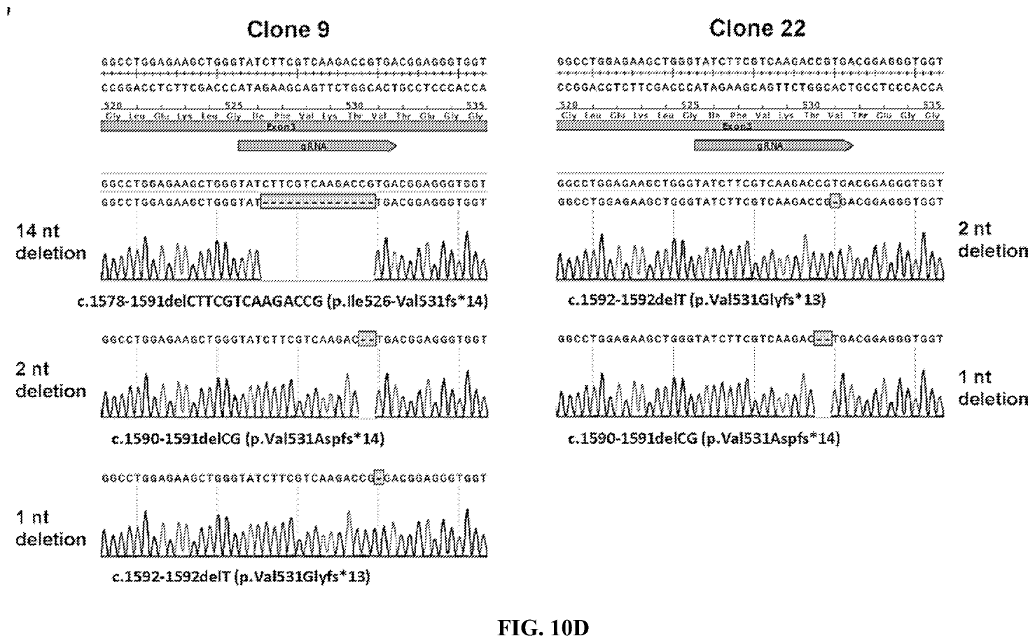

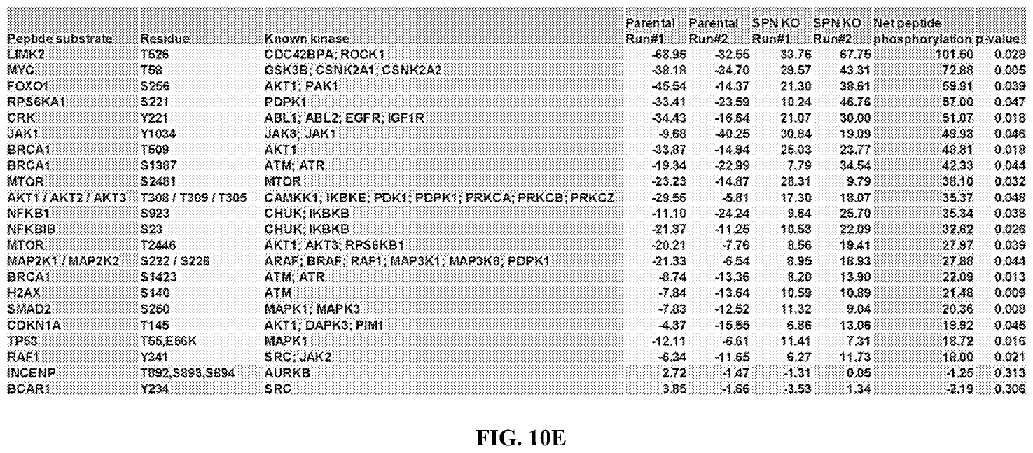

Reciprocal AP-MS was performed using 3×FLAG-tagged Spinophilin in MDA-MB-231 cells, which confirmed the interaction of Spinophilin with BRCA1 as well as with PP1 catalytic subunits (PPP1CA, PPP1CB, PPP1CC) (FIG. 9A). It was also found that Spinophilin interacts with additional proteins involved in DNA repair including WDR48 and MCM10. Without wishing to be bound by theory, these AP-MS results suggest that Spinophilin may participate in and/or regulate DNA repair by interacting with various DNA repair and replication proteins, including BRCA1. To explore this hypothesis, the effect of Spinophilin knockdown on DNA repair was analyzed by HR and single-strand annealing (SSA). In these assays, DNA Double Strand Breaks (DSBs) were induced by I-SceI endonuclease, which cleaves non-functional GFP cassettes engineered in the genome of U2OS reporter cell lines (DR-GFP and SAGFP) (Bhargava et al., 2018; Gunn and Stark, 2012). DSB repair that depends on the HR or SSA mechanism restores a functional GFP gene, yielding a readout tied to fluorescent signal intensity. Upon Spinophilin knockdown, HR activity was significantly reduced compared to NTC siRNA (FIG. 9B-C and FIG. 10A). In the same assay, knockdown of BRCA1 greatly decreased HR as expected (Anantha et al., 2017), while depletion of a protein functioning in an alternative DNA repair pathway did not (TP53BP1, nonhomologous end joining). Similarly, knockdown of Spinophilin significantly reduced SSA activity, comparable to BRCA1 depletion, while BRCA2 depletion dramatically increased SSA as seen previously (Anantha et al., 2017) (FIG. 9D-E), implying that Spinophilin promotes both HR and SSA-mediated DSB repair.

Referring to FIG. 9A, AP-MS of 3×FLAG-tagged Spinophilin (SPN, encoded by PPP1R9B) identifies BRCA1 (highlighted in a red edge) and other DDR-related proteins as well as PP1 catalytic subunits (PPP1CA, PPP1CB, and PPP1CC) in MDA-MB231 cells.

Referring to FIG. 9B, a schematic of the HR reporter assay is shown. The DR-GFP reporter contains two defective copies of the GFP gene, one disrupted by an I-SceI site and the other lacking a promoter. I-SceI cutting of the first copy generates a DSB, and repair by HR with the second copy as a template leads to restoration of a functional GFP gene. siRNA-mediated knockdown of HR-related genes leads to reduction of GFP+cells (a proxy of HR activity) compared to NTC experiments.

Referring to FIG. 9C, HR activities upon depletion of SPN relative to NTC (set to 100%). Depletion of BRCA1 and TP53BP1 was included and analyzed as controls. Data shown are the means from six independent experiments for each siRNA. Error bars represent standard deviations (SDs). **** p-value<1.0×10⁻⁵.

Referring to FIG. 9D, a schematic of the SA-GFP reporter assay is shown. The SA-GFP reporter contains a 5′-fragment of GFP (5′-GFP) and a 3′-fragment of GFP (Sce3′-GFP) that contains an I-SceI site. Repair of the DSB in Sce3′-GFP using 266 nt homology by single-strand annealing (SSA) restores a functional GFP gene.

Referring to FIG. 9E, SSA activities upon depletion of SPN relative to NTC (set to 100%) are shown. Depletion of BRCA1 and BRCA2 was included and analyzed as controls. Data shown are the means±SDs from six independent experiments for each siRNA. **** p-value<1.0×10⁻⁴, *** p-value<1.0×10⁻³.

Referring to FIG. 10A, levels of BRCA1, SPN, I-SceI and β-tubulin proteins in U2OS/DR-GFP cells following knockdown were analyzed by western blot.

Because Spinophilin is a regulatory subunit of PP1, it was hypothesized that it targets PP1 to specific DNA repair proteins, including BRCA1, for dephosphorylation. To uncover potential dephosphorylation targets under this model, a high-throughput peptide phosphorylation assay platform was used (Coppé et al., 2019a; Coppe et al., 2019; Coppé et al., 2019b). This system utilizes a collection of peptide sequences derived from biological targets of multiple kinases, which serves as phosphorylatable probes in a large-scale ATP-consumption assay (Chen and Coppé, 2012; Olow et al., 2016). In this assay, changes in phosphorylation (i.e., ATPconsumption) of peptide substrates derived from various proteins, including BRCA1 and the DSB-associated histone H2AX as well as proteins unrelated to DNA repair (e.g., INCENP, BCAR1), were measured in Spinophilin-disrupted (FIG. 10B-D) and parental cells (Key Resources Table I). Without wishing to be bound by theory, it was found that BRCA1 residues at T509, S1387, and S1423, as well as H2AX at S140 (γ-H2AX), were significantly increased in phosphorylation in Spinophilin-disrupted cell lysates compared to lysates from parental cells, and, in fact, were among the top 20 most increased sites (FIG. 9F and FIG. 10E). These results were in contrast to phosphorylation of the INCENP and BCAR1 peptides, which were not significantly changed by Spinophilin disruption. BRCA1 pT509 enhances nuclear localization and transcriptional activity of BRCA1 (Hinton et al., 2007), and pS1387 and pS1423 sites in the BRCA1 SQ-cluster region are critical for HR repair and cell-cycle checkpoint functions (Beckta et al., 2015; Cortez et al., 1999; Xu et al., 2002). γ-H2AX is a hallmark of DNA DSB (Rogakou et al., 1998) and initiates a signaling cascade to recruit various DSB repair proteins to properly repair the DNA damage (Lukas et al., 2011). Without wishing to be bound by theory, these results suggest that Spinophilin regulates BRCA1 and DDR signaling via dephosphorylation. Spinophilin knockdown may induce persistent phosphorylation of BRCA1 (and likely other DDR proteins, including H2AX), which could inhibit proper progression to subsequent repair or resetting activated DDR signaling to the initial homeostatic state following repair of damaged DNA (FIG. 9G). In agreement with these results, persistent γ-H2AX due to depletion of phosphatase PP2A has been reported to result in inefficient DSB repair (Chowdhury et al., 2005).

Referring to FIG. 9F, selective peptides derived from BRCA1 and H2AX as well as two non-DNA repair proteins (INCENP and BCAR1) were individually mixed with lysates from either SPN KO or parental cells and subsequently monitored for phosphorylation by measuring ATP consumption. Net peptide phosphorylation (difference between SPN KO and parental cells) was calculated from two independent runs as shown in the y-axis. Error bars represent standard deviations (SDs). * p-value<0.05, ** p-value<0.01.

Referring to FIG. 9G, a model for the role of SPN in regulating DDR is shown.

Referring to FIG. 10B, functional domains/motifs in the PPP1R9B gene and the location of CRISPR/Cas9-mediated cut site to introduce INDELS to generate knockout (KO) clones are shown.

Referring to FIG. 10C, confirmation of knockout by western blot analysis using u-Spinophilin (SPN) antibody is shown.

Referring to FIG. 10D, exome sequencing verified disruption of PPP1R9B alleles in two knockout clones (#9 and #22).

Referring to FIG. 10E, top 20 most increased phosphosites in SPN KO cell lysates, compared to parental MDA-MB-231 cell lysates are shown. Net phosphorylation of the peptides derived from 237 proteins including BRCA1 and H2AX was calculated based on ATP consumption between SPN KO and parental cell lysates. Peptides from INCENP and BCAR1 are shown as unaffected controls. Kinase(s) known to phosphorylate each phosphosite is also listed.

### Discussion

Here, comprehensive interaction maps were generation for 40 frequently altered breast cancer proteins. These data represent the first large-scale study of biophysical interactions in breast cancer and across multiple cell lines of human breast tissue origin, providing a useful and relevant PPI resource to study breast cancer biology. Prey proteins private to either of the two BC cell lines are more frequently mutated in breast tumors than preys from non-tumorigenic cells (FIG. 3E), implying that proteins interacting with cancer drivers may also contribute to the onset of cancer.

Approximately 79% of PPIs identified have not been previously reported (FIG. 3C), and 81% are not shared across cell lines (FIG. 3E). These findings illustrate a significant rewiring of PPIs driven by different cellular contexts, as recently seen by another large-scale AP-MS study (Huttlin et al., 2020). Without wishing to be bound by theory, these results suggest that protein abundance in a cell line is not the sole mechanism for PPI specificity (FIG. 2D). Presumably other features, such as differential posttranslational modifications (PTM), cellular compartmentalization, and/or mutational status of proteins may contribute to cell type-specificity.

S100A3 activates AKT signaling via protein interaction in MDA-MB-231 cells (FIG. 4C-D). Consistently, ≤4-fold higher protein abundance of S100A3 was observed in MDA-MB-231 cells than the other two cell lines, and it is known that chromosome 1q21.3 containing the S100A3 gene is amplified in all BC subtypes but with a higher percentage in TN breast tumors like MDA-MB-231 (31% compared to 10-12% for luminal and HER2+) (Goh et al., 2017). Consistent with these findings, higher S100A3 expression was predictive of a successful clinical response to an AKT inhibitor among BC patients in the I-SPY 2 trial (FIG. 4E). Without wishing to be bound by theory, these results suggest that S100A3 is an oncogenic driver activating the AKT signaling pathway, preferentially in TN tumors, and holds potential as a biomarker to segregate patients for AKT-directed therapy. Novel interactors of PIK3CA that negatively regulate the PI3K/AKT pathway were also identified (FIG. 6H). BPIFA1 is a lipid-binding protein with antimicrobial and immunomodulatory functions (Britto and Cohn, 2015; Ning et al., 2014). It is significantly downregulated in nasopharyngeal carcinoma (Bingle and Bingle, 2011; Lemaire et al., 2003; Zhang et al., 2003) and its single-nucleotide polymorphisms are associated with increased susceptibility to this tumor type (He et al., 2005). The BPIFA1-PIK3CA interaction identified suggests that BPIFA1 may directly modulate PI3K-AKT via PPI. BPIFA1 also increases the expression of PTEN via downregulating the miR-141 oncogene (Chen et al., 2013), thus it is also possible that knockdown of BPIFA1 may indirectly activate PI3K/AKT.

Another PIK3CA interactor, SCGB2A1, is a small secreted protein highly differentially expressed in multiple types of cancer including ovary, endometrium, and breast (Aihara et al., 1999; Bellone et al., 2013; Tassi et al., 2008). Previous studies have shown that SCGB2A1 is expressed at lower levels in luminal breast cancer compared to histologically normal breast epithelium (Zubor et al., 2015), and that overexpression of SCGB2A1 inhibits the viability of luminal BC cell lines with activated PI3K (including MCF7) via induction of apoptosis (Zhang et al., 2020). Provided that SCGB2A1 acts as a negative regulator of the PI3K-AKT pathway, elevated expression of SCGB2A1 may lead to inhibition of PI3K activity on which cell viability relies. Intriguingly, mutations in the PIK3CA kinase domain (M1043V and H1047R) significantly abolished the interaction with most of the other negative regulators identified (including BPIFA1, BPIFB1, PRR4, and ZG16B but not SCGB2A1), while these interactions were not severely affected by a helical domain mutation (E545K) (FIG. 6I). Considering that one of the steps in PIK3CA activation involves a conformational change in the kinase domain, which promotes PIK3CA membrane localization (Burke et al., 2012; Liu et al., 2014), these negative regulators may bind to the kinase domain and/or prevent the conformational change. Presumably, kinase domain mutants (e.g., H1047R) that become activated by mimicking this step may be refractory to negative regulation by these proteins. Alternatively, given that BPIFA1/B1 binds membrane lipids via the bactericidal/permeability-increasing fold (BPI) (Alva and Lupas, 2016; Beamer, 2003), some of these proteins may block the membrane localization of the PIK3CA kinase domain by interfering with its lipid binding.

In an effort to comprehensively identify BRCA1-interacting proteins in breast cancer cells, several novel interacting proteins were found, including Spinophilin, which acts as a regulatory subunit of PP1 (FIG. 7B-C and FIG. 9A). Knockdown of Spinophilin led to significant impairment in DSB repair by both HR and SSA pathways (FIG. 9C and FIG. 9E), establishing that this protein has a defined role in DNA repair. Consistent with its BRCA1 association, Spinophilin knockout revealed a net increase in phosphorylation on several phosphosites (pT509, pS1387, pS1423) (FIG. 9F), all of which are known to potentiate the DNA repair function of BRCA1 (Beckta et al., 2015; Cortez et al., 1999; Hinton et al., 2007; Xu et al., 2002). These residues are phosphorylated by AKT (T509) (Altiok et al., 1999; Nelson et al., 2010) and ATM/ATR (S1387, S1423) (Cortez et al., 1999; Gatei et al., 2000, 2001; Kim et al., 1999) but the opposing phosphatase(s) has not been identified. These results indicate that Spinophilin may dephosphorylate these residues and thus modulate BRCA1 functions via direct interaction (FIG. 9G).

A related intriguing question is how depletion of Spinophilin decreases HR and SSA repair activity. One plausible explanation is that prolonged phosphorylation of BRCA1 (and likely other DDR proteins as well) is inhibitory to multiple steps during DNA repair, including DSB-end resection, which is a prerequisite for HR and SSA. In agreement with this hypothesis, continuous DNA damage signaling and phosphorylation of several DDR proteins (including H2AX, NBN, RPA2, and CHEK2) induced by short double-stranded DNA molecules (mimicking DNA DSB) was shown to disorganize the cellular DNA repair system and inhibit DSB repair (Quanz et al., 2009). Alternatively, but not exclusively, Spinophilin may play a role in initiating the DSB repair process by removing constitutive phosphorylations that inhibit the function of DDR proteins. Supporting this scenario, a phosphoproteomic study revealed that over one-third of the captured phospho-peptides were dephosphorylated within minutes of DNA damage (Bensimon et al., 2010). Additionally, Spinophilin may be involved in counteracting DSB-induced phosphorylation events, thus promoting recycling of DDR proteins as DNA damage is being repaired. Given that somatic alterations to Spinophilin are more frequent in breast cancer than alterations to BRCA1 (approximately 8% versus 2%, respectively) (Cancer Genome Atlas, Network, 2012), this protein may be worthy of further study as a significant cancer-associated gene in DSB repair.

In summary, this study demonstrates that systematic PPI maps effectively identify new cancer susceptibility genes and recognize new druggable vulnerabilities in breast cancer. These maps provide a useful resource in contextualizing uncharacterized mutations within signaling pathways and protein complexes. Further analysis of genetic and functional interactions (gene-gene, gene-drug) of proteins in the map will help to decode their biological mechanisms and guide the development of cancer treatment strategies.

### Example 2. A Protein Network Map of Head and Neck Cancer Reveals PIK3CA Mutant Drug Sensitivity

### Experimental Methods

### Bait Cloning

Baits were cloned using the Gateway Cloning System (Life Technologies) into a doxycycline-inducible N-term or C-term 3×FLAG-Tagged vector modified to be Gateway compatible from the pLVX-Puro vector (Clontech) by the Krogan lab. Point mutant baits were cloned via site-directed mutagenesis. All expression vectors were sequence validated (Genewiz).

### Cell-Culture, Lentivirus Production, and Stable Cell Line Generation

HEK293T (ATCC, CRL-3216) and CAL-33 were maintained in DMEM (Corning) supplemented with 10% FBS (Gibco) and 1% Penicillin-Streptomycin (Corning). HET-1A was maintained in BEGM™ (Lonza), consisting of Broncho Epithelial Basal medium (BEBM) with the additives of the Bullet kit except GA-1000 (gentamycin-amphotericin B mix). SCC-25 was maintained in DMEM/F12 (Corning) with 10% FBS (Gibco), 1% Penicillin-Streptomycin (Corning) and 400 ng/mL hydrocortisone (Sigma). HET-1A was obtained from American Type Culture Collection and SCC-25 was obtained from Thomas Carey (University of Michigan), CAL-33 were provided by Gerard Milano (University of Nice, Nice, France). All cells were maintained in a humidified 37° C. incubator with 5% CO₂. Stably transduced HET-1A, SCC-25, and CAL-33 cell lines were maintained in puromycin (2 μg/mL, 2.5 μg/mL, and 0.7 μg/mL, respectively). Bait expression was induced by 1 vμg/ml doxycycline for 40 hrs. All cell lines were authenticated by the University of California, Berkeley Cell Culture Facility. Lentivirus was produced for each bait by packaging 5ug bait vector, 3.33 μg of Gag-Pol-Tat-Rev packaging vector (pJH045 from Judd Hultquist), 1.66 μg of VSV-G packaging vector (pJH046 from Judd Hultquist) with 30 μL of PolyJet (SignaGen). After incubating at room temperature for 25 min, DNA complexes were added dropwise to HEK293T cells (15 cm plate, ˜80% confluency). Lentivirus-containing supernatant was collected after 72 hrs and filtered through a 0.45 μm PVDF filter. Lentivirus particles were precipitated with PEG-6000 (8.5% final) and NaCl (0.3 M final) at 4° C. for 4-8 hrs. Particles were pelleted via centrifugation at 2,851×g for 20 min at 4° C. and resuspended in DPBS for a final volume −800-1000 μL. Stable cell lines were generated by transducing a 10 cm plate (˜80% confluency) with 200 uL of precipitated lentivirus for 24 hrs before selecting with puromycin for a minimum of 2 days.

### Affinity Purification

One 10 cm plate of cells (˜80% confluency) was washed with ice-cold DPBS and lysed with 300 μL of ice-cold lysis buffer (50 mM Tris pH 7.4, 150 mM NaCl, 1 mM EDTA, 0.5% NP40, 1 mM DTT, 1× protease inhibitor cocktail (Roche, complete mini EDTA free), 125U Benzonase/mL). Lysates were flash-frozen on dry ice for 5-10 min, followed by a 30-45 s thaw in 37° C. water bath with agitation, and rotation at 4° C. for 15 min. Lysate was clarified by centrifugation at 13000×g for 15 min at 4° C. A 30 μL lysate aliquot was saved for future BCA assay and western blot.

For FLAG purification, 25 μL of bead slurry (Anti-Flag M2 Magnetic Beads, Sigma) was washed twice with 1 mL of ice-cold wash buffer (50 mM Tris pH 7.4, 150 mM NaCl, 1 mM EDTA) and all of the remaining lysate was incubated with the anti-FLAG beads at 4° C. with rotation for 2 hrs. After incubation, flow-through was removed and beads were washed once with 500 μL of wash buffer with 0.05% NP40 and twice with 1 mL of wash buffer (no NP40). Bound proteins were eluted by incubating beads with 15 μL of 100 ug/ml 3×FLAG peptide in 0.05% RapiGest in wash buffer for 15 min at RT with shaking. Supernatants were removed and elution was repeated. Eluates were combined and 10 μL of 8 M urea, 250 mM Tris, 5 mM DTT (final concentration ˜1.7 M urea, 50 mM Tris, and 1 mM DTT) was added to give a final total volume of ˜45 μL. Samples were incubated at 60° C. for 15 min and allowed to cool to room temperature. IODO was added to a final concentration of 3 mM and incubated at room temperature for 45 min in the dark. DTT was added to a final concentration of 3 mM before adding 1 μg of sequencing-grade trypsin (Promega) and incubating at 37° C. overnight. Samples were acidified to 0.5% TFA (ph<2) with 10% TFA stock and incubated for 30 min before desalting on C18 stage tip (Rainin).

### Mass Spectrometry Data Acquisition and Analysis

For AP-MS experiments, samples were resuspended in 15 μL of MS loading buffer (4% formic acid, 2% acetonitrile) and 2 μL were separated by a reversed-phase gradient over a nanoflow 75 μm ID×25 cm long picotip column packed with 1.9 μM C18 particles (Dr. Maisch). Peptides were directly injected over the course of a 75 min acquisition into a Q-Exactive Plus mass spectrometer (Thermo), or over the course of a 90 min acquisition into a Orbitrap Elite mass spectrometer. Raw MS data were searched against the uniprot canonical isoforms of the human proteome (downloaded Mar. 21, 2018), and using the default settings in MaxQuant (version 1.6.2.10), with a match-between-runs enabled (Cox and Mann, 2008). Peptides and proteins were filtered to 1% false discovery rate in MaxQuant, and identified proteins were then subjected to protein-protein interaction scoring. To quantify changes in interactions between WT and mutant baits, a label free quantification approach was used, in which statistical analysis was performed using MSstats (Choi et al., 2014) from within the artMS Bioconductor R-package. All raw data files and search results are available from the Pride partner ProteomeXchange repository under the PXD019469 identifier (Perez-Riverol et al., 2019; Vizcaino et al., 2014).

### Targeted Proteomic Analysis

Targeted proteomic analysis of APMS samples was performed on a Thermo Q-Exactive Plus mass spectrometer using the same HPLC conditions as described for original AP-MS experiments. All peptide and fragment ion selection, as well as quantitative data extraction was performed using Skyline (MacLean et al., 2010). Quantitative values were then imported into PRISM 8 software to perform normalization by bait abundance and statistical testing (2-tailed, unpaired t-test).

### Protein-Protein Interaction Scoring

Protein spectral counts as determined by MaxQuant search results were used for PPI confidence scoring by both SAINTexpress (version 3.6.1) (Teo et al., 2014) and CompPASS (version 0.0.0.9000) (Huttlin et al., 2015; Sowa et al., 2009). All PPI scoring was performed separately for each cell line. For SAINTexpress, control samples in which bait protein was not induced by addition of doxycycline were used. For CompPASS, a stats table representing all WT baits was used. After scoring, the CompPASS WD and Z-score were normalized within a given bait for each cell line. The total list of candidate PPIs was filtered to those that met the following criteria: SAINTexpress BFDR=<0.05, WD percentile by bait>=0.95, and Z-score percentile by bait≤=0.95. PPIs passing all 3 of these criteria were considered to be high-confidence PPIs. To enable visualization and analysis of PPIs by confidence score among these 3 criteria, a PPI score was calculated: [(WD percentile by bait+Z-score percentile by bait)/2)+(1−BFDR)]/2. This score places both the PPI confidence from SAINTexpress and CompPASS on a zero to 1 scale, with 1 being the highest confidence, and then takes the weighted average of these confidence scores.

### Permutation Test

A permutation test was performed in which genes were drawn from the list of all genes detected in the global protein abundance analysis of the parental cell lines. The null distribution of the average number of samples with variation was learned from 10,000 random gene lists of equal size to the set of interacting partners. This permutation test was performed individually for mutations (excluding silent mutations), CNVs, and mRNA expression. The information for observed variation of each gene is collected from the TCGA head and neck cancer cohort (firehose legacy; downloaded from cbioportal.org/datasets).

### Protein-Protein Interaction Scoring: CompPASS

CompPASS is an acronym for Comparative Proteomic Analysis Software Suite. It relies on an unbiased comparative approach for identifying high-confidence candidate interacting proteins (HCIPs for short) from the hundreds of proteins typically identified in IP-MS/MS experiments. There are several scoring metrics calculated as part of comPASS: The Z-score, the S-score, the D-score, and the WD-score. The S-score, D-score, and WD-score were all developed empirically based on their ability to effectively discriminate known interactors from known background proteins. Each score has advantages and disadvantages, and each are used to assess distinct aspects of the dataset. However, the primary score use to determine the high-confidence protein-protein interaction dataset is the WD-score. Typically, the top 5% of the WD-score scores are taken (more information under “Determining Thresholds”).

### The Z-Score.

The first score is the conventional Z-score, which determines the number of standard deviations away from the mean (Eq. 1) at which a measurement lies (Eq. 2). In Eq. 1 & 2× is the TSC, i is the bait number, j is the interactor, n denotes which interactor is being considered, k is the total number of baits, and s is the standard deviation of the TSC mean.

$${\overline{x}}_{j} = \frac{{\sum }_{\text{?}}^{\text{?}} {x}_{i , j}}{k} : n = 1 , 2 , … m \qquad ( Eq . 1 )$$

$${z}_{i , j} = \frac{{x}_{i , j} \cdot {\tilde{x}}_{j}}{{σ}_{j}} \qquad ( Eq . 2 )$$

$$\text{?} \text{indicates text missing or illegible when filed}$$

Each interactor for each bait has a Z-score calculated and therefore, the same interactor will have a different Z-score depending on the bait (assuming the TSC is different when identified for that bait). Although the Z-score can effectively identify interactors who's TSC is significantly different from the mean, if an interactor is unique (found in association with only 1 bait), then it fails to discriminate between interactors with a single TSC (“one hit wonders”) and another that may have 20 TSC or 50 TSC, etc. In this way, the Z-score will tend to upweight unique proteins, no matter their abundance. This can be dangerous since the stochastic nature of data-dependent acquisition mass spectrometry leads to spurious identification of proteins. These would be assigned the maximal Z-score as they would be unique, however they likely do not represent bonafide interactors.

### The S-Score.

The next score is the S-score which incorporates the frequency of the observed interactor and its' abundance (TSC). Both the D- and WD-scores are based on the S-score, sharing the same fundamental formulation, but have additional terms that add increasing resolving power. The S-score (Eq. 3) is essentially a uniqueness and abundance measurement.

$${S}_{i , j} = - \sqrt{( \frac{k}{{\sum }_{\text{?}}^{\text{?}} {f}_{i , j}} ) {x}_{i , j}} ; \qquad ( Eq . 3 )$$

$${f}_{i , j} = \{ \begin{matrix}1 : {x}_{i , j} > 0 \\ {x}_{i , j}\end{matrix}$$

$$\text{?} \text{indicates text missing or illegible when filed}$$

In Eq. 3, the variables are the same as for Eq. 1 & 2.f is a term which is 0 or 1 depending on whether or not the interacting protein is found in a given bait. Placed in the summation across all baits, it is a counting term and therefore, k/Sf is the inverse ratio (or frequency) of this interactor across all baits. The smaller f, the larger this value becomes and thus upweights interactors that are rare. The term X_{i,j} is the TSC for interactor j from bait i and therefore multiplying by this value scales the S-score with increasing interactor TSC—this provides a higher score to interactors having high TSC and are therefore more abundant and less likely to be stochastically sampled. Although increasing the resolution above using the Z-score alone (the S-score can discriminate between unique one hit wonders and unique interactors with high TSC), the S-score will give its highest values to interactors that very rare and can lead to one hit wonders being scored among the top proteins. However, with a stringent cut-off value, the S-score reliably identifies HCIPs and bona fide interacting proteins but at this level, is prone to miss lower abundant likely interacting proteins. In order to address this limitation, the S-score was modified to take into account the reproducibility of the interactor for a given bait—a quantity that can be determined as a result of performing duplicate mass spectrometry runs. After adding this modification, the S-score becomes the D-score (Eq. 4).

### The D-Score.

The D-score is fundamentally the same as the S-score except with an added power term to take into account the reproducibility of the interaction. The term p can either be 1 (if the interactor was found in 1 of 2 duplicate runs) or 2 (if the interactor was found in both duplicate runs).

$${D}_{i , j} = - \sqrt{{( \frac{k}{{\sum }_{\text{?}}^{\text{?}} {f}_{i , j}} )}^{p} {x}_{i , j}} ; \qquad ( Eq . 4 )$$

$${f}_{i , j} = \{ \begin{matrix}1 : {x}_{i , j} > 0 \\ {x}_{i , j}\end{matrix}$$

$$p = \text{?}$$

$$\text{?} \text{indicates text missing or illegible when filed}$$

If p is 1 (the interactor was found in 1 of 2 duplicates) then the D-score is the same as the S-score. Adding the reproducibility term now allows for better discrimination between a true one hit wonder (a protein found with 1 peptide in a single run, not in the duplicate) which is likely a false positive versus a true interactor with low (even 1) TSC that is found in both duplicate runs. Although powerful in its ability to delineate HCIPs from background proteins, the D-score still relies heavily on the frequency term, k/Sf, and will thus assign lower scores to more frequently observed proteins. In the vast majority of the cases, this is of course a good thing since these proteins are more than likely background. However, in the event that a canonical background protein is a bonafide interactor for a specific bait, its D-score would likely be too low for passing the D-score threshold (discussed below) and would not be considered a HCIP. Another example pertains to CompPASS analysis of baits from within the same biological network or pathway. In the case of the Dub Project, most of these proteins do not share interactors as this analysis was performed across a protein family—in which case the D-score works very well. However sometimes baits do share interactors as these proteins are part of the same biological pathway and determining these share interactors (and hence the connections among these proteins) is critical for a reliable assessment of the pathway. In these cases, the D-score works fairly well for most interactors, however it can downweigh very commonly found bona fide interactors (especially when these interactors have low TSC). To address this limitation, a weighting factor to be added into the D-score was devised, and the WD-score (or Weighted D-score; Eq. 5) was created.

### The WD-Score.

Upon examination of frequently observed proteins (considered background) that are either known not to be a bona fide interactor for any bait and those that are known to be true interactors for a subset of baits, it is found that the distributions of the TSC for these groups vary in a correlated manner. In the first case, where these “background” proteins are never true interactors, the standard deviation of the TSC (s_{TSC}) is smaller than that of the latter case (“background” proteins that are known to be true interactors for specific baits). This occurs since real background protein abundance is mainly determined by the amount of resin used in the IP whereas in the case of a background protein becoming a true interactor, its TSC then rises far above this consistent level (and thus cause s_{TSC} to increase. In fact, when S_{TSC} is systematically examined across all proteins found in >50% of the IP-MS/MS datasets, the proteins that are known to be real interactors for specific baits are found to have a S_{TSC} that is >100% of the TSC mean for that protein across all IPs. Therefore, a weight factor term is introduced as wⱼ and is essentially the S_{TSC}/TSC mean for interactor j (shown below).

$${WD}_{i , j} = - \sqrt{{( \frac{k}{{\sum }_{\text{?}}^{\text{?}} {f}_{i , j}} {ω}_{j} )}^{p} {x}_{i , j}} \qquad ( Eq . 5 )$$

$${ω}_{j} = ( \frac{{σ}_{i}}{{\tilde{x}}_{i}} ) , {\tilde{x}}_{j} = \frac{{\sum }_{\text{?}}^{\text{?}} {x}_{i , j}}{k} : n = 1 , 2 , … m ,$$

$$\begin{matrix}if {ω}_{j} \times 1 \text{?} {ω}_{j} = 1 \\ if {ω}_{j} > 1 \text{?} {ω}_{j} = {ω}_{j}\end{matrix}$$

$${f}_{i , j} = \{ \begin{matrix}1 : {x}_{i , j} > 0 \\ {x}_{i , j}\end{matrix}$$

$$p = \text{?}$$

$$\text{?} \text{indicates text missing or illegible when filed}$$

The weight factor, wⱼ, is added as a multiplicative factor to the frequency term in order to offset this low value for interactors that are found frequently across baits but will only be >1 if the conditions in Eq. 5 are met. If these conditions are not met, then oⱼ is set to 1 and the WD-score is the same as the D-score. In this way, only if a frequent interactor displays the observed characteristics of a true interactor will its score increase due to the weight factor.

To determine score thresholds for determining high-confidence protein-protein interactions, randomly generated simulated run data are compared against. In order to create simulated random runs, the data from actual experiments is first used to create the proteome observed from the experiments. To do this, each protein is represented by its TSC from each run—in other words, if a protein is found with a total of 450 TSC summed across all real runs, then it is represented 450 times. Simulated runs are then created by randomly drawing from this “experimental proteome” until 300 proteins are selected and the total TSC for the simulated run is ˜1500 (these are the average values found across the actual experiments). Next, scores are calculated for the random runs to determine the distributions of the scores for random data. Finally, for each score, the corresponding value above which 5% of the random data lies is found, and that value taken to be that score's threshold. Although 5% of the random data is above this threshold value, an examination of the TSC distribution for these random data is expected to show that ≤99% have TSC<4. Therefore, although there are false positive HCIPs in real datasets, this distribution can now be used to assign a p-value for proteins passing the score thresholds. In this way, an argument can be made that a protein passing a score threshold and found to have high enough TSC (reflected in the p-value) is very likely to be a real interactor. A suitable approximation for this above described method is to simply take the minimal value of the top 5% of the scores for each metric and set that value to be the threshold for that score.

### Protein-Protein Interaction Scoring: SAINT

The aim of SAINT is to convert the label free quantification (spectral count Xᵢⱼ) for a prey protein i identified in a purification of bait j into the probability of true interaction between the two proteins, P(True|Xᵢⱼ). The spectral counts for each prey-bait pair are modeled with a mixture distribution of two components representing true and false interactions. Note that these distributions are specific to each bait-prey pair. The parameters for true and false distributions, P(Xᵢⱼ|True) and P(Xᵢⱼ|False), and the prior probability π_{T} of true interactions in the dataset, are inferred from the spectral counts for all interactions involving prey i and bait j. SAINT normalizes spectral counts to the length of the proteins and to the total number of spectra in the purification.

The spectral counts for prey i in purification with bait j are considered to be either from a Poisson distribution representing true interaction (with mean count λᵢⱼ) or from a Poisson distribution representing false interaction (with mean count κᵢⱼ). In the form of probability distribution, the following formula is written:

$$P ( {X}_{ij} \vert \text{*)} = {π}_{T} P ( {X}_{ij} \vert {λ}_{ij} ) + ( I - {π}_{T} ) P ( {X}_{ij} \vert {κ}_{ij} ) \qquad ( 1 )$$

where π_{T} is the proportion of true interactions in the data, and dot notation represents all relevant model parameters estimated from the data (here, specifically for the pair of prey i and bait j). The individual bait-prey interaction parameters λᵢⱼ and κᵢⱼ are estimated from joint modeling of the entire bait-prey association matrix, with the probability distribution (likelihood) of the form P(X|•)=Π_{i,j}P(Xᵢⱼ|•). The proportion π_{T} is also estimated from the model, which relies on latent variables in the sampling algorithm (see below).

When at least three control purifications are available, and assuming that the control purifications provide a robust representation of nonspecific interactors, the parameter κᵢⱼ can be estimated from spectral counts for prey i observed in the negative controls. This is equivalent to assuming

$$P ( {X}_{ij} \vert \text{*)} = \prod \text{?} ( {π}_{T} P ( {X}_{ij} \vert {λ}_{ij} ) + ( I - {π}_{T} ) P ( {X}_{ij} \vert {κ}_{ij} ) \times \prod \text{?} ( P ( {X}_{ij} \vert {κ}_{ij} ) ) \qquad ( 2 )$$

$$\text{?} \text{indicates text missing or illegible when filed}$$

where E and C denote the group of experimental purifications and the group of negative controls, respectively. This leads to a semi-supervised mixture model in the sense that there is a fixed assignment to false interaction distribution for negative controls. As negative controls guarantee sufficient information for inferring model parameters for false interaction distributions, Bayesian nonparametric inference using Dirichlet process mixture priors can be used to derive the posterior distribution of protein-specific abundance parameters in the model. As a result, the mean parameters in the Poisson likelihood functions follow a nonparametric posterior distribution, allowing more flexible modeling at the proteome level. Under this setting, all model parameters are estimated from an efficient Markov chain Monte Carlo algorithm.

To elaborate on the two distributions, the mean parameter for each distribution is assumed to have the following form. For false interactions, it is assumed that spectral counts follow a Poisson distribution with mean count:

$$log ( {κ}_{ij} ) = log ( {l}_{i} ) + log ( {c}_{j} ) + {γ}_{0} + {μ}_{i} \qquad ( 3 )$$

where lᵢ is the sequence length of prey i, and cⱼ is the bait coverage, the spectral count of the bait in its own purification experiment, γ₀ is the average abundance of all contaminants and μᵢ is prey i specific mean difference from γ₀. For true interactions, it is assumed that spectral counts follow a Poisson distribution with mean count:

$$log ( {λ}_{ij} ) = log ( {l}_{i} ) + log ( {c}_{j} ) + {β}_{0} + {α}_{bj} + {α}_{pi} \qquad ( 4 )$$

where β₀ is the average abundance of prey proteins in those cases where they are true interactors of the bait, α_{bj} is bait j specific abundance factor and αₚᵢ is prey i specific abundance factor. In other words, the mean spectral count for a prey protein in a true interaction is calculated using a multiplicative model combining bait- and prey-specific abundance parameters. This formulation substantially reduces the number of parameters in the model, avoiding the need to estimate every λᵢⱼ separately.

For datasets without negative control purifications, the mixture component distributions for true and false interactions have to be identified solely from experimental (non-control) purifications. In this case, a user-specified threshold is applied to divide preys into high-frequency and low-frequency groups, denoted as Yᵢ=1 or 0 if prey i belongs to the high- or low-frequency group, respectively. An arbitrary 20% threshold is applied in the case of the DUB dataset; however, the results are not expected to be very sensitive to the choice of the threshold. For preys in the high frequency group, the model considers spectral counts for the observed prey proteins (ignoring zero count data, which represent the absence of protein identification), as there are sufficient data to estimate distribution parameters. In the low-frequency group, non-detection of a prey is included to help the separation of high-count from low-count hits. The entire mixture model can then be expressed as

$$P ( {X}_{ij} \vert \text{?} ) = {\prod }_{ij} {( {π}_{T} P ( {X}_{ij} \vert {λ}_{ij} ) + ( 1 - {π}_{T} ) P ( {X}_{ij} \vert {κ}_{ij} ) )}^{Zij} \qquad ( 5 )$$

$$\text{?} \text{indicates text missing or illegible when filed}$$

where Zᵢⱼ=1(Yᵢ=0)+1(Yᵢ=1,Xᵢⱼ>0) and the false and true interaction distributions are modeled by equations (3) and (4), respectively.

The posterior probability of a true interaction given the data is computed using Bayes rule

$$P ( true \vert {X}_{ij} ) = {T}_{ij} / ( {T}_{ij} + {F}_{ij} ) \qquad ( 6 )$$

where Tᵢⱼ=π_{T} P(Xᵢⱼ|λᵢⱼ) and Fᵢⱼ=(1−π_{T}) P(Xᵢⱼ|κᵢⱼ). If there are replicate purifications for bait j, the final probability is computed as an average of individual probabilities over replicates. Note that one alternative approach is to compute the probability assuming conditional independence over replicates, that is, Π_{k∈j}P(Xᵢⱼₖ|λᵢⱼₖ) and Π_{k∈j}P(Xᵢⱼₖ|κᵢⱼₖ) for true and false interactions, with additional index k denoting replicates for bait j. Unlike average probability, this probability puts less emphasis on the degree of reproducibility, and thus may be more appropriate in datasets where replicate analysis of the same bait is performed using different experimental conditions (for example, purifications using different affinity tags) to increase the coverage of the interactome.

When probabilities have been calculated for all interaction partners, the Bayesian false discovery rate (FDR) can be estimated from the posterior probabilities as follows. For each probability threshold p*, the Bayesian FDR is approximated by

$$FDR ( {p}^{*} ) = ( {\sum }_{k} 1 ( {p}_{k} \geq {p}^{*} ) ( 1 - {p}_{k} ) ) / ( {\sum }_{k} 1 ( {p}_{k} \geq {p}^{*} ) ) \qquad ( 7 )$$

where pₖ is the posterior probability of true interaction of protein pair k. The output from SAINT allows the user to select a probability threshold to filter the data to achieve the desired FDR.

### Comparing Protein Interactions Using Hierarchical Clustering

Hierarchical clustering is performed on interactions for distinct but related proteins, including viral proteins, cancer proteins, or proteins from other diseases, which are hereout simply referred to as “conditions.” First, protein interactions that pass the master threshold (defined in “High-confidence protein interaction scoring” section above) in at least one condition are assembled. New interaction scores (K) are created by taking the average of several interaction scores. This is done to provide a single score that captures the benefits from each scoring method. Clustering is then done using this new Interaction Score (K). Clustering is performed using the ComplexHeatmap package in R, using the “average” clustering method and “euclidean” distance metric. K-means clustering is applied to capture all possible combinations of interaction patterns between conditions.

### Differential Interaction Score (DIS) Analysis

To compare PPIs across conditions (i.e., cell lines, viruses, diseases), a method for calculating a differential interaction score (DIS) was developed, and a corresponding false discovery rate (FDR) can be calculated using AP-MS data across multiple conditions. This approach uses the SAINTexpress score (G. Teo, et al., SAINTexpress: improvements and additional features in Significance Analysis of INTeractome software. J. Proteomics. 100, 37-43 (2014)), which is the probability of a PPI being bonafide in a single condition. Here, Sc(b, p) is the SAINTexpress score of a specific PPI denoted as (b, p) in a condition c. Here, an example is provided using three distinct conditions, C1, C2, and C3. Given that PPIs are independent events across different conditions, the differential interaction score is calculated for each PPI (b, p) as the product of the probability of a PPI being present in two of the conditions but absent in the third for each PPI:

$${DIS}_{A} ( b , p ) = {S}_{C 1} ( b , p ) \times {S}_{C 2} ( b , p ) \times [ 1 - {S}_{C 3} ( b , p ) ]$$

This differential interaction score highlights PPIs that are strongly conserved across two of the conditions, but not shared by the third. Additionally, PPIs that are present in the one conditions, but depleted in the other two, can be highlighted as follows:

$${DIS}_{B} ( b , p ) = [ 1 - {S}_{C 1} ( b , p ) ] \times [ 1 - {S}_{C 2} ( b , p ) ] \times {S}_{C 3} ( b , p ) ]$$

These two DIS scores can be further merged to define a single score for each PPI, where if DIS_{A}>DIS_{B}, the DIS is assigned a positive (+) sign, while if DIS_{A}<DIS_{B}, the unified DIS is assigned a negative (−) sign. In this way, the DIS for each PPI is represented by a continuum, in which negative DIS scores represent PPIs depleted in two of the three conditions, while positive DIS scores represent PPIs enriched in two of the three conditions. Additionally, for all differential interaction scores calculated, the Bayesian false discovery rate (BFDR) (G. Teo, G. Liu, J. Zhang, A. I. Nesvizhskii, A.-C. Gingras, H. Choi, SAINTexpress: improvements and additional features in Significance Analysis of INTeractome software. J. Proteomics. 100, 37-43 (2014)) estimates are also computed at all possible thresholds (p*) as follows:

$$FDR ( {p}^{*} ) = \frac{{\sum }_{i , i} ( 1 - DIS ( {p}_{i} , {p}_{j} ) ) \times I \{ DIS ( {p}_{i} , {p}_{j} ) > {p}^{*} \}}{{\sum }_{i , j} I \{ DIS ( {p}_{i} , {p}_{j} ) > {p}^{*} \}} ,$$

$$where$$

$$I \{ A \} is 1$$

$$when$$

$$A is True$$

$$and$$

$$0 otherwise .$$

Note, while these scores are used here for comparison across 3 conditions, it can also be used more simply to compare between any two conditions. Such a comparison is calculated as follows where DIS₁₁₂ results in PPIs specific to condition 1 have a positive DIS value, while PPIs specific to condition 2 results in a negative DIS value:

$${DIS}_{C 1 / C 2} ( {p}_{1} , {p}_{2} ) = {S}_{C 1} ( {p}_{1} , {p}_{2} ) \times ( 1 - {S}_{C 2} ( {p}_{1} , {p}_{2} ) )$$

$$or$$

$${DIS}_{C 3 / C 2} ( {p}_{1} , {p}_{2} ) = {S}_{C 3} ( {p}_{1} , {p}_{2} ) \times ( 1 - {S}_{C 2} ( {p}_{1} , {p}_{2} ) )$$

$$or$$

$${DIS}_{C 3 / C 1} ( {p}_{1} , {p}_{2} ) = {S}_{C 3} ( {p}_{1} , {p}_{2} ) \times ( 1 - {S}_{C 1} ( {p}_{1} , {p}_{2} ) )$$

### Differential Interaction Scoring

To compare PPIs across cell lines, a method for calculating a differential interaction score (DIS) and a corresponding false discovery rate (FDR) using AP-MS data across multiple cell lines was developed. This approach uses the SAINTexpress score (Teo et al., 2014), which is the probability of a PPI being bonafide in a single cell line. Here, Sc(b, p) was used as the SAINTexpress score of a specific PPI denoted as (b, p) in a cell line c. Given that PPIs are independent events across different cell lines, the differential interaction score was calculated for each PPI (b, p) as the product of the probability of a PPI being present in both cancer cell lines but absent in the HET-1A normal cell line as follow for each PPI:

$$DI \text{?} ( b , p ) = {S}_{CAL - 33} ( b , p ) \times \text{?} ( b , p ) \times [ I - {S}_{HET - 1 A} ( b , p ) ]$$

$$\text{?} \text{indicates text missing or illegible when filed}$$

This differential interaction score highlights PPIs that are strongly conserved across two cancer cell lines, but not shared by the normal cell line. Additionally, PPIs that are present in the control HET-1A cell line, but depleted in both cancer cell lines can be highlighted as follows:

$$DI \text{?} ( b , p ) = [ 1 - {S}_{CAL - 33} ( b , p ) ] \times [ \text{?} - \text{?} ( b , p ) ] \times {S}_{HET - 1 A} ( b , p )$$

$$\text{?} \text{indicates text missing or illegible when filed}$$

These two DIS scores were merged to define a single score for each PPI, where if DIS cancer>DISnormal, the DIS is assigned a positive (+) sign, while if DIScancer<DISnormal, the unified DIS is assigned a negative (−) sign. In this way, the DIS for each PPI is represented by a continuum, in which negative DIS scores represent PPIs depleted in HNSCC, while positive DIS scores represent PPIs enriched in HNSCC. Additionally, for all differential interaction scores that were calculated, the Bayesian false discovery rate (BFDR) (Teo et al., 2014) estimates were also computed at all possible thresholds (p*) as follows:

$$FDR ( {p}^{*} ) = \frac{\text{?}}{\text{?}} ,$$

$$where$$

$$I \{ A \} is 1$$

$$when$$

$$A is True$$

$$and$$

$$0 otherwise .$$

$$\text{?} \text{indicates text missing or illegible when filed}$$

Note, while these scores were used for comparison across 3 cell lines, it can also be used more simply to compare between any two cell lines. Such a comparison is calculated as follows where DISLineA/LineB results in PPIs specific to cell line A have a positive DIS value, while PPIs specific to cell line B results in a negative DIS value:

$$DI \text{?} ( {p}_{1} , {p}_{2} ) = {S}_{CAL - 33} ( {p}_{1} , {p}_{2} ) \times ( 1 - \text{?} ( {p}_{1} , {p}_{2} ) )$$

$$or$$

$$DI \text{?} ( {p}_{1} , {p}_{2} ) = \text{?} ( {p}_{1} , {p}_{2} ) \times ( 1 - \text{?} ( {p}_{1} , {p}_{2} ) )$$

$$or$$

$$DI \text{?} ( {p}_{1} , {p}_{2} ) = \text{?} ( {p}_{1} , {p}_{2} ) \times ( 1 - \text{?} ( {p}_{1} , {p}_{2} ) ) .$$

$$\text{?} \text{indicates text missing or illegible when filed}$$

### MAPK1 Validation Experiments

CAL-33 and HSC-6 cells were transiently transfected with 20 nM non-targeting control siRNA (Dharmacon Cat #D-001810-10) or RPS6KA1 siRNA pool (Origene Cat #SR304161). After 24 hrs, cells were seeded in 96-well plates (for viability assessment) in quadruplicate and 6-well plates (for lysate preparation). After 72 hrs, 96-well plates were stained with crystal violet for 30 min, washed with tap water, and allowed to dry for 24 hrs. Crystal violet stain was dissolved in 5% SDS solution and the resulting solution was quantified using a colorimetric plate reader at 570 nM. Lysates were procured from the 6-well plates using RIPA lysis buffer. Immunoblotting was performed to validate RPS6KA1 knockdown and PVDF membranes were probed using a RSK1/2/3 antibody (CST #9355). Total ERK1/2 (CST #4695) was used as a loading control.

### NanoBiT GAi1 Dissociation Assay

The NanoBiT G-protein dissociation assay, based on a split-luciferase system, was performed as previously described with some modifications (Inoue et al., 2019). All DNA constructs were provided by Dr. Asuka Inoue (Tohoku University, Japan). NanoBiT plasmids (pCAGGS) include Gαi1-LgBiT, Gβ1-native, and SmBiT-Gγ2 (CAAX C68S mutant). Gαi-DREADD (pcDNA3.1) was used as a synthetic Gαi-coupled GPCR. Briefly, CAL-33 and HET-1A cells were seeded on poly-D-lysine coated (Sigma, Cat #P7280), opaque, white 96-well plates (Falcon Cat #353296). The following day cells were transfected with NanoBiT and receptor plasmids using Lipofectamine 3000 (ThermoFisher Scientific, Cat #L3000008) according to manufacturer recommendations for a 12-well scale (10 μL transfection mix to each well). The NanoBiT plasmids were mixed at a ratio of 100 ng Gαi1-LgBiT, 500 ng Gβ1, 500 ng SmBiT-Gγ2, and 200 ng of receptor if needed. For gene knockdown experiments, 10 pmol of pooled siControl (Dharmacon, Cat #D-001810-10-20), siFGFR3 (Mission siRNA, Cat #SIHK0780, SIHK0781, SIHK0782), or siDaple (Dharmacon, Cat #L-033364-01-0005) was included in the plasmid mix. Media was changed the following day. Two days after transfection, media was aspirated from each well and washed once with HBSS. Cells were incubated in HBSS with a final concentration of 5 μM native coelenterazine (Biotium, Cat #10110-1) for 30 minutes at room temperature protected from light. Basal luminescence was read and ligand prepared for final concentrations of 10 ng/mL human bFGF (Roche Cat #11123149001) and 10 μM clozapine-N-oxide (Cayman Chemical, Cat #NC1044836). After ligand addition, luminescence was read in kinetic loops (each well ˜every 30 seconds) for 60 minutes total (Tecan Spark). Raw luminescent values were normalized to the corresponding basal value for each well and subsequently to the mean vehicle ratio (raw/basal) at time 0. Significance was calculated using a one-way ANOVA at the 60 minute time point.

### Scratch Migration Assay

CAL-33 cells were seeded on 12-well plates coated with 10 μg/mL fibronectin in PBS (Sigma Aldrich, Cat #F2006-1MG). Once cells reached confluence, a vertical scratch was made with a pipette tip and washed well with PBS before adding serum-free media. Cells were stimulated with vehicle, 10 ng/mL bFGF, or 1% serum for 24 hours. Images were taken at the 0 and 24 hour time points (2× magnification) and the scratch area was quantified using ImageJ. Percent scratch closure was calculated for each well and significance assessed using a one-way ANOVA.

Phosphorylated PAK, ERK, and siRNA Knockdown Confirmation Immunoblots

CAL-33 and HET-1A cells were seeded on poly-D-lysine-coated 6-well plates. Cells were transfected with siRNA using Lipofectamine RNAiMAX (Thermo Fisher Scientific, Cat #100014472) according to manufacturer recommendations. After overnight serum starvation, cells were stimulated with vehicle, 10 ng/mL bFGF, or 10 μM CNO. Cells were washed once with PBS and lysed in RIPA buffer (50 mM Tris-HCl pH 6.8, 150 mM NaCl, 1% NP-40, 0.5% sodium deoxycholate, 0.1% SDS) with protease and phosphatase inhibitors (Bimake, Cat #B14001, B15001-A/B). Lysates were briefly sonicated and cleared by centrifugation before boiling in Laemmli sample buffer (Bio-Rad Cat #1610747). After separation on 10% acrylamide gels and transfer to PVDF membranes, membranes were blocked with 2% BSA in TBST before incubating with antibodies. Primary antibodies against phospho-PAK1(S199/204)/PAK2(S192/197) (1:1000, Cell Signaling Technology, Cat #2605), PAK1 (1:2000, Cell Signaling Technology, Cat #2602), PAK2 (1:2000, Cell Signaling Technology, Cat #2608), pERK (1:2000, Cell Signaling Technology, Cat #9106), ERK (1:2000, Cell Signaling Technology, Cat #9102), FGFR3 (1:2000, OriGene, Cat #TA801078), Daple (1:1000, Millipore EMD, Cat #ABS515), and GAPDH (1:10000, Cell Signaling Technology, Cat #2118) were used. After washing with TBST, membranes were incubated in secondary goat anti-rabbit HRP (1:20000, Southern Biotech, Cat #4010-05) and goat anti-mouse HRP (1:20000, Southern Biotech, Cat #1010-05) antibodies for chemiluminescent development.

### CDX3379 Treatment In Vivo and In Vitro Experiments

All the animal studies using HNSCC tumor xenografts were approved by the University of California, San Diego Institutional Animal Care and Use Committee (IACUC), with protocol ASP #S15195. All mice were obtained from Charles River Laboratories (Worcester, MA). To establish tumor xenografts, HNSCC cells were transplanted into both flanks (2 million per tumor) of female athymic mice (nu/nu, 4-6 weeks of age and weighing 16-18 g). Mice were fed with doxycycline food (6 g/kg) from Newco Distributors (Rancho Cucamonga, CA, USA) to induce PIK3CA expression. When average tumor volume reached 100 mm³, the mice were randomized into groups and treated by intraperitoneal (IP) injection with vehicle (PBS) or CDX3379 (10 mg/kg, twice a week) for approximately 15 days. The mice were sacrificed at the indicated time points (or when mice succumbed to disease, as determined by the ASP guidelines).

### Phosphorylated HER3 Immunoblots

Wild-type (WT) or mutant PIK3CA with FLAG-tag were expressed by lentiviral transduction in SCC-25 cells. Collected cells were washed with ice-cold PBS twice and then lysed with RIPA lysis buffer (150 mM Tris, pH 7.4, 100 mM NaF, 120 mM NaCl, 100 mM sodium orthovanadate, with 1 tablet protease inhibitor cocktail (Roche 31075800) and 1 tablet phosphatase inhibitor cocktail (Roche 04906837001) added. Lysates (30 μg) were resolved by SDS-PAGE, transferred to PVDF membranes (Bio-Rad #1620177), and incubated with primary antibodies (1:1000) at 4° C. overnight. Membranes were then washed and incubated with Goat Anti-Rabbit lgG(H+L)-HRP Conjugated secondary antibodies (1:5000) (Bio-Rad #170-6515) for 1 hr at room temperature, followed by washing four times with TBST. Antibodies against P-HER3-Y1197 (#4561) and HER3 (#12708) were from Cell Signaling Technology, and anti-B-tubulin (ab6276) was from Abcam. Blots were quantified with ImageJ software, and the intensity of P-HER3-Y1197 signal was normalized to FLAG-PIK3CA intensity.

### IAS Background Network

The integrated associated stringency (IAS) network was derived from integration of five major types of protein pairwise relationships recorded in public databases: (1) physical protein-protein interaction; (2) mRNA co-expression; (3) protein co-expression; (4) co-dependence (correlation of cell line growth upon gene knockouts); and (5) sequence-based relationships. A broad survey created a compendium of 127 network features used as inputs to a random forest regression model, trained to best recover the proximity of protein pairs in the Gene Ontology (GO). The final IAS score, ranging from 0 to 1, quantifies all pairwise associations among 19035 human proteins. In this study, stringent protein interactions were displayed with IAS>0.3 when the IAS network was used in figures.

### Data Analysis

Instant Clue software was used for the generation and statistical analysis of some figures (Nolte et al., 2018). Heatmaps were generated with Morpheus (https://software.broadinstitute.org/morpheus).

### Results

### Mapping of the Head and Neck Cancer Interactome

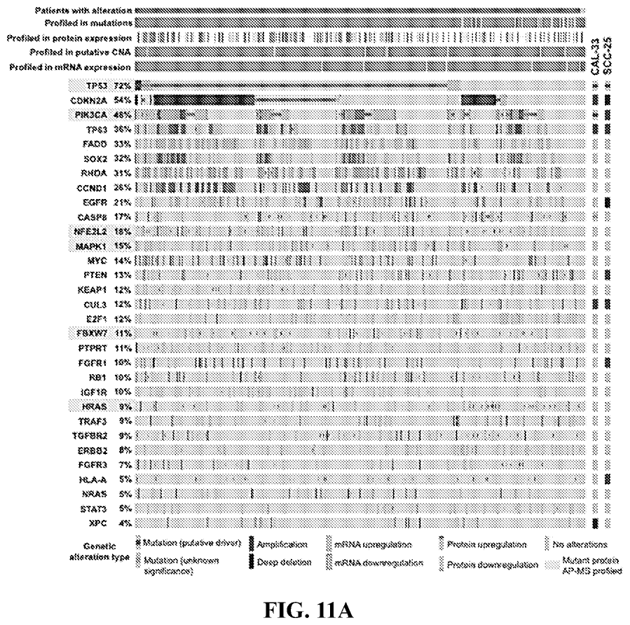

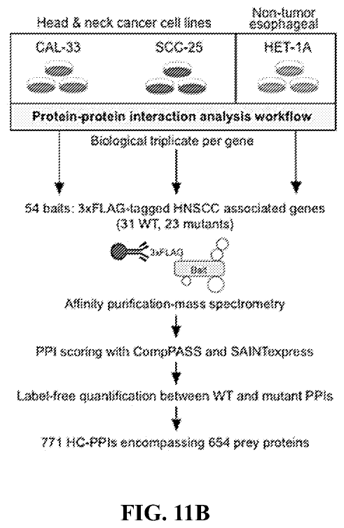

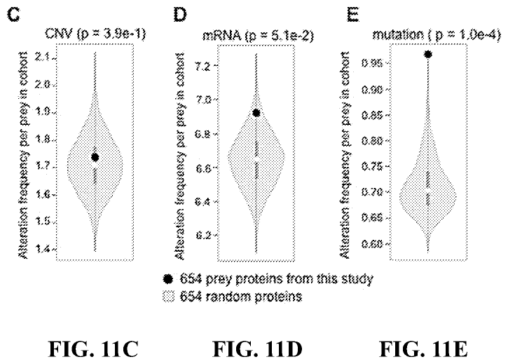

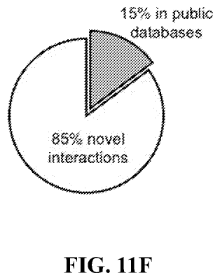

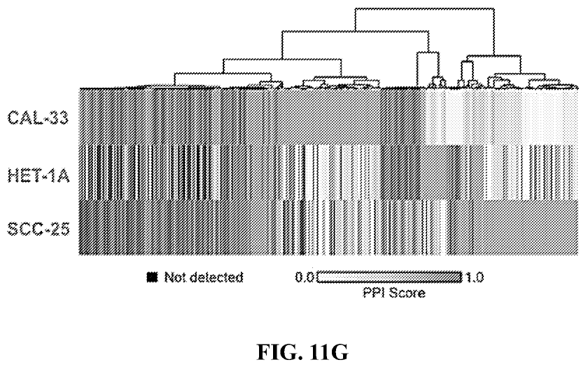

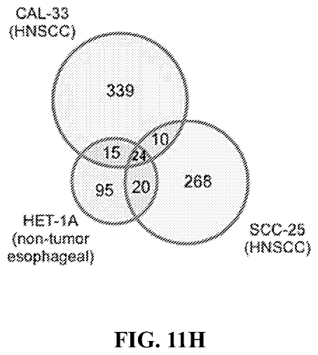

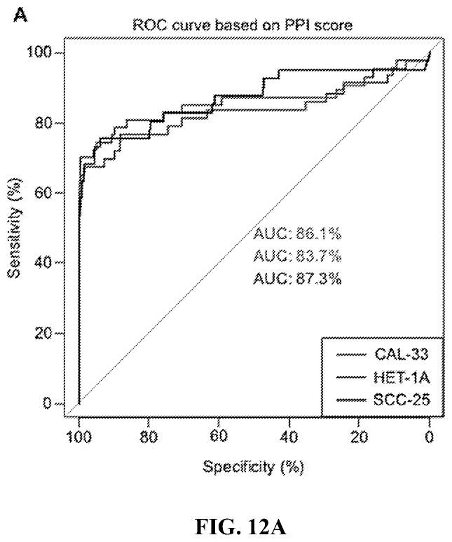

To characterize the protein-protein interaction landscape of HNSCC, proteins were selected based on altered molecular pathways identified from the TCGA analysis of HNSCC tumors FIG. 11A) (Cancer Genome Atlas, Network, 2015). Additional proteins were added based on genes with recurrent point mutations or a previously published association with HNSCC (Li et al., 2014; Martin et al., 2014; Molinolo et al., 2009; Stransky et al., 2011). In total, 33 protein baits were selected, of which 31 were found to be experimentally tractable (Key Resources Table 2). Importantly, 99% of HNSCC patients harbor an alteration in one or more of these proteins (FIG. HA).

Referring to FIG. 11A, the alteration frequencies from the HNSCC TCGA provisional dataset (n=530 patients) for the 31 experimentally tractable genes selected as AP-MS baits in this study are shown. Proteins analyzed in this study are listed, along with the percentage of patients with an alteration in that gene/protein. Each patient is represented by a grey box that is colored based on the occurrence and type of alteration(s) observed in that patient. Both the wild-type and mutant protein sequence(s) were analyzed for genes highlighted in gray. The genetic alteration types in the two cancer cell lines (CAL-33 and SCC-25) are also displayed.

Key Resources Table 2.

| Reagent or Resource | Source | Identifier |
|---|---|---|
| Cell lines | ATCC | HEK293T, HET-1A |
| Cell lines | Thomas Carey (University | SCC-25 |
| | of Michigan) | |
| Cell lines | Gerard Milano (University | CAL-33 |
| | of Nice, Nice, France) | |
| NanoBiT G-protein | Dr. Asuka Inoue (Tohoku | NanoBiT plasmids |
| dissociation assay | University, Japan) | (pCAGGS) include Gαi1- |
| | | LgBiT, Gβ1-native, and |
| | | SmBiT-Gγ2 (CAAX C68S |
| | | mutant). Gαi-DREADD |
| | | (pcDNA3.1) |
| Antibodies | | |
| RSK1/2/3 antibody | Cell Signaling Technology | 9355 |
| ERK1/2 | Cell Signaling Technology | 4695 |
| Phosphor- | Cell Signaling Technology | 2605 |
| PAK1(S199/204)/ | | |
| PAK2(S192/197) | | |
| PAK1 | Cell Signaling Technology | 2602 |
| PAK2 | Cell Signaling Technology | 2608 |
| pERK | Cell Signaling Technology | 9106 |
| FGFR3 | OriGene | TA801078 |
| Daple | Millipore EMD | ABS515 |
| GAPDH | Cell Signaling Technology | 2118 |
| Secondary goat anti-rabbit | Southern Biotech | 4010-05 |
| HRP | | |
| P-HER3-Y1197 | Cell Signaling Technology | 4561 |
| HER3 | Cell Signaling Technology | 12708 |
| Goat anti-mouse HRP | Southern Biotech | 1010-05 |
| Anti-B-tubulin | Abcam | ab6276 |
| ERK | Cell Signaling Technology | 9102 |
| Deposited data | | |
| Unprocessed peptide files | This paper | PRIDE ProteomeXchange: |
| | | PXD019469 |
| Raw data | This paper | PRIDE ProteomeXchange: |
| | | PXD019469 |
| Chemicals, Peptides, and | | |
| Recombinant Proteins | | |
| Tris | G-Biosciences | RC108 |
| Acetonitrile, HPLC grade | Thermo Fisher Scientific | A955-4 |
| (CAN) | | |
| cOmplete protease inhibitor | Roche | 11846 170 001 |
| cocktail tablets mini, | | |
| EDTA-free | | |
| Dithiothreitol (DTT) | Sigma-Aldrich | 43819 |
| Formic acid (FA) | Thermo Fisher Scientific | 28905 |
| Iodoacetamide (IAA) | Acros Organic | 122270250 |
| Sequencing-grade modified | Promega | V5111 |
| trypsin | | |
| Benzonase | Sigma | E1014-25KU |
| Trifluoroacetic acid (TFA) | Thermo Fisher Scientific | 28904 |
| Urea | Sigma-Aldrich | U5378-1kg |
| Fetal bovine serum (FBS) | Gibco | A3160502 |
| DMEM | Corning | MT10013CV |
| DMEM/F12 | Corning | MT10092CV |
| Water, HPLC grade | Sigma-Aldrich | 270733-4 L |
| Igepal (NP-40) | Sigma-Aldrich | I3021 |
| Minimal Essential Media | Corning | 10-009-CV |
| Opti-MEM | Thermo Fisher Scientific | 31985062 |
| BEGM ™ (Lonza) | Lonza | CC-3170 |
| 1% Penicillin-Streptomycin | Corning | MT30002CI |
| Paraformaldehyde, 4% | Thermo Scientific | MFCD00133991 |
| solution in PBS | | |
| PolyJet | SignaGen | SL 100688 |
| Lipfectamine 3000 | ThermoFisher Scientific | L30000008 |
| Hydrocortisone | Sigma | H6909-10ML |
| Rapigest | Waters | 186001861 |
| 3x Flag Peptide | Sigma | FA4799-4MG |
| Anti-Flag M2 Magnetic | Sigma | M8823-5ML |
| Beads | | |
| Lipofectamine RNAiMAX | Thermo Fisher Scientific | 100014472 |
| siFGFR3 | Sigma Aldrich | SIHK0780, SIHK0781, |
| | | SIHK0782 |
| Native coelenterazine | Biotium | 10110-1 |
| Pooled siControl | Dharmacon | D-001810-10-20 |
| siDaple | Dharmacon | L-033364-01-0005 |
| 10 μM clozapine-N-oxide | Cayman Chemical | NC1044836 |
| 5 μM native coelenterazine | Biotium | 10110-1 |
| RPS6KA1 siRNA pool | OriGene | SR304161 |
| Non-targeting control | Dharmacon | D-001810-10 |
| siRNA | | |
| Triton X-100 | Thermo Scientific | 9002-93-1 |
| Software and Algorithms | | |
| artMS | Bioconductor | https://www.bioconductor.org/ |
| | | packages/release/bioc/html/ |
| | | artMS.html |
| MSstats | Bioconductor | https://bioconductor.org/packages/ |
| | | release/bioc/html/MSstats/ |
| | | html |
| Skyline | MacCoss Lab | https://skyline.ms/project/home/ |
| | | begin.view? |
| The R Project for Statistical | R Core Team, 2019. R: A | http://www.r- |
| Computing | language and environment | project.org/index.html |
| | for statistical computing. R | |
| | Foundation for Statistical | |
| | Computing, Vienna, Austria. | |
| Morpheus | Broad Institute | https://software.broadinstitute.org/ |
| | | morpheus |
| MaxQuant (version | Jurgen Cox Lab | https://www.maxquant.org/ |
| 1.6.2.10) | | |
| InstantClue | | http://www.instantclue.uni- |
| | | koein.de/ |
| CompPASS (version | Github | https://github.com/dnusinow/ |
| 0.0.0.9000) | | cRomppass/blob/master/R/ |
| | | comppass.R |
| SAINTexpress (version | Sourceforge | https://sourceforge.net/projects/ |
| 3.6.1) | | saint-apms/files/ |
| Other | | |
| 1.9 μM C18 particles | Dr. Maisch | R119.aq.0001 |
| Picotip column | New Objective | PF360-75-10-N-5 |
| C18 Stage tips | Rainin | 17014047 |
| Orbitrap Elite mass | Thermo Fisher Scientific | IQLAAEGAAPFADBMAZQ |
| spectrometer | | |
| Q-Exactive Plus mass | Thermo Fisher Scientific | IQLAAEGAAPFALGMBDK |
| spectrometer | | |

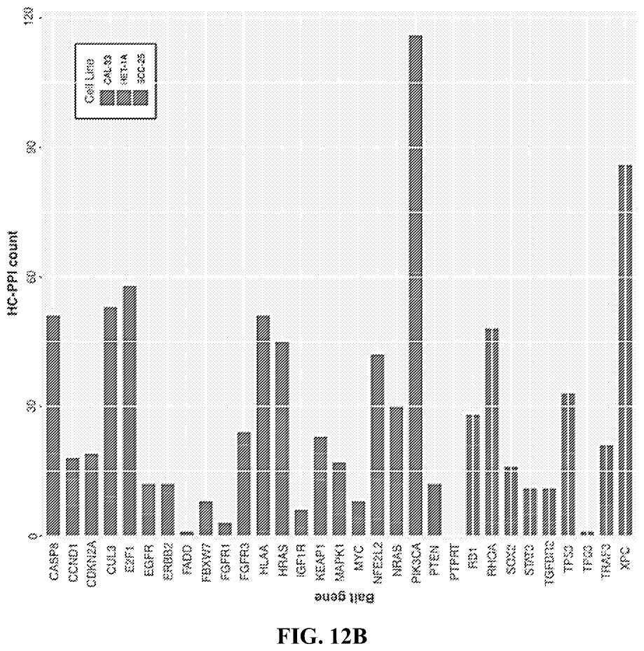

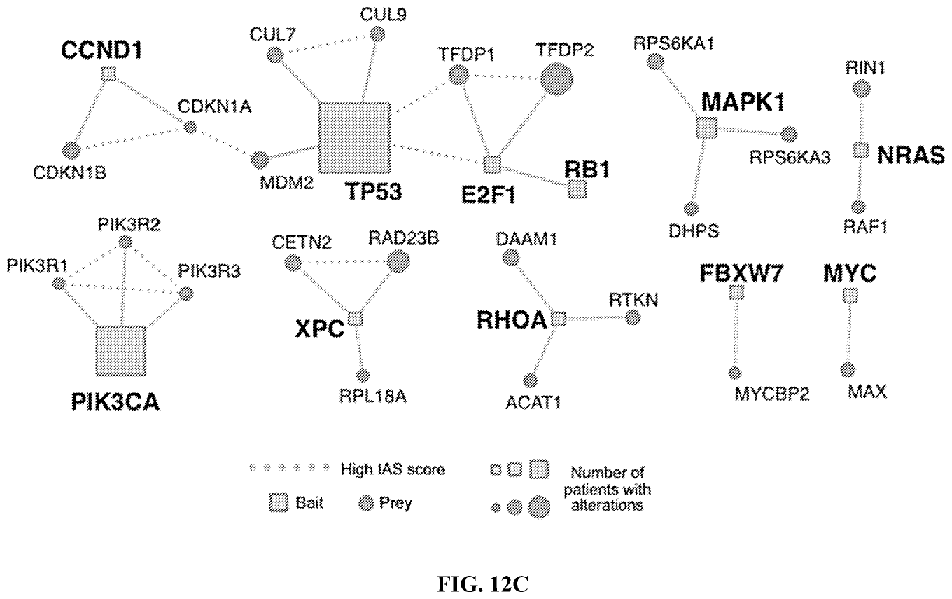

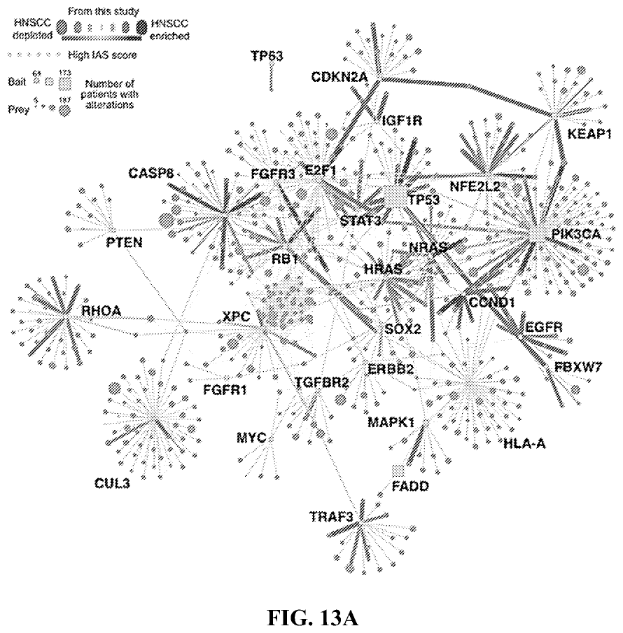

For those baits with recurrent point mutations, both the wild-type (WT) and mutant forms of the protein were tagged, purified, and analyzed. Each bait was expressed as a 3×FLAG-tagged protein under the control of a doxycycline-inducible promoter in biological triplicate in three separate cell lines (FIG. 11B). Two HPV-negative HNSCC cell lines were selected (SCC-25 and CAL-33) that harbor many genetic alterations present in the HNSCC patient population (FIG. 11A) and have previously been shown to have RNA profiles highly correlated with those of HNSCC patients (Spearman correlation=0.66 and 0.69 for CAL-33 and SCC-25, respectively) (Cancer Genome Atlas, Network, 2015; Li et al., 2014; Martin et al., 2014; Yu et al., 2019). Additionally, an immortalized non-tumorigenic cell line, HET-1A, was selected from a similar anatomical location (esophagus) for comparison. Then, a previously described AP-MS workflow was utilized to identify PPIs from these three cell lines (FIG. 11B) (Jager et al., 2011). Importantly, a conservative and high-confidence protein-protein interaction map was elected for report by requiring PPIs to pass stringent criteria by two complementary PPI scoring algorithms; SAINTexpress and CompPASS (Key Resources Table 2) (Huttlin et al., 2015; Sowa et al., 2009; Teo et al., 2014). Using this workflow, a total of 771 high-confidence PPIs (HC-PPIs) involving 654 proteins were identified (FIGS. 11B and 12A-B), for an average of 25 PPIs per bait gene.

Referring to FIG. 11B, the experimental workflow in which each bait was expressed in biological triplicate in 3 cell lines and subjected to AP-MS analysis is shown.

It has been previously shown that alteration profiles in cancer are organized into molecular networks in which the interaction partners of frequently altered proteins incur a higher rate of alteration than a random selection of genes (Bouhaddou et al., 2019; Eckhardt et al., 2018; Hofree et al., 2013; Leiserson et al., 2015). Thus, whether the HNSCC HC-PPI set was enriched was tested for different types of alterations measured in the HNSCC TCGA cohort (Key Resources Table 2). The dataset was, indeed, highly enriched for preys with point mutations; however, this enrichment was not observed for alterations in mRNA expression or for chromosomal rearrangements (FIG. 11C-E).

Referring to FIG. 11C-E, a permutation test illustrating the frequency of CNVs (FIG. 11C), mRNA alterations (FIG. 11D), or mutations (FIG. 11E) from randomly selected genes in the HNSCC TCGA data is shown. The white circle indicates the median of the random sampling, and the grey bar represents +/−1 standard deviation. The frequency of alterations found in the prey retrieved in this PPI dataset is indicated in the black circle.

Of the 771 HC-PPIs detected, the majority (85%) had not been previously reported in public PPI databases FIG. 11F). This high percentage of novel interactions is consistent with other AP-MS publications (Hein et al., 2015; Huttlin et al., 2015, 2017) and likely reflects differences across cellular contexts, as nearly all systematic PPI analyses to date have been performed in HEK293T or HeLa cell lines (Hein et al., 2015; Huttlin et al., 2015, 2017). This proportion of novel interactions is also supported by the observation of a high degree of specificity in PPIs observed even within the cell lines in this study (FIG. 11G), with only 24 PPIs being conserved as HC-PPIs across all cell lines analyzed (FIG. 11H and FIG. 12C). Notably, many well-studied cancer proteins are included in the novel interactions. For example, physical interactions were observed between the proto-oncoprotein MYC and each of two DNA repair proteins, PARP1 and TOP1. MYC has previously been shown to regulate PARP1 activity (Pyndiah et al., 2011), but this is the first evidence of a physical interaction between these two proteins. The MYC:PARP1 interaction is supported by previous studies reporting MYC:TOP1 (Kalkat et al., 2018) and PARPI:TOP1 interactions (Czubaty et al., 2005).

Referring to FIG. 11F, the percentage of HC-PPIs identified in a panel of public PPI databases (CORUM, BioPlex 2.0, or BioGRID low throughput and multivalidated) is shown.

Referring to FIG. 11G, a clustering analysis of all HC-PPIs (n=771) based on their PPI score, which is an average of the confidence scores reported from SAINTexpress and CompPASS score (see Key Resources Table 2 for details). A PPI score of 1.0 represents the highest confidence in a PPI.

Referring to FIG. 11H, a Venn diagram illustrating the overlap in HC-PPIs among the 3 cell lines is shown. For this analysis, only those PPIs passing the HC-PPI filtering criteria by both SAINTexpress and CompPASS were classified as an HC-PPI within an individual cell line.

Referring to FIG. 12A, a receiver operating characteristic (ROC) curve illustrating high recovery of gold standards (sensitivity) is shown.

Referring to FIG. 12B, the number of HC-PPIs per cell line for each bait is shown.

Referring to FIG. 12C, HC-PPIs that were detected across all cell lines (n=24) are shown. PPIs between preys from public databases having a high IAS score (see Key Resources Table 2) are also plotted as dotted lines (Zheng et al.).

Similarly, purification of tagged KEAP1 revealed an interaction with AJUBA, a scaffolding protein involved in the regulation of numerous cellular processes, including negative regulation of Wnt/β-catenin signaling (Haraguchi et al., 2008). Until recently, AJUBA was not associated with HNSCC; however, tumor genome analysis revealed it is inactivated in 7% of HPV-negative tumors (Cancer Genome Atlas, Network, 2015). The KEAPI:AJUBA interaction was further supported by the identification of a physical connection between KEAP1 and SQSTM1, a known AJUBA interactor (Copple et al., 2010; Fan et al., 2010; Feng and Longmore, 2005; Lau et al., 2010).

### A Statistical Approach to Evaluate Cell-Type Specificity of Interactions

To identify interactions with relevance to cancer biology, PPIs were compared across cell lines and those that are shared among CAL-33 and SCC-25, the two HNSCC cancer cell lines, but absent in the HET-1A non-tumorigenic cell line were prioritized. However, a simple overlap analysis of the sets of HC-PPIs identified by each cell line does not faithfully represent whether a PPI is shared. For example, a PPI might erroneously appear to be specific for a single cell line when it passes the threshold for HC-PPIs in that cell line (i.e., a true positive) while falling slightly below the threshold (i.e., false negative) in a second. Accordingly, a method for calculating differential interaction scores (DIS) for each PPI was developed, with associated Bayesian false discovery rates (BFDR). This method is based on the SAINTexpress score (Teo et al., 2014), which reports on the probability of a PPI in a single cell line given the AP-MS data. Here, quantitative SAINTexpress probabilities were combined across the three cell lines to generate the DIS (Key Resources Table 2), allowing for the identification of PPIs that are enriched in either the two cancer cell lines or the non-cancerous cells.

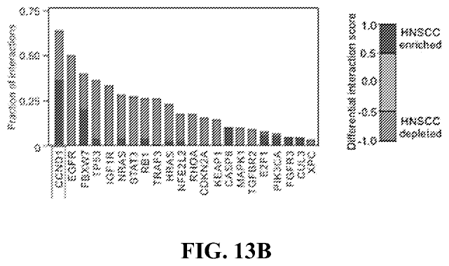

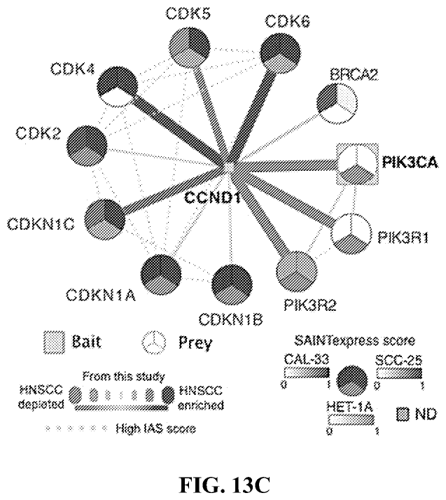

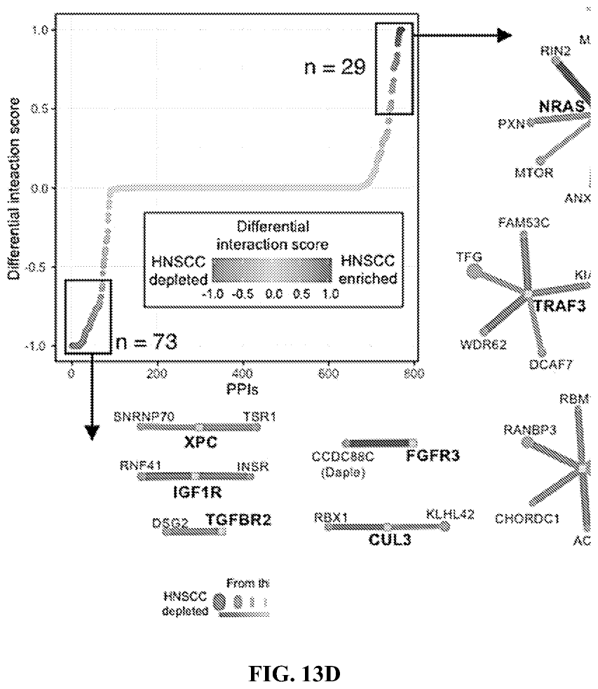

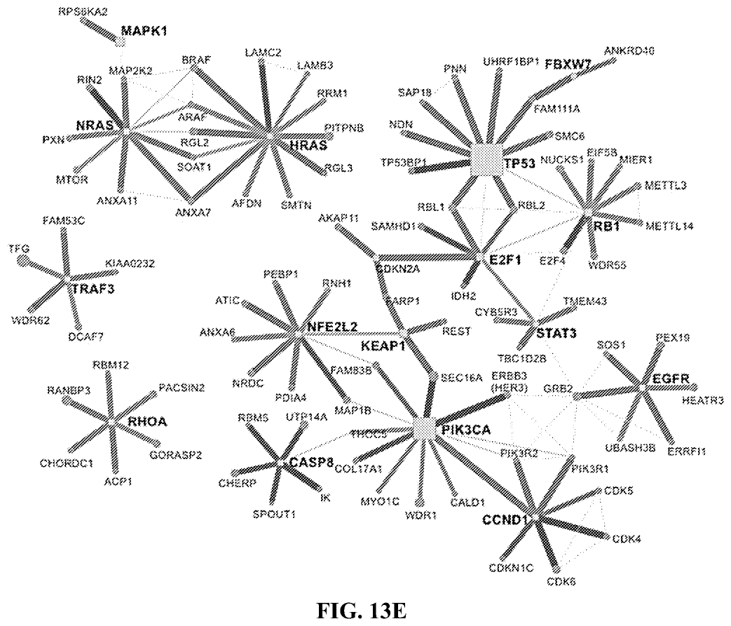

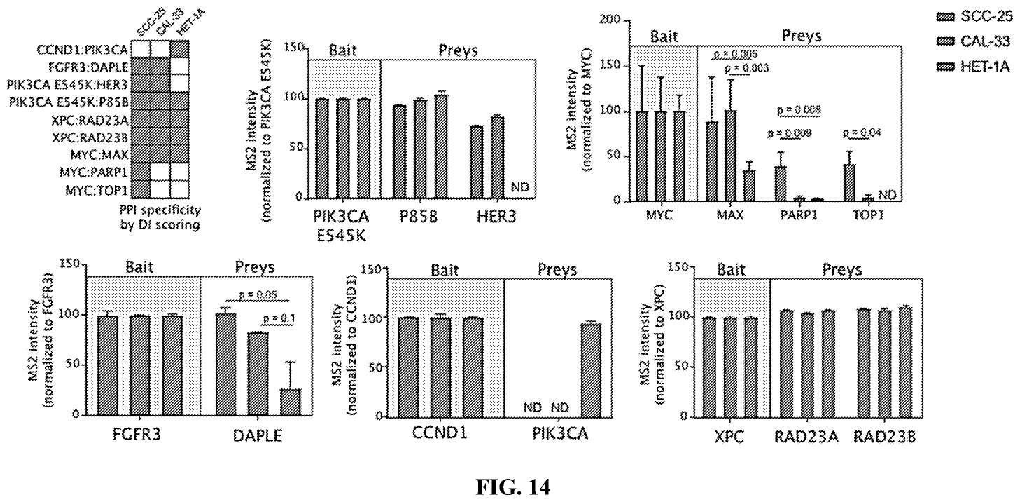

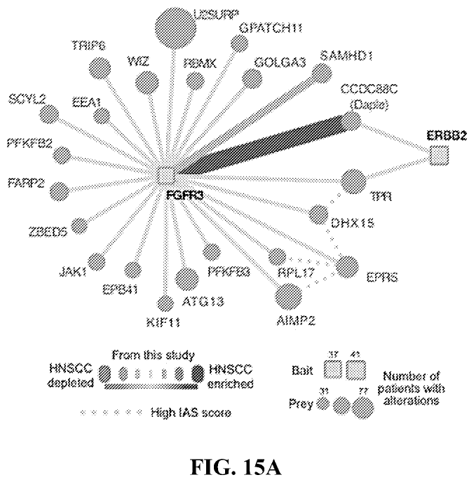

Application of the DIS method to the HC-PPIs identified numerous interactions specific to HNSCC cells as well as those specific to the HET-1A non-tumorigenic background (FIG. 13A-B). For example, the interaction profile for cyclin D1 was dramatically rewired between HNSCC and HET-1A (FIG. 13C). Cyclin D1, encoded by the CCND1 gene, is one of the most commonly altered oncogenes in HNSCC, being amplified in 31% of HPV-negative HNSCC tumors (Cancer Genome Atlas, Network, 2015). It was observed that cyclin D1 interacts with the cyclin-dependent kinase inhibitors CDKN1A (p21) and CDKN1B (p27) in all three cell lines, but preferentially interacts with multiple cyclin-dependent kinases (CDKs) only in HNSCC cells. This interaction preference was not unexpected, as CCND1:CDK4/6 interactions are known to be essential for cell proliferation and, thus, can contribute to uncontrolled cell cycle progression in cancer cells (Hamilton and Infante, 2016). A previously uncharacterized interaction of cyclin D1 was also found with components of the PI3K complex (PIK3CA, PIK3R1/2) exclusively in HET-1A cells. The specificity of this interaction, along with several others, was further supported by targeted proteomic analysis (FIG. 13C and FIG. 14). While cyclin D1 is canonically associated with the nucleus, it is also known to localize to the plasma membrane (Fusté et al., 2016). Conversely, while PI3K is often associated with cytoplasmic and membrane localization, it can also have nuclear localization (Davis et al., 2015). Without wishing to be bound by theory, the finding of a cyclin D1:PI3K kinase complex suggests that in HET-1A cells, either cyclin D1 or PI3K are present in a non-canonical subcellular location.

Referring to FIG. 13A, an interactome of the union of all HC-PPIs detected across all cell lines is shown. Edges are colored based on their differential interaction score (DIS), with pink edges representing PPIs that are enriched in HNSCC (both SCC-25 and CAL-33) as compared to HET-1A cells, and teal lines representing PPIs that are depleted from HNSCC cell lines. IAS connections represent physical protein-protein association derived from in prior studies (Zheng et al.) (see Key Resources Table 2).

Referring to FIG. 13B, for baits with |DIS|>0.5, the fraction of PPIs for that bait having HNSCC-enriched PPIs with DIS>0.5, or HNSCC-depleted DIS<−0.5 is shown.

Referring to FIG. 13C, a CCND1 interactome is shown. Here the SAINTexpress score, used for calculation of the DIS, is displayed for each cell line within the prey node, ND indicates not detected.

Referring to FIG. 14, an overview of PPI specificity as determined by DIS values for a selection of PPIs and targeted proteomic analysis for these PPIs measuring bait and prey protein abundances is shown. All prey quantification is normalized to the bait level level expression in the respective cell line. All p-values are the result of a 2-tailed unpaired t-test.

Identification of a Novel FGFR3:Daple Interaction that Regulates Gαi-Mediated Migratory Signaling

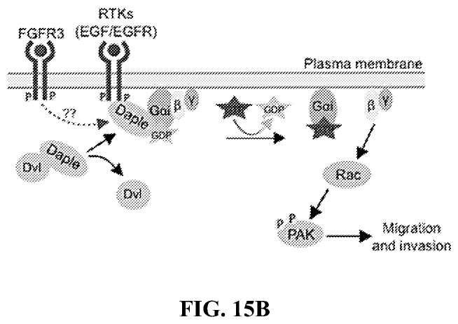

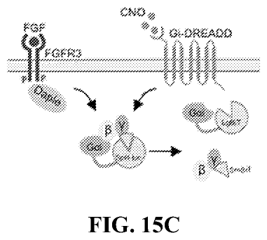

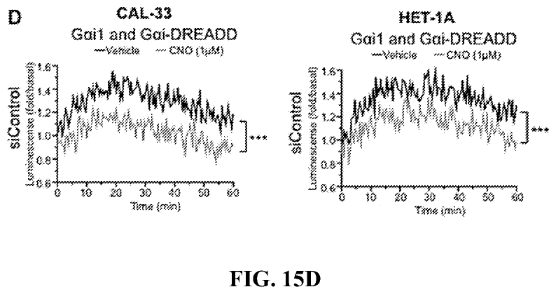

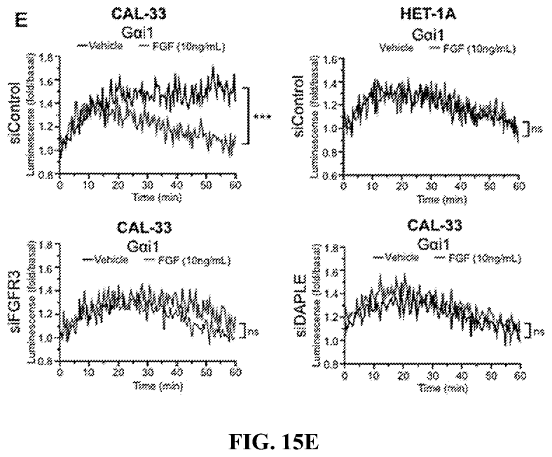

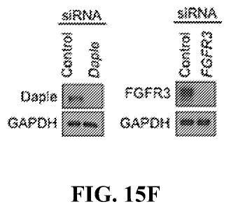

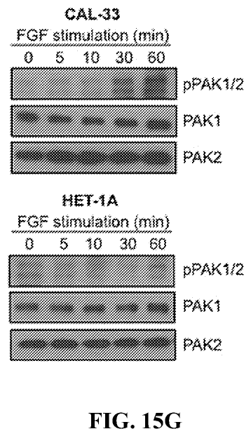

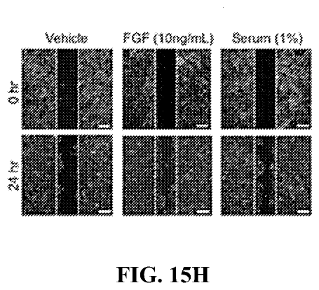

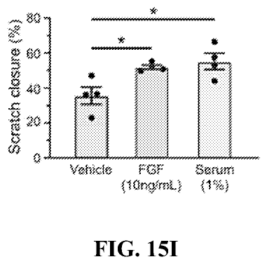

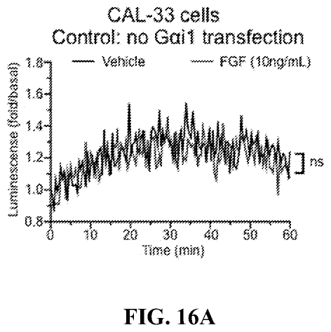

To uncover cancer-specific interactions, PPIs were ranked by their DIS (FIG. 13D), focusing on those PPIs with greatest enrichment (DIS>0.5) or depletion (DIS<−0.5) in the HNSCC cell lines (FIG. 13E). This analysis prioritized a novel interaction between FGFR3 and CCDC88C, which was strongly observed in both CAL-33 and SCC-25 cells but not in HET-1A (FIG. 15A). FGFR3 is a receptor tyrosine kinase (RTK) that recognizes fibroblast growth factor (FGF) and mediates cellular proliferation, survival and differentiation. Meanwhile, CCDC88C, also known as Daple, is a 228-kDa scaffolding protein with roles in mediating both canonical and non-canonical Wnt signaling (Aznar et al., 2017, 2018; Ishida-Takagishi et al., 2012; Oshita et al., 2003). Daple regulates Wnt through its interaction with the protein Disheveled (Dvl) (Oshita et al., 2003), and it can also interact with RTKs, including EGFR and ERBB2 (HER2) (Aznar et al., 2018), leading to its phosphorylation and dissociation from Disheveled (Aznar et al., 2018). Upon this dissociation, Daple translocates from the cytoplasm to the plasma membrane where it functions as a guanine nucleotide exchange factor (GEF) to activate G proteins (Gαi) and promote Akt signaling, cell migration, and invasion (FIG. 15B) (Aznar et al., 2015). The previously-described ERBB2:Daple interaction (Aznar et al., 2018) was detected in CAL-33 cells, as well as a novel FGFR3:Daple interaction, which was hypothesized to function to promote Gαi activation in an FGFR3-dependent manner.

Referring to FIG. 13D, DIS for the entire interactome represented in panel A ranked by DIS is shown.

Referring to FIG. 13E, a subnetwork of the interactome of the HNSCC-enriched and -depleted interactions is shown.

Referring to FIG. 15A, a differential scoring analysis of the FGFR3 interactome highlights CCDC88C (Daple) as an HNSCC-specific interaction partner to both FGFR3 and ERBB2 (HER2).

Referring to FIG. 15B, activation of RTKs can disrupt the interaction between Disheveled (Dvl) and Daple, allowing Daple to function as a GEF for Gαi. GTP binding causes dissociation of the G protein, leaving Gβγ subunits free to activate migratory signaling through Rac and PAK.

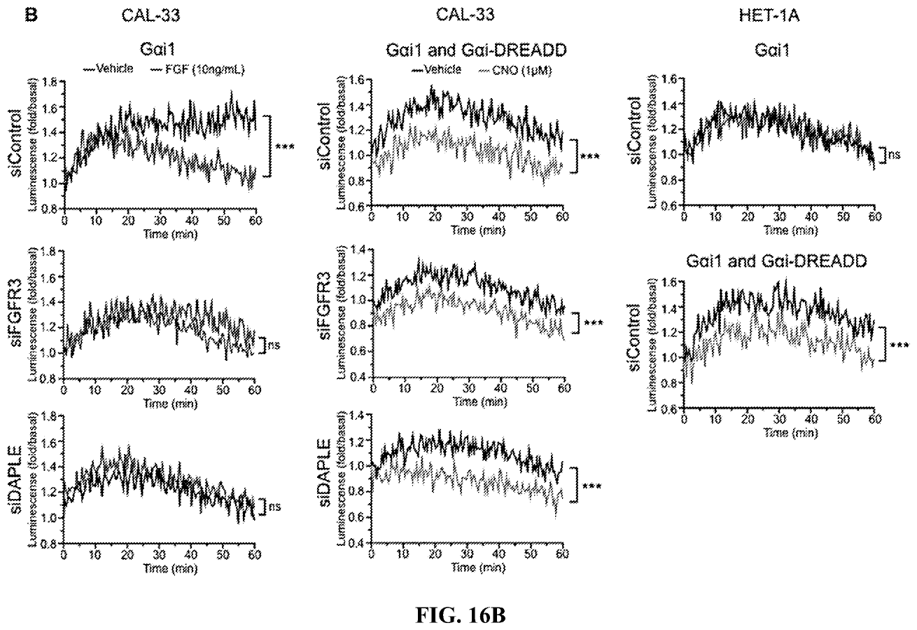

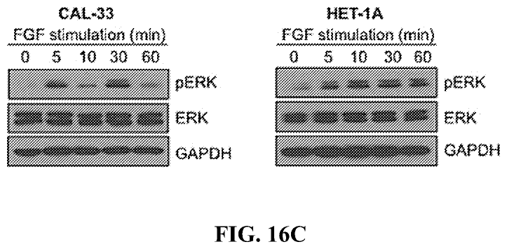

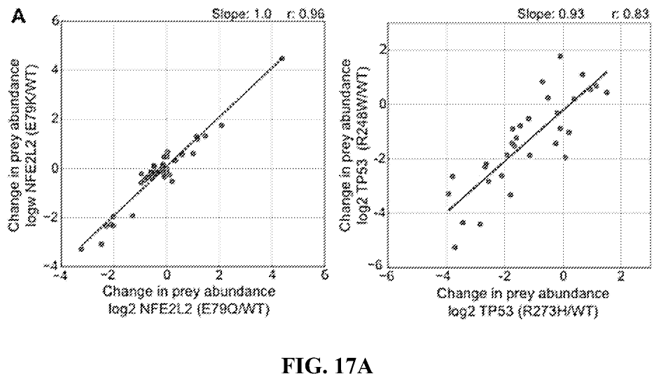

To test this idea, a split luciferase assay (Gαi NanoBiT) was used, in which signal is lost upon activation of Gαi and dissociation from Gβγ (FIG. 15C and FIG. 16A). As a control, an engineered Designer Receptor Exclusively Activated by Designer Drugs (DREADD) receptor was first transfected, and the resulting cell population stimulated with the DREADD ligand, clozapine-N-oxide (CNO). Robust Gαi activation and corresponding loss of luciferase signal was observed in both CAL-33 and HET-1A cell lines (FIG. 15D).

Next, it was observed that in the CAL-33 cells, where the interaction between FGFR3 and Daple was detected, FGF stimulation can also induce Gαi activation; however, no such activation occurred in HET-1A cells (FIG. 15E). Using siRNA knockdowns, it was found that Gαi activation in CAL-33 cells was dependent on both FGFR3 and Daple (FIG. 15E-F and FIG. 16B). Of note, FGF also rapidly induced ERK phosphorylation in both CAL-33 and HET-1A cells, in line with canonical RTK signaling (FIG. 16C). It was also observed that FGF-mediated Gαi activation in CAL-33 cells results in downstream phosphorylation of PAK1/2, an event not observed in HET-1A (FIG. 15G). PAK1/2 activity is known to promote cell migration and invasion and is associated with aggressive tumor behavior and poor patient prognosis in HNSCC (Park et al., 2015). Thus, whether FGF stimulation promoted cell migration was also evaluated. Indeed, a statistically significant increase equivalent to that of stimulation with serum was observed (FIG. 15H and FIG. 15I). Without wishing to be bound by theory, these results support a novel mechanism for regulating Gαi activity via FGFR3 and Daple, resulting in increased PAK1/2 activation and cell migration.

Referring to FIG. 15C, NanoBiT biosensor measures Gαi activation through dissociation of the luciferase split between Gα and Gβγ. CNO mediates canonical GPCR signaling through the synthetic Gαi-coupled DREADD receptor. FGF mediates HNSCC-specific signaling through FGFR3 and Daple.

Referring to FIG. 15D, CAL-33 (HNSCC) and HET-1A (normal) cells expressing the Gαi NanoBiT and DREADD receptor were stimulated with CNO (10 μM) and Gαi activity was measured by a drop in luminescence over 60 minutes (***P<0.001 when compared with the vehicle-treated group).

Referring to FIG. 15E, similarly, luminescence was measured in CAL-33 and HET-1A cells transfected with Gαi NanoBiT and siRNA (control, FGFR3, or Daple) and stimulated with FGF (1Ong/mL) (***P<0.001 when compared with the vehicle-treated group).

Referring to FIG. 15F, immunoblot analysis of CAL-33 subject to siRNA knockdown is shown.

Referring to FIG. 15G, PAK1/2 autophosphorylation was measured by immunoblot analysis over a time course of FGF stimulation (0, 5, 10, 30, 60 minutes) in CAL-33 and HET-1A cells.

Referring to FIG. 15H, a vertical scratch was introduced to fibronectin-plated CAL-33 cells. Images were taken at 0 and 24 hours after FGF stimulation (scale bar=200 μm). (I) Quantification of replicate scratch closure assays from panel H (*P<0.05 when compared with the vehicle-treated group).

Referring to FIG. 16A, luminescence was measured over 60 minutes in mock transfected CAL-33 cells stimulated with FGF (10 ng/mL).

Referring to FIG. 16B, as shown in FIG. 15E, luminescence was measured in CAL-33 cells transfected with Gαi NanoBiT, and siRNA (control, FGFR3, or Daple) and stimulated with FGF (10 ng/mL). Additionally, luminescence was measured in CAL-33 cells transfected with Gαi NanoBiT, Gαi-DREADD, and siRNA (control, FGFR3, or Daple) and stimulated with CNO (10 μM). HET-1A cells were transfected with Gαi NanoBiT alone or with the additional Gαi-DREADD and stimulated with FGF or CNO, respectively (FIG. 15D and FIG. 15E). Luminescence was measured over 60 minutes with a decrease in luminescent signal demonstrating Gαi activation (***P<0.001 when compared with the vehicle-treated group).

Referring to FIG. 16C, ERK phosphorylation over a time course was measured by immunoblotting in CAL-33 and HET-1A cells stimulated with FGF (10 ng/mL).

### Quantitative Analysis of the Effect of Mutations on the PPI Landscape

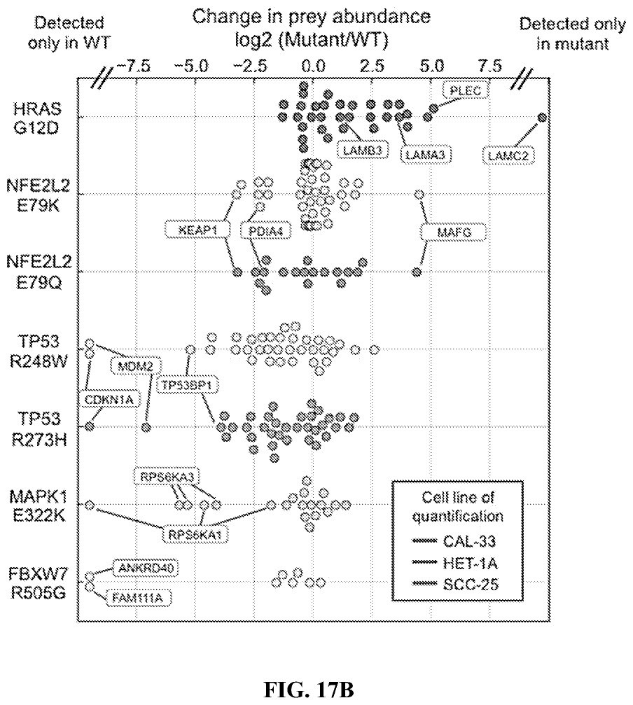

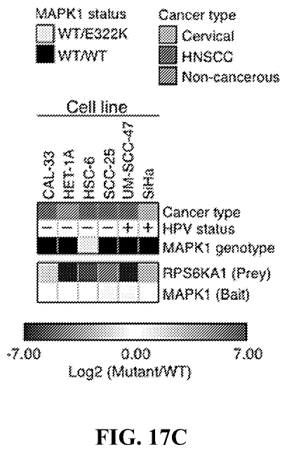

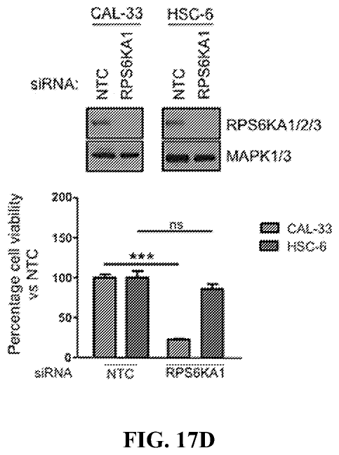

In addition to comparing the specificity of interactions across tumor and non-tumor cell lines, AP-MS data for both WT and mutant proteins was compared to identify mutant-regulated interactions. Mutations selected for this analysis were those found to be highly recurrent in HNSCC tumor genomes, considering recurrent point mutations and single amino acid deletions (Key Resources Table 2). A label-free quantitative proteomics approach was used to quantify the differential prey abundances between WT and mutant baits analyzed within the same cell line. As a negative control experiment, the correlation of prey abundances for two very similar mutations on NFE2L2, E79K, and E79Q were first examined. Very high correlation in prey abundance (r=0.96) was observed for these similar mutant isoforms (FIG. 17A). Good correlation was also seen for a second control experiment comparing R248W and R273H mutations in TP53 (r=0.83, FIG. 17A). Without wishing to be bound by theory, these results suggest not only a high degree of biological similarity between these individual point mutations on the same protein, but also a high degree of technical accuracy in our quantification of PPIs.

Referring to FIG. 17A, quantification of PPI regulation of two distinct mutations on NFE2L2 (left) or TP53 (right), respectively, is shown.

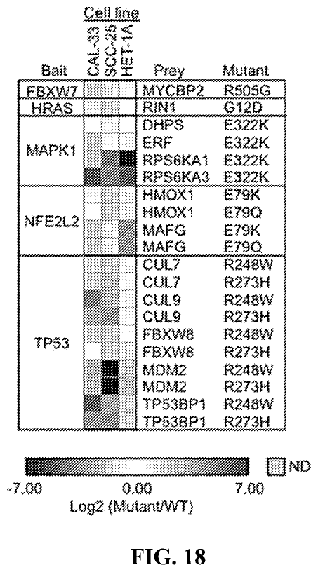

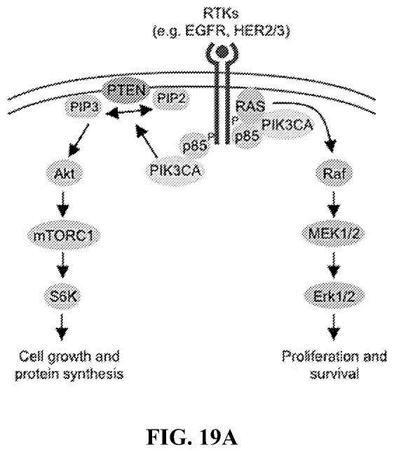

PPIs were quantitatively analyzed for missense mutations on six different proteins in total (FIG. 17B; note PIK3CA is displayed in FIG. 18). This analysis identified several previously described mutation-dependent PPIs including those involving NFE2L2, a transcriptional activator that regulates genes involved in the oxidative stress response. Under normal conditions, protein levels of NRF2, which is encoded by NFE2L2, are maintained at low levels by its association with the KEAP1 protein, which promotes its proteasome-mediated degradation. Previous work has shown that the interaction between NRF2 and KEAP1 is lost in the context of NFE2L2 E79K/Q mutations (Shibata et al., 2008), leading to increased NRF2 and promotion of carcinogenesis (Taguchi and Yamamoto, 2017). Consistent with this work, it was observed the interaction between NRF2 and KEAP1 is the most dependent on NFE2L2 mutations (FIG. 17B).

Referring to FIG. 17B, quantification of HC-PPIs for all mutants analyzed, with the exception of PIK3CA mutants, is shown. Each dot represents an individual PPI. A selection of interactions that are highly differential between WT and mutant are annotated, with the line color representing the cell line from which that PPI was quantified.

An unexpected finding was that the HRAS G12D mutant caused a general increase in the abundance of its interaction partners. Mutant HRAS is known to have increased plasma membrane localization, and, accordingly, it was found that the gained interactions included several proteins related to hemidesmosome assembly, including PLEC, LAMA3, LAMB3, and LAMC2. In particular, LAMA3 (laminin u3), LAMB3 (laminin 03), and LAMC2 (laminin γ2) are extracellular matrix proteins that function in epidermal adhesion and together form the laminin 332 heterotrimeric complex. The laminin 332 complex is highly expressed in many squamous cell carcinomas, including HNSCC where it is associated with increased tumor invasion and metastasis, and, consequently, worse prognosis (Marinkovich, 2007). Analysis of HRAS mutation and genetic alterations (mutation and CNVs) in the laminin 332 complex in HNSCC tumors revealed a statistically significant mutual exclusivity (q=0.042), suggesting functional redundancy. While this interaction between intracellular HRAS and an extracellular complex is unexpected, laminin 332 expression is known to cause clustering of RTKs and subsequent activation of Ras pathways (Tsuruta et al., 2008). It may be that the observed HRAS:laminin 332 interaction is tethered by MET, an RTK which is also find to be 2.9-fold increase in interaction with mutant HRAS.

Some of the most consistently regulated PPIs in the entire dataset were interactions of MAPK1 with RPS6KA1 and RPS6KA3, which were lost in the context of E322K mutation across all six cell lines examined (FIG. 17D). MAPK1 encodes ERK2, a protein kinase functioning directly upstream of RPS6KA1/3 (RSK1/2) in the Ras/Raf/MEK/ERK pathway that is activated in many types of cancer. The MAPK1 E322K mutation results in constitutive activation of this kinase (Arvind et al., 2005), which is associated with anchorage-independent growth (Mahalingam et al., 2008) and resistance to Raf/MEK inhibitors (Goetz et al., 2014). Structural analysis indicates that E322 coordinates a network of electrostatic interactions important for protein binding, and that mutation of this residue to a positively charged lysine destabilizes binding with RPS6KA1/3 (Alexa et al., 2015; Brenan et al., 2016; Mahalingam et al., 2008; Taylor et al., 2019). The functional consequences of the MAPK1:RPS6KA1 PPI were further tested by performing siRNA knockdown of RPS6KA1 in a cellular background of either MAPK1 WT/WT (CAL-33 cells) or MAPK1 WT/E322K (HSC-6) alleles (FIG. 17D). It was observed that in the WT/WT background, but not WT/E322K, knockdown of RPSK6A1 caused a dramatic loss in cell viability, indicating a reliance on this signaling pathway for cell survival in MAPK1 WT/WT cells. While the exact mechanism for this difference in cell viability is unclear, it may be that in the context of a WT/E322K heterozygosity, the presence of E322K can function in a dominant-negative manner, rewiring cellular signaling to maintain survival independent of RPS6KA1.

Referring to FIG. 17C, regulation of the MAPK1-interacting protein RPS6KA1 (RSK1) across a panel of six cell lines is shown.

Referring to FIG. 17D, immunoblot validation and cell viability upon siRNA knockdown of RPS6KA1 in CAL-33 cells, which endogenously harbor homozygous WT MAPK1, or the HSC-6 cell line, which is MAPK1 heterozygous (WT/E322K), are shown. MAPK1/3 (ERK1/2) total protein immunoblot is shown as a control (***P<0.001 when compared with the non-targeting control (NTC) siRNA).

### Quantitative Analysis of the Mutant PIK3CA Interactome

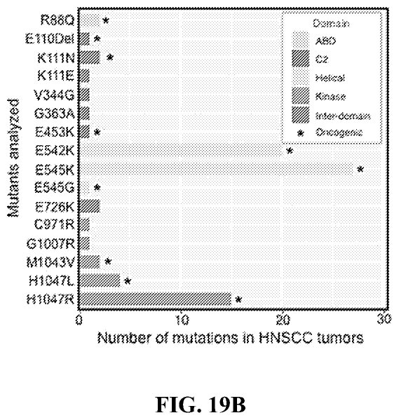

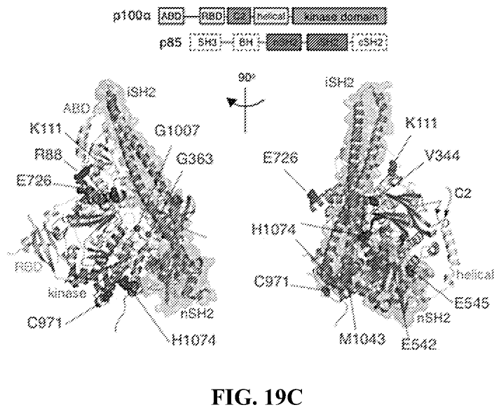

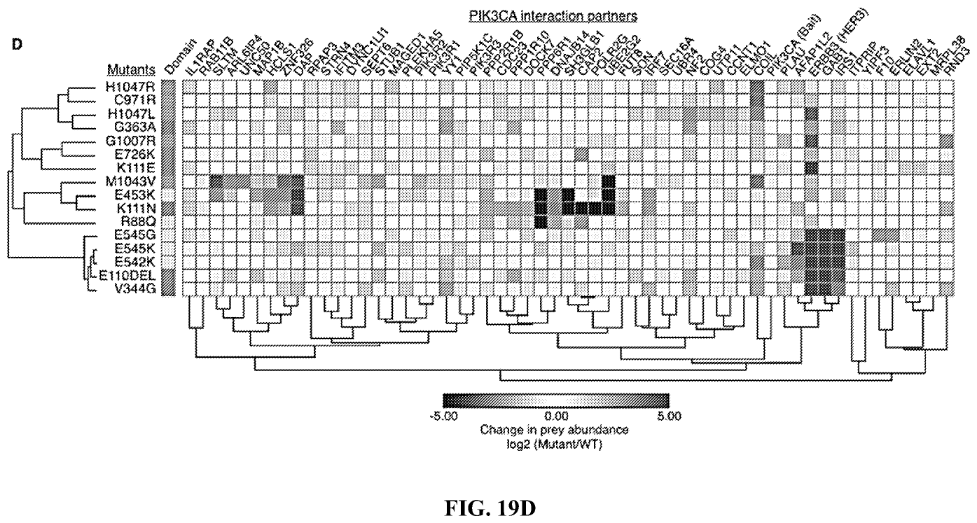

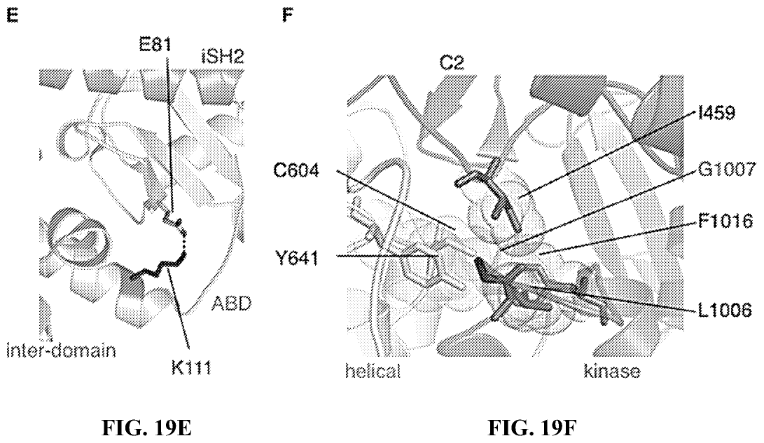

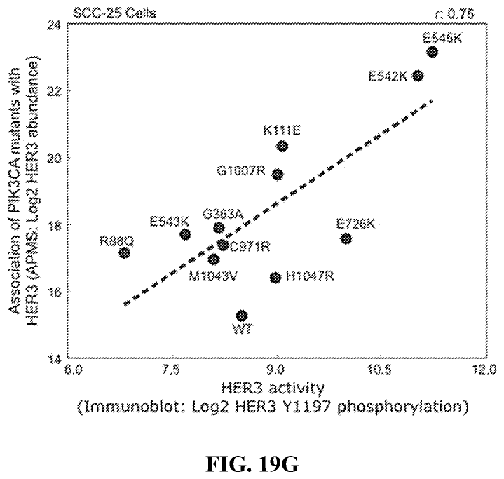

PIK3CA encodes p110alpha (p110α), the catalytic subunit of phosphatidylinositol 3-kinase (PI3K). PI3K is a potent mediator of cellular signaling, interacting with both intracellular small GTPases (e.g., RAS proteins) as well as receptor kinases (e.g., EGFR) to regulate downstream signaling via both the MAPK/ERK pathway and the Akt/mTOR pathway (FIG. 19A). Here, 16 different PIK3CA mutations observed in HNSCC patients were selected, and the effects of these mutations were quantitatively assessed on p110α interaction partners (FIG. 19B). These mutations were not limited to a particular region of the p110α structure but resided over many different surfaces (FIG. 19C). Examining the protein-protein interaction profiles of WT PIK3CA and the corresponding mutants in SCC-25 cells revealed a cluster of mutants (M1043V, E453K, and K111N) for which the same set of preys had increased interaction, particularly for DAP, death-associated protein 1 (FIG. 19D). Interestingly, a downstream component of PI3K signaling, mTOR, has been shown to phosphorylate DAP, leading to autophagy suppression (Koren et al., 2010a, 2010b). The strengthening of the DAP interaction may result in increased DAP phosphorylation and promotion of cell survival in the context of these oncogenic mutations. This same set of PIK3CA mutants exhibited a reduction of interactions with a second group of preys (FIG. 19D), including SH3GLB1 (Endophilin B1), which is known to interact with other lipid kinases, such as Class-III PIK3C3, to promote autophagy (Takahashi et al., 2007). The loss of SH3GLB1 interaction with these PIK3CA mutants may serve to reduce autophagy-promoting signals.

Referring to FIG. 19A, an overview of the PIK3CA signaling pathway, which is often stimulated by RTKs that interact with PIK3CA to stimulate RAS/Raf-mediated or Akt/mTORC1-mediated downstream signaling is shown.

Referring to FIG. 19B, analyzed PIK3CA mutants and their frequency in HNSCC tumors from TCGA is shown. Asterisk (*) denotes mutations annotated as oncogenic in OncoKB (Chakravarty et al., 2017). Graph bars corresponding to each mutation were color-coded to indicate their localization within the p110α domain (as indicated in the legend in top right corner).

Referring to FIG. 19C, selected PIK3CA mutations were mapped on the structure of PI3K (PDB: 4L23) (Zhao et al., 2014) by highlighting the mutated residues as red spheres.

Referring to FIG. 19D, quantification of PPIs for all HC-PPIs detected in the SCC-25 cell lines is shown (all cell lines displayed in FIG. 18).

Perhaps the most striking observation from the mutant PIK3CA interactome was the very high similarity in interaction patterns of five of the PIK3CA mutants (E110DEL, V344G, E542K, E545G, and E545K) (FIG. 19D), driven by a strong increase in interaction of these mutants with three proteins, ERBB3 (HER3), GAB1, and IRS1. These prey proteins all share the property of multiple YxxM motifs, representing consensus binding sites for the two SH2 domains (nSH2 and cSH2) located in the PI3K p85 regulatory subunit connected by the iSH2 coiled coil domain (Songyang et al., 1994). Engagement of phosphorylated YxxM motifs with the SH2 domains of p85 is essential for PI3K signaling by releasing p110α autoinhibition and mediating recruitment of PI3K to the plasma membrane (Dornan and Burke, 2018). The helical domain mutants (E545K, E545G, E542K) disrupt the interaction of p110α with its auto-inhibitory p85 subunits, making the p85 nSH2 domain more readily available for interaction with phosphorylated YxxM motifs. Outside of this primary cluster of mutations, other mutation sites (e.g., K111E and G1007R) were also observed with a strong increase in HER3 binding. In these cases also, mutations are expected to compromise the p85-imposed inhibition of the p110α catalytic module, either by disruption of the ABD domain relative to the inhibitory iSH2 module of p85 (K111E, FIG. 19E), or by disruption of a hydrophobic cluster coordinating amino acids from multiple p110α domains (G1007R, FIG. 19F).

Referring to FIG. 19E, a cartoon representation of a zoomed-in view of PI3K illustrating a salt bridge formed between K11 and E81 (PDB: 4L23) is shown.

Referring to FIG. 19E, a zoomed-in view depicting interactions made by G1007 in PI3K (PDB: 4L23) is shown.

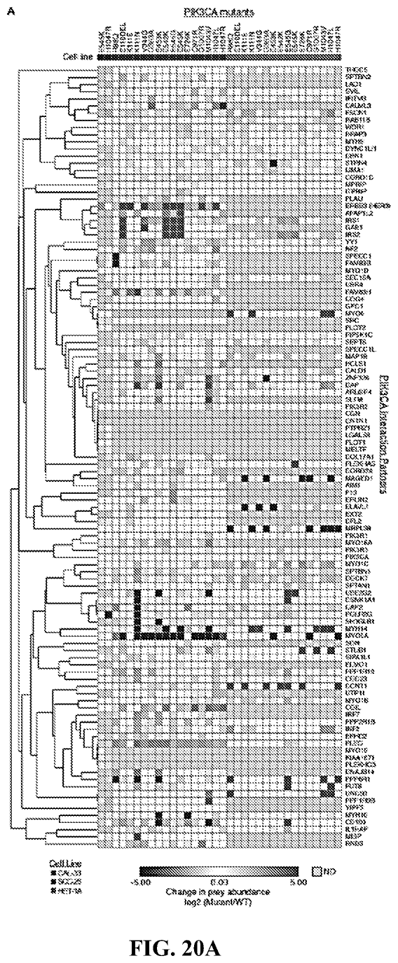

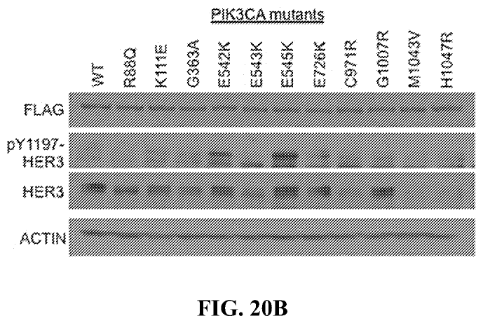

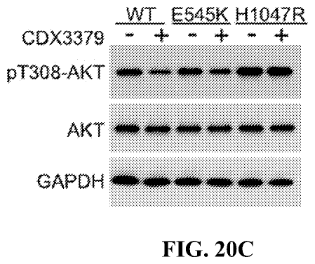

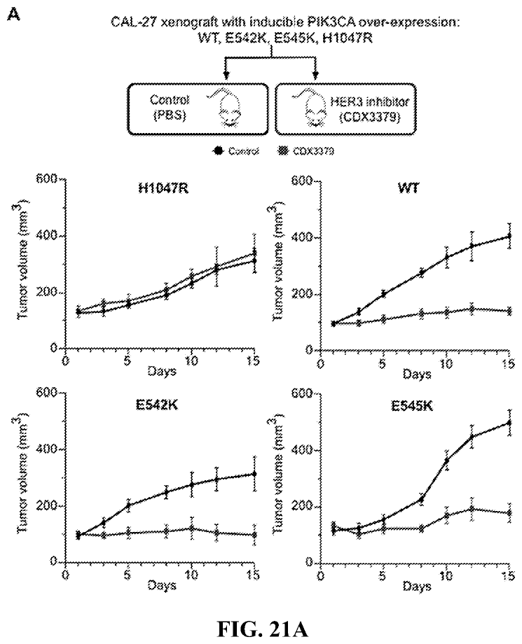

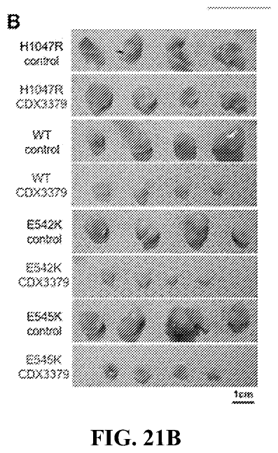

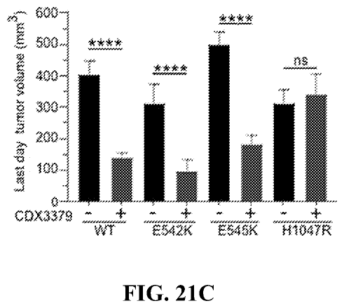

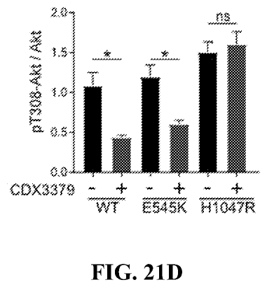

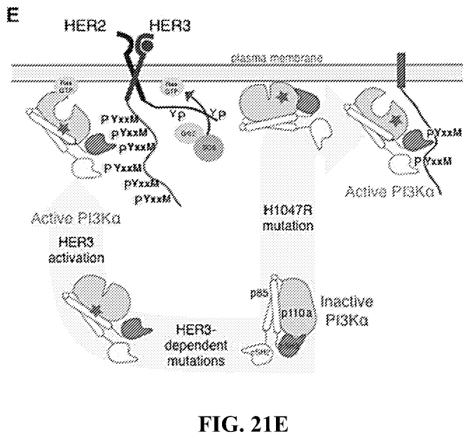

These results led to the hypothesis that the differential binding observed across PIK3CA mutants may correlate with HER3 activation. Indeed, a strong correlation between the association of individual PIK3CA mutants with HER3 was observed, as measured by AP-MS, and HER3 activation, as measured by immunoblotting of Y1197 phosphorylation (r=0.75, FIG. 19G and FIG. 20B). Activation of HER3 has previously been recognized as important in HNSCC, and clinical trials of inhibitors of HER3 signaling have been completed or are underway using a variety of agents, including the monoclonal antibody CDX3379 (Duvvuri et al., 2019). It was thus hypothesized that an HER3 inhibitor might prove particularly effective in the context of PIK3CA helical domain mutants, which show increased binding to HER3 and correlate with increased phosphorylation of HER3, in comparison to other mutants (FIG. 19D-G). To test this hypothesis, isogenic CAL-27 cell lines overexpressing either WT, E542K, E545K, or H1047R mutant isoforms of PIK3CA were generated, and injected into the flanks of athymic nude mice (Key Resources Table 2). Importantly, CAL-27 cells were used, as they are diploid for WT PIK3CA. Mice were then treated with either saline (control) or the HER3 inhibitor CDX3379 over the course of 15 days, and tumor size was monitored (FIG. 21A-C). Tumors harboring the H1047R mutant, which did not bind highly to or increase phosphorylation of HER3, were resistant to CDX3379. In contrast, CDX3379 treatment of xenograft models harboring the helical domain mutants, E542K and E545K, resulted in almost complete inhibition of tumor growth. This finding was unanticipated as all PIK3CA mutations have been thought to confer resistance to HER3 inhibition.

Referring to FIG. 19G, a correlation of Log 2 HER3 interaction levels from AP-MS experiments and Log 2 HER3 Y1197 phosphorylation levels from immunoblot analysis is shown. All values are normalized by FLAG-PIK3CA levels in their respective experiments.

Referring to FIG. 20A, quantification of PPIs for all HC-PPIs detected in all cell lines is shown.

Referring to FIG. 20B, an immunoblot of total and phosphorylated HER3 (Y1197), total HER3, Actin (loading control), and FLAG peptide in SCC-25 cells expressing a panel of FLAG-tagged PIK3CA mutants is shown.

Referring to FIG. 20C, a representative immunoblot of phosphorylated Akt (T308), total Akt, and GAPDH (loading control), in CAL-27 cells expressing WT, E545K, or H1047R PIK3CA is shown. Cells were treated in vitro with either DMSO or the CDX3379 (1 μg/ml, 1 hr).

Referring to FIG. 21A-C, CAL-27 cells expressing inducible PIK3CA variants were transplanted into athymic nude mice. Mice were fed with doxycycline to induce PIK3CA expression. When tumor volumes reached approximately 100 mm³, mice were treated with vehicle (PBS) or CDX3379 (10 mg/kg, twice a week) for approximately 15 days, as indicated. Shown are (FIG. 21A) tumor growth curves, (FIG. 21B) representative tumor images, and (FIG. 21C) last day tumor volume (****P<0.0001 when compared with the control-treated group).

To further investigate the mechanisms regulating these in vivo phenotypes, the levels of phosphorylated Akt (pAkt), a downstream mediator of PI3K signaling, were assessed in CAL-27 cells. For mutants in which CDX3379 treatment inhibited tumor growth in vivo (E542K and E545K), in vitro treatment resulted in significant downregulation of pAkt levels, whereas no such decrease was observed for the CDX3379-resistant H1047R-expressing cells (FIG. 21D and FIG. 20C).

Referring to FIG. 21D, quantification of immunoblot analysis of signaling events in the same CAL-27 cells in vitro is shown. PIK3CA variant expression was induced by doxycycline (1 μg/ml in culture medium), cells were treated with CDX3379 (1 μg/ml, 1 hr), and lysates were analyzed by immunoblotting as indicated. Densitometry analysis of western blots was performed using ImageJ. Data are represented as mean±SEM, n=3 in each group. (*P<0.05 when compared with the control-treated group).

### Discussion

In this study, the physical landscape of protein-protein interactions targeting genes genetically linked to HNSCC were examined, revealing hundreds of novel PPIs. It was observed that these interactions are highly specific to the cell line of study, with PPIs shared between cancer cell lines being no more similar than those shared between these cancer cell lines and the non-tumorigenic HET-1A cells. In support of previous observations (Huttlin et al., 2020), these results suggest the exciting premise that there remains a vast network of PPIs left to discover beyond the thousands annotated from HEK293T and HeLa cells (Hein et al., 2015; Huttlin et al., 2015, 2017). It is anticipated that developments in high-throughput protein complex determination, such as co-elution (Salas et al., 2020), proximity-labeling (Lobingier et al., 2017; Samavarchi-Tehrani et al., 2020), and cross-linking MS (Klykov et al., 2018), will enable the rapid advancement of systematic PPI mapping in a diverse array of cancer cell contexts.

An important goal of cancer therapy is to identify drug targets that are applicable across many patients and that achieve high specificity for cancer cells among a heterogeneous tumor cell population. In the context of PPIs, this goal requires moving beyond simply cataloging protein-protein interactions towards robust comparative analysis of PPIs across cellular contexts. For this purpose, a differential interaction score (DIS) was created, and the value of this DIS to statistically compare PPIs across contexts was demonstrated, which will aid in not only understanding the underlying biology behind HNSCC, but other cancers and disease in general.

### A Novel FGFR3:Daple Interaction Promotes Cell Motility

It is becoming increasingly evident that Daple has a greater diversity of cellular roles than initially appreciated. Early work established its role in mediating both canonical and non-canonical Wnt signaling via the Frizzled receptor (Ara et al., 2016; Aznar et al., 2017; Ishida-Takagishi et al., 2012; Oshita et al., 2003). Further studies have shown Daple is activated by RTK (Aznar et al., 2018) and can function as a non-receptor GEF capable of activating Gαi and Rac1 (Aznar et al., 2015). The findings disclosed herein build upon these findings by demonstrating that FGF stimulation can activate Gαi in a Daple- and FGFR3-dependent manner, which results in activation of PAK1/2 kinases and cell motility. This work also suggests that the previously undescribed connection between FGFR3 and Daple mediates Gαi and PAK1/2 activation; no such activation was observed in HET-1A cells which lack this interaction.

Importantly, PAK1 expression is highly correlated with aggressive tumor behavior and poor patient prognosis in HNSCC (Park et al., 2015; Parvathy et al., 2016). The finding that FGFR3 can mediate HNSCC-specific activation of PAK1/2 becomes increasingly important as FGFR inhibitors progress towards the clinic. Phase II clinical trials with rogaratinib, an FGFR inhibitor, are underway for HNSCC patients with FGFR1/2/3 mRNA overexpression (NCT03088059), after phase I trials demonstrated a 67% objective response rate for solid tumors with FGFR mRNA overexpression (Schuler et al., 2019). Additionally, a complete response was observed in a metastatic HNSCC tumor with multiple FGFR amplifications, including FGFR3, when treated with a pan-FGFR inhibitor (Dumbrava et al., 2018). Further work may determine if the FGFR3:Daple interaction results in frequent coupling of FGFR and PAK1/2 activity in HNSCC patients and if other cancer types exploit this novel signaling mechanism. More direct studies are necessary to determine the extent to which FGFR and PAK1/2 activity contribute to clinical outcomes, as PAK1/2 activity could serve as an additional biomarker of patients benefiting from FGFR targeted therapy.

### Tumor Response to HER3 Inhibition is Dependent Upon PIK3CA Mutation Status

These results also highlight that the oncogenic mechanisms of individual PIK3CA mutations can be influenced by differences in PPI, and these differences can be exploited for therapeutic benefit. For example, helical domain mutations activate PI3K primarily by compromising the interactions between the p85 regulatory module and the p110α catalytic module. It was found that these mutants show increased binding to HER3, increased HER3 phosphorylation, and dependence on HER3 signaling to drive tumorigenesis (FIG. 21E). In contrast, the H1047R mutant is oncogenic independently of HER3 signaling. These features of PI3K mutants seemingly contradict previous studies showing that addition of the phosphorylated YXXM motif-containing peptides increases in vitro catalytic activity of the H1047H mutant but not the helical domain mutants (Carson et al., 2008). It is postulated that phosphorylated RTK tails are necessary not for activation of the helical domain PI3K mutants, but for their recruitment to the plasma membrane where they need to interact with RasGTP for full activation (Zhao and Vogt, 2008). This strong dependence renders cells with such mutations sensitive to HER3 inhibition. These data also show that proteins with high density of YxxM motifs, such as HER3 and IRS1/2, are particularly efficient in synergizing with the PI3K helical domain mutants in which the two SH2 domains contained within the p85 regulatory module are more available. A number of other PI3K mutants that share HER3 binding features with the helical domain mutants were also identified, and it was predicted that their oncogenic potential is also HER3-dependent (FIG. 21E). In contrast, H1047R mutation increases PI3K membrane a localization (Burke et al., 2012; Carson et al., 2008; Gkeka et al., 2014; Liu et al., 2014) and confers RasGTP independence (Zhao and Vogt, 2008). While full activation of the H1047R PIK3CA mutation still requires binding of phosphorylated RTKs, the Ras independence and innate membrane localization of this mutation enables HER3-independent tumor growth and the observed resistance of this mutant to HER3-targeted therapy.

Referring to FIG. 21E, the PI3K complex is maintained in an inactive state via auto-inhibition of the p110α (PIK3CA) catalytic subunit by the p85 regulatory subunits. Mutations in p110α can promote activation of this complex by different mechanisms. Helical domain mutants relieve auto-inhibition by the p85 subunits, which in turn seek interactions with YxxM motifs, showing preference for proteins with high YxxM density, such as HER3 and IRS1/2. In contrast, the localization of the H1047R mutation blocks auto-inhibition of the kinase domain by one p85 regulatory subunit; thus, interaction with only a single YxxM motif by the remaining p85 subunit is required for PI3K activation.

Clinical inhibition of HER3 in HNSCC patients is currently being pursued in phase II clinical trials with the monoclonal antibody CDX3379 (NCT03254927). This drug locks the HER3 extracellular domain in an inactive configuration (Lee et al., 2015) and prevents not only dimerization with co-activating RTKs (e.g., HER2) but also activation of HER3 by neuregulins (e.g., NRG1). These properties make HER3 a particularly promising target, as NRG1 is expressed at higher levels in HNSCC than in any other tumor type (Alvarado et al., 2017). The results presented here further suggest that HER3 inhibitors present an opportunity to potently target specific PIK3CA mutant tumors, a utility that had not been evaluated previously. This is important, as PIK3CA is one of the most commonly mutated oncogenes in HNSCC (Cancer Genome Atlas, Network, 2015), yet targeting of PIK3CA in the clinic has been limited by toxicity (Janku et al., 2018), likely due to its pleiotropic roles in cancer and maintenance of normal cell states. In light of these findings, patient pre-selection, such as exclusion of PIK3CA H1047R mutation carriers and inclusion of those harboring helical domain mutants, may be a valuable consideration as future phases of clinical trials proceed.

In summary, this study outlines a framework for elucidating genetic complexity through multidimensional maps of cancer cell biology and demonstrates that such maps can reveal novel mechanisms of cancer pathogenesis, instructs the selection of therapeutic targets, and informs which point mutations in the tumor are most likely to respond to treatment. As such, it is anticipated that the generation and incorporation of cancer-specific physical and functional networks may represent a critical component to interpret and predict cancer biology and its clinical outcomes.

### REFERENCES

Aihara, T., Fujiwara, Y., Ooka, M., Sakita, I., Tamaki, Y., and Monden, M. (1999). Mammaglobin B as a novel marker for detection of breast cancer micrometastases in axillary lymph nodes by reverse transcriptionpolymerase chain reaction. Breast Cancer Res. Treat. 58, 137-140.
Alessi, D. R., Andjelkovic, M., Caudwell, B., Cron, P., Morrice, N., Cohen, P., and Hemmings, B. A. (1996). Mechanism of activation of protein kinase B by insulin and IGF-1. The EMBO Journal 15, 6541-6551.
Alessi, D. R., James, S. R., Downes, C. P., Holmes, A. B., Gaffney, P. R., Reese, C. B., and Cohen, P. (1997). Characterization of a 3-phosphoinositide-dependent protein kinase which phosphorylates and activates protein kinase Balpha. Curr. Biol. 7, 261-269.
Alessi, D. R., Sakamoto, K., and Bayascas, J. R. (2006). LKB1-dependent signaling pathways. Annu. Rev. Biochem. 75, 137-163.
Allen, P. B., Ouimet, C. C., and Greengard, P. (1997). Spinophilin, a novel protein phosphatase 1 binding protein localized to dendritic spines. Proc. Natl. Acad. Sci. U.S.A 94, 9956-9961.
Altiok, S., Batt, D., Altiok, N., Papautsky, A., Downward, J., Roberts, T. M., and Avraham, H. (1999). Heregulin induces phosphorylation of BRCA1 through phosphatidylinositol 3-Kinase/AKT in breast cancer cells. J. Biol. Chem. 274, 32274-32278.
Alva, V., and Lupas, A. N. (2016). The TULIP superfamily of eukaryotic lipid-binding proteins as a mediator of lipid sensing and transport. Biochim. Biophys. Acta 1861, 913-923.
American Cancer Society (2019). Cancer Facts & FIGS. 2019. American Cancer Society.
Anantha, R. W., Simhadri, S., Foo, T. K., Miao, S., Liu, J., Shen, Z., Ganesan, S., and Xia, B. (2017). Functional and mutational landscapes of BRCA1 for homology-directed repair and therapy resistance. Elife 6.
Anp, P. H. V. R. M. C., Viale, P. H., R N, M S, CNS, and ANP (2020). The American Cancer Society's Facts & Figures: 2020 Edition. Journal of the Advanced Practitioner in Oncology 11.
Apostolou, P., and Papasotiriou, I. (2017). Current perspectives on CHEK2 mutations in breast cancer. Breast Cancer 9, 331-335.
Arizti, P., Fang, L., Park, I., Yin, Y., Solomon, E., Ouchi, T., Aaronson, S. A., and Lee, S. W. (2000). Tumor suppressor p53 is required to modulate BRCA1 expression. Mol. Cell. Biol. 20, 7450-7459.
Aylon, Y., and Oren, M. (2011). New plays in the p53 theater. Curr. Opin. Genet. Dev. 21, 86-92.
Baas, A. F., Boudeau, J., Sapkota, G. P., Smit, L., Medema, R., Morrice, N. A., Alessi, D. R., and Clevers, H. C. (2003). Activation of the tumour suppressor kinase LKB1 by the STE20-like pseudokinase STRAD. EMBO J. 22, 3062-3072.
Baas, A. F., Kuipers, J., van der Wel, N. N., Batlle, E., Koerten, H. K., Peters, P. J., and Clevers, H. C. (2004). Complete polarization of single intestinal epithelial cells upon activation of LKB1 by STRAD. Cell 116, 457-466.
Barker, A. D., Sigman, C. C., Kelloff, G. J., Hylton, N. M., Berry, D. A., and Esserman, L. J. (2009). I-SPY 2: An Adaptive Breast Cancer Trial Design in the Setting of Neoadjuvant Chemotherapy. Clinical Pharmacology & Therapeutics 86, 97-100.
Beamer, L. J. (2003). Structure of human BPI (bactericidal/permeability-increasing protein) and implications for related proteins. Biochem. Soc. Trans. 31, 791-794.
Beca, F., Kensler, K., Glass, B., Schnitt, S. J., Tamimi, R. M., and Beck, A. H. (2017). EZH2 protein expression in normal breast epithelium and risk of breast cancer: results from the Nurses' Health Studies. Breast Cancer Research 19.
Beckta, J. M., Dever, S. M., Gnawali, N., Khalil, A., Sule, A., Golding, S. E., Rosenberg, E., Narayanan, A., KehnHall, K., Xu, B., et al. (2015). Mutation of the BRCA1 S Q-cluster results in aberrant mitosis, reduced homologous recombination, and a compensatory increase in non-homologous end joining. Oncotarget 6.
Bellone, S., Tassi, R., Betti, M., English, D., Cocco, E., Gasparrini, S., Bortolomai, I., Black, J. D., Todeschini, P., Romani, C., et al. (2013). Mammaglobin B (SCGB2A1) is a novel tumour antigen highly differentially expressed in all major histological types of ovarian cancer: implications for ovarian cancer immunotherapy. British Journal of Cancer 109, 462-471.
Bensimon, A., Schmidt, A., Ziv, Y., Elkon, R., Wang, S.-Y., Chen, D. J., Aebersold, R., and Shiloh, Y. (2010). ATM-Dependent and -Independent Dynamics of the Nuclear Phosphoproteome After DNA Damage. Science Signaling 3, rs3-rs3.
Bhargava, R., Sandhu, M., Muk, S., Lee, G., Vaidehi, N., and Stark, J. M. (2018). C-NHEJ without indels is robust and requires synergistic function of distinct XLF domains. Nat. Commun. 9, 2484.
Bingle, L., and Bingle, C. D. (2011). Distribution of human PLUNC/BPI fold-containing (BPIF) proteins. Biochem. Soc. Trans. 39, 1023-1027.
Bouhaddou, M., Eckhardt, M., Chi Naing, Z. Z., Kim, M., Ideker, T., and Krogan, N. J. (2019). Mapping the protein-protein and genetic interactions of cancer to guide precision medicine. Curr. Opin. Genet. Dev. 54, 110-117.
Breuleux, M., Klopfenstein, M., Stephan, C., Doughty, C. A., Barys, L., Maira, S.-M., Kwiatkowski, D., and Lane, H. A. (2009). Increased AKT S473 phosphorylation after mTORC1 inhibition is rictor dependent and does not predict tumor cell response to PI3K/mTOR inhibition. Mol. Cancer Ther. 8, 742-753.
Brieher, W. M., and Yap, A. S. (2013). Cadherin junctions and their cytoskeleton(s). Current Opinion in Cell Biology 25, 39-46.
Britto, C. J., and Cohn, L. (2015). Bactericidal/Permeability-Increasing Protein Fold-Containing Family Member Al in Airway Host Protection and Respiratory Disease. Am. J. Respir. Cell Mol. Biol. 52, 525-534.
Brognard, J., Sierecki, E., Gao, T., and Newton, A. C. (2007). PHLPP and a second isoform, PHLPP2, differentially attenuate the amplitude of Akt signaling by regulating distinct Akt isoforms. Mol. Cell 25, 917-931.
Brugge, J., Hung, M.-C., and Mills, G. B. (2007). A new mutational AKTivation in the PI3K pathway. Cancer Cell 12, 104-107.
Buller, R. E., Lallas, T. A., Shahin, M. S., Sood, A. K., Hatterman-Zogg, M., Anderson, B., Sorosky, J. I., and Kirby, P. A. (2001). The p53 mutational spectrum associated with BRCA1 mutant ovarian cancer. Clin. Cancer Res. 7, 831-838.
Burke, J. E., Perisic, O., Masson, G. R., Vadas, O., and Williams, R. L. (2012). Oncogenic mutations mimic and enhance dynamic events in the natural activation of phosphoinositide 3-kinase p110α (PIK3CA). Proc. Natl. Acad. Sci. U.S.A 109, 15259-15264.
Cairns, R. A., Harris, I. S., and Mak, T. W. (2011). Regulation of cancer cell metabolism. Nat. Rev. Cancer 11, 85-95.
Cancer Genome Atlas, Network (2012). Comprehensive molecular portraits of human breast tumours. Nature A 490, 61-70.
Canel, M., Serrels, A., Frame, M. C., and Brunton, V. G. (2013). E-cadherin-integrin crosstalk in cancer invasion and metastasis. Journal of Cell Science 126, 393-401.
Cantley, L. C., and Neel, B. G. (1999). New insights into tumor suppression: PTEN suppresses tumor formation by restraining the phosphoinositide 3-kinase/AKT pathway. Proc. Natl. Acad. Sci. U.S.A 96, 4240-4245.
Carpten, J. D., Faber, A. L., Horn, C., Donoho, G. P., Briggs, S. L., Robbins, C. M., Hostetter, G., Boguslawski, S., Moses, T. Y., Savage, S., et al. (2007). A transforming mutation in the pleckstrin homology domain of AKT1 in cancer. Nature 448, 439-444.
Chen, Z., and Coppé, J.-P. (2012). Method and System for Building and Using a Centralized and Harmonized Relational Database.
Chen, C.-C., Juan, C.-W., Chen, K.-Y., Chang, Y.-C., Lee, J.-C., and Chang, M.-C. (2017). Upregulation of RPA2 promotes NF-κB activation in breast cancer by relieving the antagonistic function of menin on NF-κB-regulated transcription. Carcinogenesis 38, 196-206.
Chen, H., Kovar, J., Sissons, S., Cox, K., Matter, W., Chadwell, F., Luan, P., Vlahos, C. J., Schutz-Geschwender, A., and Olive, D. M. (2005). A cell-based immunocytochemical assay for monitoring kinase signaling pathways and drug efficacy. Anal. Biochem. 338, 136-142.
Chen, P., Guo, X., Zhou, H., Zhang, W., Zeng, Z., Liao, Q., Li, X., Xiang, B., Yang, J., Ma, J., et al. (2013). SPLUNCI regulates cell progression and apoptosis through the miR-141-PTEN/p27 pathway, but is hindered by LMP1. PLoS One 8, e56929.
Chien, A. J., Tripathy, D., Albain, K. S., Symmans, W. F., Rugo, H. S., Melisko, M. E., Wallace, A. M., Schwab, R., Helsten, T., Forero-Torres, A., et al. (2020). MK-2206 and Standard Neoadjuvant Chemotherapy Improves Response in Patients With Human Epidermal Growth Factor Receptor 2-Positive and/or Hormone Receptor Negative Breast Cancers in the I-SPY 2 Trial. J. Clin. Oncol. 38, 1059-1069.
Cho, A., Shim, J. E., Kim, E., Supek, F., Lehner, B., and Lee, I. (2016). MUFFINN: cancer gene discovery via network analysis of somatic mutation data. Genome Biology 17.
Cho, D.-H., Jo, Y. K., Roh, S. A., Na, Y.-S., Kim, T. W., Jang, S. J., Kim, Y. S., and Kim, J. C. (2010). Upregulation of SPRR3 promotes colorectal tumorigenesis. Mol. Med. 16, 271-277.
Choi, M., Chang, C.-Y., Clough, T., Broudy, D., Killeen, T., MacLean, B., and Vitek, O. (2014). MSstats: an R package for statistical analysis of quantitative mass spectrometry-based proteomic experiments. Bioinformatics 30, 2524-2526.
Chowdhury, D., Keogh, M.-C., Ishii, H., Peterson, C. L., Buratowski, S., and Lieberman, J. (2005). gamma-H2AX dephosphorylation by protein phosphatase 2A facilitates DNA double-strand break repair. Mol. Cell 20, 801-809.
Christensen, D. E., Brzovic, P. S., and Klevit, R. E. (2007). E2-BRCA1 RING interactions dictate synthesis of mono- or specific polyubiquitin chain linkages. Nat. Struct. Mol. Biol. 14, 941-948.
Clapperton, J. A., Manke, I. A., Lowery, D. M., Ho, T., Haire, L. F., Yaffe, M. B., and Smerdon, S. J. (2004). Structure and mechanism of BRCA1 BRCT domain recognition of phosphorylated BACH1 with implications for cancer. Nat. Struct. Mol. Biol. 11, 512-518.
Coppe, J. P., Mori, M., Pan, B., Yau, C., Wolf, D. M., Ruiz-Saenz, A., Brunen, D., Prahallad, A., CornelissenSteijger, P., Kemper, K., et al. (2019). Mapping phospho-catalytic dependencies of therapy-resistant tumours reveals actionable vulnerabilities. Nat. Cell Biol. 21, 778-790.
Coppé, J.-P., Mapping, K. A., Mori, M., and Pan, B. (2019a). High-Throughput Kinase Activity Mapping (HT-KAM) system: biochemical assay. Protocol Exchange.
Coppé, J.-P., Yau, C., and Wolf, D. M. (2019b). High-Throughput Kinase Activity Mapping (HT-KAM) system: analysis of phospho-catalytic profiles. Protocol Exchange.
Cortez, D., Wang, Y., Qin, J., and Elledge, S. J. (1999). Requirement of ATM-dependent phosphorylation of brca1 in the DNA damage response to double-strand breaks. Science 286, 1162-1166.
Cox, J., and Mann, M. (2008). MaxQuant enables high peptide identification rates, individualized p.p.b.-range mass accuracies and proteome-wide protein quantification. Nat. Biotechnol. 26, 1367-1372.
Creixell, P., Reimand, J., Haider, S., Wu, G., Shibata, T., Vazquez, M., Mustonen, V., Gonzalez-Perez, A., Pearson, J., Sander, C., et al. (2015). Pathway and network analysis of cancer genomes. Nat. Methods 12, 615-621.
Davies, M. A., Stemke-Hale, K., Tellez, C., Calderone, T. L., Deng, W., Prieto, V. G., Lazar, A. J. F., Gershenwald, J. E., and Mills, G. B. (2008). A novel AKT3 mutation in melanoma tumours and cell lines. Br. J. Cancer 99, 1265-1268.
Dever, S. M., Golding, S. E., Rosenberg, E., Adams, B. R., Idowu, M. O., Quillin, J. M., Valerie, N., Xu, B., Povirk, L. F., and Valerie, K. (2011). Mutations in the BRCT binding site of BRCA1 result in hyper-recombination. Aging 3, 515-532.
Drost, R., Bouwman, P., Rottenberg, S., Boon, U., Schut, E., Klarenbeek, S., Klijn, C., van der Heijden, I., van der Gulden, H., Wientjens, E., et al. (2011). BRCA1 RING function is essential for tumor suppression but dispensable for therapy resistance. Cancer Cell 20, 797-809.
Drost, R., Dhillon, K. K., van der Gulden, H., van der Heijden, I., Brandsma, I., Cruz, C., Chondronasiou, D., Castroviejo-Bermejo, M., Boon, U., Schut, E., et al. (2016). BRCA1185delAG tumors may acquire therapy resistance through expression of RING-less BRCA1. J. Clin. Invest. 126, 2903-2918.
Eckhardt, M., Zhang, W., Gross, A. M., Von Dollen, J., Johnson, J. R., Franks-Skiba, K. E., Swaney, D. L., Johnson, T. L., Jang, G. M., Shah, P. S., et al. (2018). Multiple Routes to Oncogenesis Are Promoted by the Human Papillomavirus-Host Protein Network. Cancer Discovery 8, 1474-1489.
Epping, M. T., Meijer, L. A. T., Krijgsman, O., Bos, J. L., Pandolfi, P. P., and Bernards, R. (2011). TSPYL5 suppresses p53 levels and function by physical interaction with USP7. Nat. Cell Biol. 13, 102-108.
Escribano-Diaz, C., Orthwein, A., Fradet-Turcotte, A., Xing, M., Young, J. T., Tkac, J., Cook, M. A., Rosebrock, A. P., Munro, M., Canny, M. D., et al. (2013). A cell cycle-dependent regulatory circuit composed of 53BP1-RIF1 and BRCA1-CtIP controls DNA repair pathway choice. Mol. Cell 49, 872-883.
Feng, Z., and Levine, A. J. (2010). The regulation of energy metabolism and the IGF-1/mTOR pathways by the p53 protein. Trends Cell Biol. 20, 427-434.
Feng, J., Yan, Z., Ferreira, A., Tomizawa, K., Liauw, J. A., Zhuo, M., Allen, P. B., Ouimet, C. C., and Greengard, P. (2000). Spinophilin regulates the formation and function of dendritic spines. Proc. Natl. Acad. Sci. U.S.A 97, 9287-9292.
Forcet, C., Etienne-Manneville, S., Gaude, H., Fournier, L., Debilly, S., Salmi, M., Baas, A., Olschwang, S., Clevers, H., and Billaud, M. (2005). Functional analysis of Peutz-Jeghers mutations reveals that the LKB1 Cterminal region exerts a crucial role in regulating both the AMPK pathway and the cell polarity. Hum. Mol. Genet. 14, 1283-1292.
Fruman, D. A., Chiu, H., Hopkins, B. D., Bagrodia, S., Cantley, L. C., and Abraham, R. T. (2017). The PI3K Pathway in Human Disease. Cell 170, 605-635.
Fuqua, S. A. W., Gu, G., and Rechoum, Y. (2014). Estrogen receptor (ER) α mutations in breast cancer: hidden in plain sight. Breast Cancer Res. Treat. 144, 11-19.
Futreal, P. A., Liu, Q., Shattuck-Eidens, D., Cochran, C., Harshman, K., Tavtigian, S., Bennett, L. M., HaugenStrano, A., Swensen, J., and Miki, Y. (1994). BRCA1 mutations in primary breast and ovarian carcinomas. Science 266, 120-122.
Gatei, M., Scott, S. P., Filippovitch, I., Soronika, N., Lavin, M. F., Weber, B., and Khanna, K. K. (2000). Role for ATM in DNA damage-induced phosphorylation of BRCA1. Cancer Res. 60, 3299-3304.
Gatei, M., Zhou, B.-B., Hobson, K., Scott, S., Young, D., and Khanna, K. K. (2001). Ataxia Telangiectasia Mutated (ATM) Kinase and ATM and Rad3 Related Kinase Mediate Phosphorylation of Brca1 at Distinct and Overlapping Sites IN VIVO ASSESSMENT USING PHOSPHO-SPECIFIC ANTIBODIES. J. Biol. Chem. 276, 17276-17280.
Goh, J. Y., Feng, M., Wang, W., Oguz, G., Yatim, S. M. J. M., Lee, P. L., Bao, Y., Lim, T. H., Wang, P., Tam, W. L., et al. (2017). Chromosome 1921.3 amplification is a trackable biomarker and actionable target for breast cancer recurrence. Nat. Med. 23, 1319-1330.
Goldberg, M., Bell, K., Aronson, M., Semotiuk, K., Pond, G., Gallinger, S., and Zbuk, K. (2017). Association between the Lynch syndrome gene MSH2 and breast cancer susceptibility in a Canadian familial cancer registry. J. Med. Genet. 54, 742-746.
Górski, B., Byrski, T., Huzarski, T., Jakubowska, A., Menkiszak, J., Gronwald, J., Plużańska, A., Bçbenek, M., Fischer-Maliszewska, L., Grzybowska, E., et al. (2000). Founder Mutations in the BRCA1 Gene in Polish Families with Breast-Ovarian Cancer. Am. J. Hum. Genet. 66, 1963-1968.
Gottlieb, E., and Vousden, K. H. (2010). p⁵³ regulation of metabolic pathways. Cold Spring Harb. Perspect. Biol. 2, a001040.
Grabocka, E., Pylayeva-Gupta, Y., Jones, M. J. K., Lubkov, V., Yemanaberhan, E., Taylor, L., Jeng, H. H., and Bar-Sagi, D. (2014). Wild-type H- and N-Ras promote mutant K-Ras-driven tumorigenesis by modulating the DNA damage response. Cancer Cell 25, 243-256.
Guerrero-Zotano, A., Mayer, I. A., and Arteaga, C. L. (2016). PI3K/AKT/mTOR: role in breast cancer progression, drug resistance, and treatment. Cancer Metastasis Rev. 35, 515-524.
Gunn, A., and Stark, J. M. (2012). I-SceI-based assays to examine distinct repair outcomes of mammalian chromosomal double strand breaks. Methods Mol. Biol. 920, 379-391.
Hamilton, E., and Infante, J. R. (2016). Targeting CDK4/6 in patients with cancer. Cancer Treat. Rev. 45, 129-138.
Hardie, D. G., Grahame Hardie, D., and Alessi, D. R. (2013). LKB1 and AMPK and the cancer-metabolism link-ten years after. BMC Biology 11.
Harkness, E. F., Barrow, E., Newton, K., Green, K., Clancy, T., Lalloo, F., Hill, J., and Evans, D. G. (2015). Lynch syndrome caused by MLH1 mutations is associated with an increased risk of breast cancer: a cohort study. J. Med. Genet. 52, 553-556.
Hatchi, E., Skourti-Stathaki, K., Ventz, S., Pinello, L., Yen, A., Kamieniarz-Gdula, K., Dimitrov, S., Pathania, S., McKinney, K. M., Eaton, M. L., et al. (2015). BRCA1 recruitment to transcriptional pause sites is required for Rloop-driven DNA damage repair. Mol. Cell 57, 636-647.
He, X., Zhu, Z., Johnson, C., Stoops, J., Eaker, A. E., Bowen, W., and DeFrances, M. C. (2008). PIK3IP1, a negative regulator of PI3K, suppresses the development of hepatocellular carcinoma. Cancer Res. 68, 5591-5598.
A He, Y., Zhou, G., Zhai, Y., Dong, X., Lv, L., He, F., and Yao, K. (2005). Association of PLUNC gene polymorphisms with susceptibility to nasopharyngeal carcinoma in a Chinese population. J. Med. Genet. 42, 172-176.
Hein, M. Y., Hubner, N. C., Poser, I., Cox, J., Nagaraj, N., Toyoda, Y., Gak, I. A., Weisswange, I., Mansfeld, J., Buchholz, F., et al. (2015). A human interactome in three quantitative dimensions organized by stoichiometries and abundances. Cell 163, 712-723.
Higashiguchi, M., Nagatomo, I., Kijima, T., Morimura, O., Miyake, K., Minami, T., Koyama, S., Hirata, H., Iwahori, K., Takimoto, T., et al. (2016). Clarifying the biological significance of the CHK 2 K373E somatic mutationdiscovered in The Cancer Genome Atlas database. FEBS Lett. 590, 4275-4286.
Hill, S. J., Rolland, T., Adelmant, G., Xia, X., Owen, M. S., Dricot, A., Zack, T. I., Sahni, N., Jacob, Y., Hao, T., et al. (2014). Systematic screening reveals a role for BRCA1 in the response to transcription-associated DNA damage. Genes Dev. 28, 1957-1975.
Hinton, C. V., Fitzgerald, L. D., and Thompson, M. E. (2007). Phosphatidylinositol 3-kinase/Akt signaling enhances nuclear localization and transcriptional activity of BRCA1. Exp. Cell Res. 313, 1735-1744.
Hoenerhoff, M. J., Chu, I., Barkan, D., Liu, Z.-Y., Datta, S., Dimri, G. P., and Green, J. E. (2009). BMI1 cooperates with H-RAS to induce an aggressive breast cancer phenotype with brain metastases. Oncogene 28, 3022-3032.
Hofree, M., Shen, J. P., Carter, H., Gross, A., and Ideker, T. (2013). Network-based stratification of tumor mutations. Nat. Methods 10, 1108-1115.
Hollstein, P. E., Eichner, L. J., Brun, S. N., Kamireddy, A., Svensson, R. U., Vera, L. I., Ross, D. S., Rymoff, T. J., Hutchins, A., Galvez, H. M., et al. (2019). The AMPK-Related Kinases SIK1 and SIK3 Mediate Key TumorSuppressive Effects of LKB1 in NSCLC. Cancer Discov. 9, 1606-1627.
Holstege, H., Joosse, S. A., van Oostrom, C. T. M., Nederlof, P. M., de Vries, A., and Jonkers, J. (2009). High incidence of protein-truncating TP53 mutations in BRCA1-related breast cancer. Cancer Res. 69, 3625-3633.
Hong, Y., Shi, J., Ge, Z., and Wu, H. (2017). Associations between mutations of the cell cycle checkpoint kinase 2 gene and gastric carcinogenesis. Mol. Med. Rep. 16, 4287-4292.
Huttlin, E. L., Ting, L., Bruckner, R. J., Gebreab, F., Gygi, M. P., Szpyt, J., Tam, S., Zarraga, G., Colby, G., Baltier, K., et al. (2015a). The BioPlex Network: A Systematic Exploration of the Human Interactome. Cell 162, 425-440.
Huttlin, E. L., Ting, L., Bruckner, R. J., Gebreab, F., Gygi, M. P., Szpyt, J., Tam, S., Zarraga, G., Colby, G., Baltier, K., et al. (2015b). The BioPlex Network: A Systematic Exploration of the Human Interactome. Cell 162, 425-440.
Huttlin, E. L., Bruckner, R. J., Paulo, J. A., Cannon, J. R., Ting, L., Baltier, K., Colby, G., Gebreab, F., Gygi, M. P., Parzen, H., et al. (2017). Architecture of the human interactome defines protein communities and disease networks. Nature 545, 505-509.
Huttlin, E. L., Bruckner, R. J., Navarrete-Perea, J., Cannon, J. R., Baltier, K., Gebreab, F., Gygi, M. P., Thornock, A., Zarraga, G., Tam, S., et al. (2020). Dual Proteome-scale Networks Reveal Cell-specific Remodeling of the Human Interactome.
Iorio, F., Knijnenburg, T. A., Vis, D. J., Bignell, G. R., Menden, M. P., Schubert, M., Aben, N., Gonçalves, E., Barthorpe, S., Lightfoot, H., et al. (2016). A Landscape of Pharmacogenomic Interactions in Cancer. Cell 166, 740-754.
Johnson, W. E., Li, C., and Rabinovic, A. (2007). Adjusting batch effects in microarray expression data using empirical Bayes methods. Biostatistics 8, 118-127.
Kim, E. M., Jung, C.-H., Kim, J., Hwang, S.-G., Park, J. K., and Um, H.-D. (2017). The p53/p21 Complex Regulates Cancer Cell Invasion and Apoptosis by Targeting Bcl-2 Family Proteins. Cancer Res. 77, 3092-3100.
Kim, H., Huang, J., and Chen, J. (2007a). CCDC98 is a BRCA1-BRCT domain-binding protein involved in the DNA damage response. Nature Structural & Molecular Biology 14, 710-715.
Kim, H., Chen, J., and Yu, X. (2007b). Ubiquitin-binding protein RAP80 mediates BRCA1-dependent DNA damage response. Science 316, 1202-1205.
Kim, S. T., Lim, D. S., Canman, C. E., and Kastan, M. B. (1999). Substrate specificities and identification of putative substrates of ATM kinase family members. J. Biol. Chem. 274, 37538-37543.
Knijnenburg, T. A., Wang, L., Zimmermann, M. T., Chambwe, N., Gao, G. F., Cherniack, A. D., Fan, H., Shen, H., Way, G. P., Greene, C. S., et al. (2018). Genomic and Molecular Landscape of DNA Damage Repair Deficiency across The Cancer Genome Atlas. Cell Rep. 23, 239-254 e6.
Kumar, R. D., and Bose, R. (2017). Analysis of somatic mutations across the kinome reveals loss-of-function mutations in multiple cancer types. Sci. Rep. 7, 6418.
Landgraf, K. E., Pilling, C., and Falke, J. J. (2008). Molecular Mechanism of an Oncogenic Mutation That Alters Membrane Targeting: Glu17Lys Modifies the PIP Lipid Specificity of the AKT1 P H Domaint. Biochemistry 47, 12260-12269.
Lee, S.-W., Li, C.-F., Jin, G., Cai, Z., Han, F., Chan, C.-H., Yang, W.-L., Li, B.-K., Rezaeian, A. H., Li, H.-Y., et al. (2015). Skp2-dependent ubiquitination and activation of LKB1 is essential for cancer cell survival under energy stress. Mol. Cell 57, 1022-1033.
Leiserson, M. D. M., Vandin, F., Wu, H.-T., Dobson, J. R., Eldridge, J. V., Thomas, J. L., Papoutsaki, A., Kim, Y., Niu, B., McLellan, M., et al. (2015). Pan-cancer network analysis identifies combinations of rare somatic mutations across pathways and protein complexes. Nat. Genet. 47, 106-114.
Lemaire, F., Millon, R., Young, J., Cromer, A., Wasylyk, C., Schultz, I., Muller, D., Marchal, P., Zhao, C., Melle, D., et al. (2003). Differential expression profiling of head and neck squamous cell carcinoma (HNSCC). Br. J. Cancer 89, 1940-1949.
Leung, C. C. Y., and Glover, J. N. M. (2011). BRCT domains: easy as one, two, three. Cell Cycle 10, 2461-2470.
Levy-Lahad, E., Catane, R., Eisenberg, S., Kaufman, B., Hornreich, G., Lishinsky, E., Shohat, M., Weber, B. L., Beller, U., Lahad, A., et al. (1997). Founder BRCA1 and BRCA2 mutations in Ashkenazi Jews in Israel: frequency and differential penetrance in ovarian cancer and in breast-ovarian cancer families. Am. J. Hum. Genet. 60, 1059-1067.
Li, M. L., and Greenberg, R. A. (2012). Links between genome integrity and BRCA1 tumor suppression. Trends Biochem. Sci. 37, 418-424.
Li, C., Xiao, L., Jia, J., Li, F., Wang, X., Duan, Q., Jing, H., Yang, P., Chen, C., Wang, Q., et al. (2019). Cornulin Is Induced in Psoriasis Lesions and Promotes Keratinocyte Proliferation via Phosphoinositide 3-Kinase/Akt Pathways. J. Invest. Dermatol. 139, 71-80.
Li, D., Marchenko, N. D., Schulz, R., Fischer, V., Velasco-Hernandez, T., Talos, F., and Moll, U. M. (2011). Functional inactivation of endogenous MDM2 and CHIP by HSP90 causes aberrant stabilization of mutant p53 in human cancer cells. Mol. Cancer Res. 9, 577-588.
Lim, J. S. J., Turner, N. C., and Yap, T. A. (2016). CDK4/6 Inhibitors: Promising Opportunities beyond Breast Cancer. Cancer Discovery 6, 697-699.
Lin, Y.-C., Lee, Y.-C., Li, L.-H., Cheng, C.-J., and Yang, R.-B. (2014). Tumor suppressor SCUBE2 inhibits breast cancer cell migration and invasion through the reversal of epithelial-mesenchymal transition. J. Cell Sci. 127, 85-100.
Lindhurst, M. J., Sapp, J. C., Teer, J. K., Johnston, J. J., Finn, E. M., Peters, K., Turner, J., Cannons, J. L., Bick, D., Blakemore, L., et al. (2011). A mosaic activating mutation in AKT1 associated with the Proteus syndrome. N. Engl. J. Med. 365, 611-619.
Liu, S., Knapp, S., and Ahmed, A. A. (2014). The structural basis of PI3K cancer mutations: from mechanism to therapy. Cancer Res. 74, 641-646.
Liu, Z., Wu, J., and Yu, X. (2007). CCDC98 targets BRCA1 to DNA damage sites. Nat. Struct. Mol. Biol. 14, 716-720.
Lombaerts, M., van Wezel, T., Philippo, K., Dierssen, J. W. F., Zimmerman, R. M. E., Oosting, J., van Eijk, R., Eilers, P. H., van de Water, B., Cornelisse, C. J., et al. (2006). E-cadherin transcriptional downregulation by promoter methylation but not mutation is related to epithelial-to-mesenchymal transition in breast cancer cell lines. Br. J. Cancer 94, 661-671.
Lukas, J., Lukas, C., and Bartek, J. (2011). More than just a focus: The chromatin response to DNA damage and its role in genome integrity maintenance. Nat. Cell Biol. 13, 1161-1169.
Malumbres, M., and Barbacid, M. (2001). To cycle or not to cycle: a critical decision in cancer. Nat. Rev. Cancer 1, 222-231.
Manning, B. D., and Cantley, L. C. (2007). AKT/PKB signaling: navigating downstream. Cell 129, 1261-1274.
Manning, B. D., and Toker, A. (2017). AKT/PKB Signaling: Navigating the Network. Cell 169, 381-405.
McAllister, K. A., and Wiseman, R. W. (2002). Are Trp53 rescue of Brcal embryonic lethality and Trp53/Brcal breast cancer association related? Breast Cancer Res. 4, 54-57.
McCubrey, J. A., Steelman, L. S., Chappell, W. H., Abrams, S. L., Franklin, R. A., Montalto, G., Cervello, M., Libra, M., Candido, S., Malaponte, G., et al. (2012).
Ras/Raf/MEK/ERK and PI3K/PTEN/Akt/mTOR cascade inhibitors: how mutations can result in therapy resistance and how to overcome resistance. Oncotarget 3, 1068-1111.
Miki, Y., Swensen, J., Shattuck-Eidens, D., Futreal, P. A., Harshman, K., Tavtigian, S., Liu, Q., Cochran, C., Bennett, L. M., and Ding, W. (1994). A strong candidate for the breast and ovarian cancer susceptibility gene BRCA1. Science 266, 66-71.
Mimori, K., Inoue, H., Shiraishi, T., Ueo, H., Mafune, K.-I., Tanaka, Y., and Mori, M. (2002). A single-nucleotide polymorphism of SMARCB1 in human breast cancers. Genomics 80, 254-258.
Morales, J. C., Richard, P., Patidar, P. L., Motea, E. A., Dang, T. T., Manley, J. L., and Boothman, D. A. (2016). XRN2 Links Transcription Termination to DNA Damage and Replication Stress. PLoS Genet. 12, e1006107.
Moynahan, M. E., and Jasin, M. (2010). Mitotic homologous recombination maintains genomic stability and suppresses tumorigenesis. Nat. Rev. Mol. Cell Biol. 11, 196-207.
Mullan, P. B., Quinn, J. E., and Harkin, D. P. (2006). The role of BRCA1 in transcriptional regulation and cell cycle control. Oncogene 25, 5854-5863.
Nelson, A. C., and Holt, J. T. (2010). Impact of RING and BRCT domain mutations on BRCA1 protein stability, localization and recruitment to DNA damage. Radiat. Res. 174, 1-13.
Nelson, A. C., Lyons, T. R., Young, C. D., Hansen, K. C., Anderson, S. M., and Holt, J. T. (2010). AKT regulates BRCA1 stability in response to hormone signaling. Mol. Cell. Endocrinol. 319, 129-142.
Ning, F., Wang, C., Berry, K. Z., Kandasamy, P., Liu, H., Murphy, R. C., Voelker, D. R., Nho, C. W., Pan, C.-H., A Dai, S., et al. (2014). Structural characterization of the pulmonary innate immune protein SPLUNC1 and identification of lipid ligands. FASEB J. 28, 5349-5360.
Niu, Y., Xu, J., and Sun, T. (2019). Cyclin-Dependent Kinases 4/6 Inhibitors in Breast Cancer: Current Status, Resistance, and Combination Strategies. J. Cancer 10, 5504-5517.
Olow, A., Chen, Z., Niedner, R. H., Wolf, D. M., Yau, C., Pankov, A., Lee, E. P., Brown-Swigart, L., van't Veer, L. J., and Coppe, J. P. (2016). An Atlas of the Human Kinome Reveals the Mutational Landscape Underlying Dysregulated Phosphorylation Cascades in Cancer. Cancer Res. 76, 1733-1745.
Paczkowska, M., Barenboim, J., Sintupisut, N., Fox, N. S., Zhu, H., Abd-Rabbo, D., Mee, M. W., Boutros, P. C., PCAWG Drivers and Functional Interpretation Working Group, Reimand, J., et al. (2020). Integrative pathway enrichment analysis of multivariate omics data. Nat. Commun. 11, 735.
Pal, S. K., Reckamp, K., Yu, H., and Figlin, R. A. (2010). Akt inhibitors in clinical development for the treatment of cancer. Expert Opin. Investig. Drugs 19, 1355-1366.
Papatheodorou, I., Moreno, P., Manning, J., Fuentes, A. M.-P., George, N., Fexova, S., Fonseca, N. A., Ftillgrabe, A., Green, M., Huang, N., et al. (2020). Expression Atlas update: from tissues to single cells. Nucleic Acids Res. 48, D77-D83.
Parrales, A., Ranjan, A., Iyer, S. V., Padhye, S., Weir, S. J., Roy, A., and Iwakuma, T. (2016). DNAJA1 controls the fate of misfolded mutant p53 through the mevalonate pathway. Nat. Cell Biol. 18, 1233-1243.
Prakash, R., Zhang, Y., Feng, W., and Jasin, M. Homologous recombination and human health: the roles of BRCA1, BRCA2, and associated proteins. Cold Spring Harb Perspect Biol. 2015; 7: a016600.
Qi, M., Zhang, J., Zeng, W., and Chen, X. (2014). DNAJB1 stabilizes MDM2 and contributes to cancer cell proliferation in a p53-dependent manner. Biochim. Biophys. Acta 1839, 62-69.
Quanz, M., Chassoux, D., Berthault, N., Agrario, C., Sun, J.-S., and Dutreix, M. (2009). Hyperactivation of DNAPK by double-strand break mimicking molecules disorganizes DNA damage response. PLoS One 4, e6298.
Reyna, M. A., Haan, D., Paczkowska, M., Verbeke, L. P. C., Vazquez, M., Kahraman, A., Pulido-Tamayo, S., Barenboim, J., Wadi, L., Dhingra, P., et al. (2020). Pathway and network analysis of more than 2500 whole cancer genomes. Nat. Commun. 11, 729.
Reynet, C., and Kahn, C. R. (1993). Rad: a member of the Ras family overexpressed in muscle of type II diabetic humans. Science 262, 1441-1444.
Rogakou, E. P., Pilch, D. R., Orr, A. H., Ivanova, V. S., and Bonner, W. M. (1998). DNA double-stranded breaks induce histone H2AX phosphorylation on serine 139. J. Biol. Chem. 273, 5858-5868.
van Roy, F., and Berx, G. (2008). The cell-cell adhesion molecule E-cadherin. Cell. Mol. Life Sci. 65, 3756-3788. Rudolph, M., Anzeneder, T., Schulz, A., Beckmann, G., Byrne, A. T., Jeffers, M., Pena, C., Politz, O., Köchert, K., Vonk, R., et al. (2016). AKT1 E17K mutation profiling in breast cancer: prevalence, concurrent oncogenic alterations, and blood-based detection. BMC Cancer 16, 622.
Rugo, H. S., Olopade, O. I., DeMichele, A., Yau, C., van't Veer, L. J., Buxton, M. B., Hogarth, M., Hylton, N. M., Paoloni, M., Perlmutter, J., et al. (2016). Adaptive Randomization of Veliparib-Carboplatin Treatment in Breast Cancer. N. Engl. J. Med. 375, 23-34.
Sanchez-Vega, F., Mina, M., Armenia, J., Chatila, W. K., Luna, A., La, K. C., Dimitriadoy, S., Liu, D. L., Kantheti, H. S., Saghafinia, S., et al. (2018). Oncogenic Signaling Pathways in The Cancer Genome Atlas. Cell 173, 321-337.e10.
Santagata, S., Thakkar, A., Ergonul, A., Wang, B., Woo, T., Hu, R., Harrell, J. C., McNamara, G., Schwede, M., Culhane, A. C., et al. (2014). Taxonomy of breast cancer based on normal cell phenotype predicts outcome.
Santo, L., Siu, K. T., and Raje, N. (2015). Targeting Cyclin-Dependent Kinases and Cell Cycle Progression in Human Cancers. Semin. Oncol. 42, 788-800.
Sarbassov, D. D., Guertin, D. A., Ali, S. M., and Sabatini, D. M. (2005). Phosphorylation and regulation of Akt/PKB by the rictor-mTOR complex. Science 307, 1098-1101.
Sarrouilhe, D., di Tommaso, A., M6tay6, T., and Ladeveze, V. (2006). Spinophilin: from partners to functions. Biochimie 88, 1099-1113.
Savage, K. I., Gorski, J. J., Barros, E. M., Irwin, G. W., Manti, L., Powell, A. J., Pellagatti, A., Lukashchuk, N., McCance, D. J., McCluggage, W. G., et al. (2014). Identification of a BRCA1-mRNA splicing complex required for efficient DNA repair and maintenance of genomic stability. Mol. Cell 54, 445-459.
Schulz-Heddergott, R., and Moll, U. M. (2018). Gain-of-Function (GOF) Mutant p53 as Actionable Therapeutic Target. Cancers 10.
Shakya, R., Reid, L. J., Reczek, C. R., Cole, F., Egli, D., Lin, C.-S., deRooij, D. G., Hirsch, S., Ravi, K., Hicks, J. B., et al. (2011). BRCA1 tumor suppression depends on BRCT phosphoprotein binding, but not its E3 ligase activity. Science 334, 525-528.
Sobhian, B., Shao, G., Lilli, D. R., Culhane, A. C., Moreau, L. A., Xia, B., Livingston, D. M., and Greenberg, R. A. (2007). RAP80 targets BRCA1 to specific ubiquitin structures at DNA damage sites. Science 316, 1198-1202.
Society, A. C. (2019). Breast Cancer Facts and Figures 2019-2020. Am. Cancer Soc 1-44.
Sowa, M. E., Bennett, E. J., Gygi, S. P., and Harper, J. W. (2009a). Defining the human deubiquitinating enzyme interaction landscape. Cell 138, 389-403.
Sowa, M. E., Bennett, E. J., Gygi, S. P., and Harper, J. W. (2009b). Defining the Human Deubiquitinating Enzyme Interaction Landscape. Cell 138, 389-403.
Stephens, P. J., Tarpey, P. S., Davies, H., Van Loo, P., Greenman, C., Wedge, D. C., Nik-Zainal, S., Martin, S., Varela, I., Bignell, G. R., et al. (2012). The landscape of cancer genes and mutational processes in breast cancer. Nature 486, 400-404.
Stokoe, D., Stephens, L. R., Copeland, T., Gaffney, P. R. J., Reese, C. B., Painter, G. F., Holmes, A. B., McCormick, F., and Hawkins, P. T. (1997). Dual Role of Phosphatidylinositol-3,4,5-trisphosphate in the Activation of Protein Kinase B. Science 277, 567-570.
Suzuki, M., Shigematsu, H., Shames, D. S., Sunaga, N., Takahashi, T., Shivapurkar, N., Iizasa, T., Minna, J. D., Fujisawa, T., and Gazdar, A. F. (2007). Methylation and gene silencing of the Ras-related GTPase gene in lung and breast cancers. Ann. Surg. Oncol. 14, 1397-1404.
Tassi, R. A., Bignotti, E., Falchetti, M., Calza, S., Ravaggi, A., Rossi, E., Martinelli, F., Bandiera, E., Pecorelli, S., and Santin, A. D. (2008). Mammaglobin B expression in human endometrial cancer. Int. J. Gynecol. Cancer 18, 1090-1096.
Tate, C. R., Rhodes, L. V., Segar, H. C., Driver, J. L., Pounder, F. N., Burow, M. E., and Collins-Burow, B. M. (2012). Targeting triple-negative breast cancer cells with the histone deacetylase inhibitor panobinostat. Breast Cancer Res. 14, R79.
Teo, G., Liu, G., Zhang, J., Nesvizhskii, A. I., Gingras, A. C., and Choi, H. (2014a). SAINTexpress: improvements and additional features in Significance Analysis of INTeractome software. J. Proteomics 100, 37-43.
Teo, G., Liu, G., Zhang, J., Nesvizhskii, A. I., Gingras, A.-C., and Choi, H. (2014b). SAINTexpress: improvements and additional features in Significance Analysis of INTeractome software. J. Proteomics 100, 37-43.
Thompson, E. R., Doyle, M. A., Ryland, G. L., Rowley, S. M., Choong, D. Y. H., Tothill, R. W., Thorne, H., kConFab, Barnes, D. R., Li, J., et al. (2012). Exome sequencing identifies rare deleterious mutations in DNA repair genes FANCC and BLM as potential breast cancer susceptibility alleles. PLoS Genet. 8, e1002894.
Tokunaga, E., Nakashima, Y., Yamashita, N., Hisamatsu, Y., Okada, S., Akiyoshi, S., Aishima, S., Kitao, H., Morita, M., and Maehara, Y. (2014). Overexpression of metadherin/MTDH is associated with an aggressive phenotype and a poor prognosis in invasive breast cancer. Breast Cancer 21, 341-349.
Uche, U. N., and Kane, L. P. (2016). PIK3IP1—A novel negative regulator of PI3K. The Journal of Immunology 196, 57.9-57.9.
Vega, A., Campos, B., Bressac-de-Paillerets, B., Bond, P. M., Janin, N., Douglas, F. S., Domenech, M., Baena, M., Pericay, C., Alonso, C., et al. (2001). The R71GBRCAlis a founder Spanish mutation and leads to aberrant splicing of the transcript. Human Mutation 17, 520-521.
Venkitaraman, A. R. (2014). Cancer suppression by the chromosome custodians, BRCA1 and BRCA2. Science 343, 1470-1475.
Vivanco, I., and Sawyers, C. L. (2002). The phosphatidylinositol 3-Kinase AKT pathway in human cancer. Nat. Rev. Cancer 2, 489-501.
Vizcaíno, J. A., Deutsch, E. W., Wang, R., Csordas, A., Reisinger, F., Rios, D., Dianes, J. A., Sun, Z., Farrah, T., Bandeira, N., et al. (2014). ProteomeXchange provides globally coordinated proteomics data submission and dissemination. Nat. Biotechnol. 32, 223-226.
Wang, B., Hurov, K., Hofmann, K., and Elledge, S. J. (2009). NBA1, a new player in the Brcal A complex, is required for DNA damage resistance and checkpoint control. Genes Dev. 23, 729-739.
Wang, Y., Cortez, D., Yazdi, P., Neff, N., Elledge, S. J., and Qin, J. (2000). BASC, a super complex of BRCA1-associated proteins involved in the recognition and repair of aberrant DNA structures. Genes Dev. 14, 927-939.
Wang, Y., Bernhardy, A. J., Cruz, C., Krais, J. J., Nacson, J., Nicolas, E., Peri, S., van der Gulden, H., van der Heijden, I., O'Brien, S. W., et al. (2016a). The BRCA1-Δ11q Alternative Splice Isoform Bypasses Germline Mutations and Promotes Therapeutic Resistance to PARP Inhibition and Cisplatin. Cancer Res. 76, 2778-2790.
Wang, Y., Krais, J. J., Bernhardy, A. J., Nicolas, E., Cai, K. Q., Harrell, M. I., Kim, H. H., George, E., Swisher, E. M., Simpkins, F., et al. (2016b). RING domain-deficient BRCA1 promotes PARP inhibitor and platinum resistance. J. Clin. Invest. 126, 3145-3157.
Warburg, O. (1956). On the origin of cancer cells. Science 123, 309-314.
Wolf, D. M., Yau, C., Sanil, A., Glas, A., Petricoin, E., Wulfkuhle, J., Severson, T. M., Linn, S., Brown-Swigart, L., Hirst, G., et al. (2017). DNA repair deficiency biomarkers and the 70-gene ultra-high risk signature as predictors of veliparib/carboplatin response in the I-SPY 2 breast cancer trial. NPJ Breast Cancer 3, 31.
Wood, L. D., Parsons, D. W., Jones, S., Lin, J., Sjöblom, T., Leary, R. J., Shen, D., Boca, S. M., Barber, T., Ptak, J., et al. (2007). The genomic landscapes of human breast and colorectal cancers. Science 318, 1108-1113.
Woods, N. T., Mesquita, R. D., Sweet, M., Carvalho, M. A., Li, X., Liu, Y., Nguyen, H., Thomas, C. E., Iversen, E. S., Marsillac, S., et al. (2012). Charting the Landscape of Tandem BRCT Domain-Mediated Protein Interactions. Science Signaling 5, rs6-rs6.
Wu, L. C., Wang, Z. W., Tsan, J. T., Spillman, M. A., Phung, A., Xu, X. L., Yang, M. C., Hwang, L. Y., Bowcock, A. M., and Baer, R. (1996). Identification of a RING protein that can interact in vivo with the BRCA1 gene product. Nat. a Genet. 14, 430-440.
Wu, Q., Paul, A., Su, D., Mehmood, S., Foo, T. K., Ochi, T., Bunting, E. L., Xia, B., Robinson, C. V., Wang, B., et al. (2016). Structure of BRCA1-BRCT/Abraxas Complex Reveals Phosphorylation-Dependent BRCT Dimerization at DNA Damage Sites. Mol. Cell 61, 434-448.
Wulfkuhle, J. D., Yau, C., Wolf, D. M., Vis, D. J., Gallagher, R. I., Brown-Swigart, L., Hirst, G., Voest, E. E., DeMichele, A., Hylton, N., et al. (2018). Evaluation of the HER/PI3K/AKT Family Signaling Network as a Predictive Biomarker of Pathologic Complete Response for Patients With Breast Cancer Treated With Neratinib in the I-SPY 2 TRIAL. JCO Precision Oncology 1-20.
Xu, B., O'Donnell, A. H., Kim, S.-T., and Kastan, M. B. (2002). Phosphorylation of serine 1387 in Brcal is specifically required for the Atm-mediated S-phase checkpoint after ionizing irradiation. Cancer Res. 62, 4588-4591.
Xu, X., Omelchenko, T., and Hall, A. (2010). LKB1 tumor suppressor protein regulates actin filament assembly through Rho and its exchange factor Dbl independently of kinase activity. BMC Cell Biol. 11, 77.
Yap, T. A., Yan, L., Patnaik, A., Fearen, I., Olmos, D., Papadopoulos, K., Baird, R. D., Delgado, L., Taylor, A., Lupinacci, L., et al. (2011). First-in-man clinical trial of the oral pan-AKT inhibitor MK-2206 in patients with advanced solid tumors. J. Clin. Oncol. 29, 4688-4695.
Yu, X., and Chen, J. (2004). DNA damage-induced cell cycle checkpoint control requires CtIP, a phosphorylationdependent binding partner of BRCA1 C-terminal domains. Mol. Cell. Biol. 24, 9478-9486.
Yu, K., Chen, B., Aran, D., Charalel, J., Yau, C., Wolf, D. M., van't Veer, L. J., Butte, A. J., Goldstein, T., and Sirota, M. (2019). Comprehensive transcriptomic analysis of cell lines as models of primary tumors across 22 tumor types. Nature Communications 10.
Yu, X., Chini, C. C., He, M., Mer, G., and Chen, J. (2003). The BRCT domain is a phospho-protein binding domain. Science 302, 639-642.
Yuan, T. L., and Cantley, L. C. (2008). PI3K pathway alterations in cancer: variations on a theme. Oncogene 27, 5497-5510.
Yue, X., Zhao, Y., Huang, G., Li, J., Zhu, J., Feng, Z., and Hu, W. (2016). A novel mutant p53 binding partner BAG5 stabilizes mutant p53 and promotes mutant p53 GOFs in tumorigenesis. Cell Discov 2, 16039.
Yun, M. H., and Hiom, K. (2009). CtIP-BRCA1 modulates the choice of DNA double-strand-break repair pathway throughout the cell cycle. Nature 459, 460-463.
Zeqiraj, E., Filippi, B. M., Goldie, S., Navratilova, I., Boudeau, J., Deak, M., Alessi, D. R., and van Aalten, D. M. F. (2009a). ATP and MO25alpha regulate the conformational state of the STRADalpha pseudokinase and activation of the LKB1 tumour suppressor. PLoS Biol. 7, e1000126.
Zeqiraj, E., Filippi, B. M., Deak, M., Alessi, D. R., and van Aalten, D. M. F. (2009b). Structure of the LKB1-STRADMO25 complex reveals an allosteric mechanism of kinase activation. Science 326, 1707-1711.
Zhang, B., Nie, X., Xiao, B., Xiang, J., Shen, S., Gong, J., Zhou, M., Zhu, S., Zhou, J., Qian, J., et al. (2003). Identification of tissue-specific genes in nasopharyngeal epithelial tissue and differentially expressed genes in nasopharyngeal carcinoma by suppression subtractive hybridization and cDNA microarray. Genes Chromosomes Cancer 38, 80-90.
Zhang, C., Liu, J., Wu, R., Liang, Y., Lin, M., Liu, J., Chan, C. S., Hu, W., and Feng, Z. (2014). Tumor suppressor p53 negatively regulates glycolysis stimulated by hypoxia through its target RRAD. Oncotarget 5, 5535-5546.
Zhang, L., Yan, X., Yu, S., Zhong, X., Tian, R., Xu, L., Bian, X., and Su, J. (2020). LINC00365-SCGB2A1 axis A inhibits the viability of breast cancer through targeting NF-cB signaling. Oncol. Lett. 19, 753-762.
Zhang, S., Schafer-Hales, K., Khuri, F. R., Zhou, W., Vertino, P. M., and Marcus, A. I. (2008). The tumor suppressor LKB1 regulates lung cancer cell polarity by mediating cdc42 recruitment and activity. Cancer Res. 68, 740-748.
Zhao, G. Y., Sonoda, E., Barber, L. J., Oka, H., Murakawa, Y., Yamada, K., Ikura, T., Wang, X., Kobayashi, M., Yamamoto, K., et al. (2007). A critical role for the ubiquitin-conjugating enzyme Ubcl3 in initiating homologous recombination. Mol. Cell 25, 663-675.
Zheng, W., Cong, X.-F., Cai, W.-H., Yang, S., Mao, C., and Zou, H.-W. (2011). Current evidences on XPC polymorphisms and breast cancer susceptibility: a meta-analysis. Breast Cancer Res. Treat. 128, 811-815.
Zubor, P., Hatok, J., Moricova, P., Kajo, K., Kapustova, I., Mendelova, A., Racay, P., and Danko, J. (2015). Gene expression abnormalities in histologically normal breast epithelium from patients with luminal type of breast cancer. Mol. Biol. Rep. 42, 977-988.
Akavia, U. D., Litvin, O., Kim, J., Sanchez-Garcia, F., Kotliar, D., Causton, H. C., Pochanard, P., Mozes, E., Garraway, L. A., and Pe'er, D. (2010). An integrated approach to uncover drivers of cancer. Cell 143, 1005-1017.
Alexa, A., Gógl, G., Glatz, G., Garai, A., Zeke, A., Varga, J., Dudás, E., Jeszenöi, N., Bodor, A., Hetényi, C., et al. (2015). Structural assembly of the signaling competent ERK2-RSK1 heterodimeric protein kinase complex. Proc. Natl. Acad. Sci. U.S.A 112, 2711-2716.
Alvarado, D., Ligon, G. F., Lillquist, J. S., Seibel, S. B., Wallweber, G., Neumeister, V. M., Rimm, D. L., McMahon, G., and LaVallee, T. M. (2017). ErbB activation signatures as potential biomarkers for anti-ErbB3 treatment in HNSCC. PLoS One 12, e0181356.
Ara, H., Takagishi, M., Enomoto, A., Asai, M., Ushida, K., Asai, N., Shimoyama, Y., Kaibuchi, K., Kodera, Y., A and Takahashi, M. (2016). Role for Daple in non-canonical Wnt signaling during gastric cancer invasion and metastasis. Cancer Sci. 107, 133-139.
Arvind, R., Shimamoto, H., Momose, F., Amagasa, T., Omura, K., and Tsuchida, N. (2005). A mutation in the common docking domain of ERK2 in a human cancer cell line, which was associated with its constitutive phosphorylation. Int. J. Oncol. 27, 1499-1504.
Aznar, N., Midde, K. K., Dunkel, Y., Lopez-Sanchez, I., Pavlova, Y., Marivin, A., Barbazán, J., Murray, F., Nitsche, U., Janssen, K.-P., et al. (2015). Daple is a novel non-receptor GEF required for trimeric G protein activation in Wnt signaling. Elife 4, e07091.
Aznar, N., Sun, N., Dunkel, Y., Ear, J., Buschman, M. D., and Ghosh, P. (2017). A Daple-Akt feed-forward loop enhances noncanonical Wnt signals by compartmentalizing (3-catenin. Mol. Biol. Cell 28, 3709-3723.
Aznar, N., Ear, J., Dunkel, Y., Sun, N., Satterfield, K., He, F., Kalogriopoulos, N. A., Lopez-Sanchez, I., Ghassemian, M., Sahoo, D., et al. (2018). Convergence of Wnt, growth factor, and heterotrimeric G protein signals on the guanine nucleotide exchange factor Daple. Sci. Signal. 11.
Bailey, M. H., Tokheim, C., Porta-Pardo, E., Sengupta, S., Bertrand, D., Weerasinghe, A., Colaprico, A., Wendl, M. C., Kim, J., Reardon, B., et al. (2018). Comprehensive Characterization of Cancer Driver Genes and Mutations. Cell 174, 1034-1035.
Biankin, A. V., Waddell, N., Kassahn, K. S., Gingras, M. C., Muthuswamy, L. B., Johns, A. L., Miller, D. K., Wilson, P. J., Patch, A. M., Wu, J., et al. (2012). Pancreatic cancer genomes reveal aberrations in axon guidance pathway genes. Nature 491, 399-405.
Bouhaddou, M., Eckhardt, M., Chi Naing, Z. Z., Kim, M., Ideker, T., and Krogan, N. J. (2019). Mapping the protein-protein and genetic interactions of cancer to guide precision medicine. Curr. Opin. Genet. Dev. 54, 110-117.
Brenan, L., Andreev, A., Cohen, O., Pantel, S., Kamburov, A., Cacchiarelli, D., Persky, N. S., Zhu, C., Bagul, M., Goetz, E. M., et al. (2016). Phenotypic Characterization of a Comprehensive Set of MAPK1/ERK2 Missense Mutants. Cell Rep. 17, 1171-1183.
Burke, J. E., Perisic, O., Masson, G. R., Vadas, O., and Williams, R. L. (2012). Oncogenic mutations mimic and enhance dynamic events in the natural activation of phosphoinositide 3-kinase p110α (PIK3CA). Proc. Natl. Acad. Sci. U.S.A 109, 15259-15264.
Cancer Genome Atlas, Network (2012). Comprehensive molecular portraits of human breast tumours. Nature 490, 61-70.
Cancer Genome Atlas, Network (2015). Comprehensive genomic characterization of head and neck squamous cell carcinomas. Nature 517, 576-582.
Cancer Genome Atlas Network (2015). Comprehensive genomic characterization of head and neck squamous cell carcinomas. Nature 517, 576-582.
Cancer Genome Atlas Research, Network (2008). Comprehensive genomic characterization defines human glioblastoma genes and core pathways. Nature 455, 1061-1068.
Cancer Genome Atlas Research, Network (2011). Integrated genomic analyses of ovarian carcinoma. Nature 474, 609-615.
Carson, J. D., Van Aller, G., Lehr, R., Sinnamon, R. H., Kirkpatrick, R. B., Auger, K. R., Dhanak, D., Copeland, R. A., Gontarek, R. R., Tummino, P. J., et al. (2008). Effects of oncogenic p1Oalpha subunit mutations on the A lipid kinase activity of phosphoinositide 3-kinase. Biochem. J 409, 519-524.
Cerami, E., Demir, E., Schultz, N., Taylor, B. S., and Sander, C. (2010). Automated network analysis identifies core pathways in glioblastoma. PLoS One 5, e8918.
Chakravarty, D., Gao, J., Phillips, S. M., Kundra, R., Zhang, H., Wang, J., Rudolph, J. E., Yaeger, R., Soumerai, T., Nissan, M. H., et al. (2017). OncoKB: A Precision Oncology Knowledge Base. JCO Precis Oncol 2017.
Choi, M., Chang, C.-Y., Clough, T., Broudy, D., Killeen, T., MacLean, B., and Vitek, O. (2014). MSstats: an R package for statistical analysis of quantitative mass spectrometry-based proteomic experiments. Bioinformatics 30, 2524-2526.
Consequences, T. M., and Consortium, Pathway Analysis working group of the International Cancer Genome (2015). Pathway and network analysis of cancer genomes. Nat. Methods 12, 615-621.
Copple, I. M., Lister, A., Obeng, A. D., Kitteringham, N. R., Jenkins, R. E., Layfield, R., Foster, B. J., Goldring, C. E., and Park, B. K. (2010). Physical and functional interaction of sequestosome 1 with KeapI regulates the Keap1-Nrf2 cell defense pathway. J. Biol. Chem. 285, 16782-16788.
Cox, J., and Mann, M. (2008). MaxQuant enables high peptide identification rates, individualized p.p.b.-range mass accuracies and proteome-wide protein quantification. Nat. Biotechnol. 26, 1367-1372.
Czubaty, A., Girstun, A., Kowalska-Loth, B., Trzcinska, A. M., Purta, E., Winczura, A., Grajkowski, W., and Staron, K. (2005). Proteomic analysis of complexes formed by human topoisomerase I. Biochim. Biophys. Acta 1749, 133-141.
Davis, W. J., Lehmann, P. Z., and Li, W. (2015). Nuclear PI3K signaling in cell growth and tumorigenesis. Front Cell Dev Biol 3, 24.
Dogruluk, T., Tsang, Y. H., Espitia, M., Chen, F., Chen, T., Chong, Z., Appadurai, V., Dogruluk, A., Eterovic, A. K., Bonnen, P. E., et al. (2015). Identification of Variant-Specific Functions of PIK3CA by Rapid Phenotyping of Rare Mutations. Cancer Res. 75, 5341-5354.
Dornan, G. L., and Burke, J. E. (2018). Molecular Mechanisms of Human Disease Mediated by Oncogenic and Primary Immunodeficiency Mutations in Class IA Phosphoinositide 3-Kinases. Front. Immunol. 9, 575.
Drier, Y., Sheffer, M., and Domany, E. (2013). Pathway-based personalized analysis of cancer. Proc. Natl. Acad. Sci. U.S.A 110, 6388-6393.
Dumbrava, E. I., Alfattal, R., Miller, V. A., and Tsimberidou, A. M. (2018). Complete Response to a Fibroblast Growth Factor Receptor Inhibitor in a Patient With Head and Neck Squamous Cell Carcinoma Harboring FGF Amplifications. JCO Precis Oncol 2.
Duvvuri, U., George, J., Kim, S., Alvarado, D., Neumeister, V. M., Chenna, A., Gedrich, R., Hawthorne, T., LaVallee, T., Grandis, J. R., et al. (2019). Molecular and Clinical Activity of CDX-3379, an Anti-ErbB3 Monoclonal Antibody, in Head and Neck Squamous Cell Carcinoma Patients. Clin. Cancer Res. 25, 5752-5758.
Eckhardt, M., Zhang, W., Gross, A. M., Von Dollen, J., Johnson, J. R., Franks-Skiba, K. E., Swaney, D. L., Johnson, T. L., Jang, G. M., Shah, P. S., et al. (2018). Multiple Routes to Oncogenesis Are Promoted by the Human Papillomavirus-Host Protein Network. Cancer Discov. 8, 1474-1489.
Fan, W., Tang, Z., Chen, D., Moughon, D., Ding, X., Chen, S., Zhu, M., and Zhong, Q. (2010). KeapI facilitates p62-mediated ubiquitin aggregate clearance via autophagy. Autophagy 6, 614-621.
Feng, Y., and Longmore, G. D. (2005). The LIM protein Ajuba influences interleukin-1-induced NF-kappaB A activation by affecting the assembly and activity of the protein kinase Czeta/p62/TRAF6 signaling complex. Mol. Cell. Biol. 25, 4010-4022.
Fusté, N. P., Castelblanco, E., Felip, I., Santacana, M., Fernández-Hernández, R., Gatius, S., Pedraza, N., Pallards, J., Cemeli, T., Valls, J., et al. (2016). Characterization of cytoplasmic cyclin D1 as a marker of invasiveness in cancer. Oncotarget 7, 26979-26991.
Gkeka, P., Evangelidis, T., Pavlaki, M., Lazani, V., Christoforidis, S., Agianian, B., and Cournia, Z. (2014). Investigating the structure and dynamics of the PIK3C A wild-type and H1047R oncogenic mutant. PloS Comput. Biol. 10, e1003895.
Goetz, E. M., Ghandi, M., Treacy, D. J., Wagle, N., and Garraway, L. A. (2014). ERK mutations confer resistance to mitogen-activated protein kinase pathway inhibitors. Cancer Res. 74, 7079-7089.
Hamilton, E., and Infante, J. R. (2016). Targeting CDK4/6 in patients with cancer. Cancer Treat. Rev. 45, 129-138.
Hanahan, D., and Weinberg, R. A. (2011). Hallmarks of cancer: the next generation. Cell 144, 646-674.
Hanahan, D., Douglas, H., and Weinberg, R. A. (2000). The Hallmarks of Cancer. Cell 100, 57-70.
Haraguchi, K., Ohsugi, M., Abe, Y., Semba, K., Akiyama, T., and Yamamoto, T. (2008). Ajuba negatively regulates the Wnt signaling pathway by promoting GSK-3beta-mediated phosphorylation of beta-catenin. Oncogene 27, 274-284.
Hein, M. Y., Hubner, N. C., Poser, I., Cox, J., Nagaraj, N., Toyoda, Y., Gak, I. A., Weisswange, I., Mansfeld, J., Buchholz, F., et al. (2015). A Human Interactome in Three Quantitative Dimensions Organized by Stoichiometries and Abundances. Cell 163, 712-723.
Hoadley, K. A., Yau, C., Hinoue, T., Wolf, D. M., Lazar, A. J., Drill, E., Shen, R., Taylor, A. M., Cherniack, A. D., Thorsson, V., et al. (2018). Cell-of-Origin Patterns Dominate the Molecular Classification of 10,000 Tumors from 33 Types of Cancer. Cell 173, 291-304.e6.
Hofree, M., Shen, J. P., Carter, H., Gross, A., and Ideker, T. (2013). Network-based stratification of tumor mutations. Nat. Methods 10, 1108-1115.
Horn, H., Lawrence, M. S., Chouinard, C. R., Shrestha, Y., Hu, J. X., Worstell, E., Shea, E., Ilic, N., Kim, E., Kamburov, A., et al. (2018). NetSig: network-based discovery from cancer genomes. Nat. Methods 15, 61-66.
Huttlin, E. L., Ting, L., Bruckner, R. J., Gebreab, F., Gygi, M. P., Szpyt, J., Tam, S., Zarraga, G., Colby, G., Baltier, K., et al. (2015). The BioPlex Network: A Systematic Exploration of the Human Interactome. Cell 162, 425-440.
Huttlin, E. L., Bruckner, R. J., Paulo, J. A., Cannon, J. R., Ting, L., Baltier, K., Colby, G., Gebreab, F., Gygi, M. P., Parzen, H., et al. (2017). Architecture of the human interactome defines protein communities and disease networks. Nature 545, 505-509.
Huttlin, E. L., Bruckner, R. J., Navarrete-Perea, J., Cannon, J. R., Baltier, K., Gebreab, F., Gygi, M. P., Thornock, A., Zarraga, G., Tam, S., et al. (2020). Dual Proteome-scale Networks Reveal Cell-specific Remodeling of the Human Interactome.
Inoue, A., Raimondi, F., Kadji, F. M. N., Singh, G., Kishi, T., Uwamizu, A., Ono, Y., Shinjo, Y., Ishida, S., Arang, N., et al. (2019). Illuminating G-Protein-Coupling Selectivity of GPCRs. Cell 177, 1933-1947.e25.
Ishida-Takagishi, M., Enomoto, A., Asai, N., Ushida, K., Watanabe, T., Hashimoto, T., Kato, T., Weng, L., Matsumoto, S., Asai, M., et al. (2012). The Dishevelled-associating protein Daple controls the non-canonical A Wnt/Rac pathway and cell motility. Nat. Commun. 3, 859.
Jäger, S., Cimermancic, P., Gulbahce, N., Johnson, J. R., McGovern, K. E., Clarke, S. C., Shales, M., Mercenne, G., Pache, L., Li, K., et al. (2011). Global landscape of HIV-human protein complexes. Nature 481, 365-370.
Janku, F., Yap, T. A., and Meric-Bernstam, F. (2018). Targeting the PI3K pathway in cancer: are we making headway? Nat. Rev. Clin. Oncol. 15, 273-291.
Kalkat, M., Resetca, D., Lourenco, C., Chan, P.-K., Wei, Y., Shiah, Y.-J., Vitkin, N., Tong, Y., Sunnerhagen, M., Done, S. J., et al. (2018). MYC Protein Interactome Profiling Reveals Functionally Distinct Regions that Cooperate to Drive Tumorigenesis. Mol. Cell 72, 836-848.e7.
Klykov, O., Steigenberger, B., Pekta, S., Fasci, D., Heck, A. J. R., and Scheltema, R. A. (2018). Efficient and robust proteome-wide approaches for cross-linking mass spectrometry. Nat. Protoc.
Koren, I., Reem, E., and Kimchi, A. (2010a). Autophagy gets a brake: DAPi, a novel mTOR substrate, is activated to suppress the autophagic process. Autophagy 6, 1179-1180.
Koren, I., Reem, E., and Kimchi, A. (2010b). DAP1, a novel substrate of mTOR, negatively regulates autophagy. Curr. Biol. 20, 1093-1098.
Krogan, N. J., Lippman, S., Agard, D. A., Ashworth, A., and Ideker, T. (2015). The cancer cell map initiative: defining the hallmark networks of cancer. Mol. Cell 58, 690-698.
Lau, A., Wang, X.-J., Zhao, F., Villeneuve, N. F., Wu, T., Jiang, T., Sun, Z., White, E., and Zhang, D. D. (2010). A noncanonical mechanism of Nrf2 activation by autophagy deficiency: direct interaction between Keap1 and p62. Mol. Cell. Biol. 30, 3275-3285.
Lee, S., Greenlee, E. B., Amick, J. R., Ligon, G. F., Lillquist, J. S., Natoli, E. J., Jr, Hadari, Y., Alvarado, D., and Schlessinger, J. (2015). Inhibition of ErbB3 by a monoclonal antibody that locks the extracellular domain in an inactive configuration. Proc. Natl. Acad. Sci. U.S.A 112, 13225-13230.
Leiserson, M. D. M., Vandin, F., Wu, H.-T., Dobson, J. R., Eldridge, J. V., Thomas, J. L., Papoutsaki, A., Kim, Y., Niu, B., McLellan, M., et al. (2015). Pan-cancer network analysis identifies combinations of rare somatic mutations across pathways and protein complexes. Nat. Genet. 47, 106-114.
Li, H., Wawrose, J. S., Gooding, W. E., Garraway, L. A., Lui, V. W. Y., Peyser, N. D., and Grandis, J. R. (2014). Genomic Analysis of Head and Neck Squamous Cell Carcinoma Cell Lines and Human Tumors: A Rational Approach to Preclinical Model Selection. Mol. Cancer Res. 12, 571-582.
Li, T., Wernersson, R., Hansen, R. B., Horn, H., Mercer, J., Slodkowicz, G., Workman, C. T., Rigina, O., Rapacki, K., Storfeldt, H. H., et al. (2016). A scored human protein-protein interaction network to catalyze genomic interpretation. Nat. Methods.
Liu, S., Knapp, S., and Ahmed, A. A. (2014). The structural basis of PI3K cancer mutations: from mechanism to therapy. Cancer Res. 74, 641-646.
Lobingier, B. T., Htittenhain, R., Eichel, K., Miller, K. B., Ting, A. Y., von Zastrow, M., and Krogan, N. J. (2017). An Approach to Spatiotemporally Resolve Protein Interaction Networks in Living Cells. Cell 169, 350-360.e12.
Luck, K., Kim, D.-K., Lambourne, L., Spirohn, K., Begg, B. E., Bian, W., Brignall, R., Cafarelli, T., Campos-Laborie, F. J., Charloteaux, B., et al. (2020). A reference map of the human binary protein interactome. Nature 580, 402-408.
Lui, V. W. Y., Hedberg, M. L., Li, H., Vangara, B. S., Pendleton, K., Zeng, Y., Lu, Y., Zhang, Q., Du, Y., Gilbert, B. R., et al. (2013). Frequent Mutation of the PI3K Pathway in Head and Neck Cancer Defines Predictive A Biomarkers. Cancer Discov. 3, 761-769.
MacLean, B., Tomazela, D. M., Shulman, N., Chambers, M., Finney, G. L., Frewen, B., Kern, R., Tabb, D. L., Liebler, D. C., and MacCoss, M. J. (2010). Skyline: an open source document editor for creating and analyzing targeted proteomics experiments. Bioinformatics 26, 966-968.
Mahalingam, M., Arvind, R., and Ida, H. (2008). ERK2 C D domain mutation from a human cancer cell line enhanced anchorage-independent cell growth and abnormality in Drosophila. Oncology.
Marinkovich, M. P. (2007). Tumour microenvironment: laminin 332 in squamous-cell carcinoma. Nat. Rev. Cancer 7, 370-380.
Martin, D., Abba, M. C., Molinolo, A. A., Vitale-Cross, L., Wang, Z., Zaida, M., Delic, N. C., Samuels, Y., Lyons, J. G., and Gutkind, J. S. (2014). The head and neck cancer cell oncogenome: a platform for the development of precision molecular therapies. Oncotarget 5, 8906-8923.
Miled, N., Yan, Y., Hon, W.-C., Perisic, O., Zvelebil, M., Inbar, Y., Schneidman-Duhovny, D., Wolfson, H. J., Backer, J. M., and Williams, R. L. (2007). Mechanism of two classes of cancer mutations in the phosphoinositide 3-kinase catalytic subunit. Science 317, 239-242.
Molinolo, A. A., Amornphimoltham, P., Squarize, C. H., Castilho, R. M., Patel, V., and Gutkind, J. S. (2009). Dysregulated molecular networks in head and neck carcinogenesis. Oral Oncol. 45, 324-334.
Nolte, H., MacVicar, T. D., Tellkamp, F., and Kruger, M. (2018). Instant Clue: A Software Suite for Interactive Data Visualization and Analysis. Sci. Rep. 8, 12648.
Oshita, A., Kishida, S., Kobayashi, H., Michiue, T., Asahara, T., Asashima, M., and Kikuchi, A. (2003). Identification and characterization of a novel Dvl-binding protein that suppresses Wnt signalling pathway. Genes Cells 8, 1005-1017.
Paczkowska, M., Barenboim, J., Sintupisut, N., Fox, N. S., Zhu, H., Abd-Rabbo, D., Mee, M. W., Boutros, P. C., PCAWG Drivers and Functional Interpretation Working Group, Reimand, J., et al. (2020). Integrative pathway enrichment analysis of multivariate omics data. Nat. Commun. 11, 735.
Park, J., Kim, J.-M., Park, J. K., Huang, S., Kwak, S. Y., Ryu, K. A., Kong, G., Park, J., and Koo, B. S. (2015). Association of p21-activated kinase-1 activity with aggressive tumor behavior and poor prognosis of head and neck cancer. Head Neck 37, 953-963.
Parvathy, M., Sreeja, S., Kumar, R., and Pillai, M. R. (2016). Potential role of p21 Activated Kinase 1 (PAKI) in the invasion and motility of oral cancer cells. BMC Cancer 16 Suppl 1, 293.
Paull, E. O., Carlin, D. E., Niepel, M., Sorger, P. K., Haussler, D., and Stuart, J. M. (2013). Discovering causal pathways linking genomic events to transcriptional states using Tied Diffusion Through Interacting Events (TieDIE). Bioinformatics 29, 2757-2764.
Perez-Riverol, Y., Csordas, A., Bai, J., Bernal-Llinares, M., Hewapathirana, S., Kundu, D. J., Inuganti, A., Griss, J., Mayer, G., Eisenacher, M., et al. (2019). The PRIDE database and related tools and resources in 2019: improving support for quantification data. Nucleic Acids Res. 47, D442-D450.
Pyndiah, S., Tanida, S., Ahmed, K. M., Cassimere, E. K., Choe, C., and Sakamuro, D. (2011). c-MYC Suppresses BIN1 to Release Poly(ADP-Ribose) Polymerase 1: A Mechanism by Which Cancer Cells Acquire Cisplatin Resistance. Sci. Signal. 4, ra19-ra19.
Reyna, M. A., Haan, D., Paczkowska, M., Verbeke, L. P. C., Vazquez, M., Kahraman, A., Pulido-Tamayo, S., Barenboim, J., Wadi, L., Dhingra, P., et al. (2020). Pathway and network analysis of more than 2500 whole A cancer genomes. Nat. Commun. 11, 729.
Riaz, N., Morris, L. G., Lee, W., and Chan, T. A. (2014). Unraveling the molecular genetics of head and neck cancer through genome-wide approaches. Genes Dis 1, 75-86.
Robinson, D., Van Allen, E. M., Wu, Y. M., Schultz, N., Lonigro, R. J., Mosquera, J. M., Montgomery, B., Taplin, M. E., Pritchard, C. C., Attard, G., et al. (2015). Integrative Clinical Genomics of Advanced Prostate Cancer. Cell 162, 454.
Rolland, T., Ta an, M., Charloteaux, B., Pevzner, S. J., Zhong, Q., Sahni, N., Yᵢ, S., Lemmens, I., Fontanillo, C., Mosca, R., et al. (2014). A Proteome-Scale Map of the Human Interactome Network. Cell 159, 1212-1226.
Rudd, M. L., Price, J. C., Fogoros, S., Godwin, A. K., Sgroi, D. C., Merino, M. J., and Bell, D. W. (2011). A unique spectrum of somatic PIK3C A (p110alpha) mutations within primary endometrial carcinomas. Clin. Cancer Res. 17, 1331-1340.
Salas, D., Stacey, R. G., Akinlaja, M., and Foster, L. J. (2020). Next-generation Interactomics: Considerations for the Use of Co-elution to Measure Protein Interaction Networks. Mol. Cell. Proteomics 19, 1-10.
Samavarchi-Tehrani, P., Samson, R., and Gingras, A.-C. (2020). Proximity dependent biotinylation: key enzymes and adaptation to proteomics approaches. Mol. Cell. Proteomics.
Schuler, M., Cho, B. C., Sayehli, C. M., Navarro, A., Soo, R. A., Richly, H., Cassier, P. A., Tai, D., Penel, N., Nogova, L., et al. (2019). Rogaratinib in patients with advanced cancers selected by FGFR mRNA expression: a phase 1 dose-escalation and dose-expansion study. Lancet Oncol. 20, 1454-1466.
Shekar, S. C., Wu, H., Fu, Z., Yip, S.-C., Nagajyothi, Cahill, S. M., Girvin, M. E., and Backer, J. M. (2005). Mechanism of constitutive phosphoinositide 3-kinase activation by oncogenic mutants of the p85 regulatory subunit. J. Biol. Chem. 280, 27850-27855.
Shibata, T., Ohta, T., Tong, K. I., Kokubu, A., Odogawa, R., Tsuta, K., Asamura, H., Yamamoto, M., and Hirohashi, S. (2008). Cancer related mutations in NRF2 impair its recognition by Keap1-Cul3 E3 ligase and promote malignancy. Proc. Natl. Acad. Sci. U.S.A. 105, 13568-13573.
Songyang, Z., Shoelson, S. E., McGlade, J., Olivier, P., Pawson, T., Bustelo, X. R., Barbacid, M., Sabe, H., Hanafusa, H., and Yᵢ, T. (1994). Specific motifs recognized by the SH2 domains of Csk, 3BP2, fps/fes, GRB-2, HCP, SHC, Syk, and Vav. Mol. Cell. Biol. 14, 2777-2785.
Sowa, M. E., Bennett, E. J., Gygi, S. P., and Harper, J. W. (2009). Defining the Human Deubiquitinating Enzyme Interaction Landscape. Cell 138, 389-403.
Stephens, P. J., Tarpey, P. S., Davies, H., Van Loo, P., Greenman, C., Wedge, D. C., Nik-Zainal, S., Martin, S., Varela, I., Bignell, G. R., et al. (2012). The landscape of cancer genes and mutational processes in breast cancer. Nature 486, 400-404.
Stransky, N., Egloff, A. M., Tward, A. D., Kostic, A. D., Cibulskis, K., Sivachenko, A., Kryukov, G. V., Lawrence, M. S., Sougnez, C., McKenna, A., et al. (2011). The mutational landscape of head and neck squamous cell carcinoma. Science 333, 1157-1160.
Taguchi, K., and Yamamoto, M. (2017). The KEAP1-NRF2 System in Cancer. Front. Oncol. 7, 85.
Takahashi, Y., Coppola, D., Matsushita, N., Cualing, H. D., Sun, M., Sato, Y., Liang, C., Jung, J. U., Cheng, J. Q., Muld, J. J., et al. (2007). Bif-1 interacts with Beclin 1 through UVRAG and regulates autophagy and tumorigenesis. Nat. Cell Biol. 9, 1142-1151.
Taylor, C. A., 4th, Cormier, K. W., Keenan, S. E., Earnest, S., Stippec, S., Wichaidit, C., Juang, Y.-C., Wang, J., A Shvartsman, S. Y., Goldsmith, E. J., et al. (2019). Functional divergence caused by mutations in an energetic hotspot in ERK2. Proc. Natl. Acad. Sci. U.S.A. 116, 15514-15523.
Teo, G., Liu, G., Zhang, J., Nesvizhskii, A. I., Gingras, A.-C., and Choi, H. (2014). SAINTexpress: improvements and additional features in Significance Analysis of INTeractome software. J. Proteomics 100, 37-43.
Tsuruta, D., Kobayashi, H., Imanishi, H., Sugawara, K., Ishii, M., and Jones, J. C. R. (2008).
Laminin-332-integrin interaction: a target for cancer therapy? Curr. Med. Chem. 15, 1968-1975.
Vizcaíno, J. A., Deutsch, E. W., Wang, R., Csordas, A., Reisinger, F., Ríos, D., Dianes, J. A., Sun, Z., Farrah, T., Bandeira, N., et al. (2014). ProteomeXchange provides globally coordinated proteomics data submission and dissemination. Nat. Biotechnol. 32, 223-226.
Vogelstein, B., Bert, V., and Kinzler, K. W. (2004). Cancer genes and the pathways they control. Nat. Med. 10, 789-799.
Yu, K., Chen, B., Aran, D., Charalel, J., Yau, C., Wolf, D. M., van't Veer, L. J., Butte, A. J., Goldstein, T., and Sirota, M. (2019). Comprehensive transcriptomic analysis of cell lines as models of primary tumors across 22 tumor types. Nat. Commun. 10, 3574.
Zhao, L., and Vogt, P. K. (2008). Helical domain and kinase domain mutations in p110alpha of phosphatidylinositol 3-kinase induce gain of function by different mechanisms. Proc. Natl. Acad. Sci. U.S.A 105, 2652-2657.
Zhao, Y., Zhang, X., Chen, Y., Lu, S., Peng, Y., Wang, X., Guo, C., Zhou, A., Zhang, J., Luo, Y., et al. (2014). Crystal Structures of PI3Kα Complexed with PI103 and Its Derivatives: New Directions for Inhibitors Design. ACS Med. Chem. Lett. 5, 138-142.
Zheng, F., Tutuncuoglu, B., Ono, K., Swaney, D. L., Kim, M., Silva, E., Liu, S., Park, J., Kratz, A., Yu, M. K., et al. Convergence of cancer mutation on a hierarchy of protein systems. Submitted.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of identifying a subject likely to respond to a hyperproliferative disorder treatment, the method comprising:
(a) compiling genetic data about a population of subjects that has a mutation candidate that causes a hyperproliferative disorder, wherein the population of subjects includes the subject;
(b) performing a mass spectrometry analysis on a sample associated with the hyperproliferative disorder to identify dysfunctional protein-protein interactions associated with the hyperproliferative disorder;
(c) obtaining a first set of rules that define dysfunctional protein-protein interactions as a function of a differential interaction score (DIS);
(c) calculating a differential interaction score (DIS);
(d) correlating the DIS with the likelihood that the dysfunctional protein-protein interaction is a causal agent of the hyperproliferative disorder by evaluating the DIS against the first set of rules, thereby generating a list of one or more causal agents to which a hyperproliferative disorder treatment for the subject should be targeted.

2.-3. (canceled)

4. The method of claim 1, wherein the mass spectrometry analysis is performed on a plurality of samples.

5. (canceled)

6. The method of claim 1, wherein the calculating comprises calculating one or more of a SAINTexpress algorithm score and a CompPASS algorithm score.

7. The method of claim 6, wherein the SAINTexpress algorithm score is calculated by a formula:
$$P ( {X}_{ij} \vert \text{?} ) = {π}_{T} P ( {X}_{ij} \vert {λ}_{ij} ) + ( 1 - {π}_{T} ) P ( {X}_{ij} \vert {κ}_{ij} ) ) \qquad ( 1 )$$
$$\text{?} \text{indicates text missing or illegible when filed}$$
wherein Xᵢⱼ is the spectral count for a prey protein i identified in a purification of bait j;
wherein λᵢⱼ is the mean count from a Poisson distribution representing true interaction;
wherein κᵢⱼ is the mean count from a Poisson distribution representing false interaction;
wherein π_{T} is the proportion of true interactions in the data; and
wherein dot notation represents all relevant model parameters estimated from the data for the pair of prey i and bait j.

8. (canceled)

9. The method of claim 1, wherein the DIS is calculated by a first formula:
$${DIS}_{A} ( b , p ) = {S}_{C 1} ( b , p ) \times {S}_{C 2} ( b , p ) \times [ 1 - {S}_{C 3} ( b , p ) ]$$
wherein DIS_{A}(b,p) is the DIS for each PPI (b, p) that is conserved in a first cell line and a second cell line, but not shared by a third cell line;
wherein S_{C1}(b,p) is the probability of a PPI being present in the first cell line;
wherein S_{C2}(b,p) is the probability of a PPI being present in the second cell line; and
wherein S_{c3}(b,p) is the probability of a PPI being present in the third cell line; and a second formula:
$${DIS}_{B} ( b , p ) = [ 1 - {S}_{C 1} ( b , p ) ] \times [ 1 - {S}_{C 2} ( b , p ) ] \times {S}_{C 3} ( b , p$$
wherein DIS_{B}(b,p) is the DIS score for each PPI (b, p) that is conserved in the third cell line, but not shared by the first cell line and the second cell line;
wherein a (+) sign is assigned if DIS_{A}(b,p)>DIS_{B}(b,p); and
wherein a (−) sign is assigned if DIS_{A}(b,p)<DIS_{B}(b,p).

10. The method of claim 1, wherein the DIS is an average of a SAINTexpress algorithm score and a CompPASS algorithm score.

11. The method of claim 1, wherein the DIS is a SAINTexpress algorithm score.

12. (canceled)

13. The method of claim 1, wherein a DIS of greater than 0.5 indicates that the dysfunctional protein-protein interaction is likely a causal agent of the hyperproliferative disorder: wherein a DIS of less than 0.5 indicates that the dysfunctional protein-protein interaction is not likely a causal agent of the hyperproliferative disorder.

14. (canceled)

15. The method of claim 1, wherein the mass spectrometry analysis is performed on a plurality of samples, wherein calculating comprises calculating a SAINTexpress algorithm score for each sample, and averaging the SAINTexpress algorithm scores.

16. The method of claim 1, wherein the hyperproliferative disorder is a cancer.

17-47. (canceled)

48. A method of identifying a subject likely to respond to a hyperproliferative disorder treatment, the method comprising:
a. calculating a differential interaction score (DIS); and
b. correlating the DIS with a likelihood that a dysfunctional protein-protein interaction is a causal agent of the hyperproliferative disorder,
wherein if the DIS score is above a first threshold, then the subject is likely to respond to a hyperproliferative disorder treatment based upon the causal agent, and
wherein if the DIS score is below the first threshold, then the subject is not likely to respond to the hyperproliferative disorder treatment based upon the causal agent.

49. The method of claim 0, further comprising:
a. compiling genetic data about a population of subjects comprising the subject, wherein the population of subjects has a mutation candidate that causes the hyperproliferative disorder; and
b. performing a mass spectrometry analysis on a sample associated with the hyperproliferative disorder to identify dysfunctional protein-protein interactions associated with the hyperproliferative disorder.

50. A method of predicting a likelihood that a subject does or does not respond to a hyperproliferative disorder treatment, the method comprising:
a. compiling genetic data about a population of subjects that has a mutation candidate that causes a hyperproliferative disorder, wherein the population of subjects includes the subject;
b. performing a mass spectrometry analysis on a sample associated with the hyperproliferative disorder to identify dysfunctional protein-protein interactions associated with the hyperproliferative disorder;
c. calculating a differential interaction score (DIS);
d. correlating the DIS with the likelihood that the dysfunctional protein-protein interaction is the causal agent of the cancer; and
e. selecting a cancer treatment for the subject based upon the causal agent.

51. The method of claim 50, further comprising:
(f) comparing the DIS score to a first threshold; and
(g) classifying the subject as being likely to respond to a hyperproliferative disorder treatment,
wherein each of steps (f) and (g) are performed after step (c), and
wherein the first threshold is calculated relative to a first control dataset.

52. A computer program product encoded on a computer-readable storage medium, wherein the computer program product comprises instructions for:
a. performing a mass spectrometry analysis on a sample from a subject that has a mutation candidate that causes a hyperproliferative disorder;
b. identifying dysfunctional protein-protein interactions associated with the hyperproliferative disorder; and
c. calculating a differential interaction score (DIS).

53. The computer program product of claim 52, further comprising a step of correlating the DIS with the likelihood that the dysfunctional protein-protein interaction is a causal agent of the hyperproliferative disorder.

54. The computer program product of claim 53, further comprising instructions for selecting a hyperproliferative treatment for the subject based upon the causal agent.

55. The computer program product of claim 52, further comprising instructions for:
(d) comparing the DIS score to a first threshold; and
(e) classifying the subject as being likely to respond to a hyperproliferative disorder treatment,
wherein each of steps (d) and (e) are performed after step (c), and
wherein the first threshold is calculated relative to a first control dataset.

56. A system comprising the computer program product of any of claims 52 through 55, and one or more of:
a. a processor operable to execute programs; and
b. a memory associated with the processor.

57.-61. (canceled)

62. A method of selecting a hyperproliferative disorder treatment for a subject in need thereof, the method comprising:
a. identifying genetic data from the subject in need of treatment;
b. comparing the genetic data from the subject to a compilation of genetic data from population of subjects that has a mutation candidate that causes a hyperproliferative disorder, wherein the population of subjects includes the subject in need thereof;
c. performing a mass spectrometry analysis on a sample from the subject associated with the hyperproliferative disorder to identify dysfunctional protein-protein interactions associated with the hyperproliferative disorder;
d. calculating a differential interaction score (DIS);
e. correlating the DIS with the likelihood that the dysfunctional protein-protein interaction is a causal agent of the hyperproliferative disorder; and
f. selecting a hyperproliferative disorder treatment for the subject based upon the causal agent.

63-65. (canceled)
